(19) United States
(12) Patent Application Publication
(10) Pub. No.: US 2008/0312298 A1
(43) Pub. Date: Dec. 18, 2008

(54) Methods for Identification of Modulators of Carm1 Methyl Transferase Activity

(76) Inventors: Kenneth William Foreman, Farmingdale, NY (US); Salam Shaaban, Westborough, MA (US); Frances E. Park, San Diego, CA (US); Michael Sauder, San Diego, CA (US)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(21) Appl. No.: 12/101,631

(22) Filed: Apr. 11, 2008

Related U.S. Application Data
(60) Provisional application No. 60/911,210, filed on Apr. 11, 2007.

(51) Int. Cl. A61K 31/4406; C12N 9/10; C12Q 1/48; C07C 215/30; A61P 29/00; G06F 17/50; G06F 19/00; A61P 35/00; A61P 3/10; C07D 213/52; A61K 31/138

(52) U.S. Cl. 514/357; 435/193; 435/15; 564/355; 546/338; 514/653; 703/1; 702/19

(57) ABSTRACT

This invention relates to CARM1, CARM1 binding pockets, or CARM1-like binding pockets. The invention relates to a computer comprising a data storage medium encoded with the structure coordinates of such binding pockets. The invention also relates to methods of using the structure coordinates to solve the structure of homologous proteins or protein complexes. The invention relates to methods of using the structure coordinates to screen for and design compounds that bind to CARM1 protein, complexes of CARM1 protein, homologs thereof, or CARM1-like protein or protein complexes. The invention also relates to crystallizable compositions and crystals comprising a CARM1 protein or homologs thereof. The invention also relates to methods of identifying binders of CARM1 proteins. The invention also relates to methods for determining the intracellular activity of CARM1 methyltransferase and methods for identifying an agent that inhibits the intracellular activity of CARM1 methyltransferase.

### RELATED APPLICATIONS

This application claims the benefit of U.S. Ser. No. 60/911,210, filed Apr. 11, 2007 the contents of which is incorporated herein by reference in its entirety.

### TECHNICAL FIELD OF THE INVENTION

The present invention relates to human CARM1, CARM1 binding pockets or CARM1-like binding pockets. The present invention provides a computer comprising a data storage medium encoded with the structure coordinates of such binding pockets. This invention also relates to methods of using the structure coordinates to solve the structure of homologous proteins or protein complexes. In addition, this invention relates to methods of using the structure coordinates to screen for and design compounds, including compounds, that bind to CARM1 protein, CARM1 protein complexes, homologues thereof, or CARM1-like protein or CARM1-like protein complexes. The invention also relates to crystallizable compositions and crystals comprising CARM1.

### BACKGROUND OF THE INVENTION

Protein arginine methylation is a post-translational modification that was first documented in 1967 but the discovery of the first PRMT enzymes that catalyze the modification (PRMT=Protein Arginine Methyltransferase) happened only in 1996 with the identification of human PRMT1 and its yeast homolog HMT1/RMT1 (Bedford, M. T., and Richard, S. (2005). Arginine methylation an emerging regulator of protein function. Molecular cell 18, 263-272). PRMT1 is responsible for at least 50% of the methylarginines within the cell and is essential for survival of mouse embryos (knockout embryos die at embryonic day 6.5). CARM1 is likewise essential for murine survival, but embryos survive to term and instead die just after birth. Eight other human PRMTs have since been identified but of the known PRMTs only human PRMT1, CARM1/PRMT4, and PRMT5 have been studied biologically in any detail. Structural studies have also been performed on PRMTs, generating crystal structures for HMT1 [2FYT], PRMT1 [1ORH, 1OR8 1ORI], and PRMT3 [1F3L] (N.B. The RCSB Protein Data Bank (http://home.rcsb.org/) coordinates codes are indicated in parentheses), as well as the SH2 domain of PRMT2 and the C2h2 zinc finger domain of mouse PRMT3.

CARM1 was first isolated through its ability to interact with GRIP1, a p160 steroid receptor coactivator, and was found to synergize with GRIP1 in transcriptional co-activation of nuclear receptors (Chen, D., Ma, H., Hong, H., Koh, S. S., Huang, S. M., Schurter, B. T., Aswad, D. W., and Stallcup, M. R. (1999). Regulation of transcription by a protein methyltransferase. Science 284, 2174-2177.). CARM1 also synergizes with other nuclear receptor co-activators such as AIB1, PRMT1, CBP, among others (Lee, D. Y., Northrop, J. P., Kuo, M. H., and Stallcup, M. R. (2006). Histone H3 lysine 9 methyltransferase G9a is a transcriptional coactivator for nuclear receptors. The Journal of Biological Chemistry 281, 8476-8485.). In addition to co-activation of nuclear receptors, CARM1 co-activates other transcription factors, such as the myocyte enhancer factor-2C (MEF2C) (Chen, S. L., Loffler, K. A., Chen, D., Stallcup, M. R., and Muscat, G. E. (2002). The coactivator-associated arginine methyltransferase is necessary for muscle differentiation: CARM1 coactivates myocyte enhancer factor-2. The Journal of Biological Chemistry 277, 4324-4333.), β-catenin (Koh, S. S., Li, H., Lee, Y. H., Widelitz, R. B., Chuong, C. M., and Stallcup, M. R. (2002). Synergistic coactivator function by coactivator-associated arginine methyltransferase (CARM) 1 and beta-catenin with two different classes of DNA-binding transcriptional activators. The Journal of Biological Chemistry 277, 26031-26035.), the tumor suppressor p53 (An, W., Kim, J., and Roeder, R. G. (2004). Ordered cooperative functions of PRMT1, p300, and CARM1 in transcriptional activation by p53. Cell 117, 735-748.), CREB (Krones-Herzig, A., Mesaros, A., Metzger, D., Ziegler, A., Lemke, U., Bruning, J. C., and Herzig, S. (2006). Signal-dependent control of gluconeogenic key enzyme genes through coactivator-associated arginine methyltransferase 1. The Journal of Biological Chemistry 281, 3025-3029.), and NF-κB (Covic, M., Hassa, P. O., Saccani, S., Buerki, C., Meier, N. I., Lombardi, C., Imhof, R., Bedford, M. T., Natoli, G., and Hottiger, M. O. (2005). Arginine methyltransferase CARM1 is a promoter-specific regulator of NF-kappaB-dependent gene expression. EMBO J 24, 85-96.). CARM1's co-activation function is mediated in part through its ability to methylate histone H3 and histone acetyltransferase CBP. A non-transcriptional role for CARM1 is becoming evident from other identified substrates (PABP1, TARPP, HuR, HuD, SmB, SAP49, U1C and CA150) that are mainly RNA binding protein involved in splicing, RNA stability, and protein translation.

CARM1 therefore can impact several signaling pathway through its enzymatic activity. Up-regulation or down-regulation of CARM1 is likely to affect several human pathologies. Indications for such an involvement derive from several studies. CARM1 is important for estrogen and androgen-dependent transcription in breast and prostate cancer cells respectively which make it a good target for hormone-dependent types of these cancers. Moreover CARM1 was shown to be up-regulated in androgen-independent prostate tumors (Hong, H., Kao, C., Jeng, M. H., Eble, J. N., Koch, M. O., Gardner, T. A., Zhang, S., Li, L., Pan, C. X., Hu, Z., et al. (2004). Aberrant expression of CARM1, a transcriptional coactivator of androgen receptor, in the development of prostate carcinoma and androgen-independent status. Cancer 101, 83-89; Majumder, S., Liu, Y., Ford, O. H., 3rd, Mohler, J. L., and Whang, Y. E. (2006). Involvement of arginine methyltransferase CARM1 in androgen receptor function and prostate cancer cell viability. Prostate 66, 1292-1301). CARM1 was shown to augment the function of the transcription factor β-catenin both in its role as a co-activator of androgen receptor and TCF/LEF1 (Koh, S. S., Li, H., Lee, Y. H., Widelitz, R. B., Chuong, C. M., and Stallcup, M. R. (2002). Synergistic coactivator function by coactivator-associated arginine methyltransferase (CARM) 1 and beta-catenin with two different classes of DNA-binding transcriptional activators. The Journal of biological chemistry 277, 26031-26035.) and in its role as a co-repressor of glucocorticoid receptor function in wound healing (Stojadinovic, O., Brem, H., Vouthounis, C., Lee, B., Fallon, J., Stallcup, M., Merchant, A., Galiano, R. D., and Tomic-Canic, M. (2005). Molecular pathogenesis of chronic wounds: the role of beta-catenin and c-myc in the inhibition of epithelialization and wound healing. The American journal of pathology 167, 59-69). CARM1's participation in transcriptional activation of some NF-κB-regulated genes (Covic, M., Hassa, P. O., Saccani, S., Buerki, C., Meier, N. I., Lombardi, C., Imhof, R., Bedford, M. T., Natoli, G., and Hottiger, M. O. (2005). Arginine methyltransferase CARM1 is a promoter-specific regulator of NF-kappaB-dependent gene expression. EMBO J 24, 85-96.) and of MHC-II genes in response to Interferon gamma (Zika, E., Fauquier, L., Vandel, L., and Ting, J. P. (2005). Interplay among coactivator-associated arginine methyltransferase 1, CBP, and CIITA in IFN-gamma-inducible MHC-II gene expression. Proceedings of the National Academy of Sciences of the United States of America 102, 16321-16326.) potentially links it to inflammatory diseases, autoimmunity, and cancer. CARM1 works in conjunction with CREB to up-regulate the expression of hepatic gluconeogenesis enzymes such as phosphoenolpyruvate carboxykinase (PEPCK) and glucose-6-phosphatase (G6 Pase) (Krones-Herzig, A., Mesaros, A., Metzger, D., Ziegler, A., Lemke, U., Bruning, J. C., and Herzig, S. (2006). Signal-dependent control of gluconeogenic key enzyme genes through coactivator-associated arginine methyltransferase 1. The Journal of Biological Chemistry 281, 3025-3029.). PEPCK and G6 Pase are overexpressed under diabetic conditions, thereby promoting diabetic hyperglycemia. CARM1 coactivates the framesoid X-receptor FXR (Ananthanarayanan, M., Li, S., Balasubramaniyan, N., Suchy, F. J., and Walsh, M. J. (2004). Ligand-dependent activation of the framesoid X-receptor directs arginine methylation of histone H3 by CARM1. The Journal of Biological Chemistry 279, 54348-54357.) which would make CARM1 small-molecule drugs promising therapies for diseases resulting from lipid, cholesterol and bile acid abnormalities. In a subset of schizophrenic patients CARM1 histone methyltransferase activity was upregulated in the prefrontal cortex and this upregulation was associated with downregulation of four metabolic genes (Akbarian, S., Ruehl, M. G., Bliven, E., Luiz, L. A., Peranelli, A. C., Baker, S. P., Roberts, R. C., Bunney, W. E., Jr., Conley, R. C., Jones, E. G., et al. (2005). Chromatin alterations associated with down-regulated metabolic gene expression in the prefrontal cortex of subjects with schizophrenia. Archives of General Psychiatry 62, 829-840).

CARM1 from the protozoan parasite Toxoplasma gondii, the causative agent of ‘toxoplasmosis’ disease, was characterized and shown to regulate the parasite's life cycle (Saksouk, N., Bhatti, M. M., Kieffer, S., Smith, A. T., Musset, K., Garin, J., Sullivan, W. J., Jr., Cesbron-Delauw, M. F., and Hakimi, M. A. (2005). Histone-modifying complexes regulate gene expression pertinent to the differentiation of the protozoan parasite Toxoplasma gondii. Molecular and Cellular Biology 25, 10301-10314.). CARM1 genes from Toxoplasma gondii and other infectious parasites could therefore be suitable targets for drug therapy.

### SUMMARY OF THE INVENTION

The present invention provides the first time the crystal structure of the CARM1 methyltransferase domain. This structure elucidates the key residues for S-adenosyl-methionine (SAM) binding and the binding region for its substrates. The structure also presents a rationale for the structure-based design of small molecule CARM1 binders as therapeutic agents, thus addressing the need for novel drugs for the treatment of inflammation, cancer, diabetes, heart disease, schizophrenia, wound healing, and/or parasitic infections and related diseases.

The present invention also provides molecules comprising CARM1 binding pockets, or CARM1-like binding pockets that have similar three-dimensional shapes. In one embodiment, the molecules are CARM1 or CARM1-like proteins, protein complexes, or homologues thereof. In another embodiment, the molecules are CARM1 domains or homologues thereof. In another embodiment, the molecules are in crystalline form.

The invention provides crystallizable compositions and crystal compositions comprising human CARM1 or a homologue thereof with or without a chemical entity.

The invention provides a computer comprising a machine-readable storage medium, comprising a data storage material encoded with machine-readable data, wherein the data defines the binding pockets or domains according to the structure coordinates of molecules or molecular complexes of CARM1 or CARM1-like proteins, protein complexes or homologues thereof. The invention also provides a computer comprising the data storage medium. Such storage medium when read and utilized by a computer programmed with appropriate software can display, on a computer screen or similar viewing device, a three-dimensional graphical representation of such binding pockets or domains. In one embodiment, the structure coordinates of said molecules or molecular complexes are produced by homology modeling of the coordinates of FIG. 1A.

The invention also provides methods for designing, selecting, evaluating and identifying and/or optimizing compounds that bind to the molecules or molecular complexes or their binding pockets. Such compounds are potential binders of CARM1, CARM1-like proteins or their homologues.

The invention also provides a method for determining at least a portion of the three-dimensional structure of molecules or molecular complexes which contain at least some structurally similar features to CARM1, particularly CARM1 homologues. This is achieved by using at least some of the structure coordinates obtained from a CARM1 domain.

The invention provides a crystal comprising a domain of a CARM1 protein or a homologue thereof, wherein the domain of the CARM1 protein is selected from the group consisting of amino acid residues X-Y of SEQ ID NO:1, where X is one of 27, 60, 93, 128, 133, or 140, and Y is one of 472, 480, 521, or 608, and optionally additional chemical entities are present. Alternatively, the domain of the CARM1 protein comprises amino acid residues 128-480 of SEQ ID NO:1, and optionally other chemical entities are present.

The invention provides a crystallizable composition comprising a domain of a CARM1 protein or a homologue thereof, wherein the domain of the CARM1 protein is selected from the group consisting of amino acid residues X-Y of SEQ ID NO:1, where X is one of 27, 60, 93, 128, 133, or 140, and Y is one of 472, 480, 521, or 608. Preferably. the domain of the CARM1 protein comprises amino acid residues 128-480 of SEQ ID NO:1, and optionally other chemical entities are present.

The invention provides a computer comprising:

(a) a machine-readable data storage medium, comprising a data storage material encoded with machine-readable data, wherein the data defines a binding pocket or domain selected from the group consisting of:

(i) a set of amino acid residues which are identical to human CARM1 amino acid residues R168, E214, and E243 according to FIG. 1A, wherein the root mean square deviation of the backbone atoms between the set of amino acid residues and the CARM1 amino acid residues is not greater than about 2.0 Å;

(ii) a set of amino acid residues comprising at least three amino acid residues which are identical to human CARM1 amino acid residues F150, R168, D190, C193, L198, A212, E214, V242 and E243 according to FIG. 1A, wherein the root mean square deviation of the backbone atoms between the at least three amino acid residues and the CARM1 amino acid residues which are identical is not greater than about 2.0 Å;

(iii) a set of amino acid residues comprising at least five amino acid residues which are identical to human CARM1 amino acid residues F150, R168, D190, C193, L198, A212, E214, V242 and E243 according to FIG. 1A, wherein the root mean square deviation of the backbone atoms between the at least five amino acid residues and the CARM1 amino acid residues which are identical is not greater than about 2.0 Å;

(iv) a set of amino acid residues comprising at least five amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least five amino acid residues and the CARM1 amino acid residues which are identical is not greater than about 2.0 Å;

(v) a set of amino acid residues comprising at least six amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least six amino acid residues and the CARM1 amino acid residues which are identical is not greater than about 2.0 Å; and

(vi) a set of amino acid residues that are identical to CARM1 amino acid residues according to FIG. 1A, wherein the root mean square deviation between the set of amino acid residues and the CARM1 amino acid residues is not more than about 2.0 Å;

(vii) a set of amino acid residues that are identical to CARM1 amino acid residues according to FIG. 1A, wherein the root mean square deviation between the set of amino acid residues and the CARM1 amino acid residues is not more than about 3.0 Å;

(b) a working memory for storing instructions for processing the machine-readable data;

(c) a central processing unit coupled to the working memory and to the machine-readable data storage medium for processing the machine-readable data and a means for generating three-dimensional structural information of the binding pocket or domain; and

(d) output hardware coupled to the central processing unit for outputting said three-dimensional structural information of the binding pocket or domain, or information produced using the three-dimensional structural information of the binding pocket or domain.

The binding pocket is produced by homology modeling of the structure coordinates of the CARM1 amino acid residues according to FIG. 1A. Optionally the means for generating three-dimensional structural information is provided by means for generating a three-dimensional graphical representation of the binding pocket or domain.

The output hardware is for example a ZIP™ or JAZ™ drive, a disk drive, or other machine-readable data storage device.

The invention provides a method of using a computer for selecting an orientation of a chemical entity that may interact favorably with a binding pocket or domain selected from the group consisting of:

(i) a set of amino acid residues which are identical to human CARM1 amino acid residues R168, E214, and E243 according to FIG. 1A, wherein the root mean square deviation of the backbone atoms between the set of amino acid residues and the CARM1 amino acid residues is not greater than about 2.0 Å;

(ii) a set of amino acid residues comprising at least three amino acid residues which are identical to human CARM1 amino acid residues F150, R168, D190, C193, L198, A212, E214, V242 and E243 according to FIG. 1A, wherein the root mean square deviation of the backbone atoms between the at least three amino acid residues and the CARM1 amino acid residues which are identical is not greater than about 2.0 Å;

(iii) a set of amino acid residues comprising at least five amino acid residues which are identical to human CARM1 amino acid residues F150, R168, D190, C193, L198, A212, E214, V242 and E243 according to FIG. 1A, wherein the root mean square deviation of the backbone atoms between the at least five amino acid residues and the CARM1 amino acid residues which are identical is not greater than about 2.0 Å;

(iv) a set of amino acid residues comprising at least five amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least five amino acid residues and the CARM1 amino acid residues which are identical is not greater than about 2.0 Å;

(v) a set of amino acid residues comprising at least six amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least six amino acid residues and the CARM1 amino acid residues which are identical is not greater than about 2.0 Å; and

(vi) a set of amino acid residues that are identical to CARM1 amino acid residues according to FIG. 1A, wherein the root mean square deviation between the set of amino acid residues and the CARM1 amino acid residues is not more than about 2.0 Å;

(vii) a set of amino acid residues that are identical to CARM1 amino acid residues according to FIG. 1A, wherein the root mean square deviation between the set of amino acid residues and the CARM1 amino acid residues is not more than about 3.0 Å;

the method comprising the steps of:

(a) providing the structure coordinates of the binding pocket or domain on a computer comprising means for generating three-dimensional structural information from the structure coordinates;

(b) employing computational means to dock a first chemical entity in the binding pocket or domain;

(c) quantifying the association between the chemical entity and all or part of the binding pocket or domain for different orientations of the chemical entity; and

(d) selecting the orientation of the chemical entity with the most favorable interaction based on the quantified association.

In another embodiment the method further comprises generating a three-dimensional graphical representation of the binding pocket or domain prior to step (b). In another aspect the energy minimization, molecular dynamics simulations, rigid-body minimizations, combinations thereof, or similar induced-fit manipulations are performed simultaneously with or following step (b). The method according further comprises the steps of:

(e) repeating steps (b) through (d) with a second chemical entity; and

(f) selecting at least one of said first or second chemical entity that interacts more favorably with said-binding pocket or domain based on said quantified association of said first or second chemical entity.

The invention provides a method of using a computer for selecting an orientation of a chemical entity with a favorable shape complementarity in a binding pocket selected from the group consisting of:

(i) a set of amino acid residues which are identical to human CARM1 amino acid residues R168, E214, and E243 according to FIG. 1A, wherein the root mean square deviation of the backbone atoms between the set of amino acid residues and the CARM1 amino acid residues is not greater than about 2.0 Å;

(ii) a set of amino acid residues comprising at least three amino acid residues which are identical to human CARM1 amino acid residues F150, R168, D190, C193, L198, A212, E214, V242 and E243 according to FIG. 1A, wherein the root mean square deviation of the backbone atoms between the at least three amino acid residues and the CARM1 amino acid residues which are identical is not greater than about 2.0 Å;

(iii) a set of amino acid residues comprising at least five amino acid residues which are identical to human CARM1 amino acid residues F150, R168, D190, C193, L198, A212, E214, V242 and E243 according to FIG. 1A, wherein the root mean square deviation of the backbone atoms between the at least five amino acid residues and the CARM1 amino acid residues which are identical is not greater than about 2.0 Å;

(iv) a set of amino acid residues comprising at least five amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least five amino acid residues and the CARM1 amino acid residues which are identical is not greater than about 2.0 Å;

(v) a set of amino acid residues comprising at least six amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least six amino acid residues and the CARM1 amino acid residues which are identical is not greater than about 2.0 Å; and

(vi) a set of amino acid residues that are identical to CARM1 amino acid residues according to FIG. 1A, wherein the root mean square deviation between the set of amino acid residues and the CARM1 amino acid residues is not more than about 2.0 Å;

(vii) a set of amino acid residues that are identical to CARM1 amino acid residues according to FIG. 1A, wherein the root mean square deviation between the set of amino acid residues and the CARM1 amino acid residues is not more than about 3.0 Å;

The method comprising the steps of:

(a) providing the structure coordinates of the binding pocket and all or part of the substrate binding pocket therein on a computer comprising means for generating three-dimensional structural information from the structure coordinates;

(b) employing computational means to dock a first chemical entity in the binding pocket;

(c) quantitating the contact score of the chemical entity in different orientations; and

(d) selecting the orientation with the highest contact score.

In a further embodiment the method, further comprises the step of:

(e) generating a three-dimensional graphical representation of the binding pocket and all or part of the substrate binding pocket therein prior to step (b). In another embodiment, the method, further comprises the steps of:

(e) repeating steps (b) through (d) with a second chemical entity; and

(f) selecting at least one of said first or second chemical entity that has a higher contact score based on the quantitated contact score of the first or second chemical entity.

The invention provides a method for identifying a candidate binder of a molecule or molecular complex comprising a binding pocket or domain selected from the group consisting of:

(i) a set of amino acid residues which are identical to human CARM1 amino acid residues R168, E214, and E243 according to FIG. 1A, wherein the root mean square deviation of the backbone atoms between the set of amino acid residues and the CARM1 amino acid residues is not greater than about 2.0 Å;

(ii) a set of amino acid residues comprising at least three amino acid residues which are identical to human CARM1 amino acid residues F150, R168, D190, C193, L198, A212, E214, V242 and E243 according to FIG. 1A, wherein the root mean square deviation of the backbone atoms between the at least three amino acid residues and the CARM1 amino acid residues which are identical is not greater than about 2.0 Å;

(iii) a set of amino acid residues comprising at least five amino acid residues which are identical to human CARM1 amino acid residues F150, R168, D190, C193, L198, A212, E214, V242 and E243 according to FIG. 1A, wherein the root mean square deviation of the backbone atoms between the at least five amino acid residues and the CARM1 amino acid residues which are identical is not greater than about 2.0 Å;

(iv) a set of amino acid residues comprising at least five amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least five amino acid residues and the CARM1 amino acid residues which are identical is not greater than about 2.0 Å;

(v) a set of amino acid residues comprising at least six amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least six amino acid residues and the CARM1 amino acid residues which are identical is not greater than about 2.0 Å; and

(vi) a set of amino acid residues that are identical to CARM1 amino acid residues according to FIG. 1A, wherein the root mean square deviation between the set of amino acid residues and the CARM1 amino acid residues is not more than about 2.0 Å;

(vii) a set of amino acid residues that are identical to CARM1 amino acid residues according to FIG. 1A, wherein the root mean square deviation between the set of amino acid residues and the CARM1 amino acid residues is not more than about 3.0 Å;

comprising the steps of:

(a) using a three-dimensional structure of the binding pocket or domain to design, select or optimize a plurality of chemical entities;

(b) contacting each chemical entity with the molecule or the molecular complex;

(c) monitoring the inhibitory or stimulatory effect on the catalytic activity of the molecule or molecular complex by each chemical entity; and

(d) selecting a chemical entity based on the inhibitory or stimulatory effect of the chemical entity on the catalytic activity of the molecule or molecular complex.

Whether one monitors and selects a chemical with an inhibitory or stimulatory effect on the catalytic activity will depend on the intended use of the selected chemical. For example, an inhibitor may be desirable as a treatment for certain cancers.

The invention provides a method of designing a compound or complex that interacts with a binding pocket or domain selected from the group consisting of:

(i) a set of amino acid residues which are identical to human CARM1 amino acid residues R168, E214, and E243 according to FIG. 1A, wherein the root mean square deviation of the backbone atoms between the set of amino acid residues and the CARM1 amino acid residues is not greater than about 2.0 Å;

(ii) a set of amino acid residues comprising at least three amino acid residues which are identical to human CARM1 amino acid residues F150, R168, D190, C193, L198, A212, E214, V242 and E243 according to FIG. 1A, wherein the root mean square deviation of the backbone atoms between the at least three amino acid residues and the CARM1 amino acid residues which are identical is not greater than about 2.0 Å;

(iii) a set of amino acid residues comprising at least five amino acid residues which are identical to human CARM1 amino acid residues F150, R168, D190, C193, L198, A212, E214, V242 and E243 according to FIG. 1A, wherein the root mean square deviation of the backbone atoms between the at least five amino acid residues and the CARM1 amino acid residues which are identical is not greater than about 2.0 Å;

(iv) a set of amino acid residues comprising at least five amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least five amino acid residues and the CARM1 amino acid residues which are identical is not greater than about 2.0 Å;

(v) a set of amino acid residues comprising at least six amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least six amino acid residues and the CARM1 amino acid residues which are identical is not greater than about 2.0 Å; and

(vi) a set of amino acid residues that are identical to CARM1 amino acid residues according to FIG. 1A, wherein the root mean square deviation between the set of amino acid residues and the CARM1 amino acid residues is not more than about 2.0 Å;

(vii) a set of amino acid residues that are identical to CARM1 amino acid residues according to FIG. 1A, wherein the root mean square deviation between the set of amino acid residues and the CARM1 amino acid residues is not more than about 3.0 Å;

comprising the steps of:

(a) providing the structure coordinates of the binding pocket or domain on a computer comprising means for generating three-dimensional structural information from the structure coordinates;

(b) using the computer to dock a first chemical entity in part of the binding pocket or domain;

(c) docking at least a second chemical entity in another part of the binding pocket or domain;

(d) quantifying the association between the first or second chemical entity and part of the binding pocket or domain;

(e) repeating steps (b) to (d) with another first and second chemical entity,

(f) selecting a first and a second chemical entity based on the quantified association of both the first and second chemical entity;

(g) optionally, visually inspecting the relationship of the selected first and second chemical entity to each other in relation to the binding pocket or domain on a computer screen using the three-dimensional graphical representation of the binding pocket or domain and the first and second chemical entity; and

(h) assembling the selected first and second chemical entity into a compound or complex that interacts with said binding pocket or domain by model building.

The method provides a method of utilizing molecular replacement to obtain structural information about a molecule or a molecular complex of unknown structure,

wherein the molecule is sufficiently homologous to a domain of a CARM1 protein, comprising the steps of:

(a) crystallizing the molecule or molecular complex;

(b) generating an X-ray diffraction pattern from the crystallized molecule or molecular complex; and

(c) applying at least a portion of the structure coordinates set forth in FIG. 1A or a homology model thereof to the X-ray diffraction pattern to generate a three-dimensional electron density map of at least a portion of the molecule or molecular complex of unknown structure; and

(d) generating a structural model of the molecule or molecular complex from the three-dimensional electron density map.

The molecule is for example, a CARM1 protein, a domain of CARM1 protein, or a homologue of a domain of CARM1 protein.

The molecular complex is for example, a CARM1 protein complex or a homologue of the domain of CARM1 complex.

The invention provides a method for identifying a candidate binder that interacts with a binding site of a CARM1 protein or a homologue thereof, comprising the steps of:

(a) obtaining a crystal comprising a domain of said CARM1 protein or said homologue thereof, wherein the crystal is characterized with space group P_{21 21 2} and has unit cell parameters of a=74.852, b=98.629 Å, c=207.316 Å;

(b) obtaining the structure coordinates of amino acids of the crystal of step (a), wherein the structure coordinates are set forth in FIG. 1A-1 to 1A-240;

(c) generating a three-dimensional model of the domain of said CARM1 protein or said homologue thereof using the structure coordinates of the amino acids obtained in step (b), a root mean square deviation from backbone atoms of said amino acids of not more than ±2.0 Å;

(d) determining a binding site of the domain of said CARM1 protein or said homologue thereof from said three-dimensional model; and

(e) performing computer fitting analysis to identify the candidate binder which interacts with said binding site.

In another embodiment the method further comprises the step of: (f) contacting the identified candidate binder with the domain of said CARM1 protein or said homologue thereof in order to determine the effect of the binder on CARM1 protein activity.

The binding site of the domain of said CARM1 protein or said homologue thereof determined in step (d) comprises the structure coordinates according to FIG. 1A-1 to 1A-240 of amino acid residues R168, E214, and E243, wherein the root mean square deviation from the backbone atoms of said amino acids is not more than ±2.0 Å.

Alternatively the binding site of the domain of said CARM1 protein or said homologue thereof determined in step (d) comprises the structure coordinates according to FIG. 1A-1 to 1A-240 of amino acid residues F150, R168, D190, C193, L198, A212, E214, V242 and E243, wherein the root mean square deviation from the backbone atoms of said amino acids is not more than ±2.0 Å.

The invention provides a method for identifying a candidate binder that interacts with a binding site of a domain of a CARM1 protein or a homologue thereof, comprising the steps of:

(a) obtaining a crystal comprising the domain of said CARM1 protein or said homologue thereof, wherein the crystal is characterized with space group P_{21 21 2} and has unit cell parameters of a=74.852, b=98.629 Å, c=207.316 Å;

(b) obtaining the structure coordinates of amino acids of the crystal of step (a);

(c) generating a three-dimensional model of said CARM1 protein or said homologue thereof using the structure coordinates of the amino acids generated in step (b), a root mean square deviation from backbone atoms of said amino acids of not more than ±2.0 Å;

(d) determining a binding site of the domain of said CARM1 protein or said homologue thereof from said three-dimensional model; and

(e) performing computer fitting analysis to identify the candidate binder which interacts with said binding site.

In further embodiment the method further comprises the step of:

(f) contacting the identified candidate binder with the domain of said CARM1 protein or said homologue thereof in order to determine the effect of the binder on CARM1 protein activity.

The binding site of the domain of said CARM1 protein or said homologue thereof determined in step (d) comprises the structure coordinates according to FIG. 1A-1 to 1A-240 of amino acid residues R168, E214, and E243, wherein the root mean square deviation from the backbone atoms of said amino acids is not more than ±2.0 Å.

Alternatively, the binding site of the domain of said CARM1 protein or said homologue thereof determined in step (d) comprises the structure coordinates according to FIG. 1A-1 to 1A-240 of amino acid residues F150, R168, D190, C193, L198, A212, E214, V242 and E243, wherein the root mean square deviation from the backbone atoms of said amino acids is not more than ±2.0 Å or the structure coordinates according to FIG. 1A-1 to 1A-240 of amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415, wherein the root mean square deviation from the backbone atoms of said amino acids is not more than ±2.0 Å.

The invention provides a method for identifying a candidate binder that interacts with a binding site of a domain of a CARM1 protein or a homologue thereof, comprising the step of determining a binding site of the domain of said CARM1 protein or the homologue thereof from a three-dimensional model to design or identify the candidate binder which interacts with said binding site.

The binding site of the domain of said CARM1 protein or said homologue thereof determined comprises the structure coordinates according to FIG. 1A-1 to 1A-240 of amino acid residues R168, E214, and E243, wherein the root mean square deviation from the backbone atoms of said amino acids is not more than ±2.0 Å.

In various embodiments the binding site of the domain of said CARM1 protein or said homologue thereof determined comprises the structure coordinates according to FIG. 1A-1 to 1A-240 of amino acid residues F150, R168, D190, C193, L198, A212, E214, V242 and E243, wherein the root mean square deviation from the backbone atoms of said amino acids is not more than ±2.0 Å.

Alternatively, the binding site of the domain of said CARM1 protein or said homologue thereof determined comprises the structure coordinates according to FIG. 1A-1 to 1A-240 of amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415, wherein the root mean square deviation from the backbone atoms of said amino acids is not more than ±2.0 Å.

The invention provides a method for identifying a candidate binder of a molecule or molecular complex comprising a binding pocket or domain selected from the group consisting of:

(i) a set of amino acid residues which are identical to human CARM1 amino acid residues R168, E214, and E243 according to FIG. 1A, wherein the root mean square deviation of the backbone atoms between the set of amino acid residues and the CARM1 amino acid residues is not greater than about 2.0 Å;

(ii) a set of amino acid residues comprising at least three amino acid residues which are identical to human CARM1 amino acid residues F150, R168, D190, C193, L198, A212, E214, V242 and E243 according to FIG. 1A, wherein the root mean square deviation of the backbone atoms between the at least three amino acid residues and the CARM1 amino acid residues which are identical is not greater than about 2.0 Å;

(iii) a set of amino acid residues comprising at least five amino acid residues which are identical to human CARM1 amino acid residues F150, R168, D190, C193, L198, A212, E214, V242 and E243 according to FIG. 1A, wherein the root mean square deviation of the backbone atoms between the at least five amino acid residues and the CARM1 amino acid residues which are identical is not greater than about 2.0 Å;

(iv) a set of amino acid residues comprising at least five amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least five amino acid residues and the CARM1 amino acid residues which are identical is not greater than about 2.0 Å;

(v) a set of amino acid residues comprising at least six amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least six amino acid residues and the CARM1 amino acid residues which are identical is not greater than about 2.0 Å; and

(vi) a set of amino acid residues that are identical to CARM1 amino acid residues according to FIG. 1A, wherein the root mean square deviation between the set of amino acid residues and the CARM1 amino acid residues is not more than about 2.0 Å;

(vii) a set of amino acid residues that are identical to CARM1 amino acid residues according to FIG. 1A, wherein the root mean square deviation between the set of amino acid residues and the CARM1 amino acid residues is not more than about 3.0 Å;

comprising the steps of:

(a) using a three-dimensional structure of the binding pocket or domain to design, select or optimize a plurality of chemical entities; and

(b) selecting said candidate binder based on the binding effect of said chemical entities on a domain of a CARM1 protein or a domain of a CARM1 protein homologue on the catalytic activity of the molecule or molecular complex.

The invention also provided methods of using the crystal in a binder screening assay comprising: (a) selecting a potential binder by performing rational drug design with a three-dimensional structure determined for the crystal, wherein said selecting is performed in conjunction with computer modeling; (b) contacting the potential binder with a methyltransferase; and (c) detecting the ability of the potential binder to modulate the activity of the methyltransferase.

The invention also relates to a method of obtaining a crystal of a CARM1-like methyltransferase protein or homologue thereof, comprising the steps of a) optionally producing and purifying a CARM1-like methyltransferase protein or homologue thereof; b) combining a crystallization solution with said CARM1-like methyltransferase protein or homologue thereof to produce a crystallizable composition; and c) subjecting the composition to conditions which promote crystallization and obtaining said crystal. Other chemical entities that bind CARM1-like methyltransferases may optionally be present at any stage.

The invention provides a composition comprising an isolated fragment of the protein CARM1 comprising the amino acid residues 140-472 of CARM1 (Seq. I.D. No. 1; FIG. 4) that comprises a 3-dimensional structure defined by the set of atomic coordinates in FIG. 1A-1 to 1A-240. In one embodiment of this composition the isolated fragment of the protein CARM1 comprising the amino acid residues 140-472 of CARM1 comprises residues 128-480 of CARM1. In one embodiment of this composition the isolated fragment of the protein CARM1 is present in a crystalline form.

The invention provides example compounds, as depicted in Example 5, that have been identified by the methods described herein.

The invention provides a method of treating inflammation, cancer, diabetes, heart disease, schizophrenia, wound healing, and/or parasitic infections in a patient by administering one or more of the compounds identified by the methods described herein, such as those depicted in FIG. 2, with or without additional formulation or administration of other treatments (e.g. anticancer treatments, anti-diabetics).

The present invention provides a method for determining the intracellular activity of CARM1 methyltransferase comprising, providing a sample of cells to be tested for CARM1 methyltransferase activity, wherein the cells have been engineered to express a CARM1 methyltransferase peptide substrate that is specific for CARM1 methyltransferase, determining the degree of methylation of the peptide substrate by CARM1 methyltransferase in the sample, and thus determining the intracellular activity of CARM1 methyltransferase in the sample of cells.

The invention further provides a method for identifying an agent that inhibits the intracellular activity of CARM1 methyltransferase comprising, providing a sample of cells having CARM1 methyltransferase activity, wherein the cells have been engineered to express a CARM1 methyltransferase peptide substrate that is specific for CARM1 methyltransferase, determining the degree of reduction of methylation of the peptide substrate by CARM1 methyltransferase by contacting the sample of cells with a test agent and comparing the peptide substrate methylation level with the methylation level of peptide substrate in an identical control sample of cells that was not contacted with the test agent, determining the degree of inhibition of intracellular activity of CARM1 methyltransferase in the sample of cells contacted with the agent, and thus determining whether the test agent is an agent that inhibits the intracellular activity of CARM1 methyltransferase.

### BRIEF DESCRIPTION OF THE DRAWINGS

FIG. 1A: (1A-1 to 1A-240) lists the atomic coordinates for human CARM1 [amino acid residues 128-480 of the methyltransferase domain of human CARM1 protein (GenBank accession no. NP_{—}954592; SEQ ID NO:1)] as derived from X-ray diffraction. Residues 128-135 and, in chains A, B, C, and D, residues 477-480, 475-480, 475-480, and 476-480, respectively, were not included in the final model. The coordinates are shown in Protein Data Bank (PDB) format. Residues “SAH W” and “HOH W” represent S-Adenosyl-L-Homocysteine (SAH) and water molecules, respectively. The following abbreviations are used in FIG. 1A: “Atom type” refers to the element whose coordinates are measured. The first letter in the column defines the element. “Resid” refers to the amino acid residue in the molecular model. “X, Y, Z” define the atomic position of the element measured. “B” is a thermal factor that measures movement of the atom around its atomic center. “Occ” is an occupancy factor that refers to the fraction of the molecules in which each atom occupies the position specified by the coordinates. A value of “1” indicates that each atom has the same conformation, i.e., the same position, in the molecules.

FIG. 2: FIG. 2A depicts the CARM1 structure as a ribbon diagram. The crystals yielded a dimer of dimers in the unit cell. The biologically active arrangement is putatively a single dimer. The line demarks the junction between the pair of dimers. FIG. 2B depicts a single dimer of CARM1 proteins as a ribbon diagram. An arm (indicated by the oval) of the one protein reaches over to touch a region not too distant from the completely buried SAM binding pocket (indicated by the circle) of the other. FIGS. 2C and 2D show the CARM1 monomer as a ribbon diagram and as a surface, respectively. FIGS. 2E and 2F show rigidly rotated views of FIGS. 2C and 2D.

FIG. 3: FIG. 3A depicts the SAM binding site with SAH bound. The Cα trace is represented by a ribbon diagram, while crystallographically resolved atoms from the protein within 5 Å of SAH are depicted in a ball-and-stick representation. SAH is depicted with capped sticks. FIG. 3A provides the same binding site in the same orientation, except without SAH present. Hydrogen bonds are denoted with a dashed line and residues making key interactions with SAH are labeled.

FIG. 4 shows the amino acid sequence of human CARM1 (SEQ ID NO:1).

FIG. 5 shows a diagram of a system used to carry out the instructions encoded by the storage media of FIG. 6.

FIG. 6 shows cross sections of magnetic (A) and optically-readable (B) data storage media.

### DETAILED DESCRIPTION OF THE INVENTION

In order that the invention described herein may be more fully understood, the following detailed description is set forth.

Throughout the specification, the word “comprise” or variations such as “comprises” or “comprising” will be understood to imply the inclusion of a stated integer or groups of integers but not the exclusion of any other integer or groups of integers.

The following abbreviations are used throughout the application:

A=Ala=Alanine T=Thr=Threonine V=Val=Valine C=Cys=Cysteine L=Leu=Leucine Y=Tyr=Tyrosine I=Ile=Isoleucine N=Asn=Asparagine P=Pro=Proline Q=Gln=Glutamine F=Phe=Phenylalanine D=Asp=Aspartic Acid W=Trp=Tryptophan E=Glu=Glutamic Acid M=Met=Methionine K=Lys=Lysine G=Gly=Glycine R=Arg=Arginine S=Ser=Serine H=His=Histidine

As used herein, the following definitions shall apply unless otherwise indicated.

The term “about” when used in the context of root mean square deviation (RMSD) values takes into consideration the standard error of the RMSD value, which is ±0.1 Å.

The term “associating with” refers to a condition of proximity between a chemical entity or compound, or portions thereof, and a binding pocket or binding site on a protein. The association may be non-covalent—wherein hydrogen bonding, hydrophobic, Van der Waals and electrostatic interactions, taken together, favor the juxtaposition—or it may be covalent.

The term “binding pocket” refers to a region of a molecule or molecular complex, which, as a result of its shape, favorably associates with a chemical entity. The term “pocket” includes, but is not limited to, cleft, channel or site. CARM1, CARM1-like molecules or homologues thereof may have binding pockets that include, but are not limited to, peptide or substrate binding and SAM-binding sites. The shape of a first binding pocket may be largely pre-formed before binding of a chemical entity, may be formed simultaneously with binding of a chemical entity, or may be formed by the binding of another chemical entity to a different binding pocket of the molecule, which in turn induces a change in shape of the first binding pocket

The term “catalytic active site” or “active site” refers to the portion of the protein to which nucleotide substrates bind. For example, the catalytic active site of CARM1 is at the interface between the β-strand- and α-helical-rich portions of the protein.

The term “chemical entity” refers to chemical compounds, complexes of at least two chemical compounds, and fragments of such compounds or complexes. The chemical entity can be, for example, a ligand, substrate, nucleotide amino acid, non-naturally occurring nucleotide amino acid, amino acid, nucleotide, agonist, antagonist, binder, antibody, peptide, protein or drug. In one embodiment, the chemical entity is a binder or substrate for the active site of CARM1 proteins or protein complexes, or homologues thereof. The first and second chemical entities referred to in the present invention may be identical or distinct from each other. When iterative steps of using first and second chemical entities are carried out, taken as a pair, the first and second chemical entities used in repeated steps should be different from the first and second chemical entities of the prior steps.

The term “complex” or “molecular complex” refers to a protein associated with a chemical entity.

The term “conservative substitutions” refers to residues that are physically or functionally similar to the corresponding reference residues. That is, a conservative substitution and its reference residue have similar size, shape, electric charge, chemical properties including the ability to form covalent or hydrogen bonds, or the like. Preferred conservative substitutions are those fulfilling the criteria defined for an accepted point mutation in Dayhoff et al., Atlas of Protein Sequence and Structure, 5: 345-352 (1978 & Supp.), which is incorporated herein by reference. Examples of conservative substitutions are substitutions including but not limited to the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine.

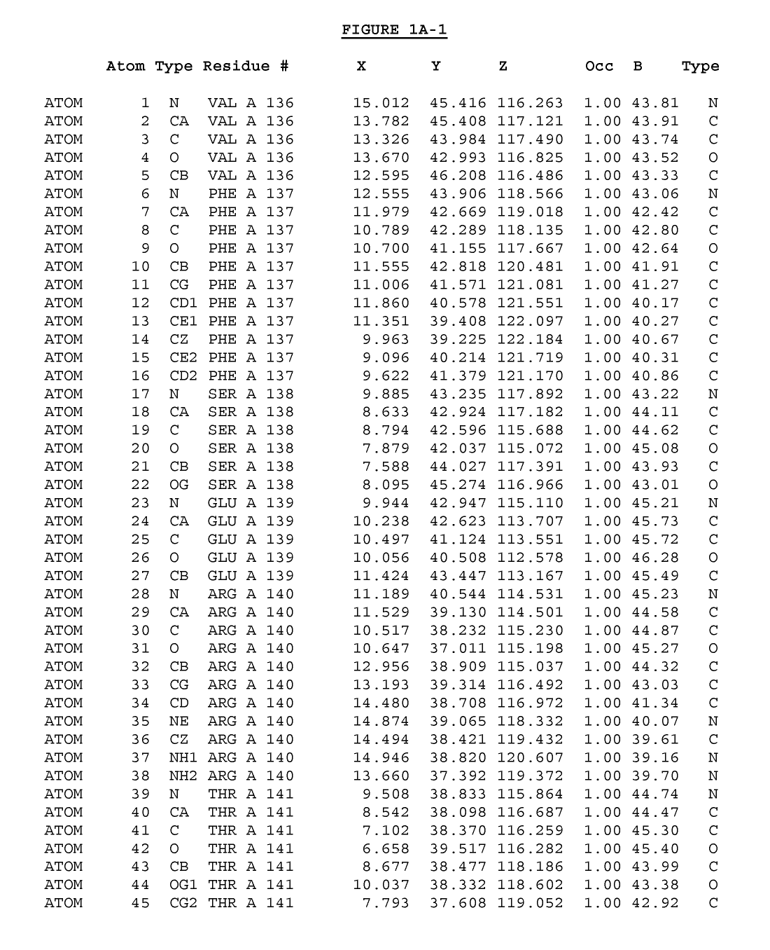

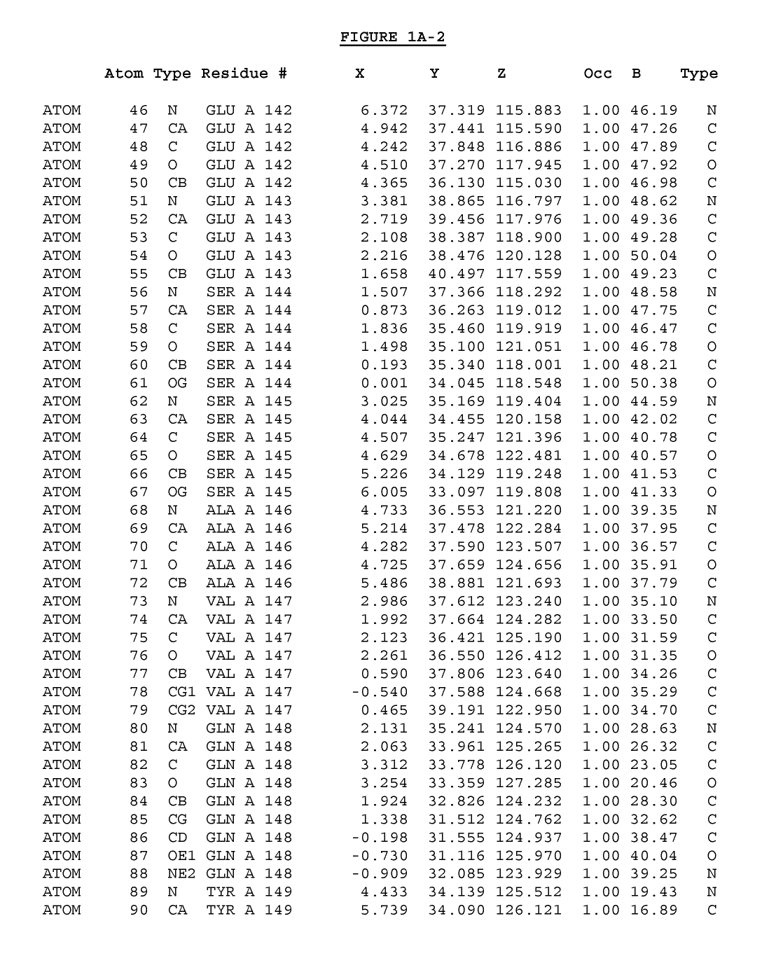

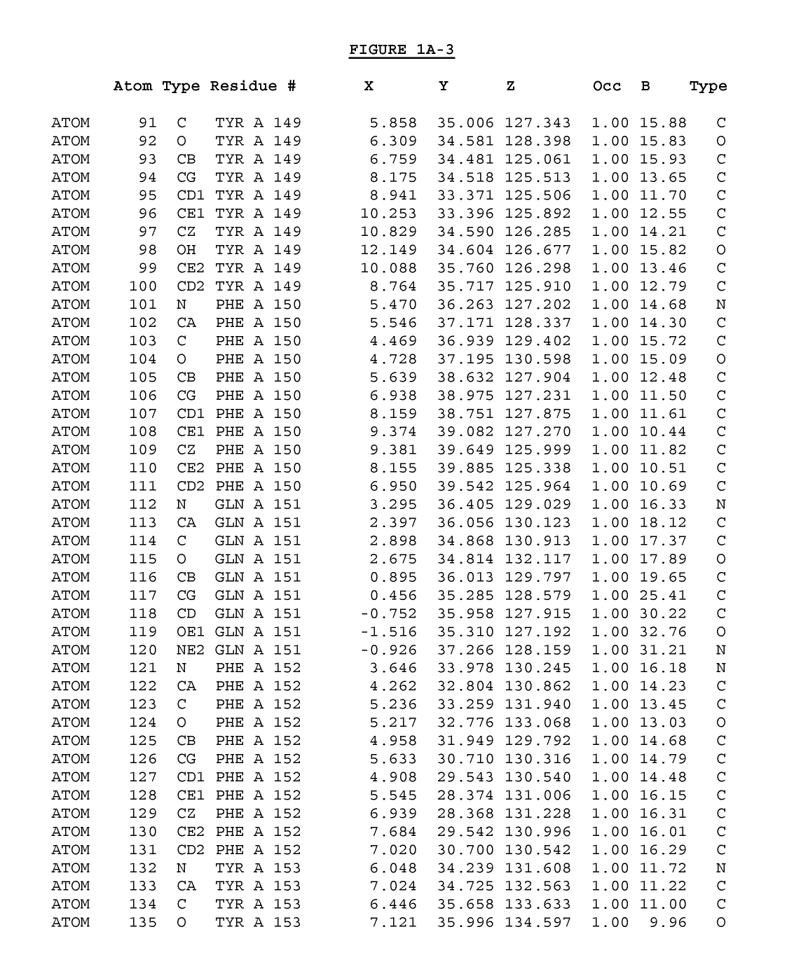

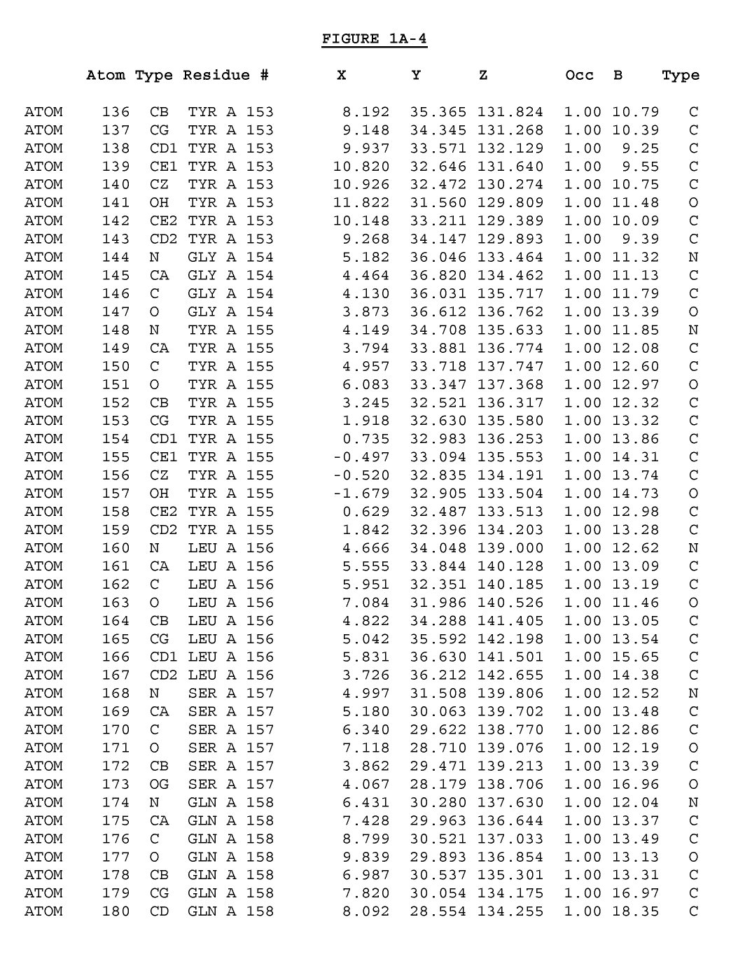

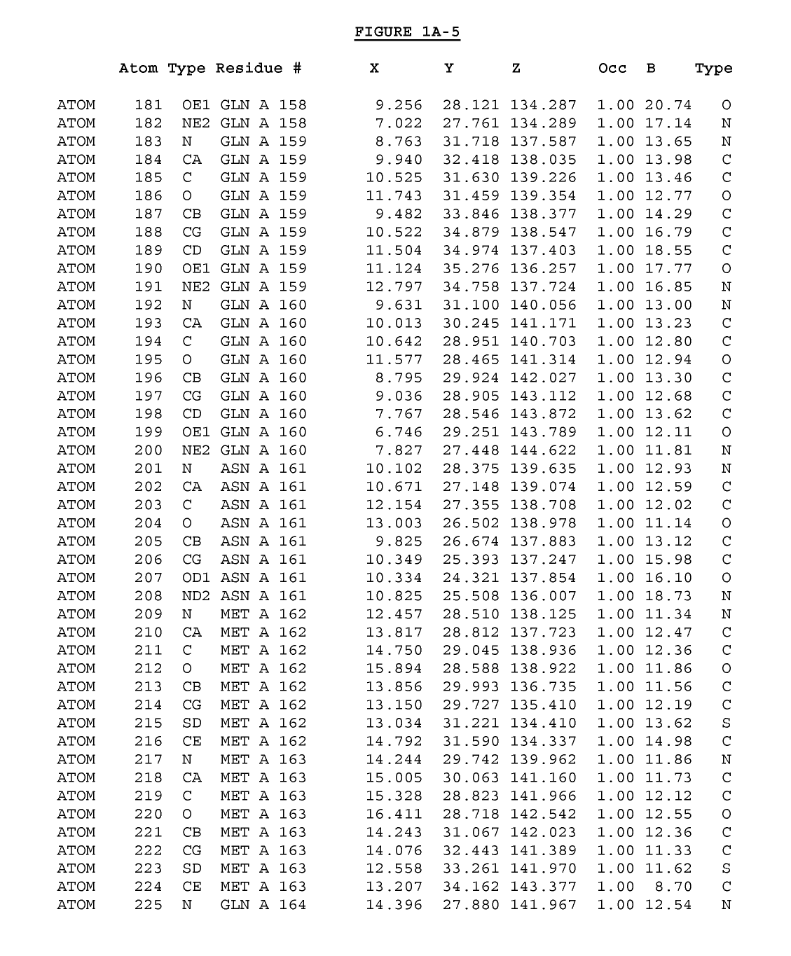

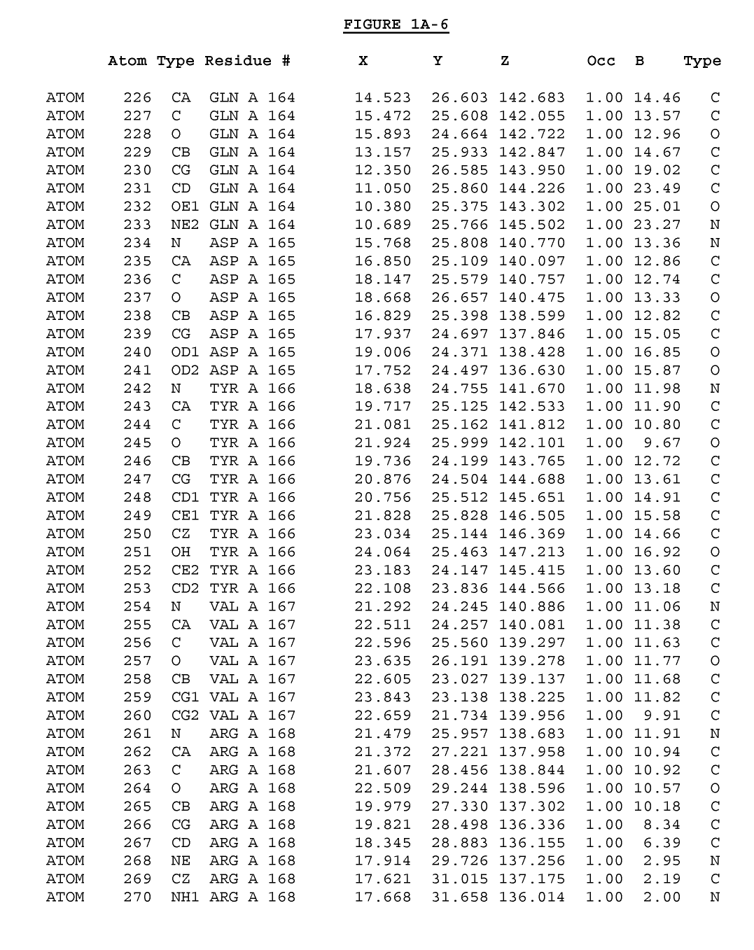

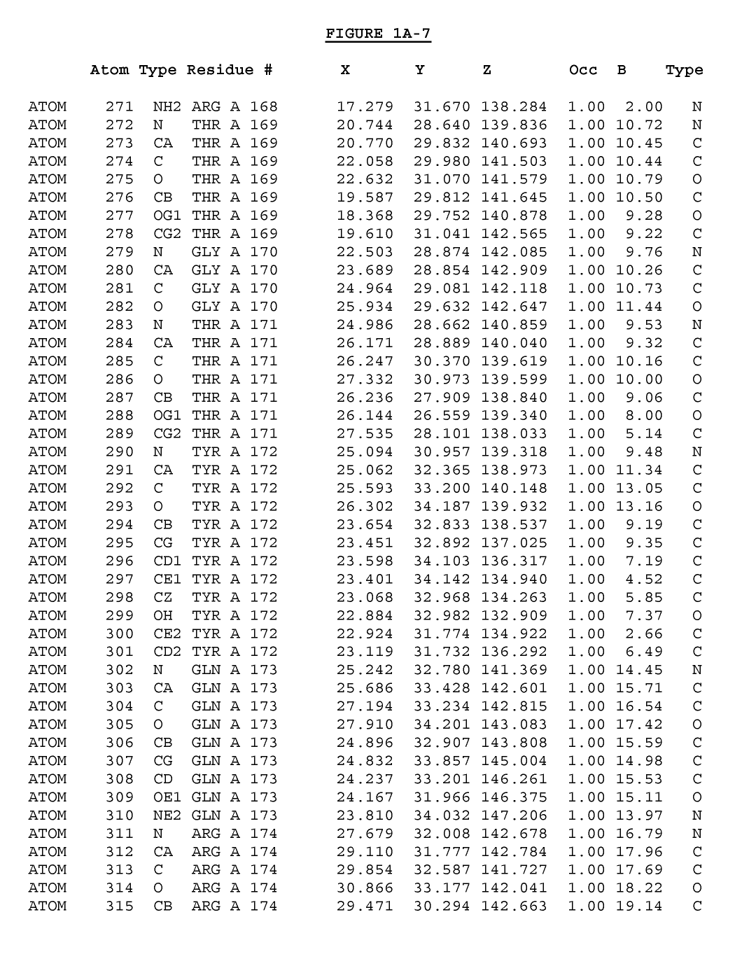

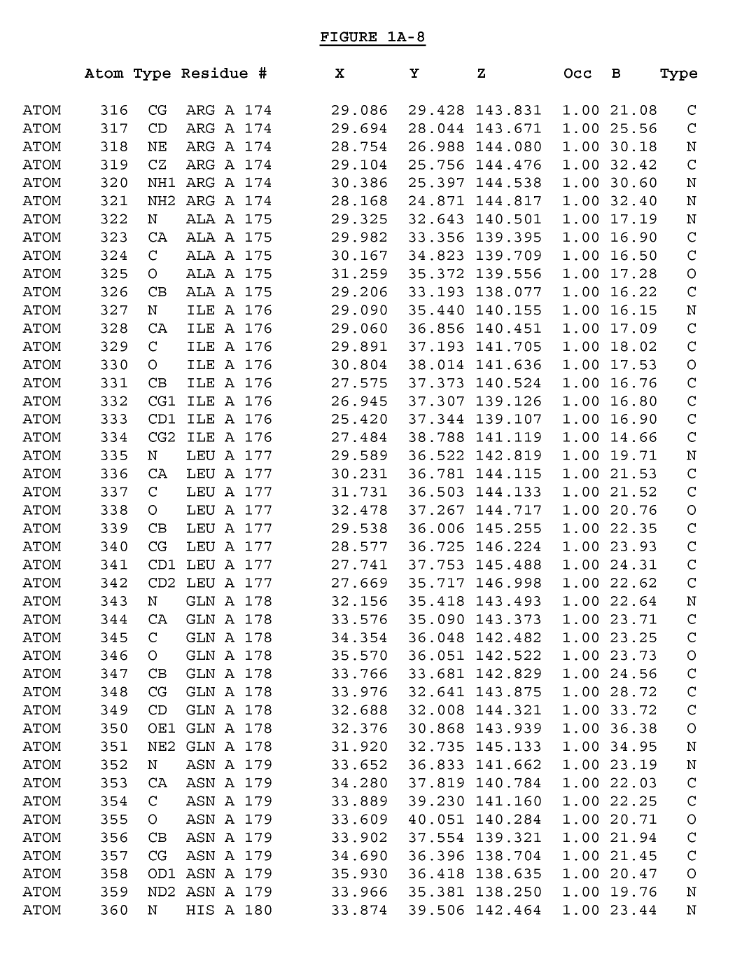

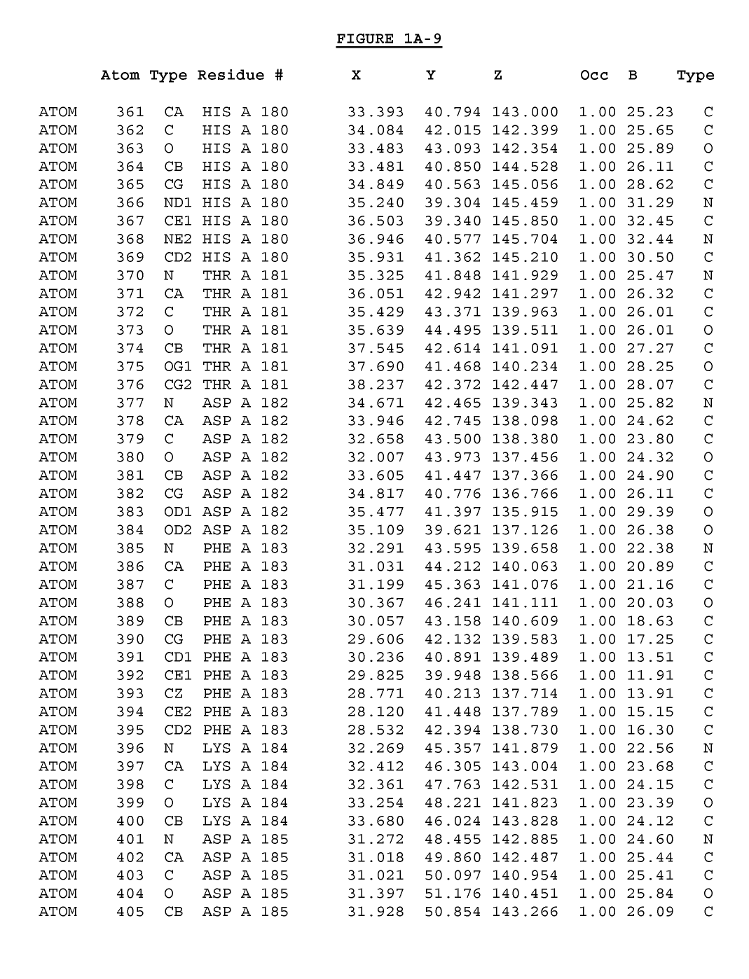

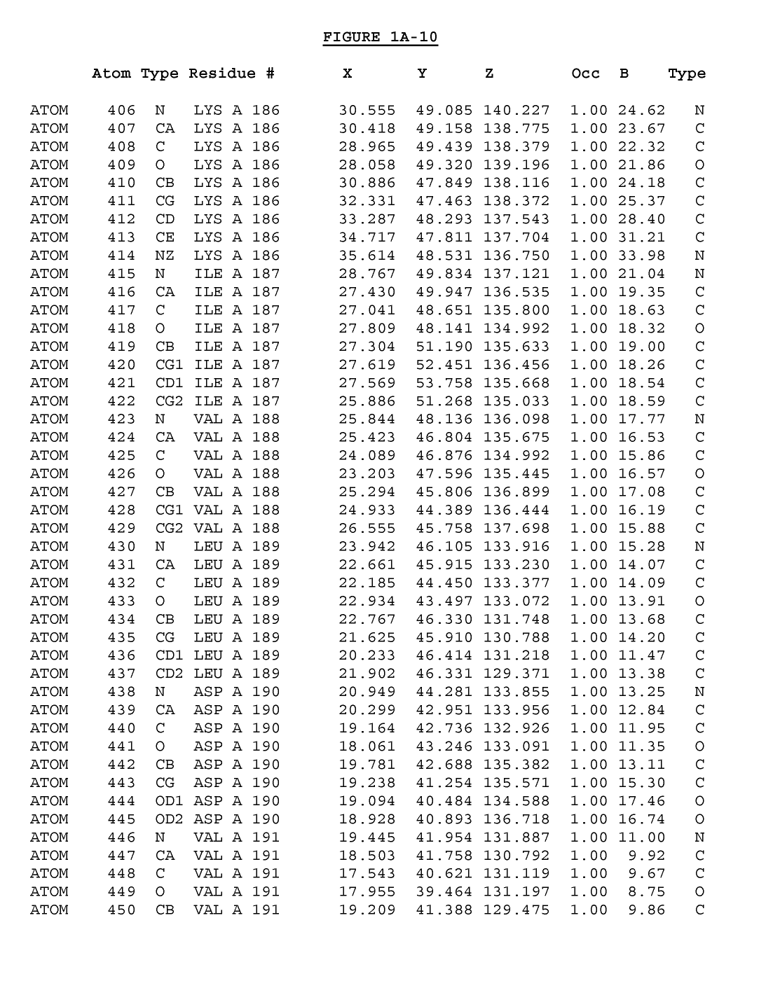

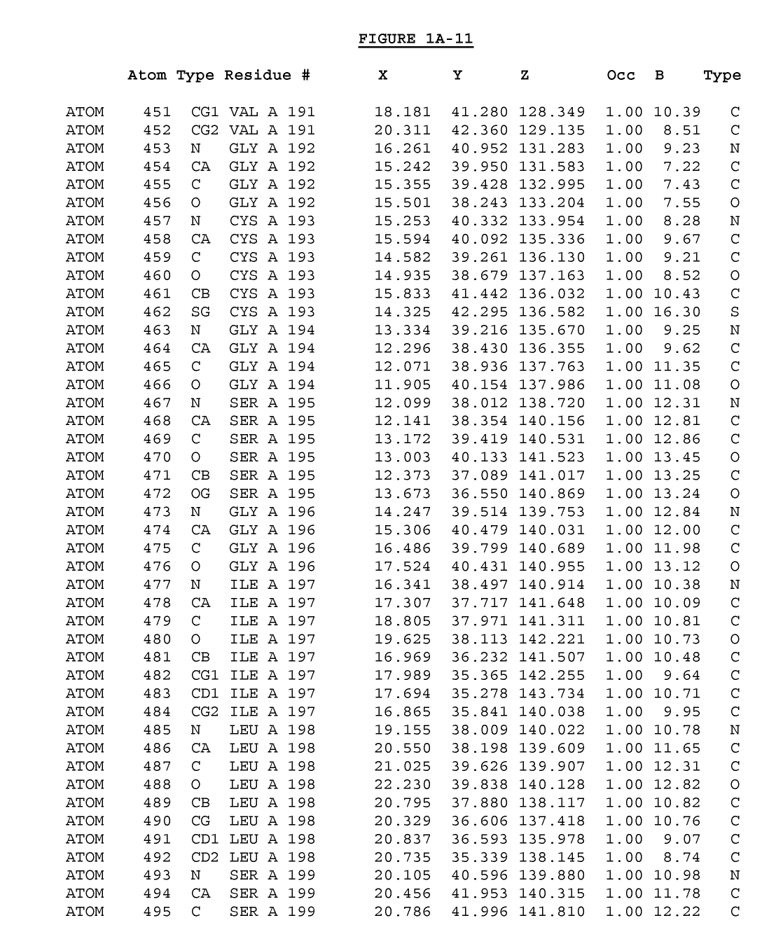

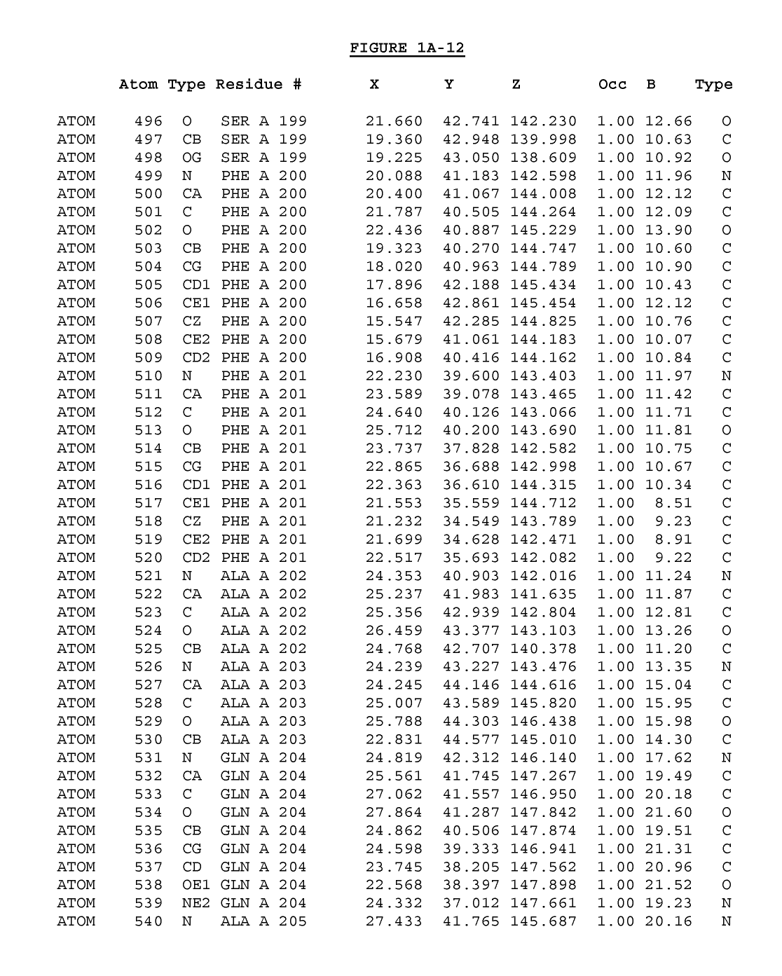

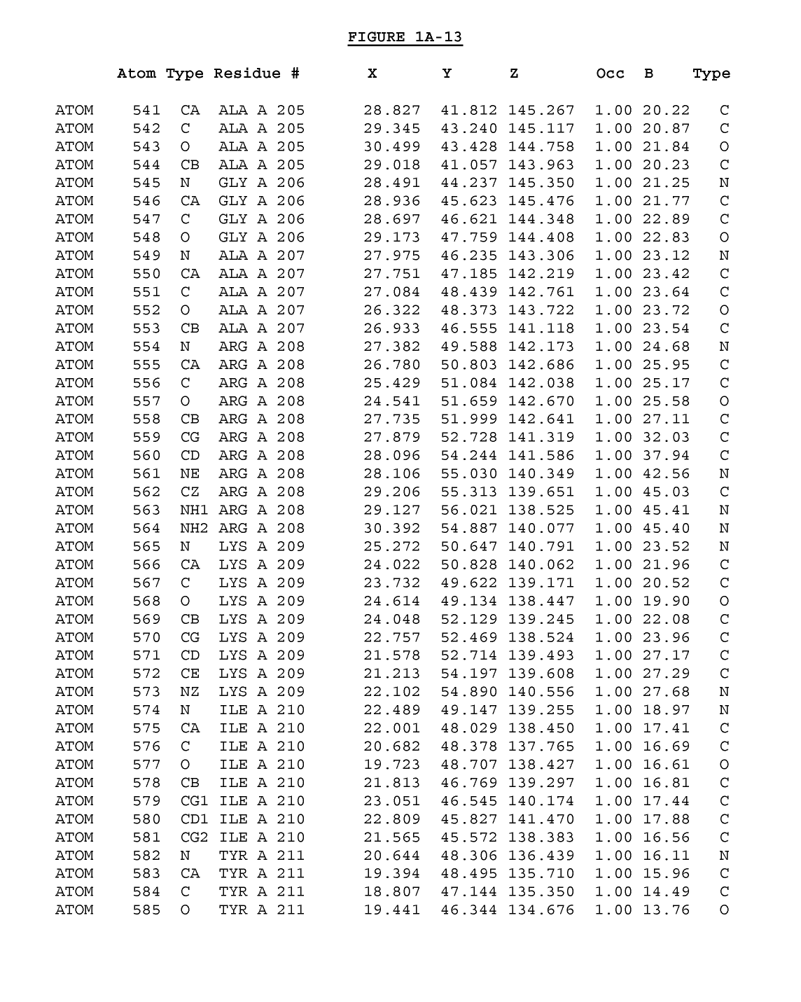

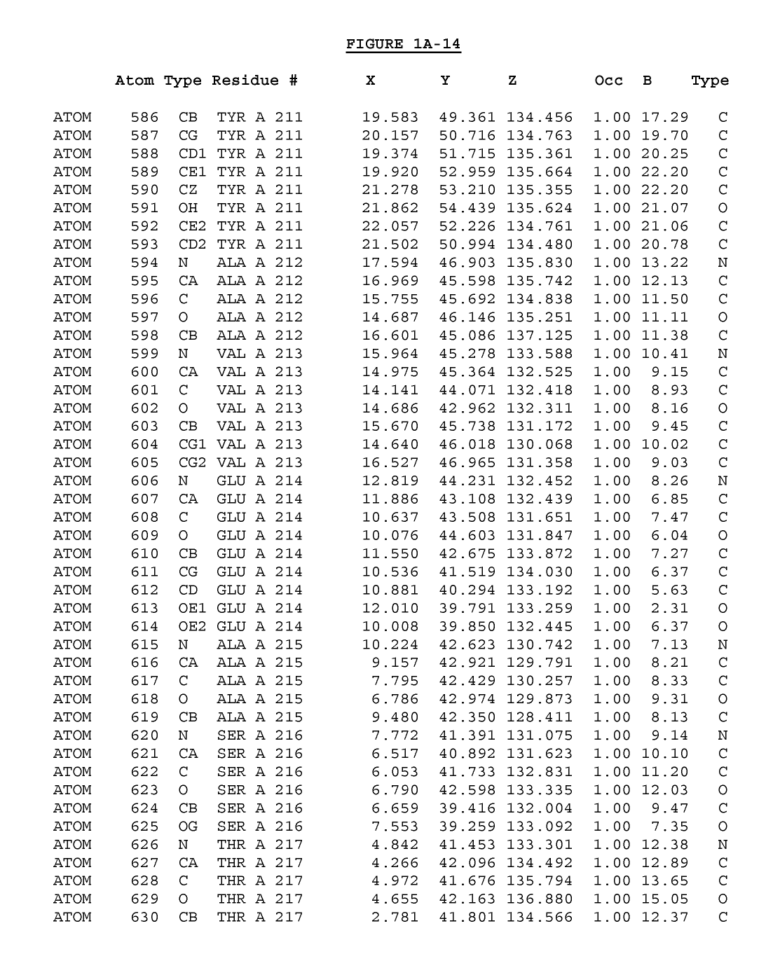

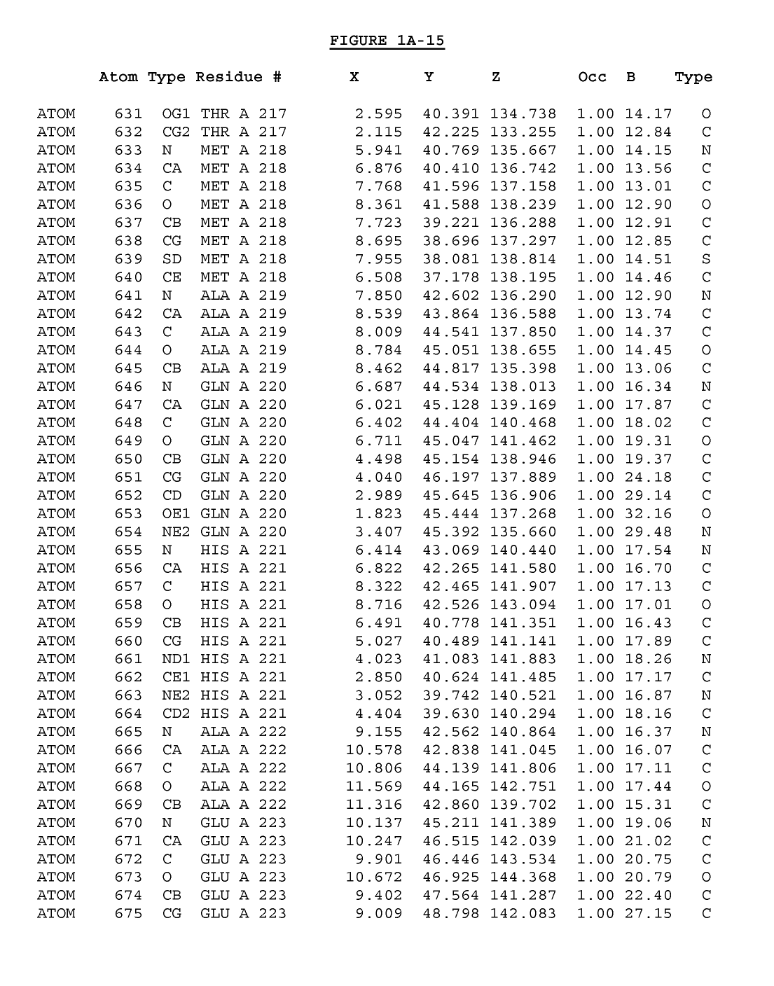

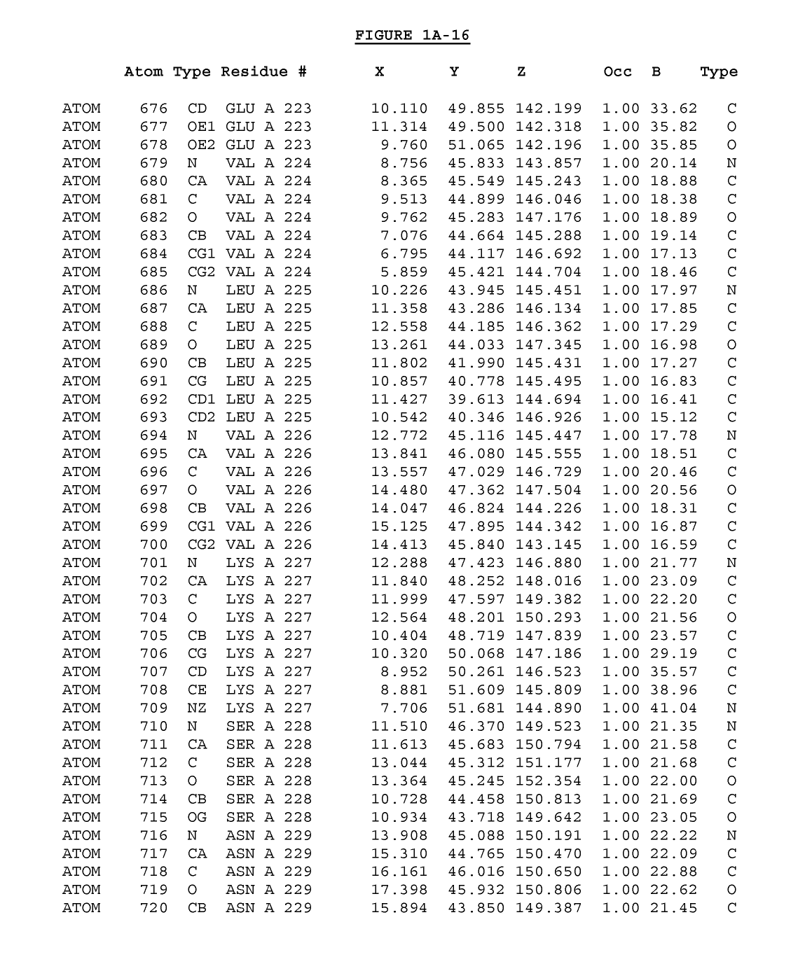

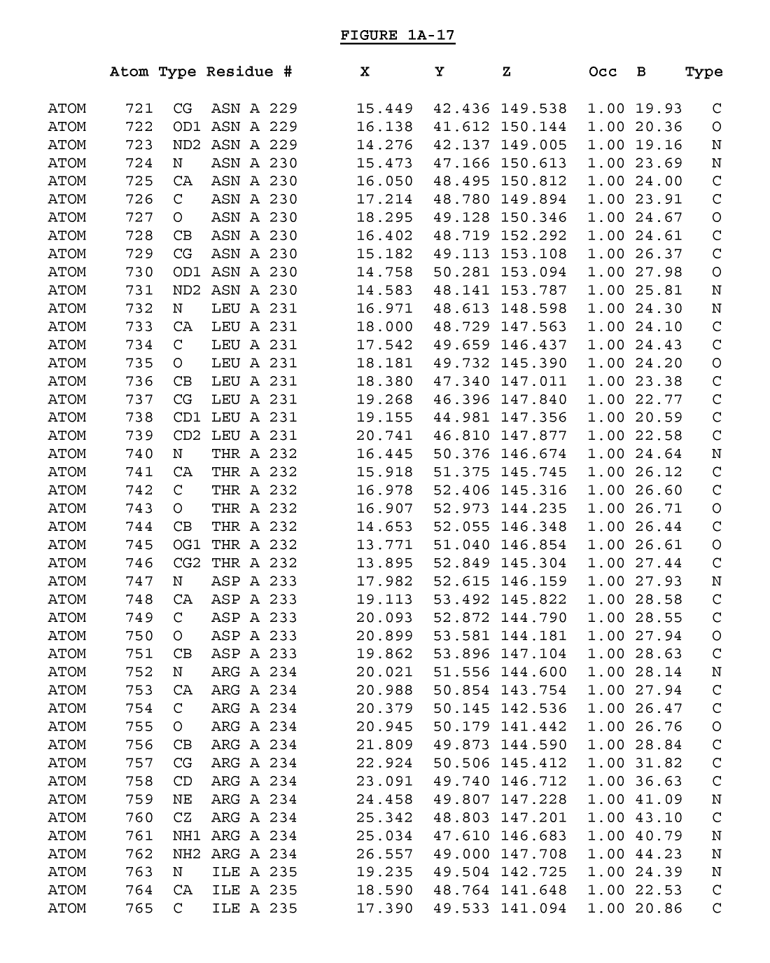

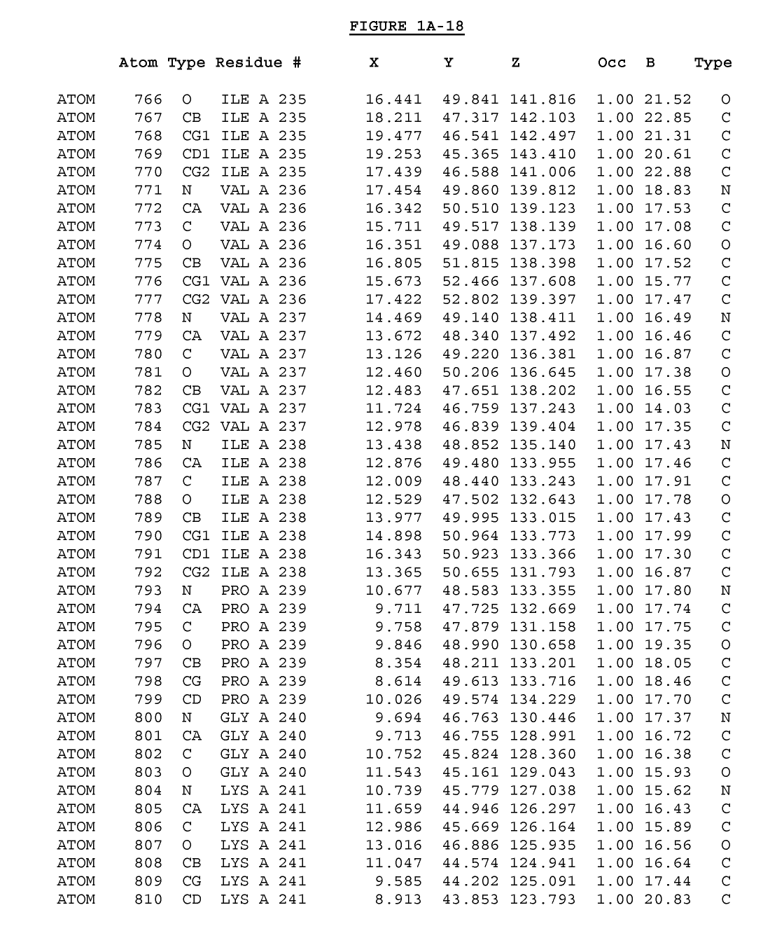

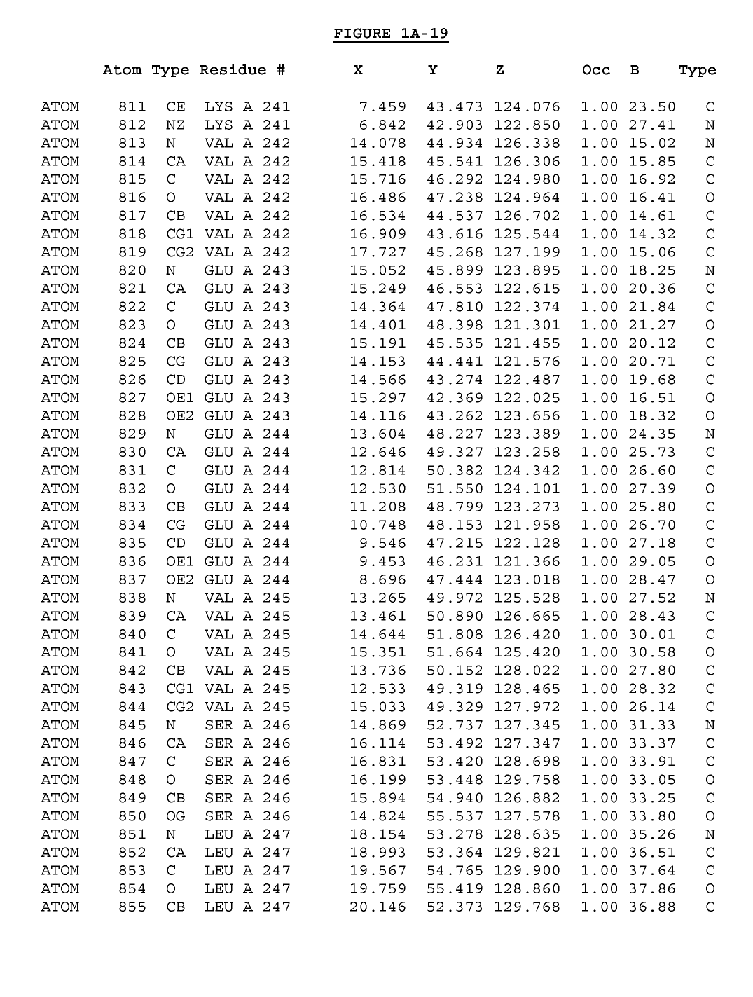

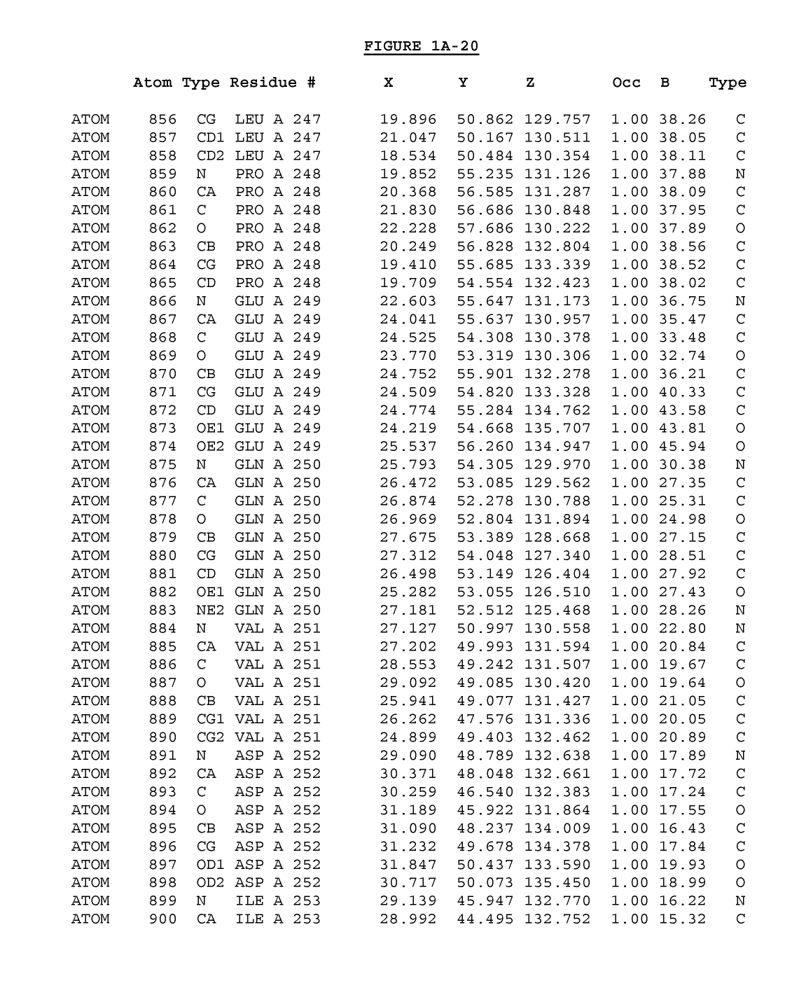

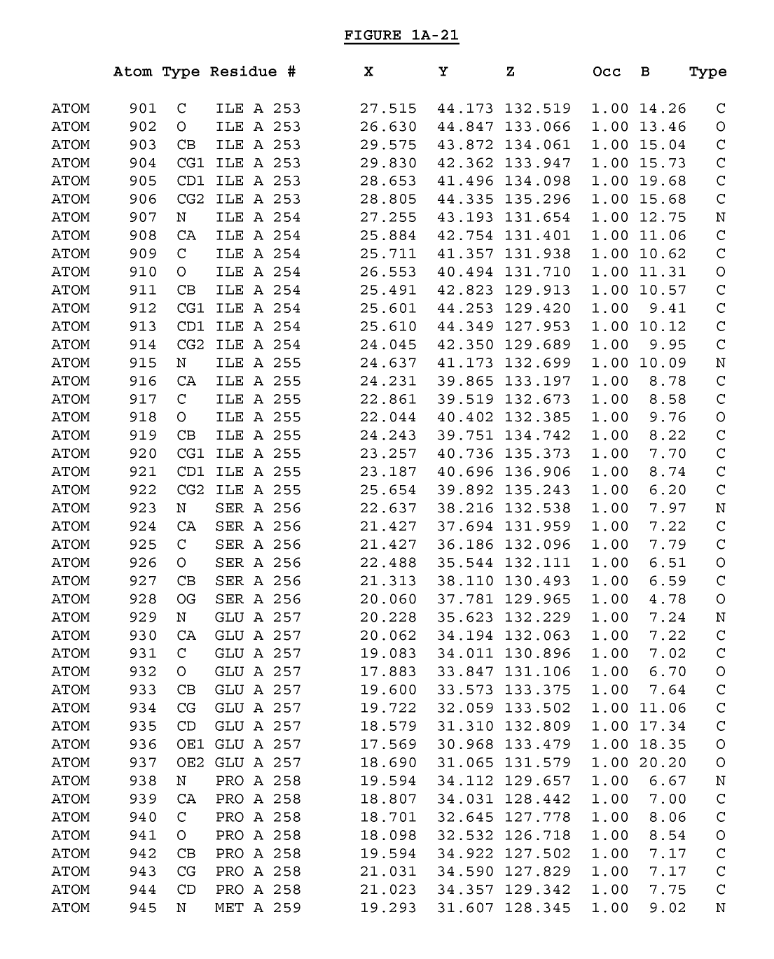

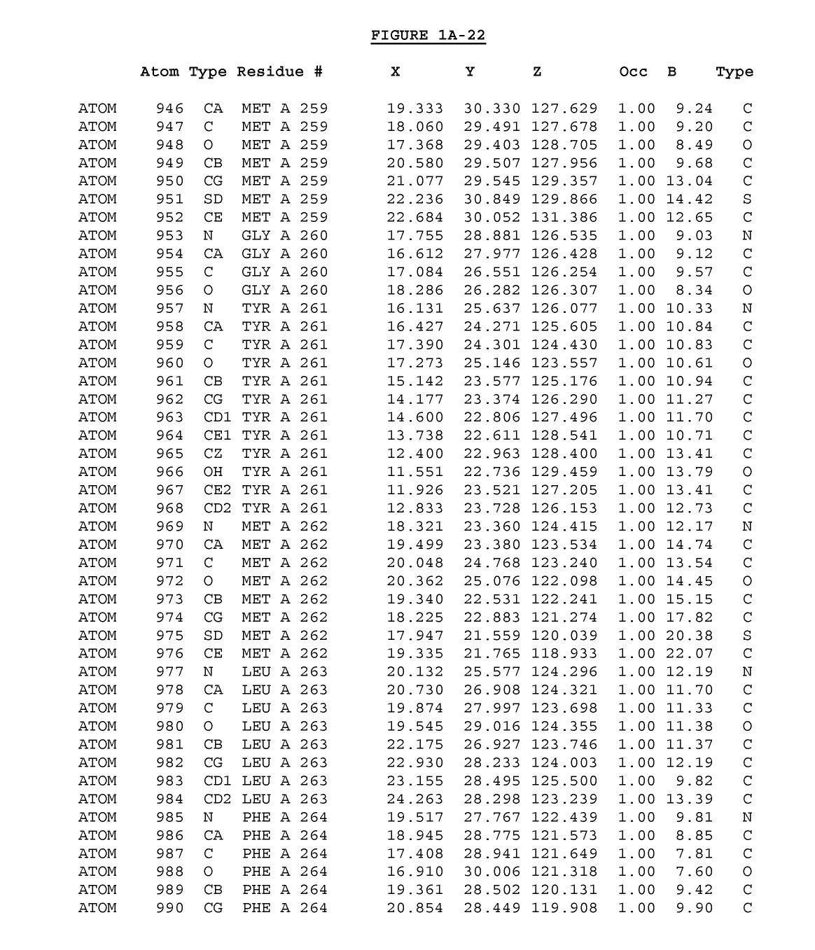

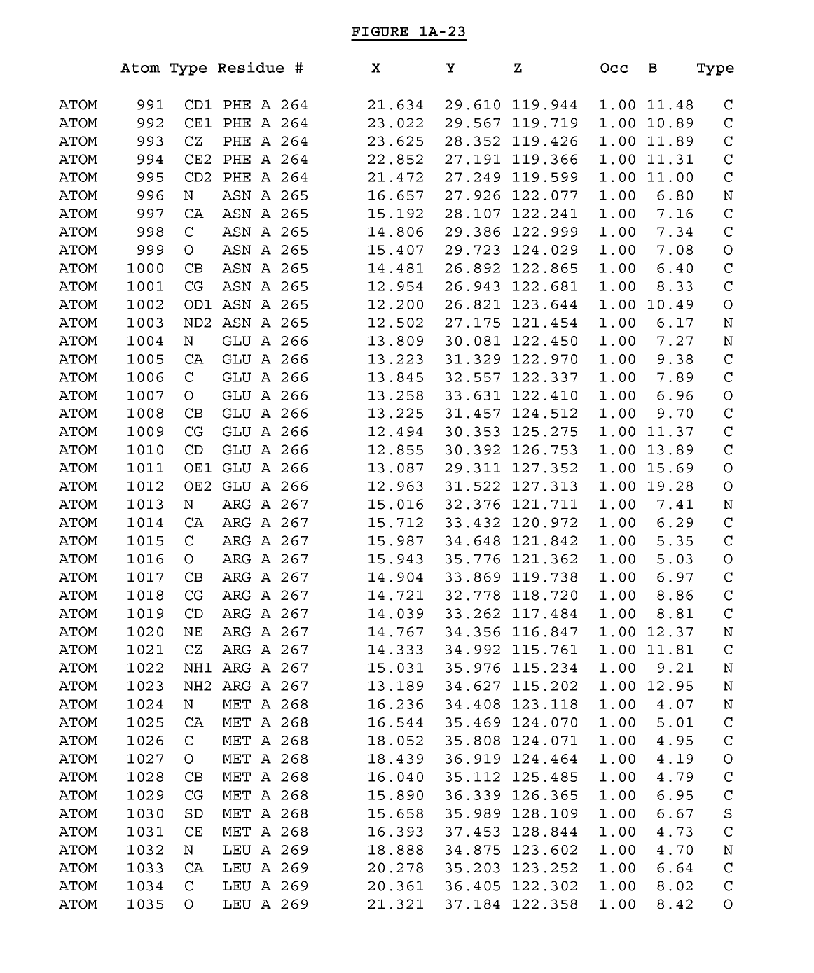

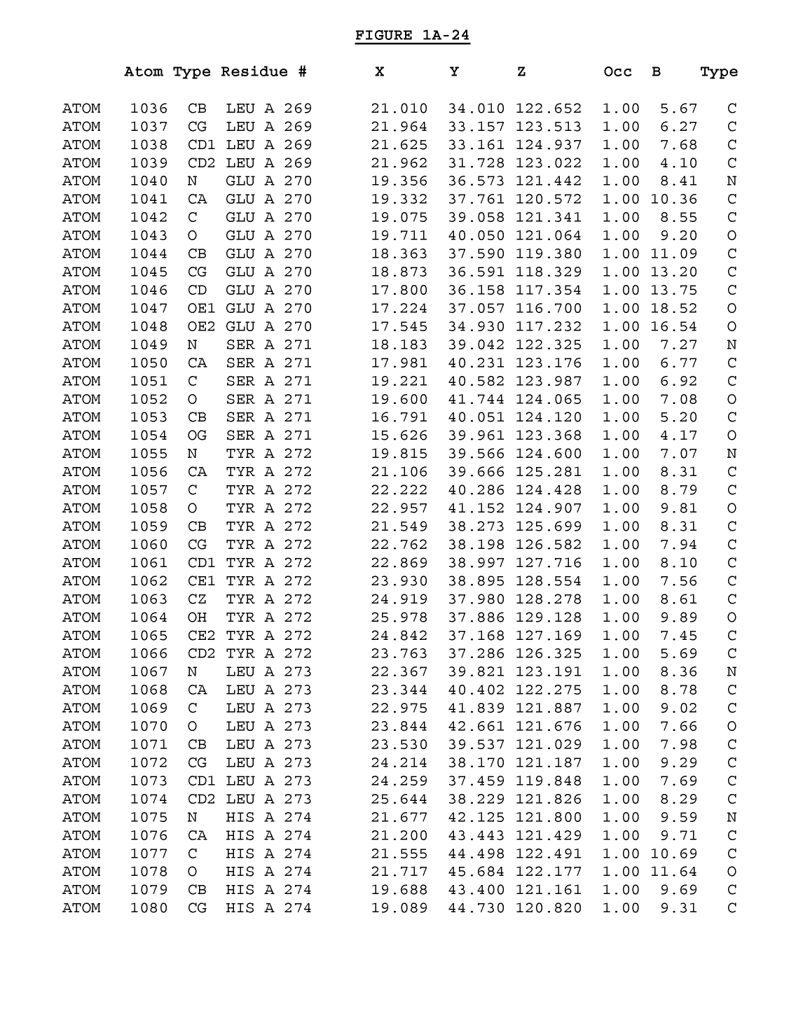

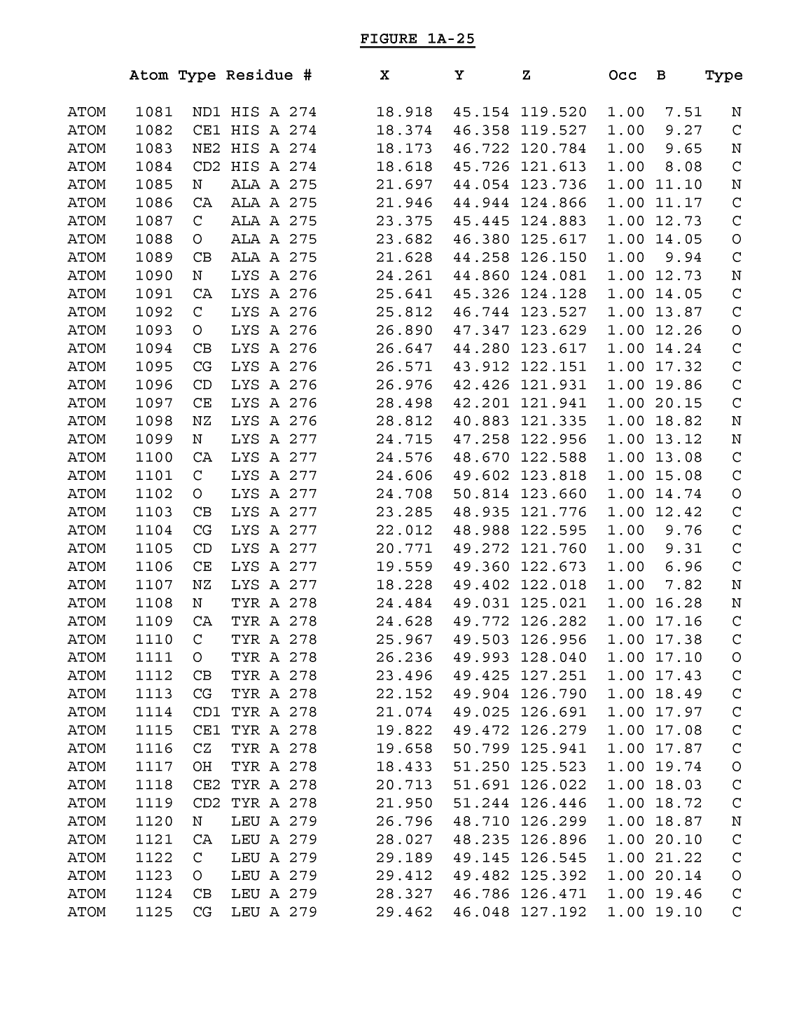

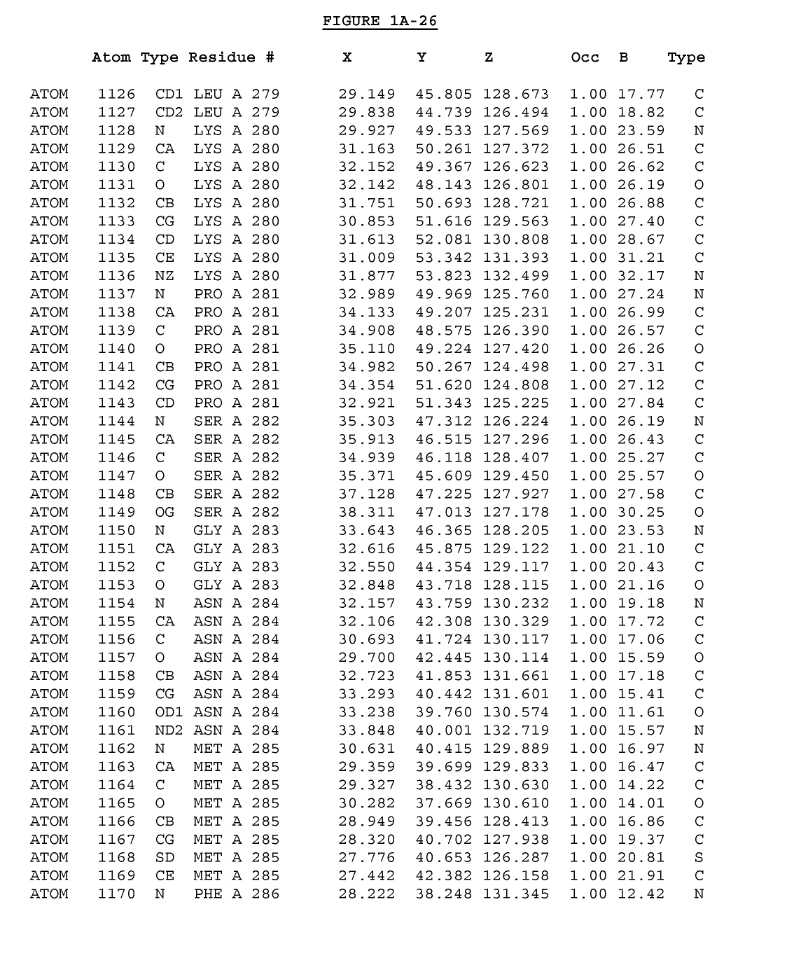

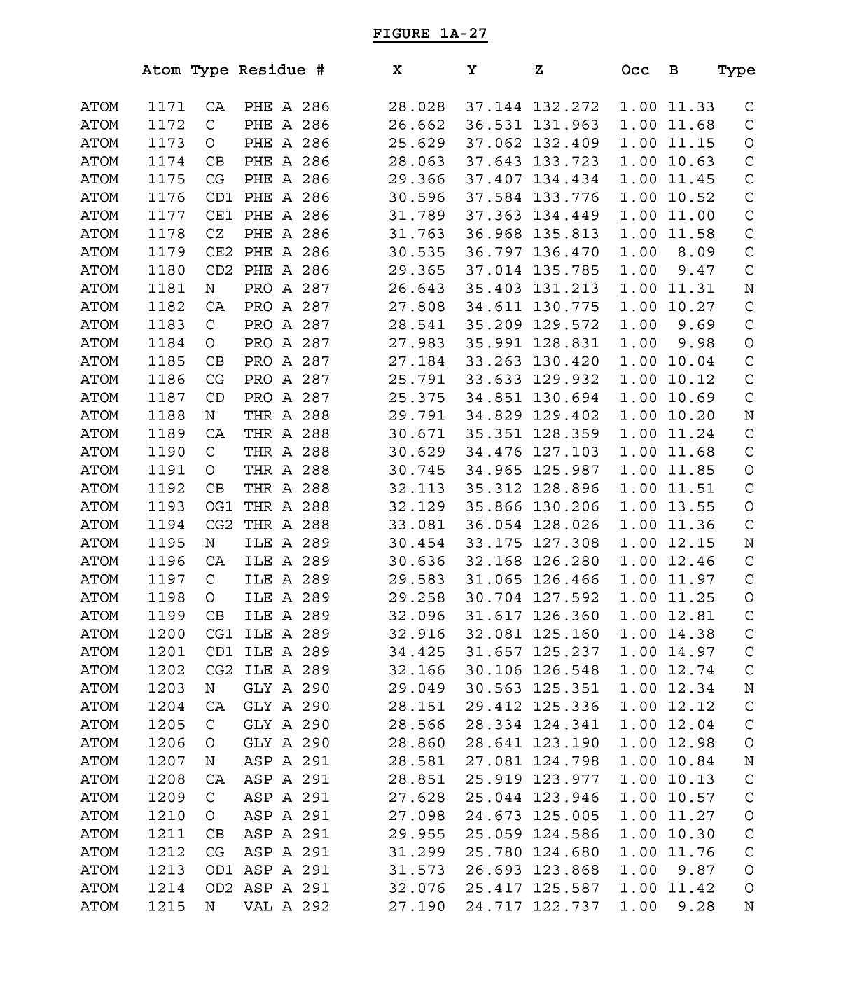

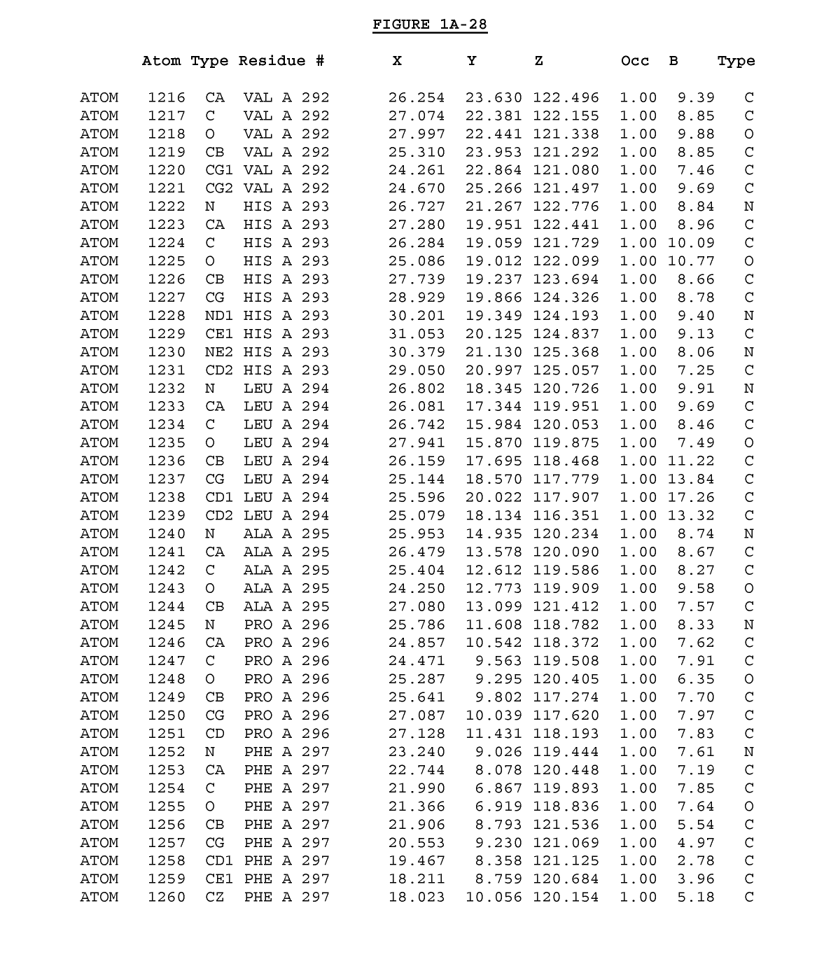

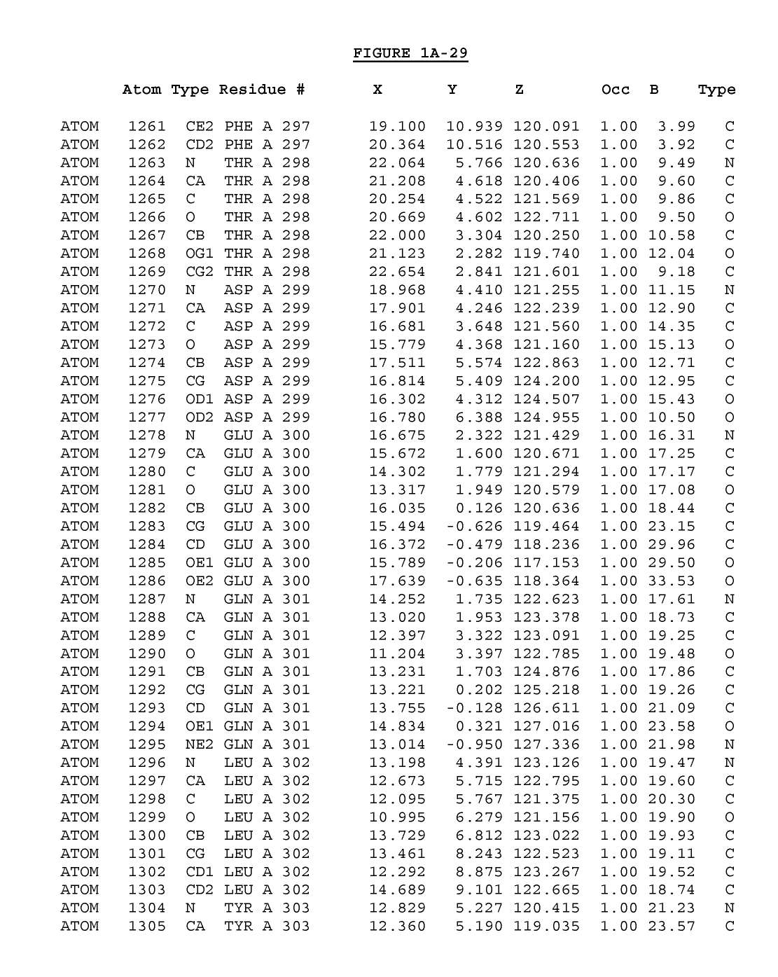

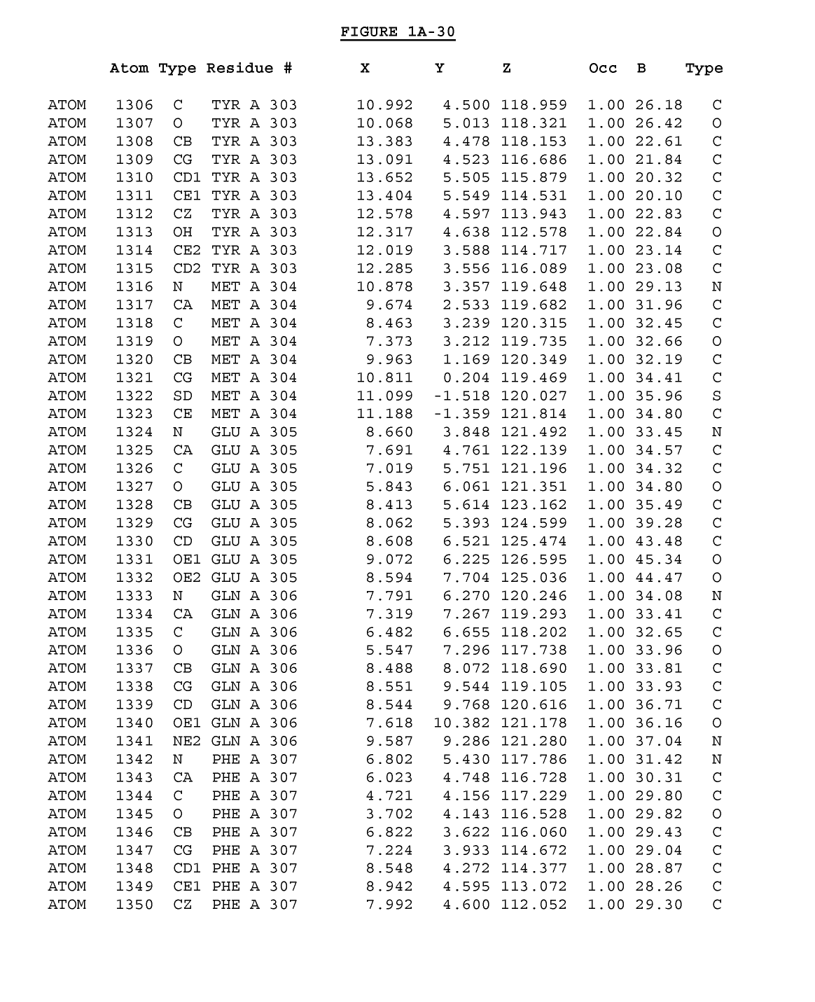

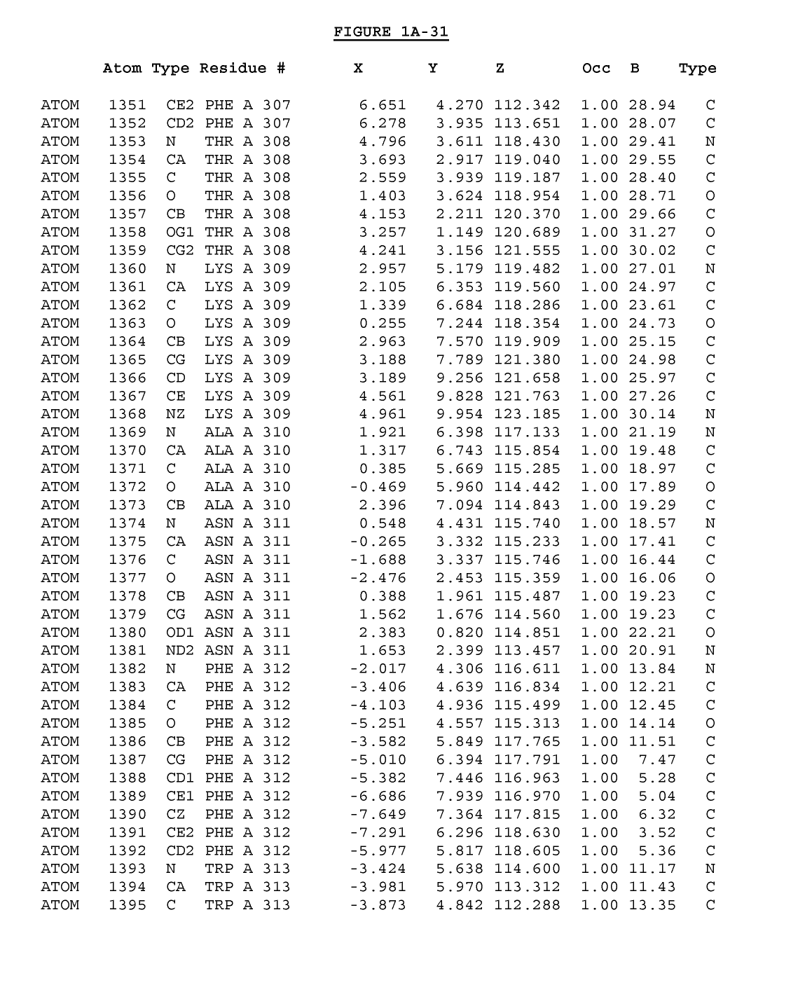

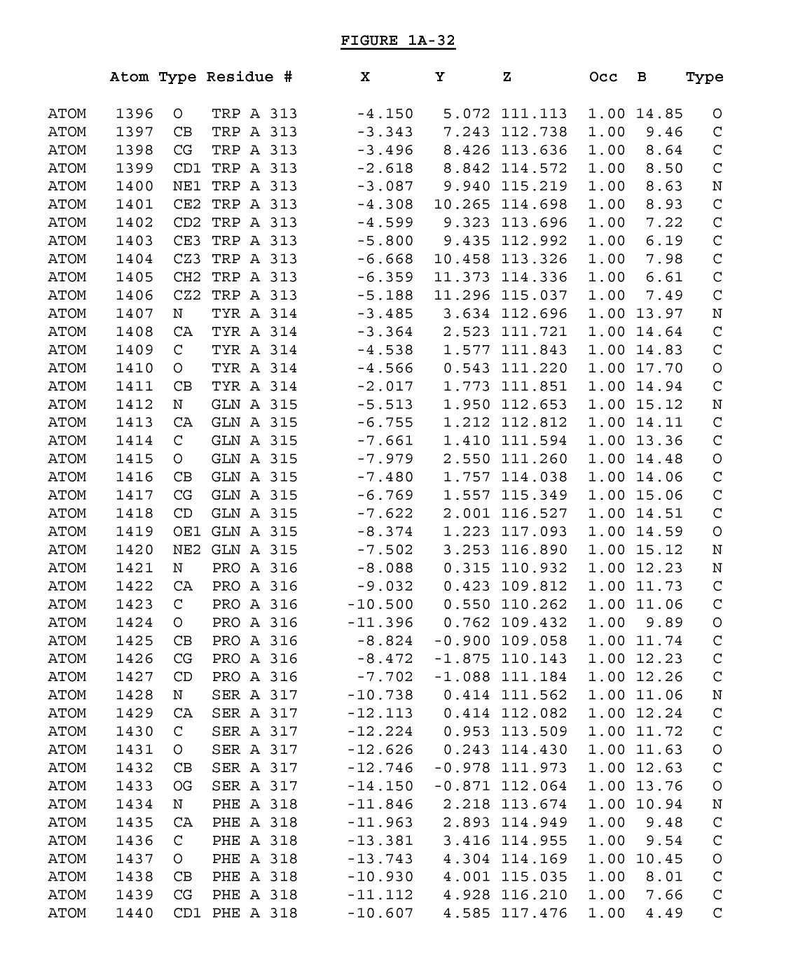

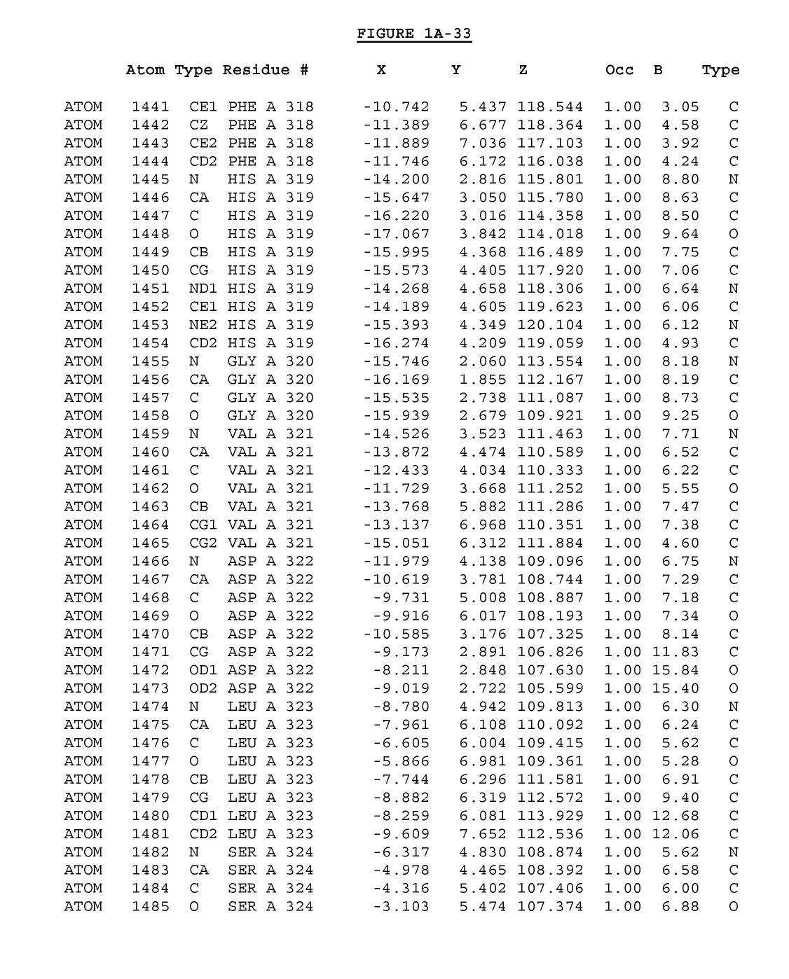

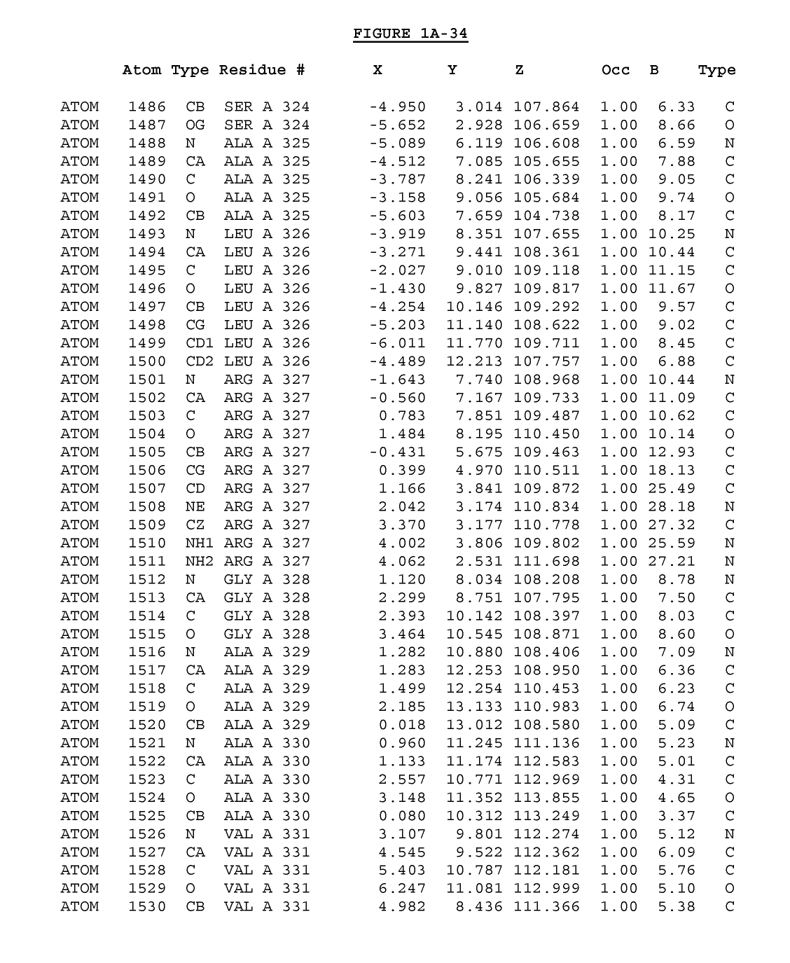

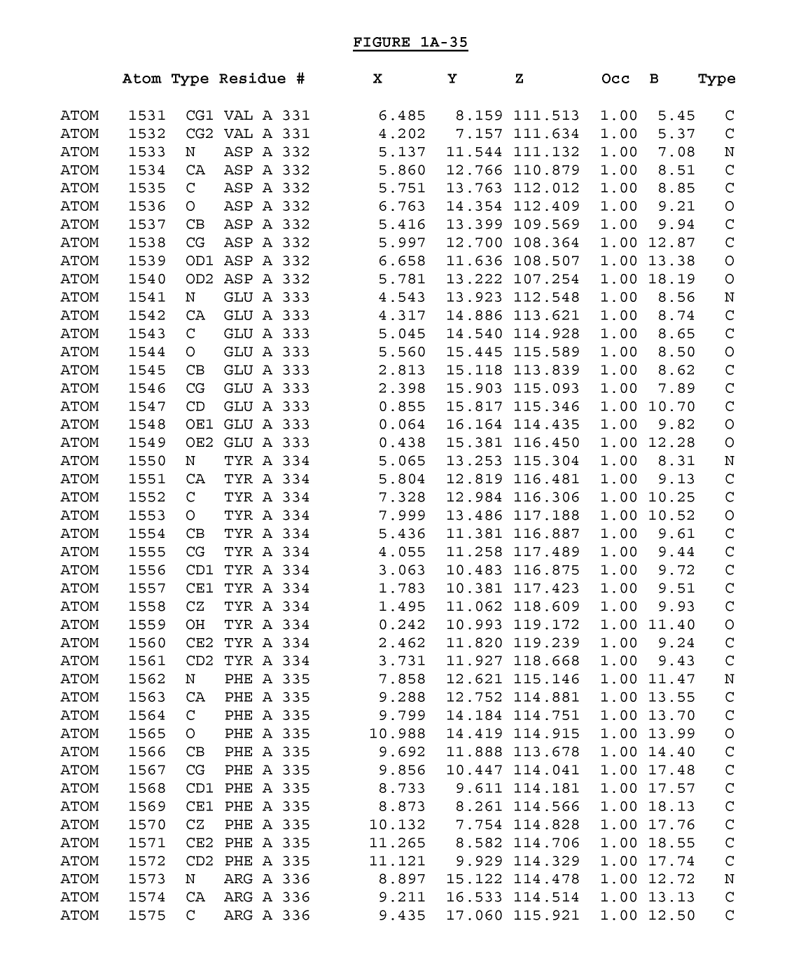

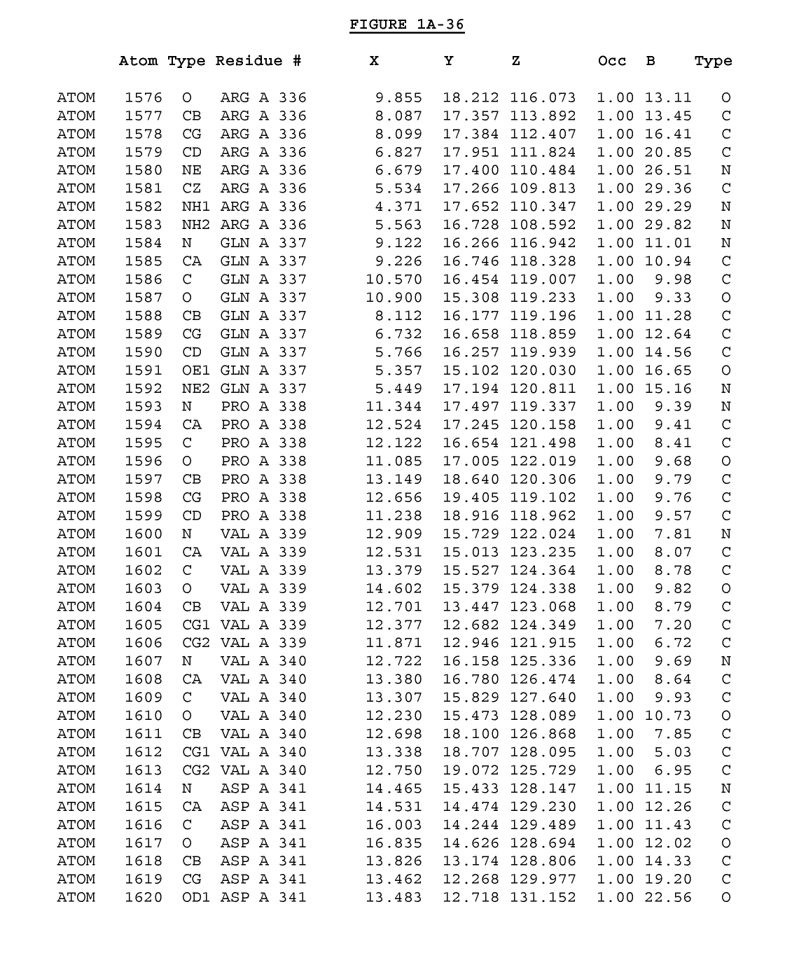

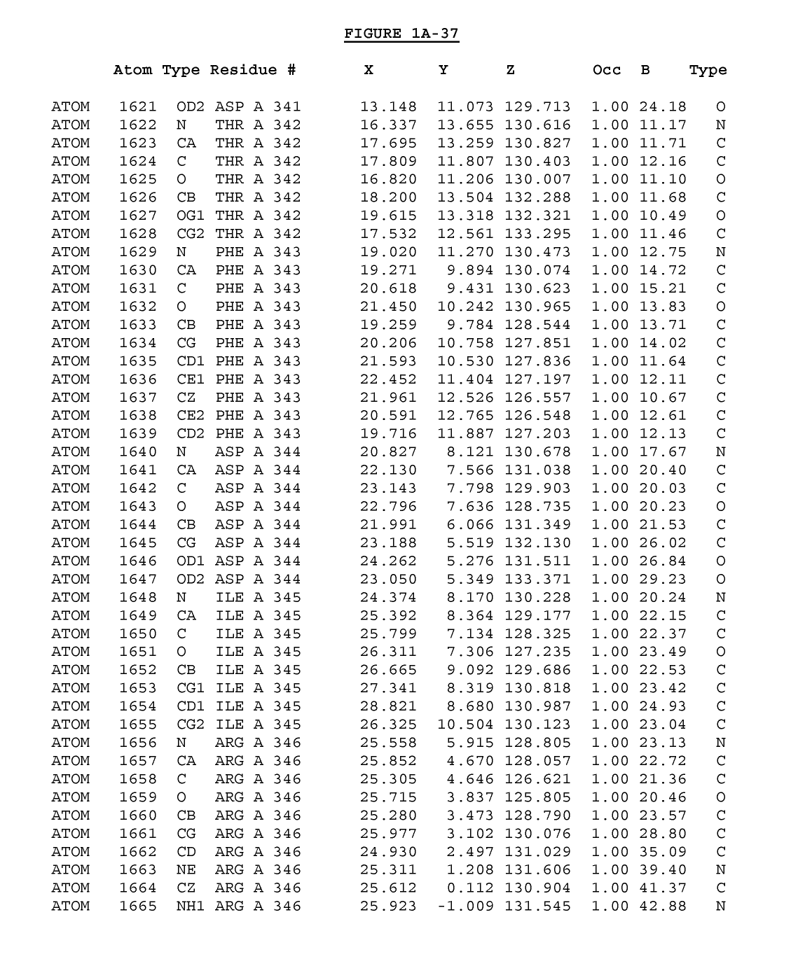

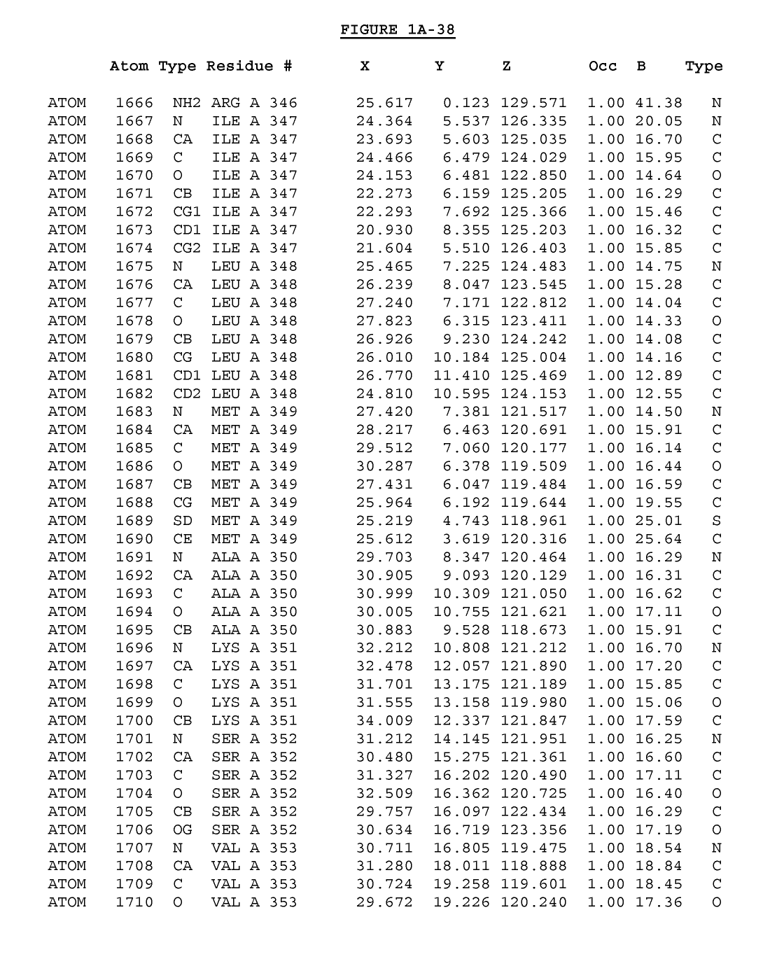

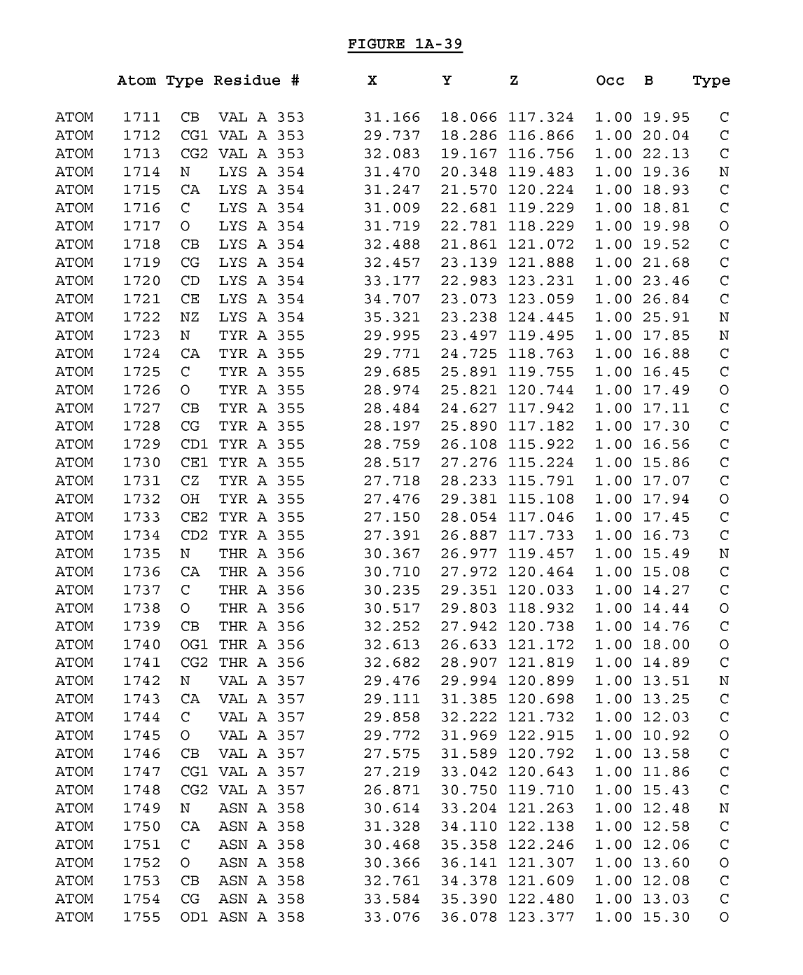

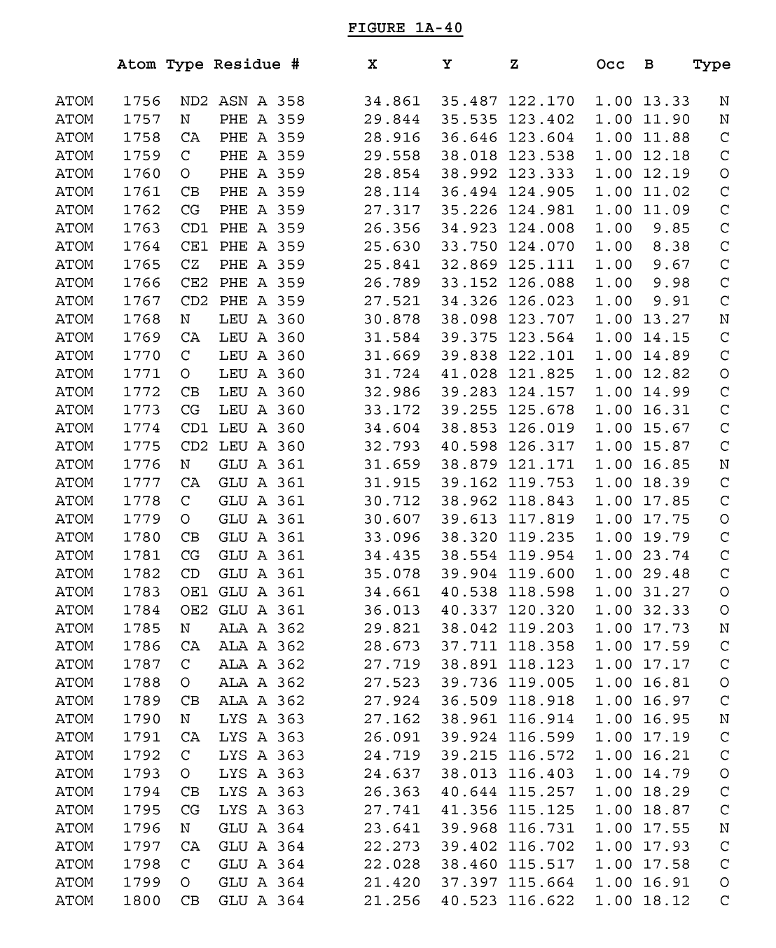

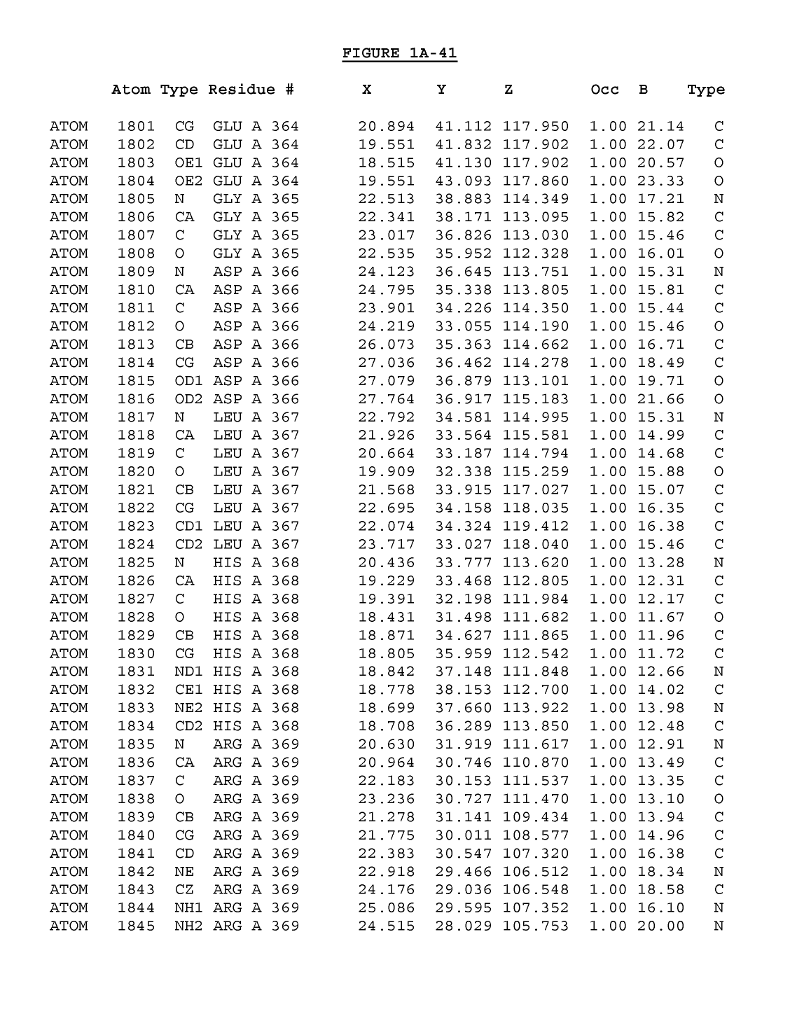

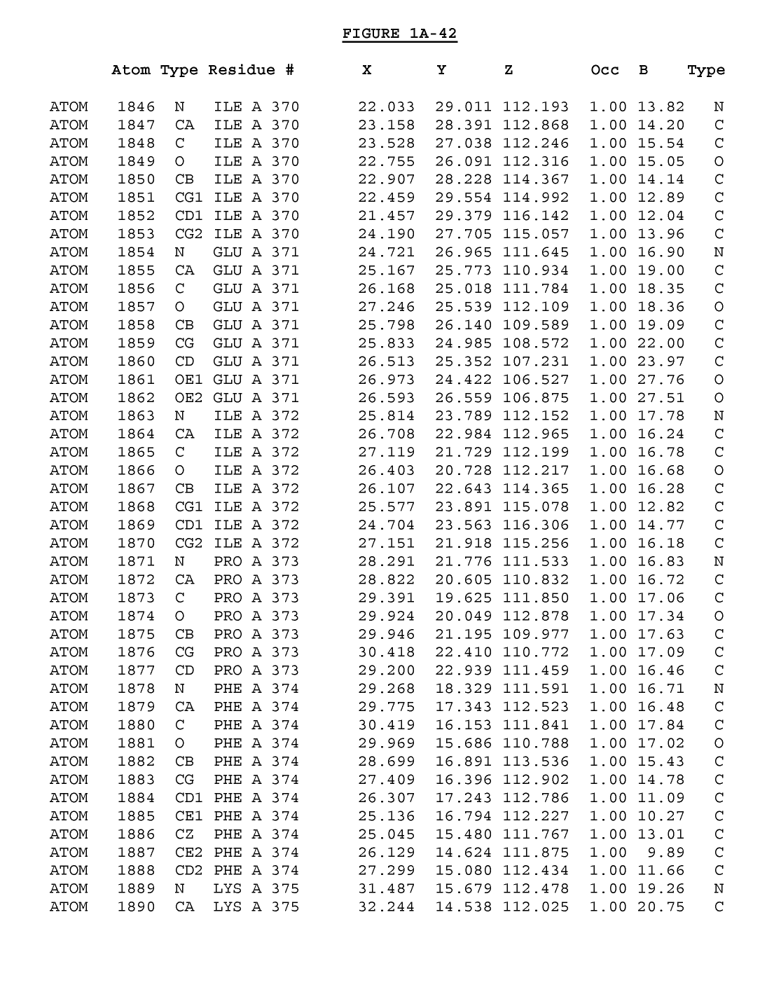

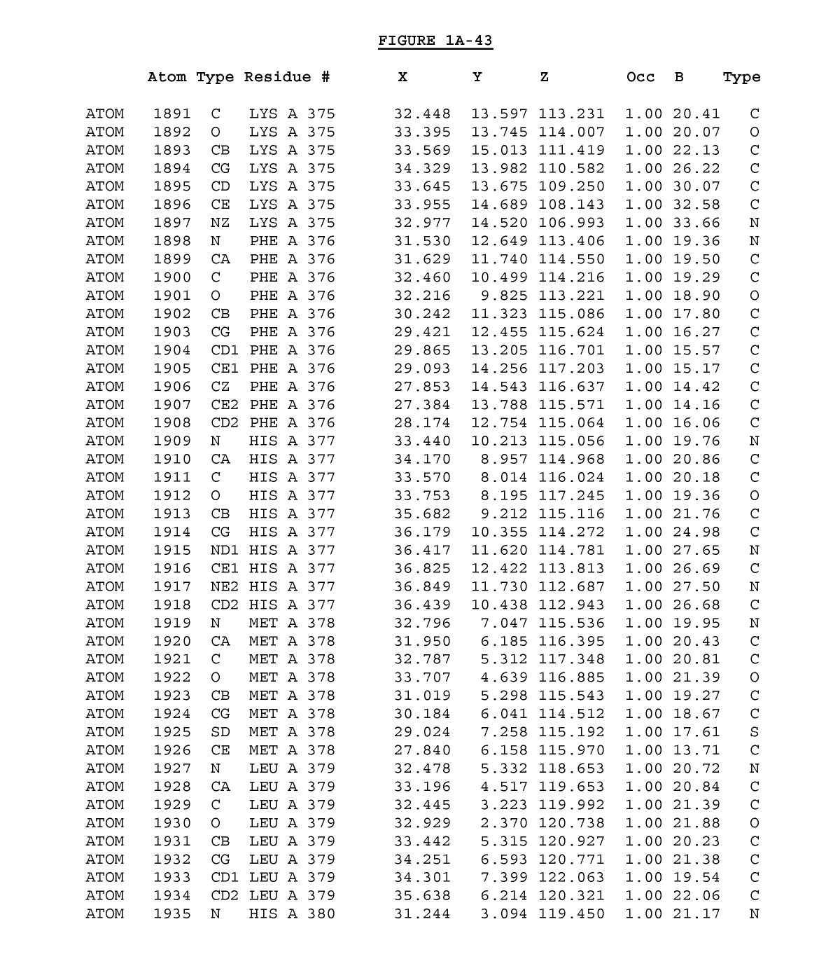

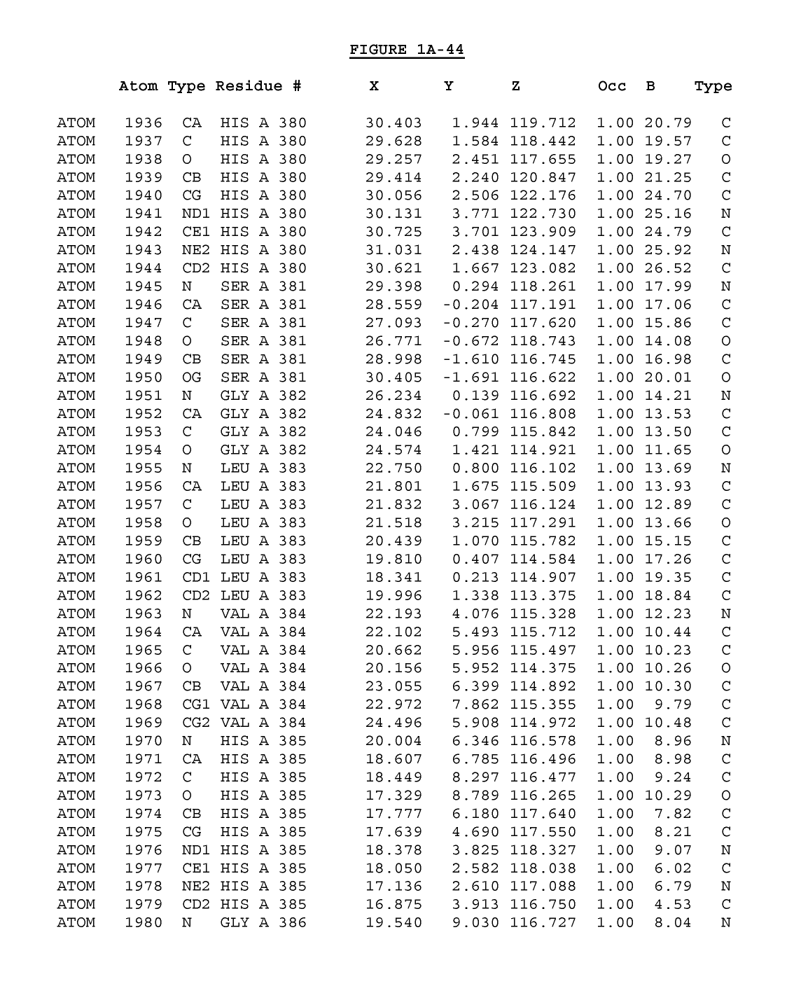

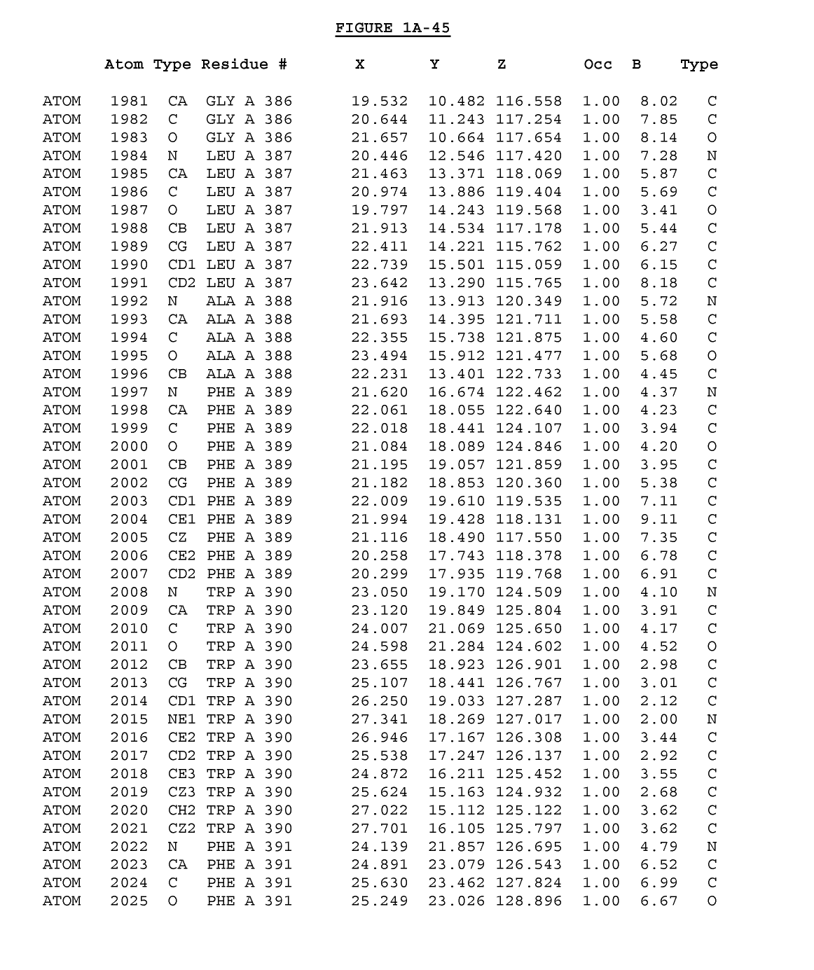

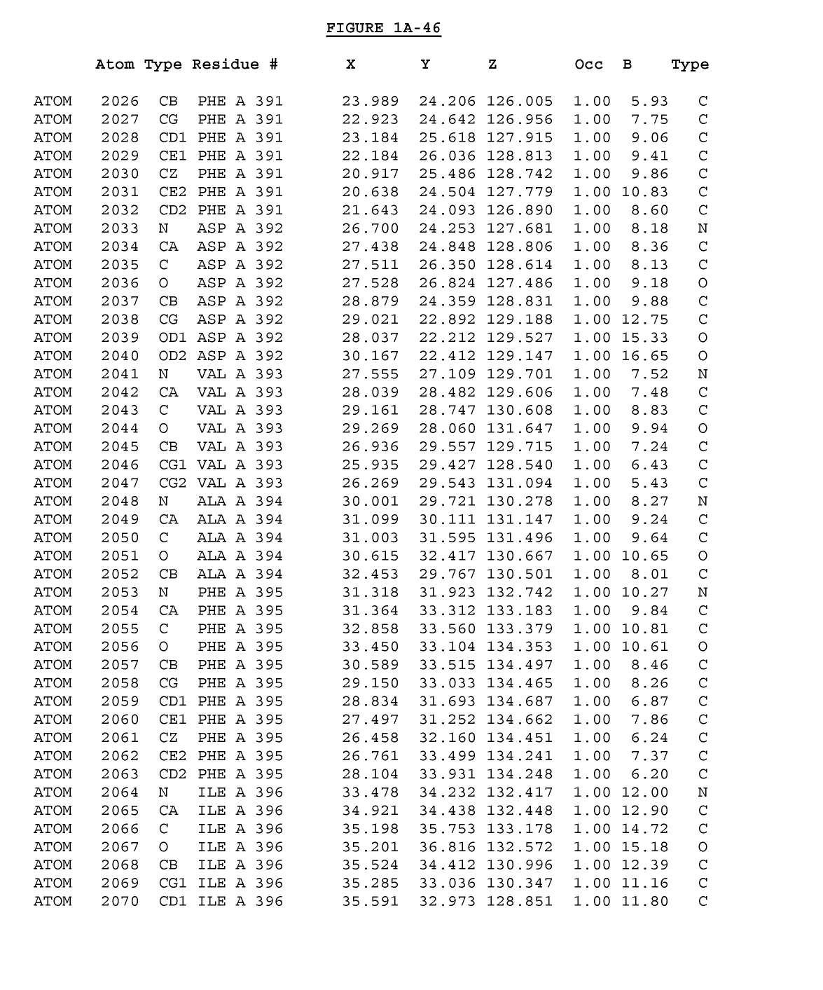

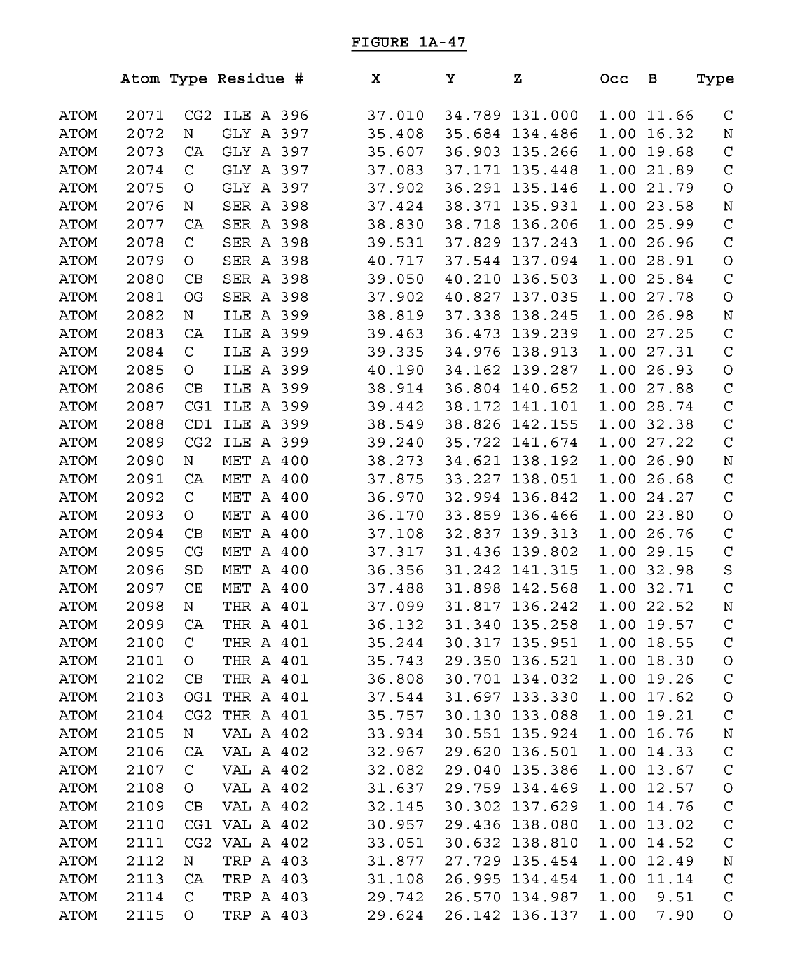

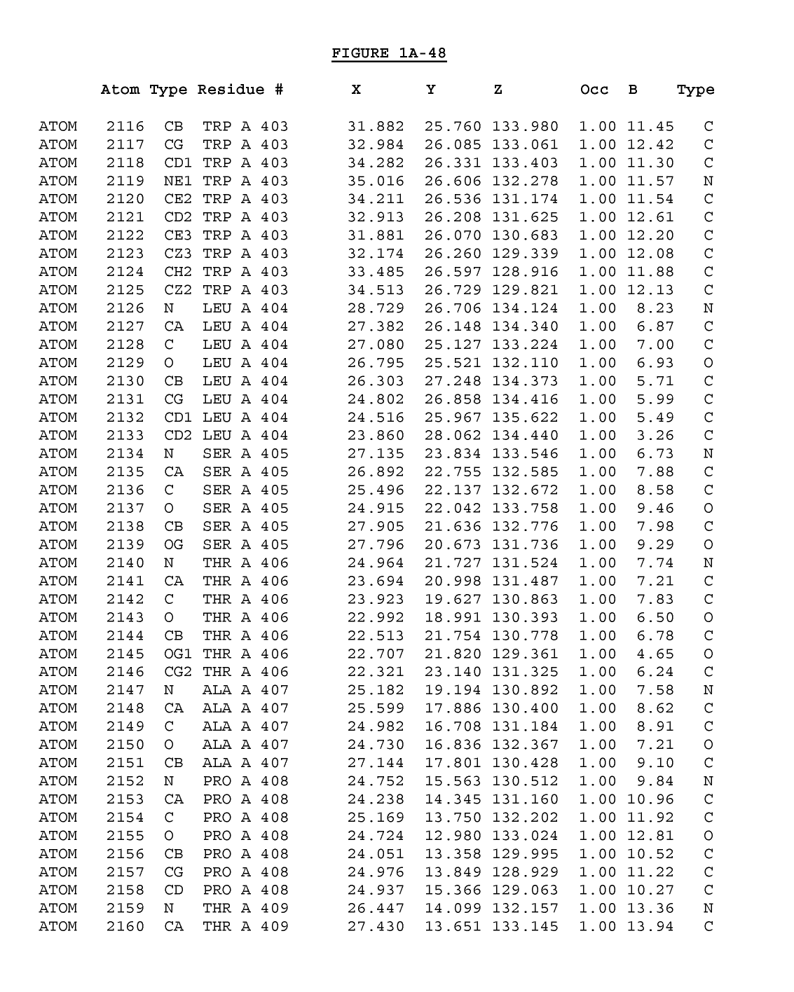

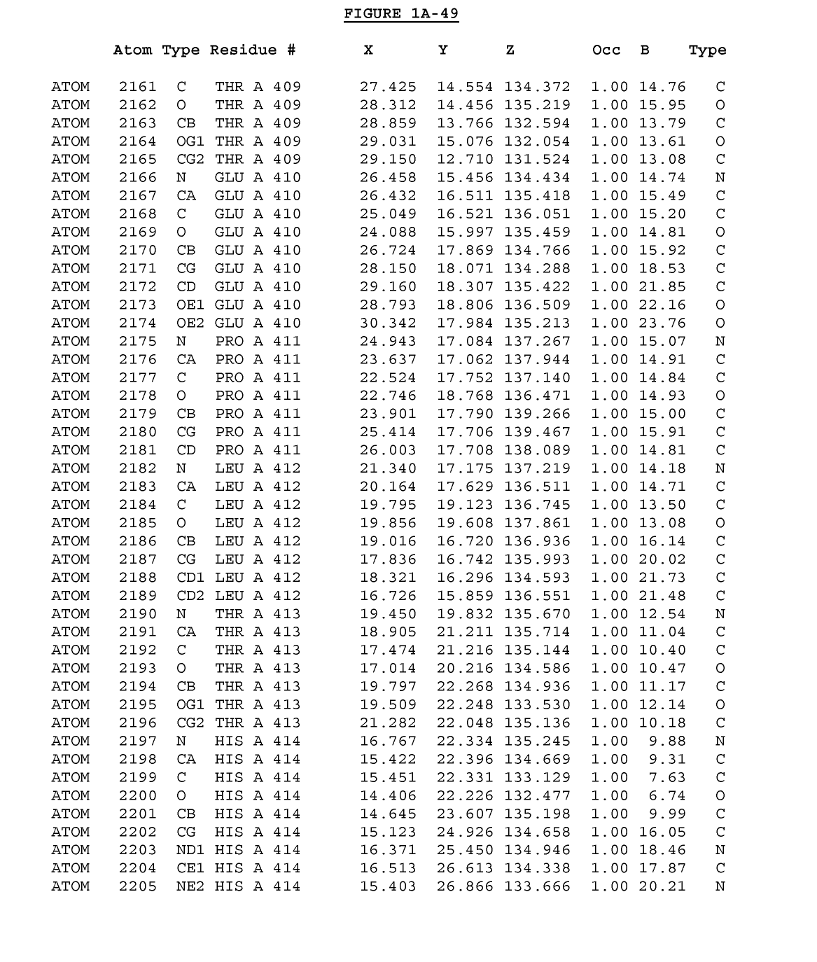

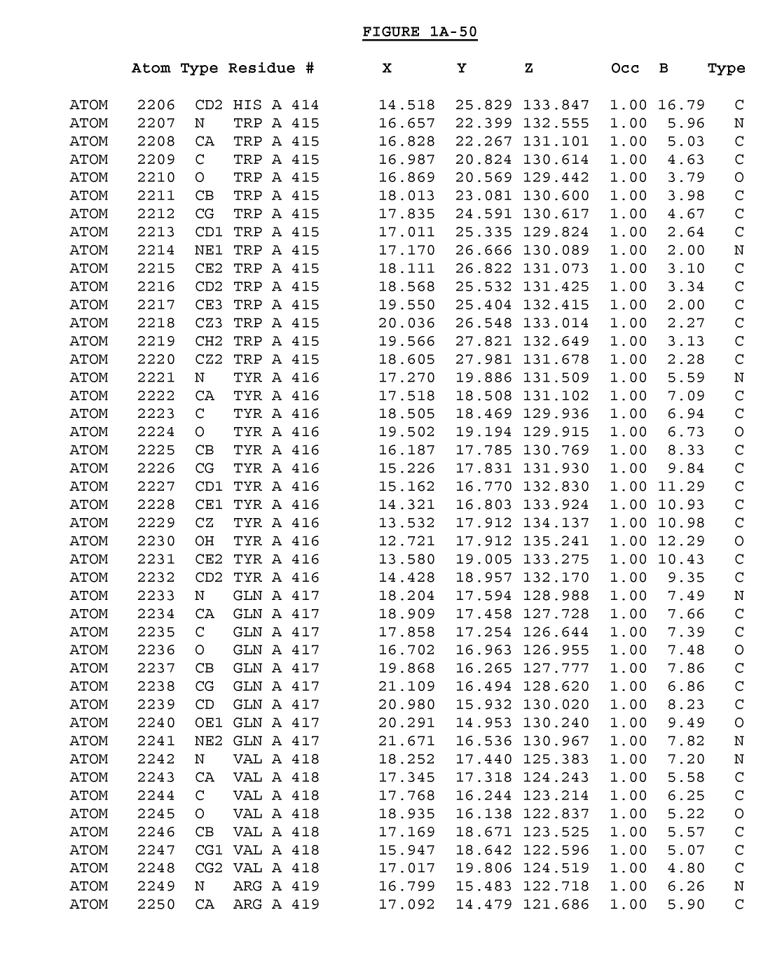

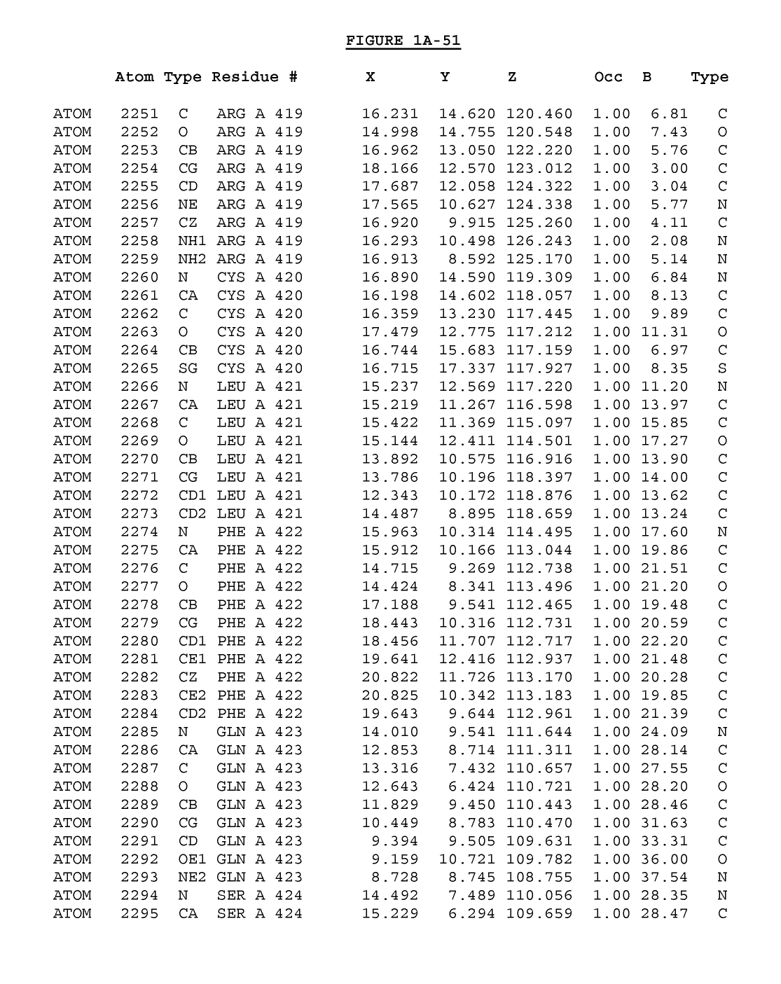

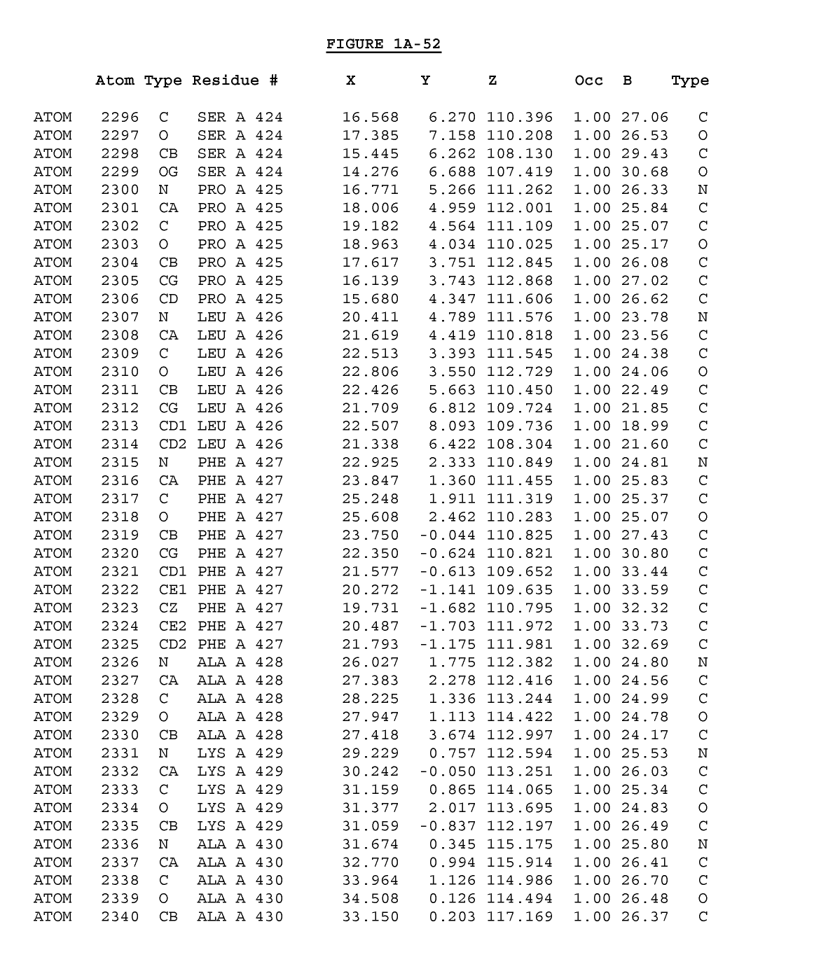

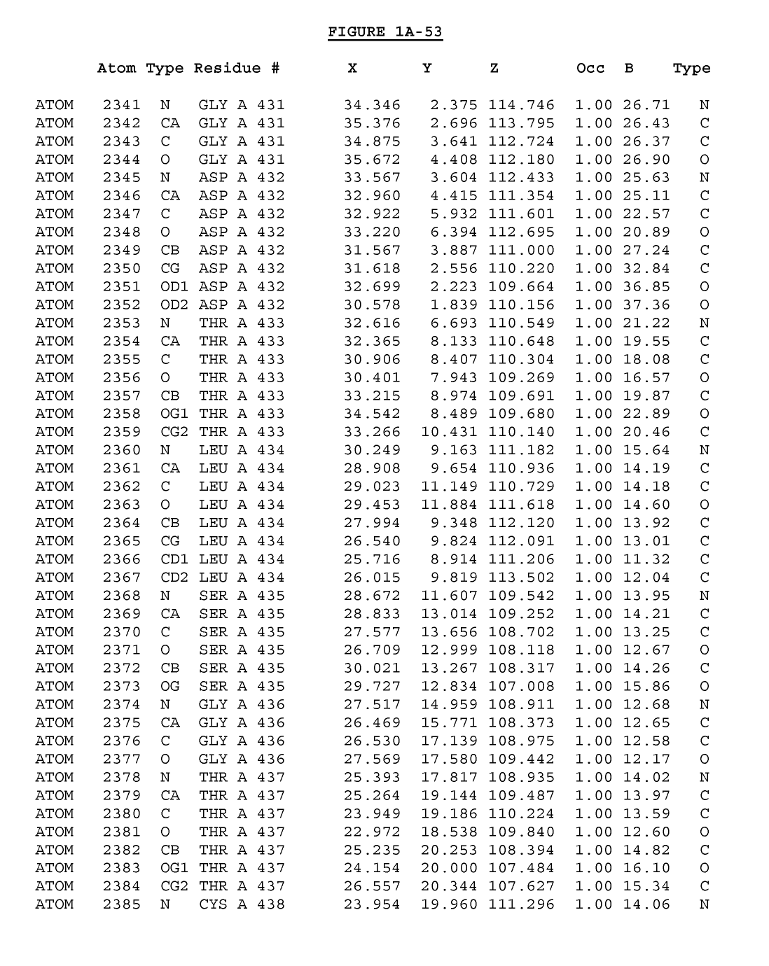

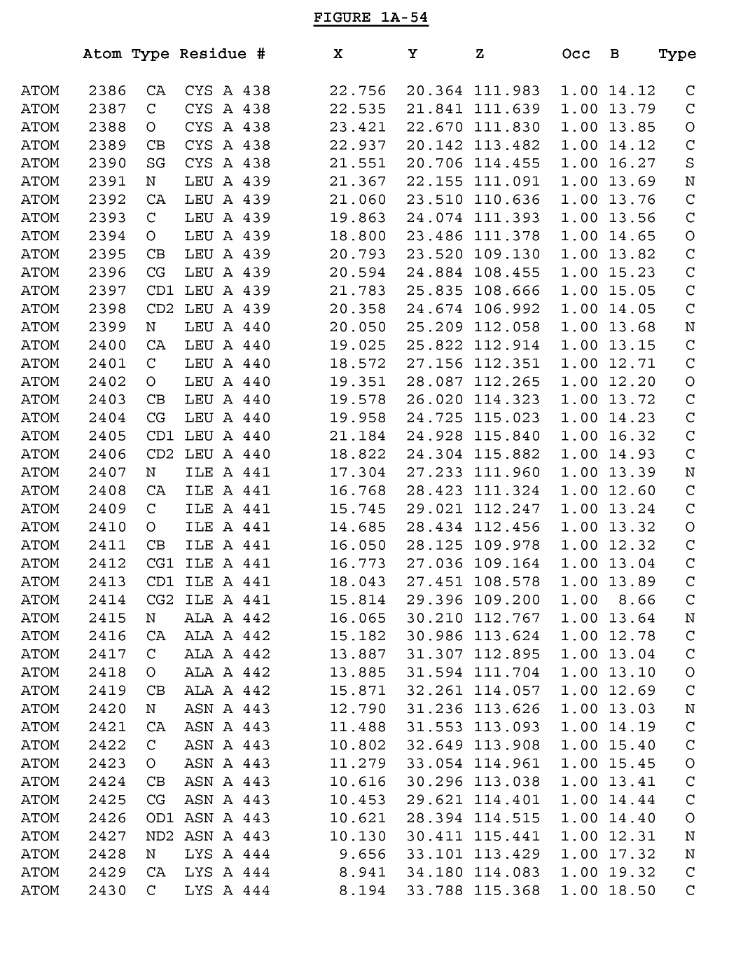

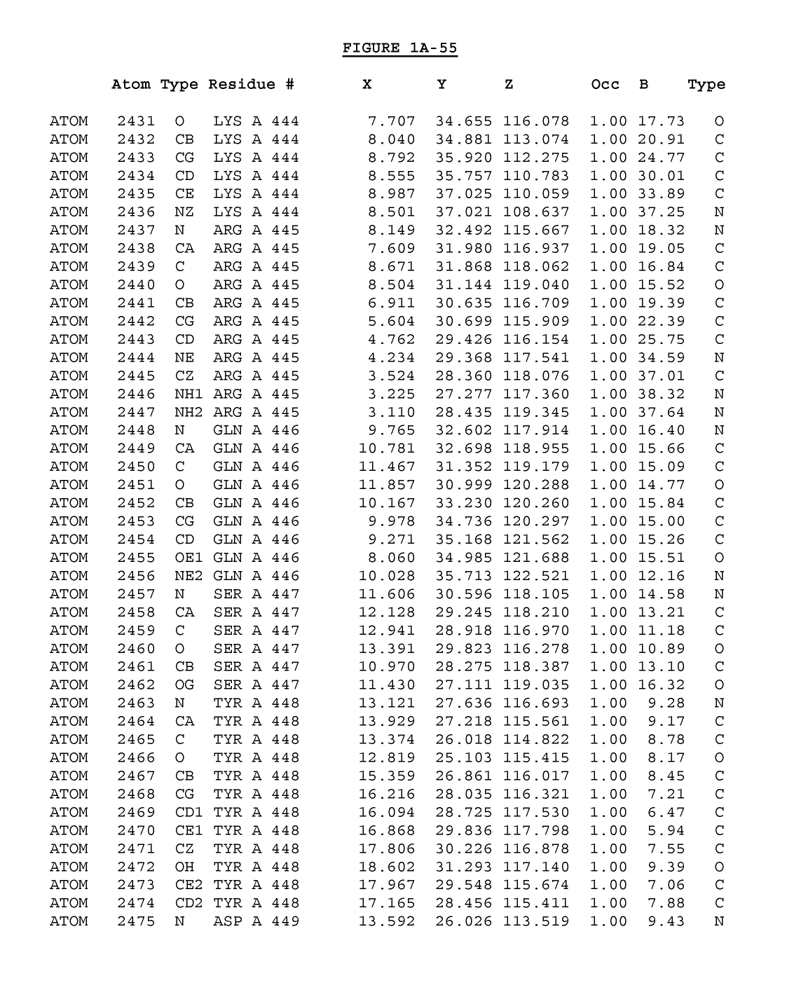

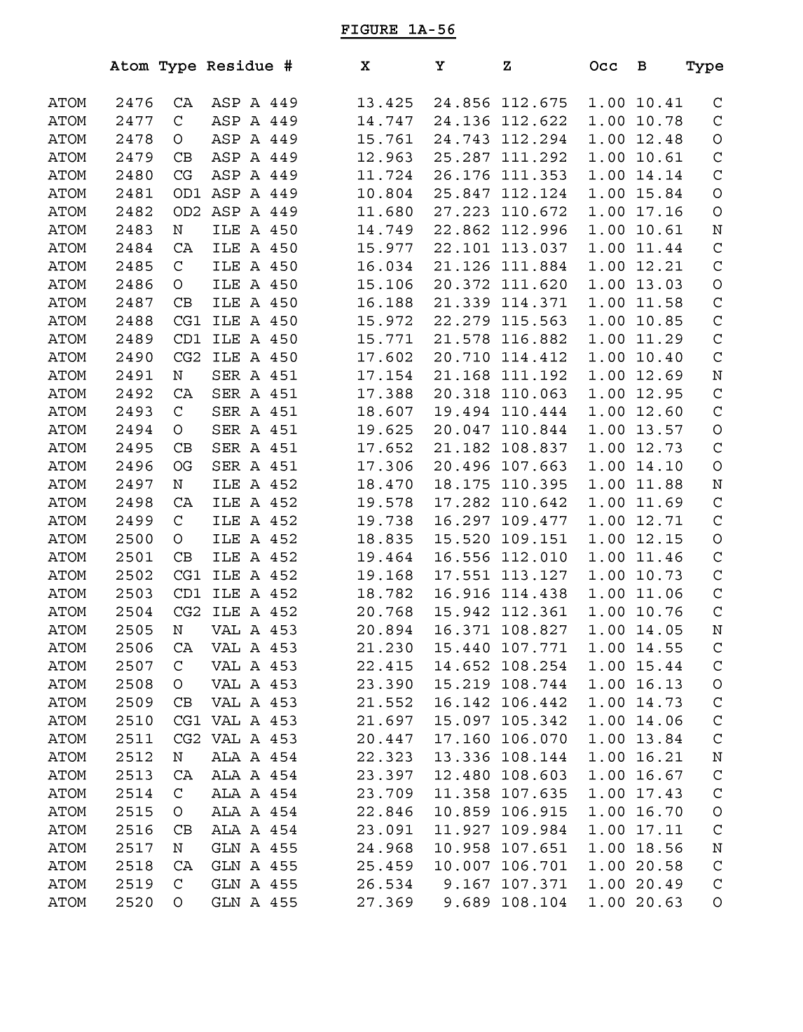

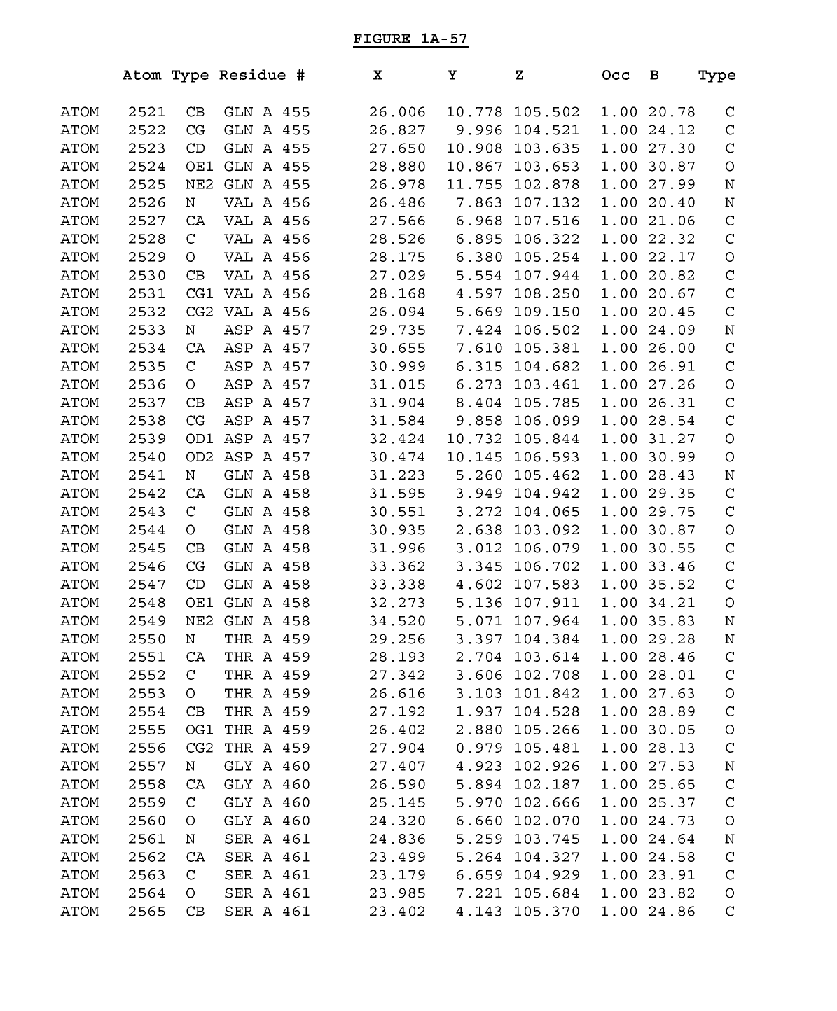

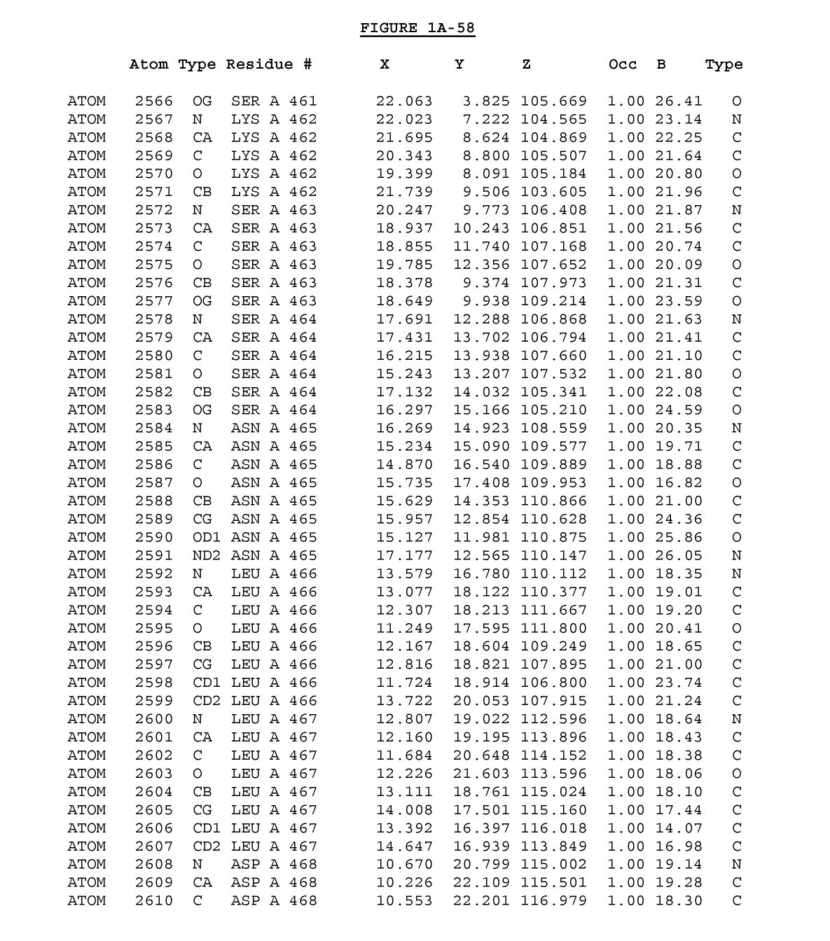

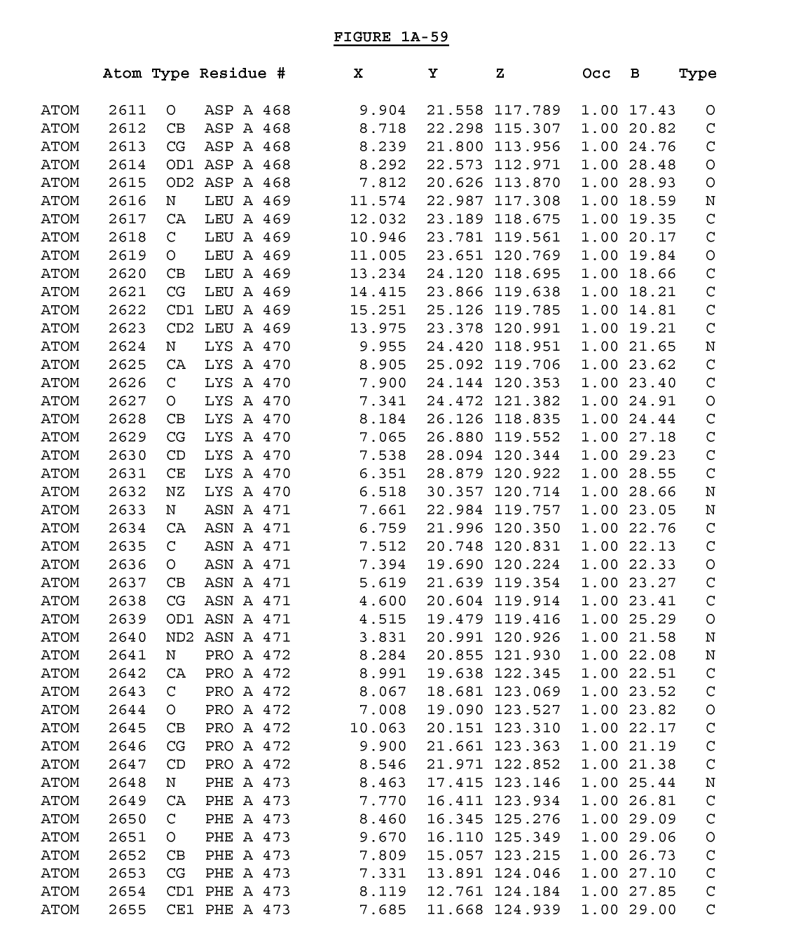

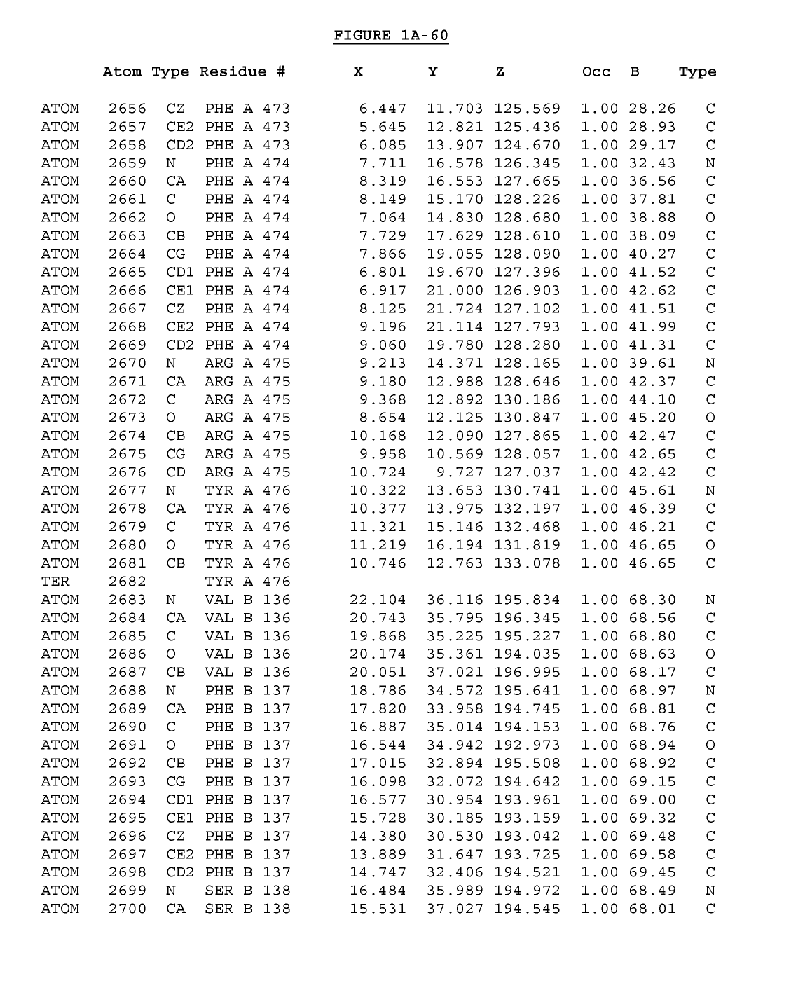

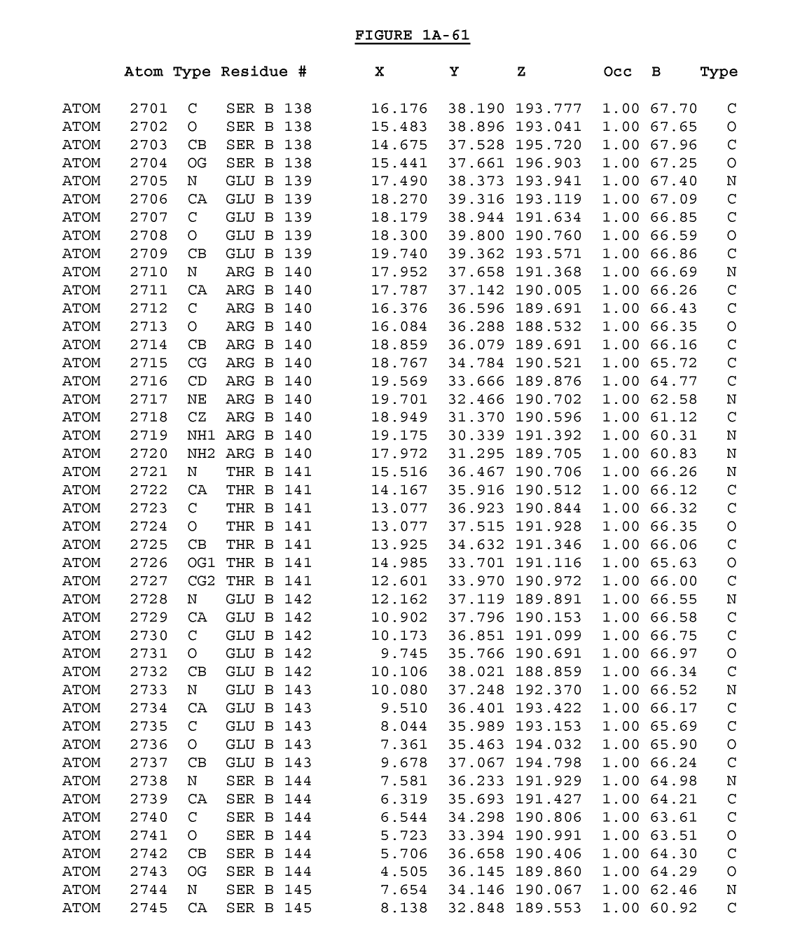

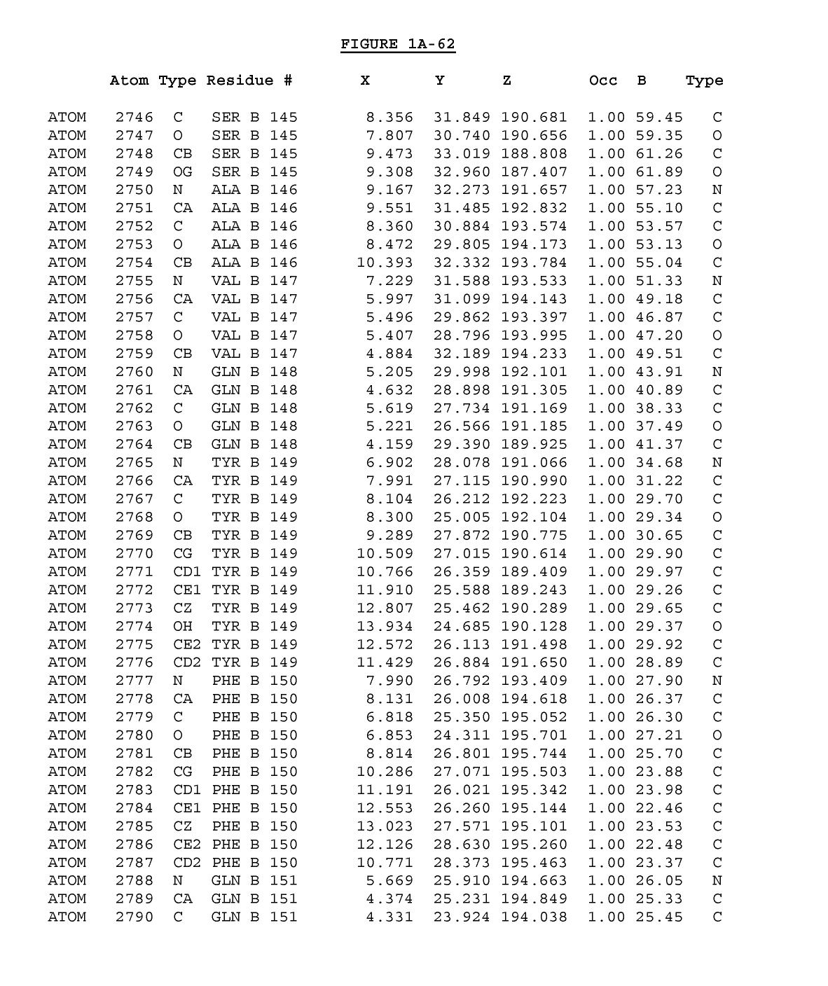

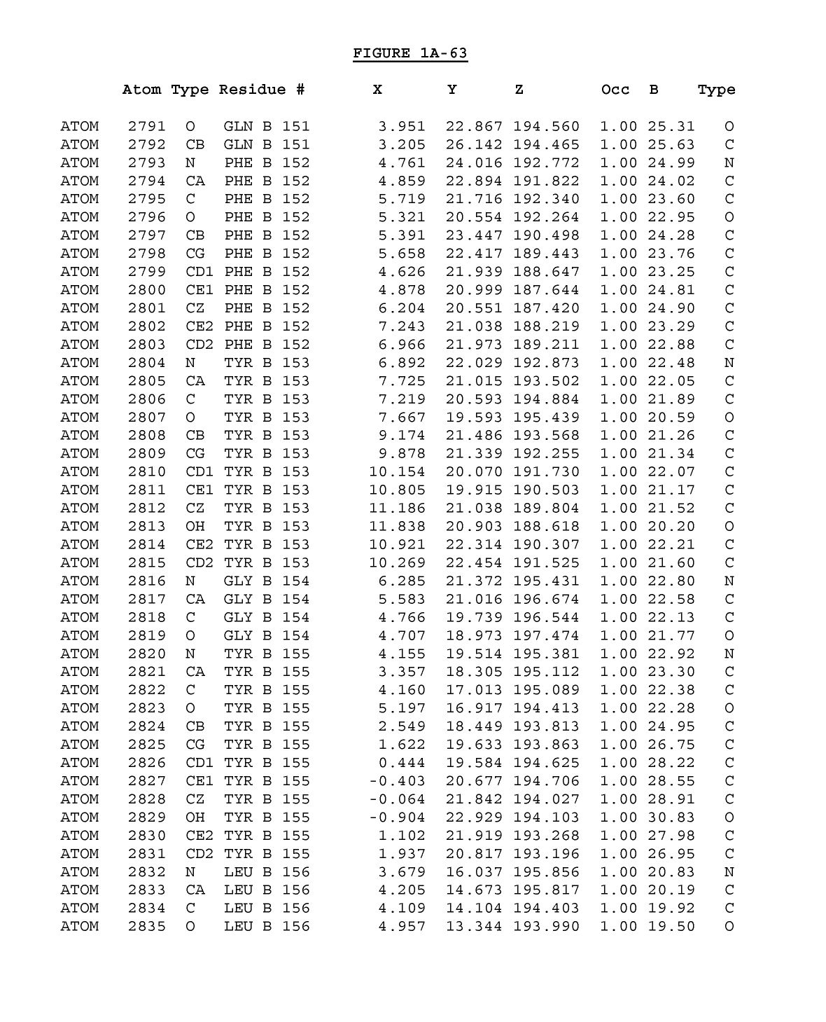

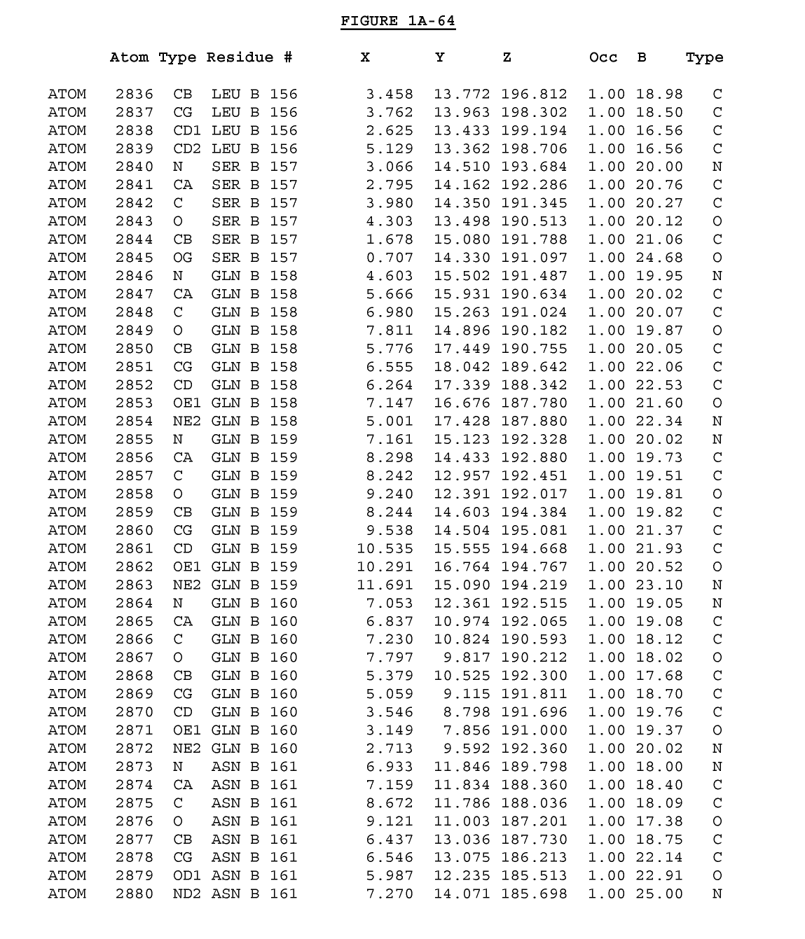

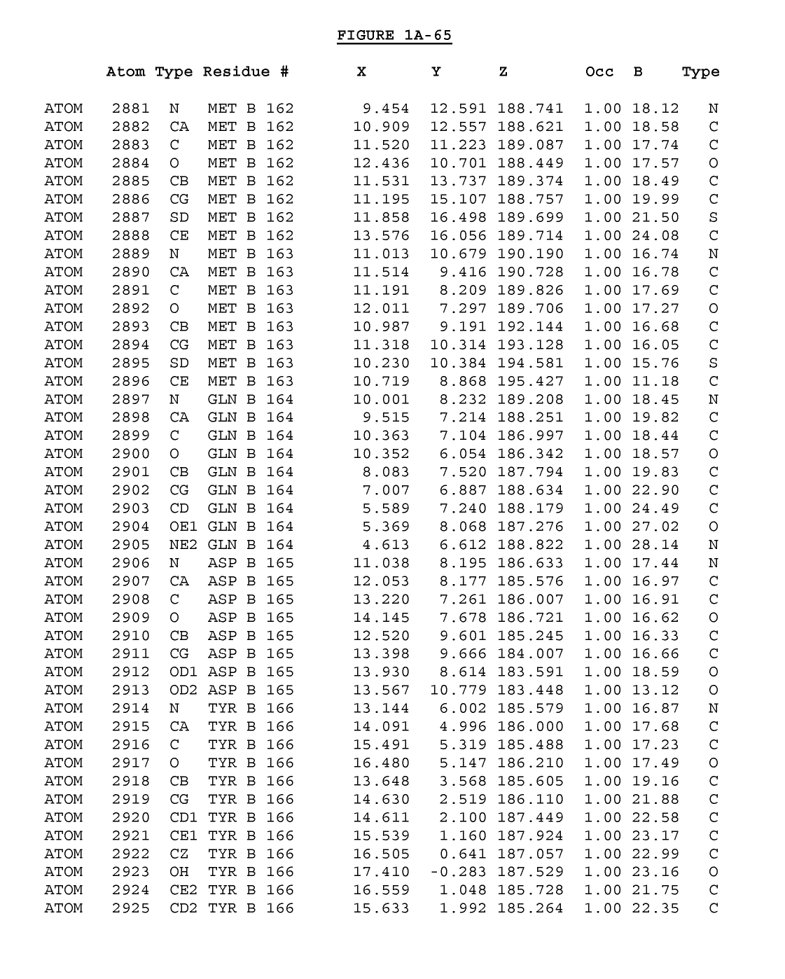

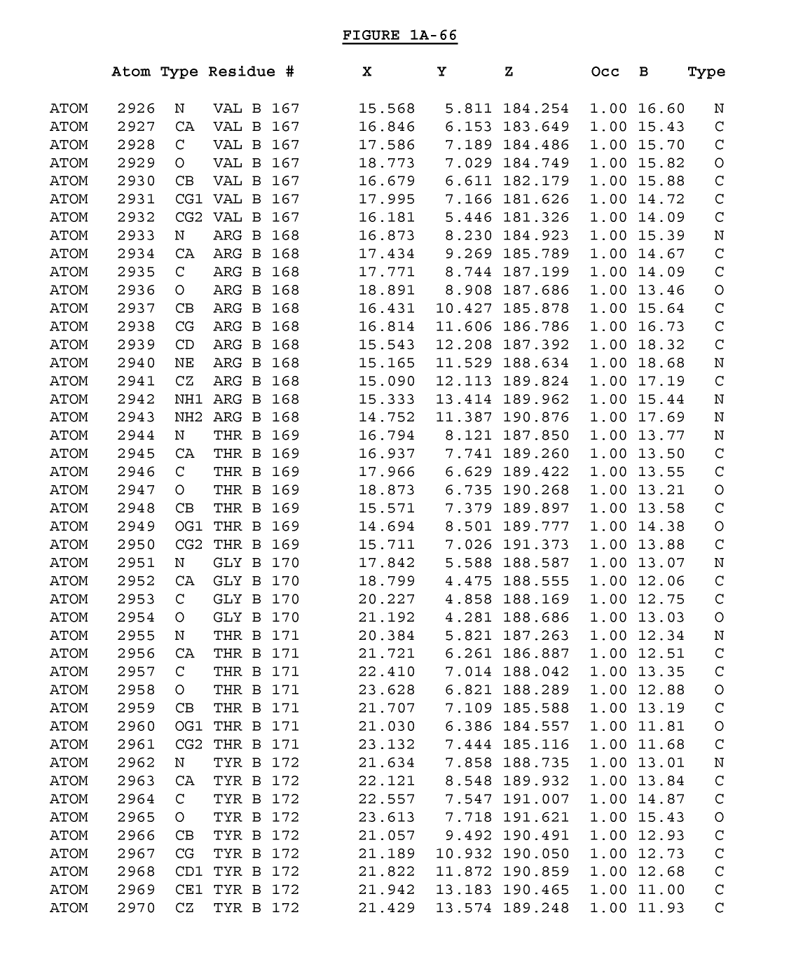

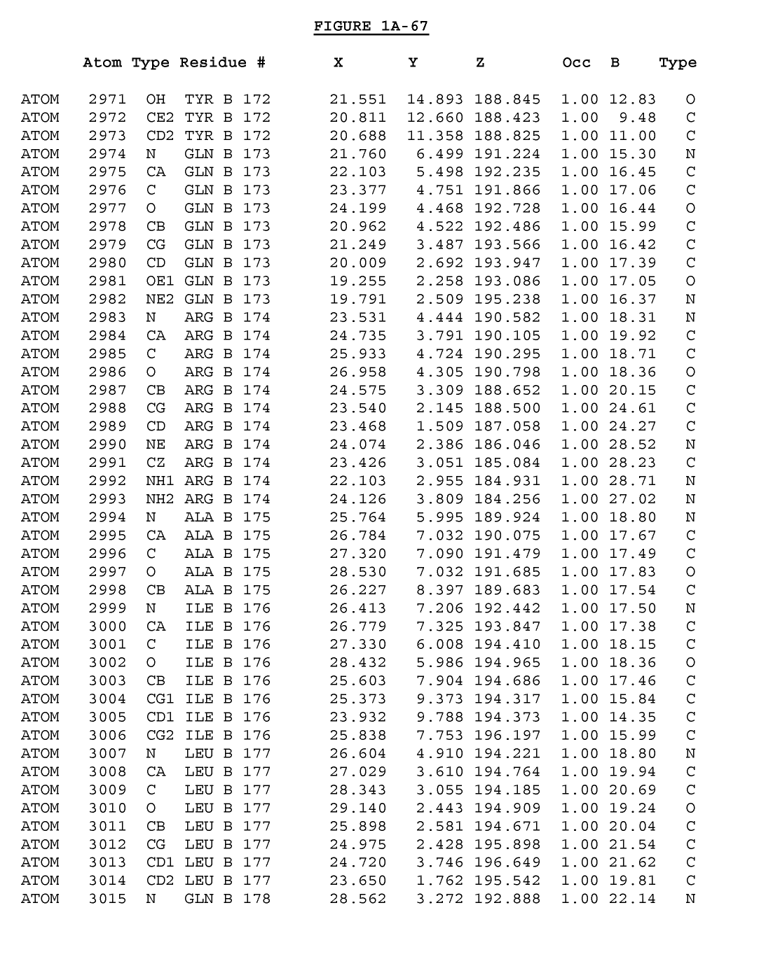

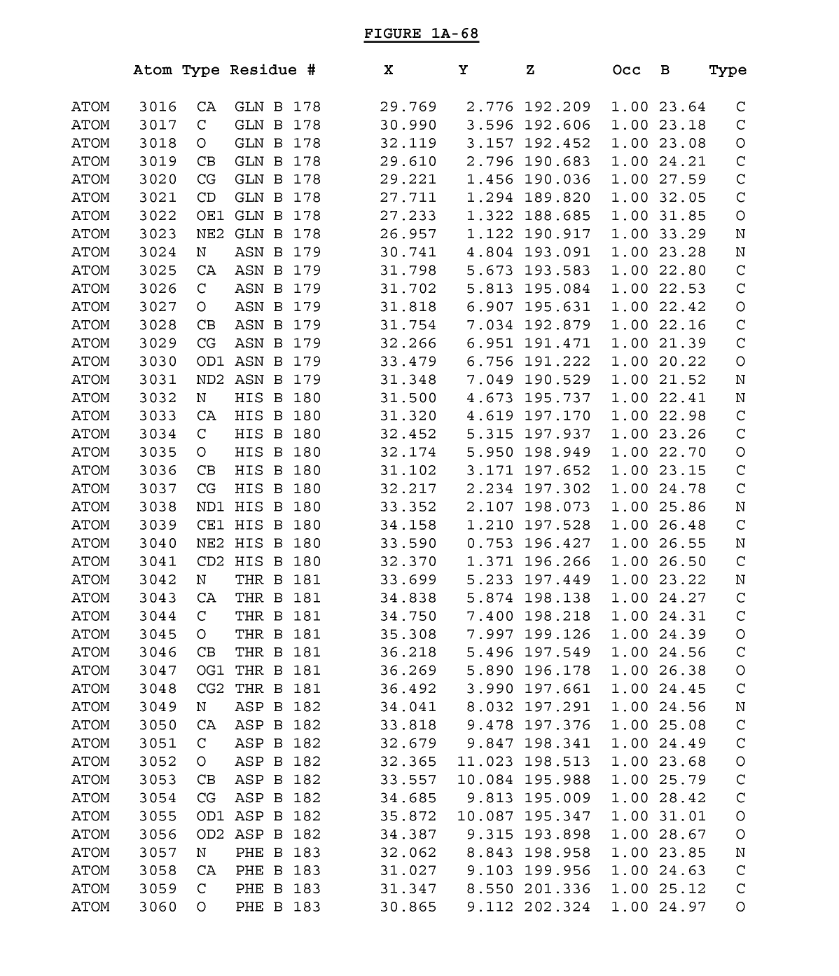

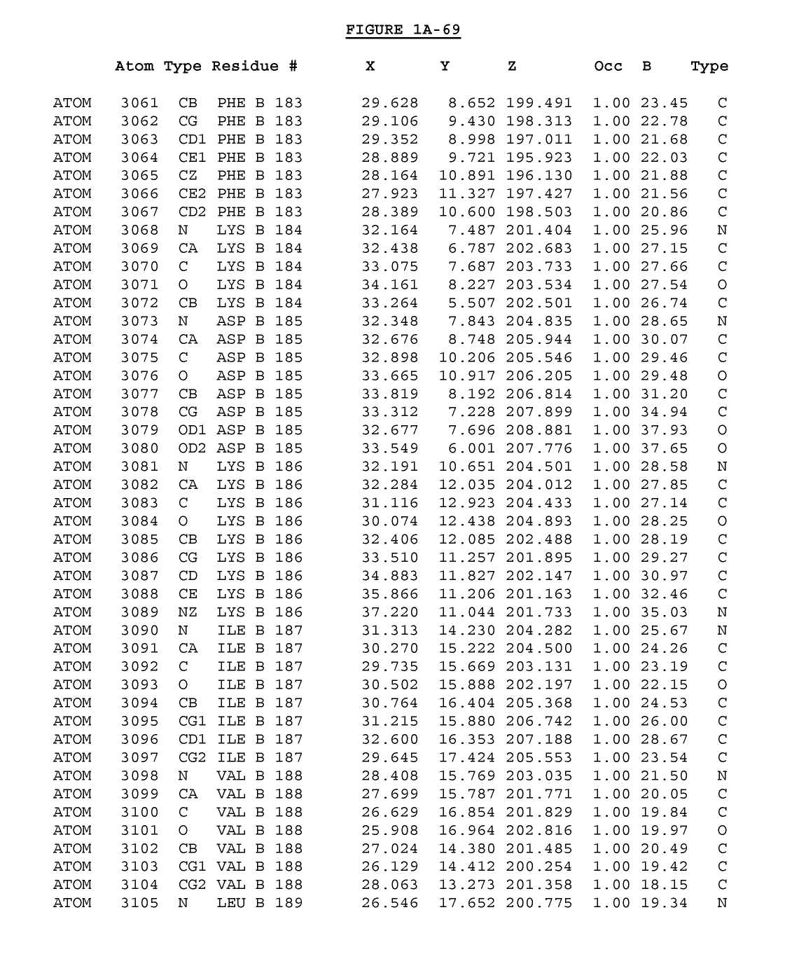

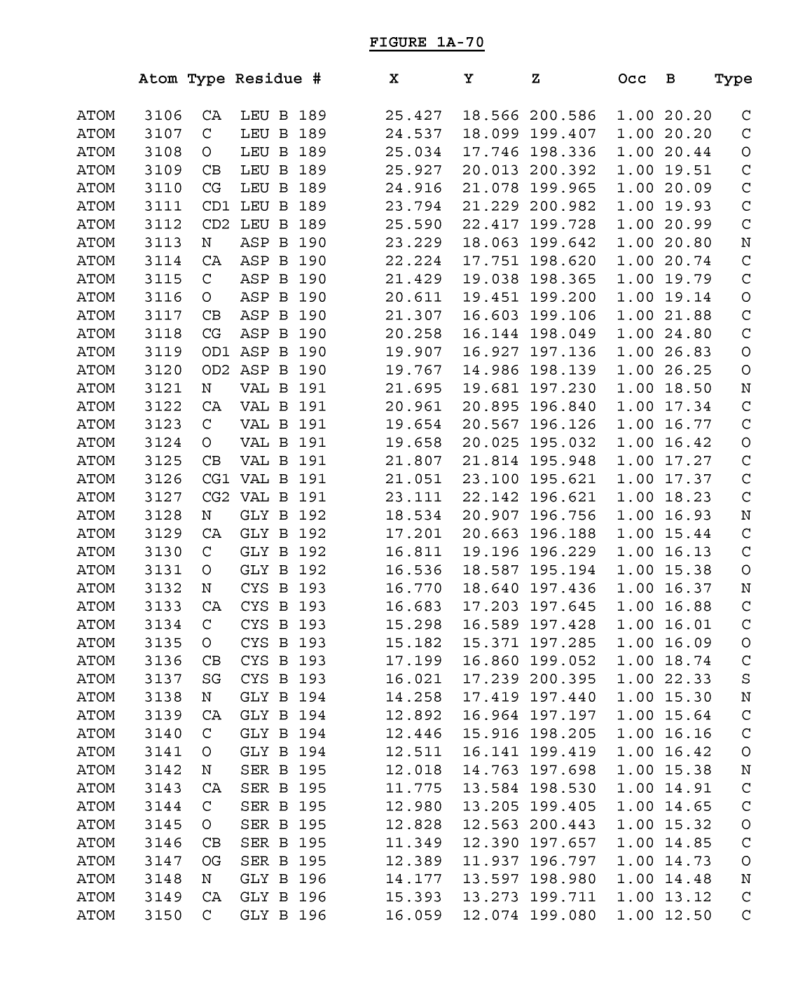

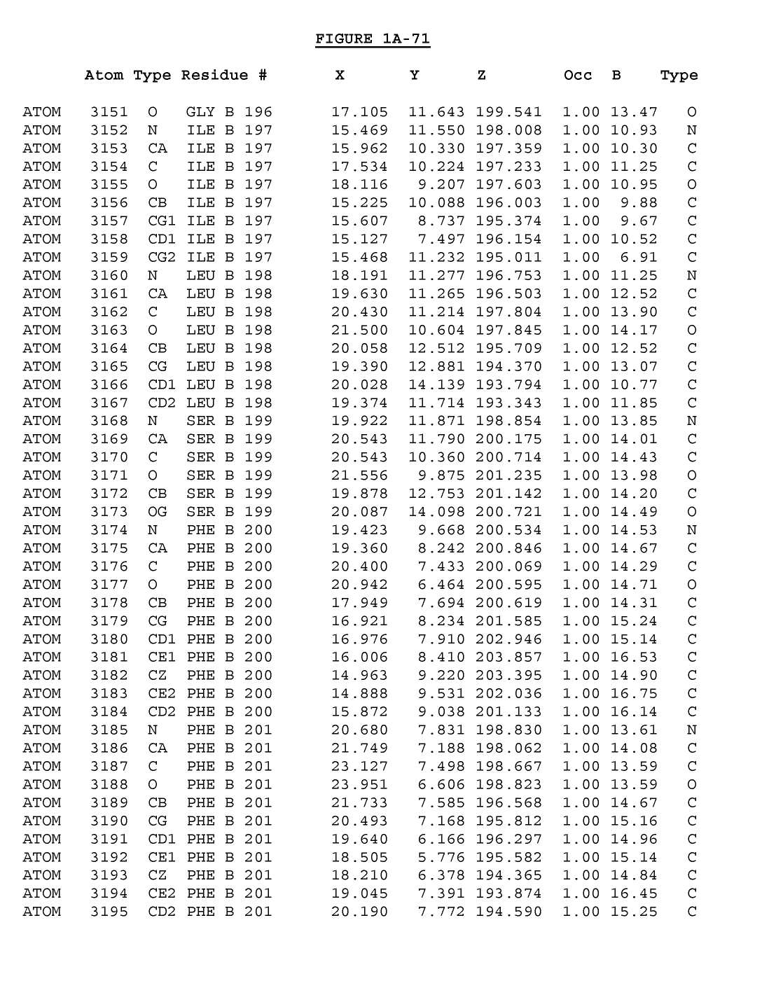

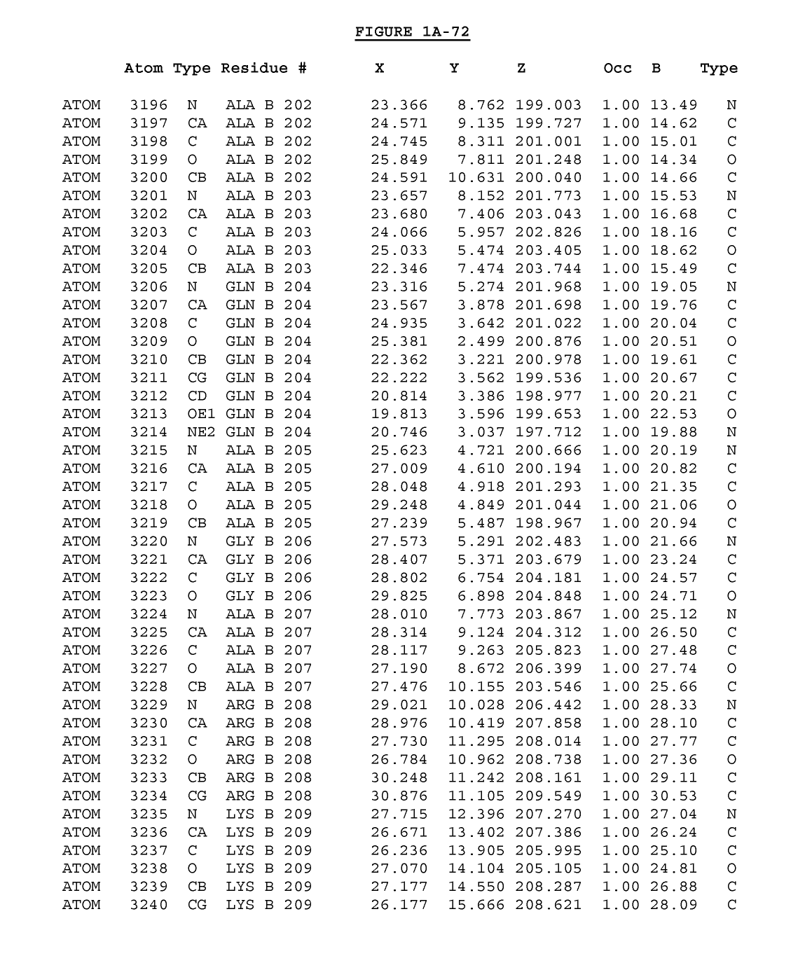

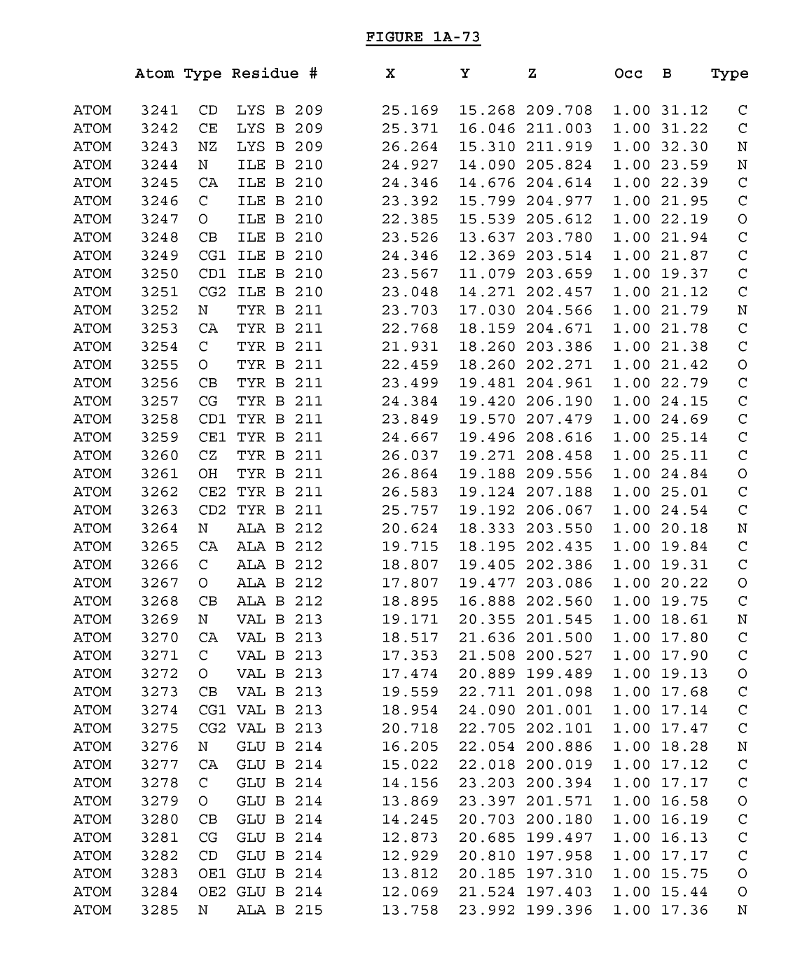

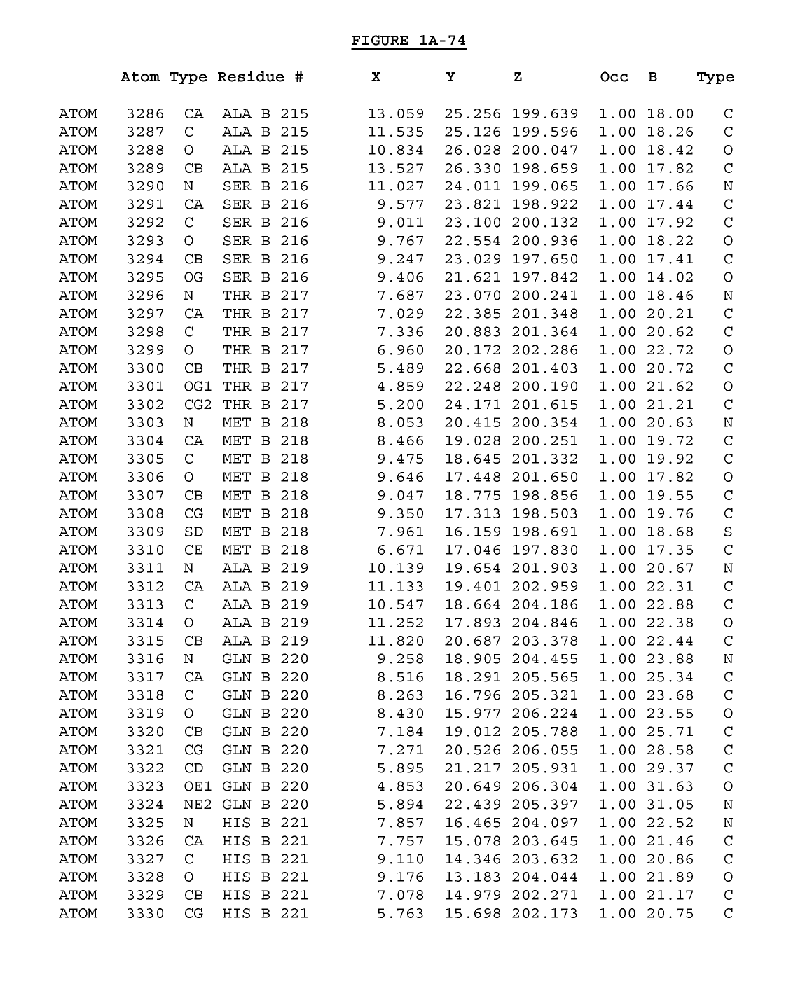

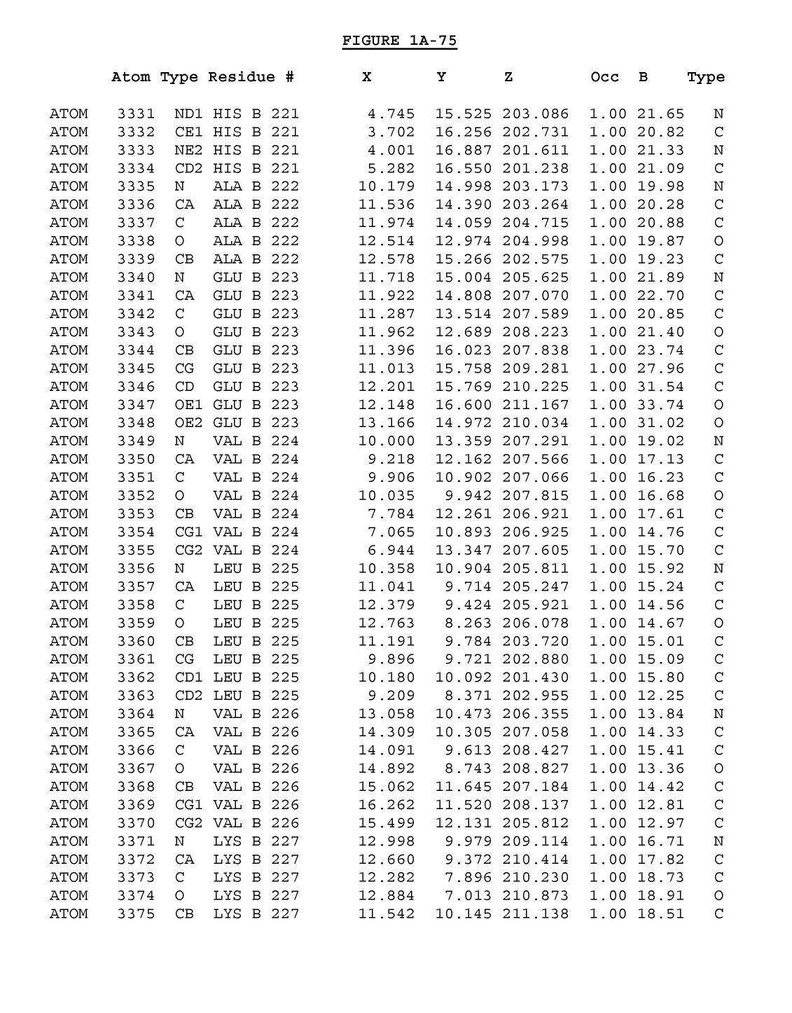

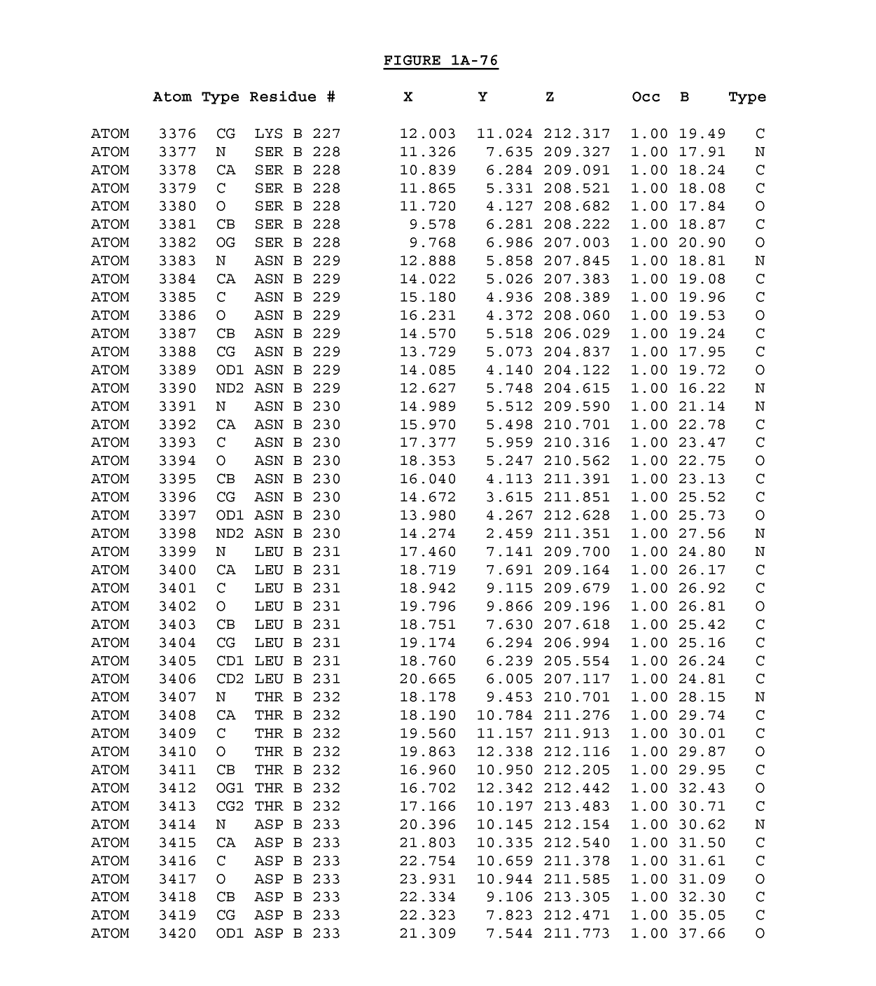

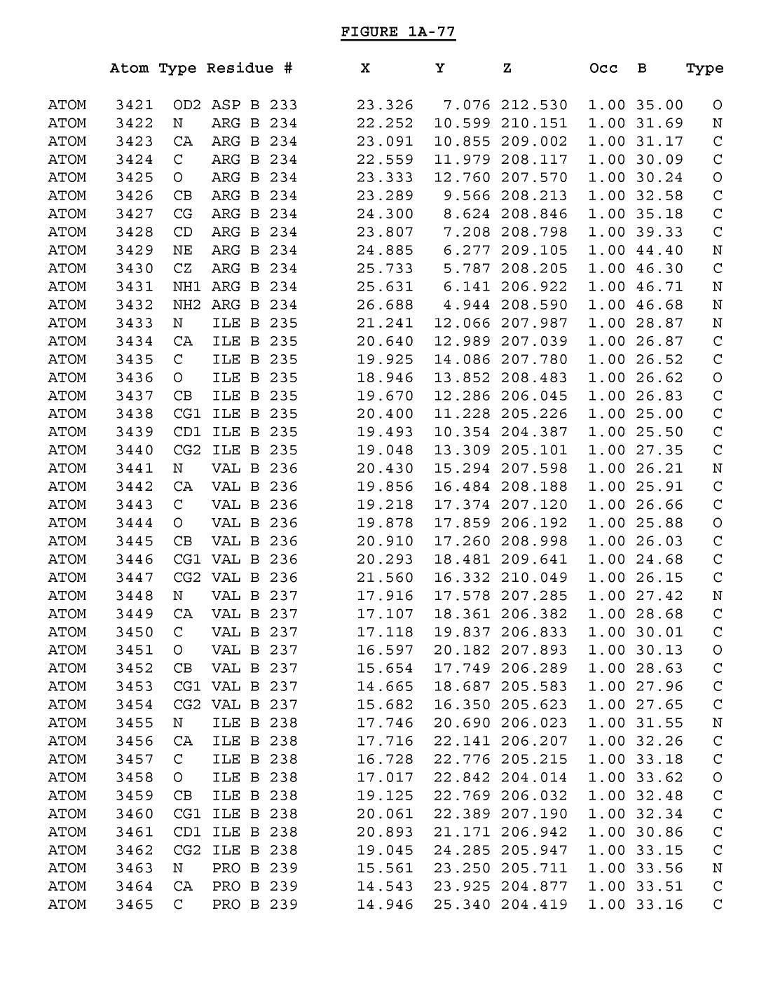

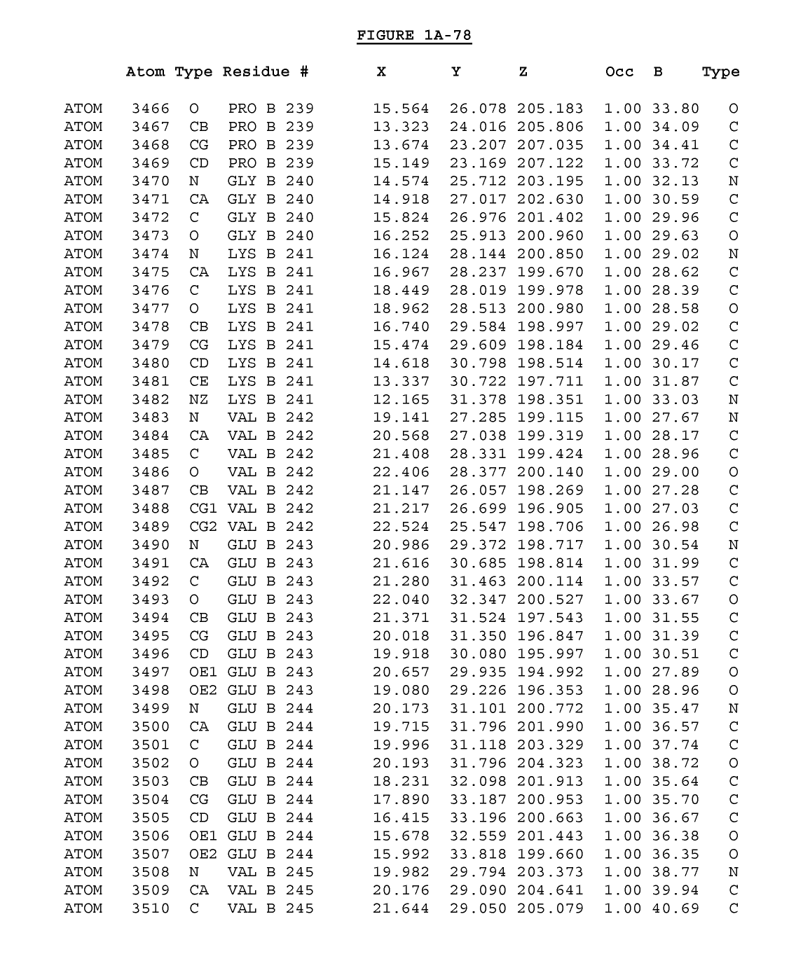

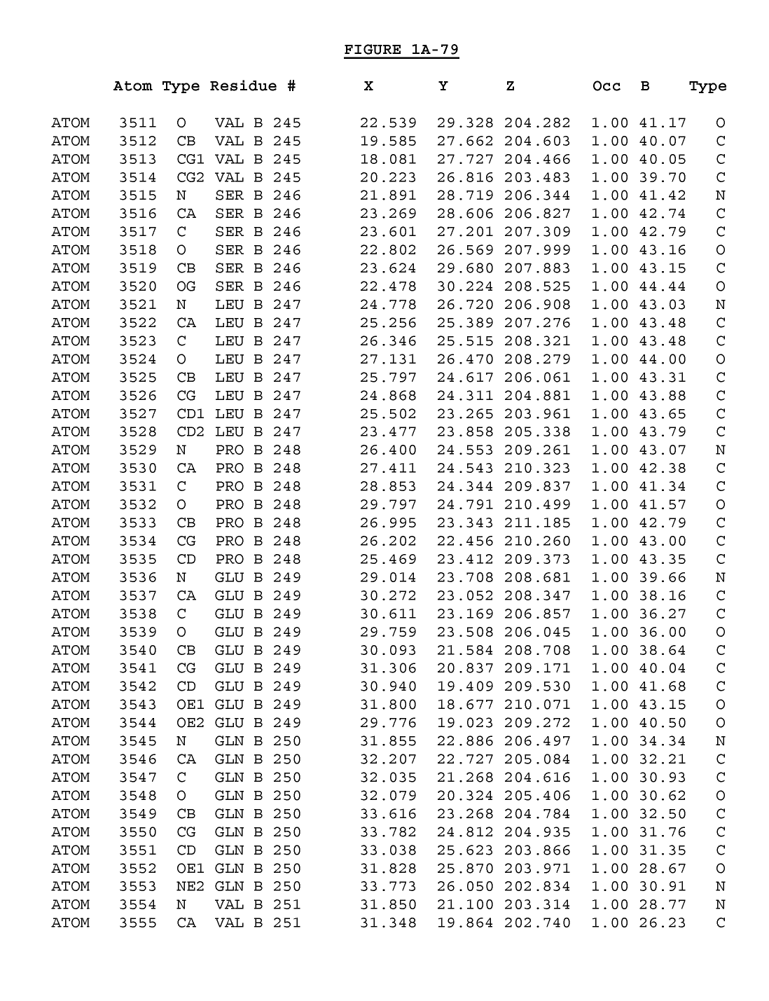

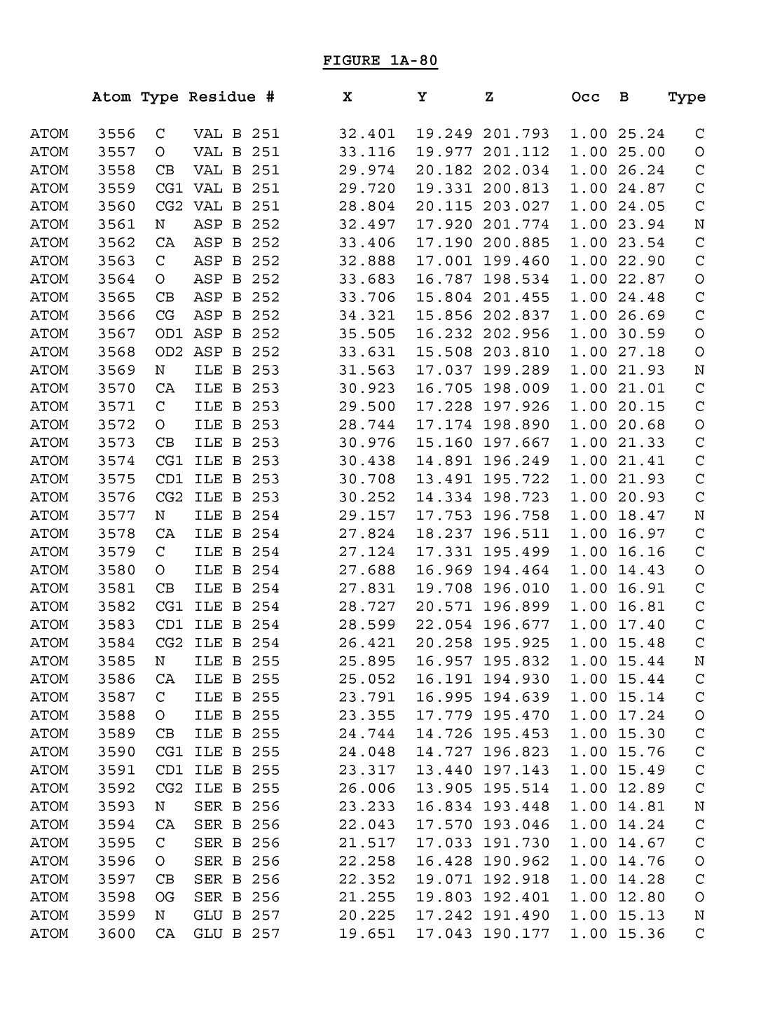

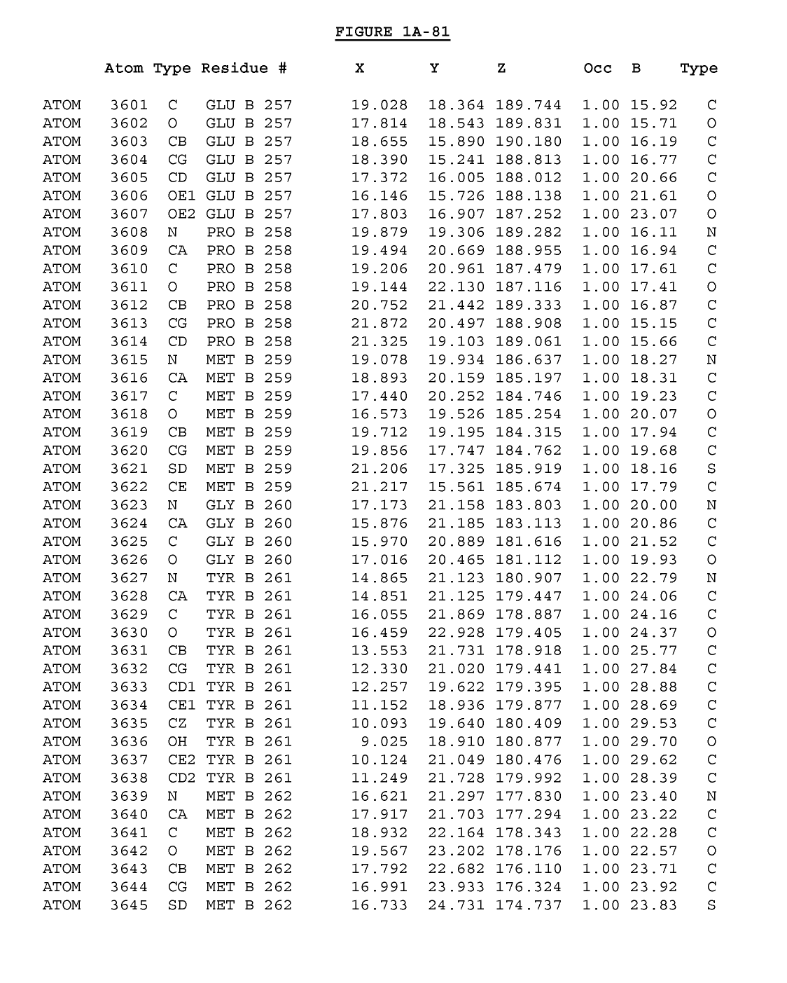

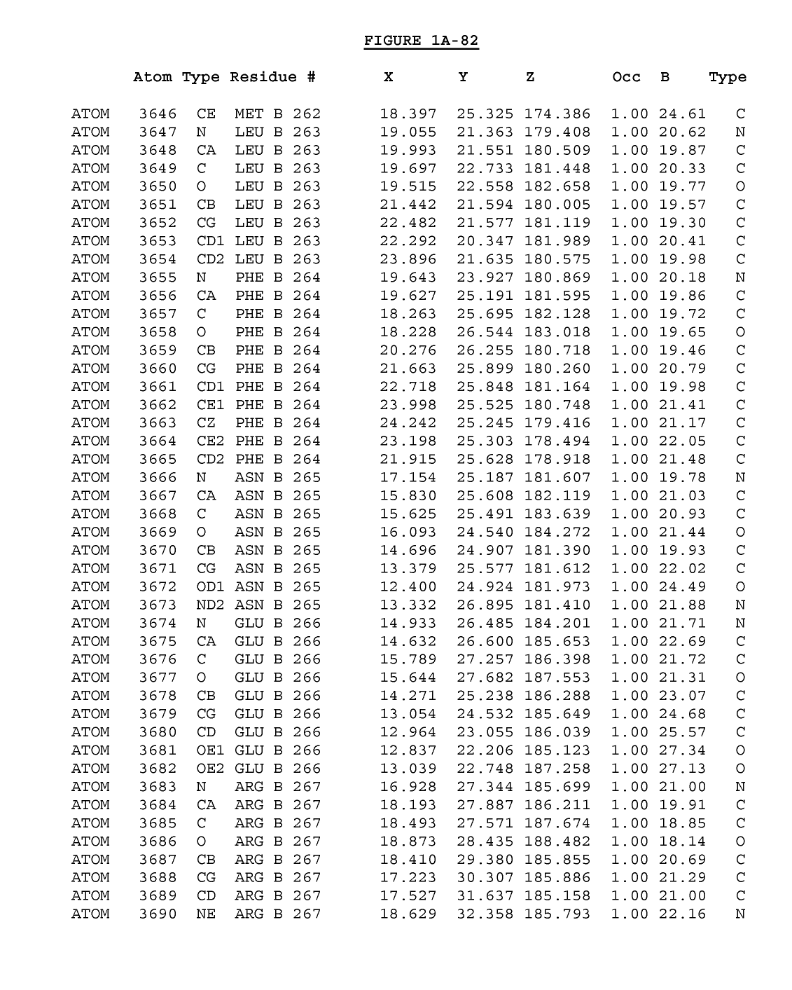

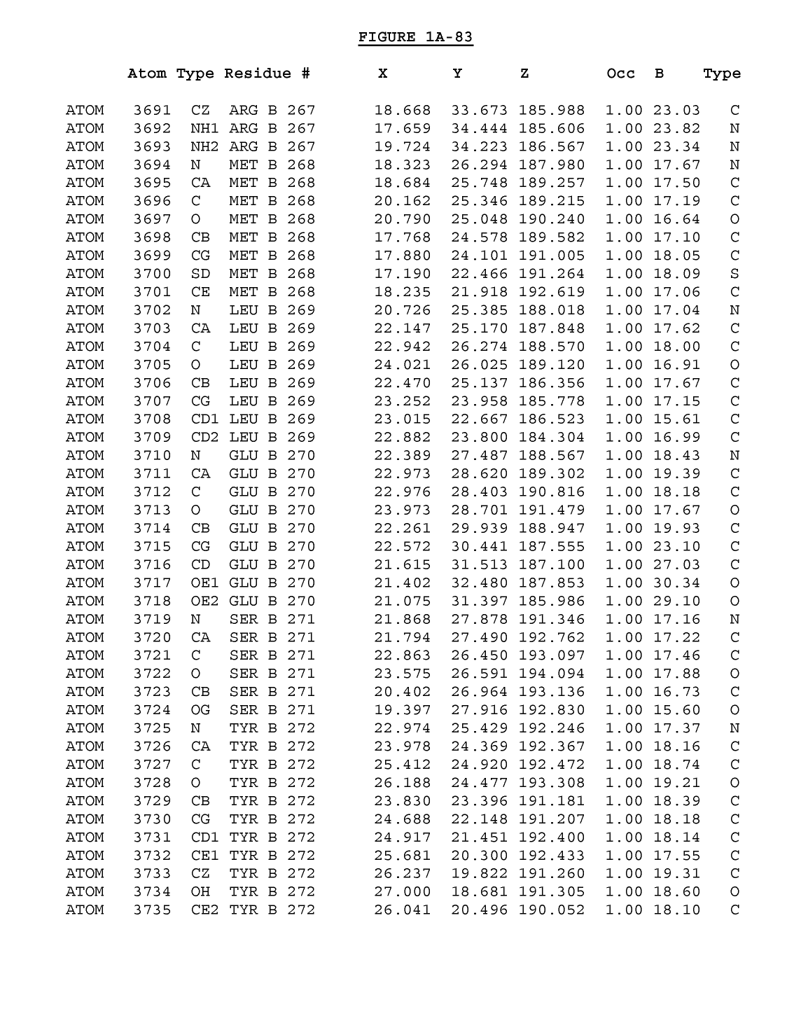

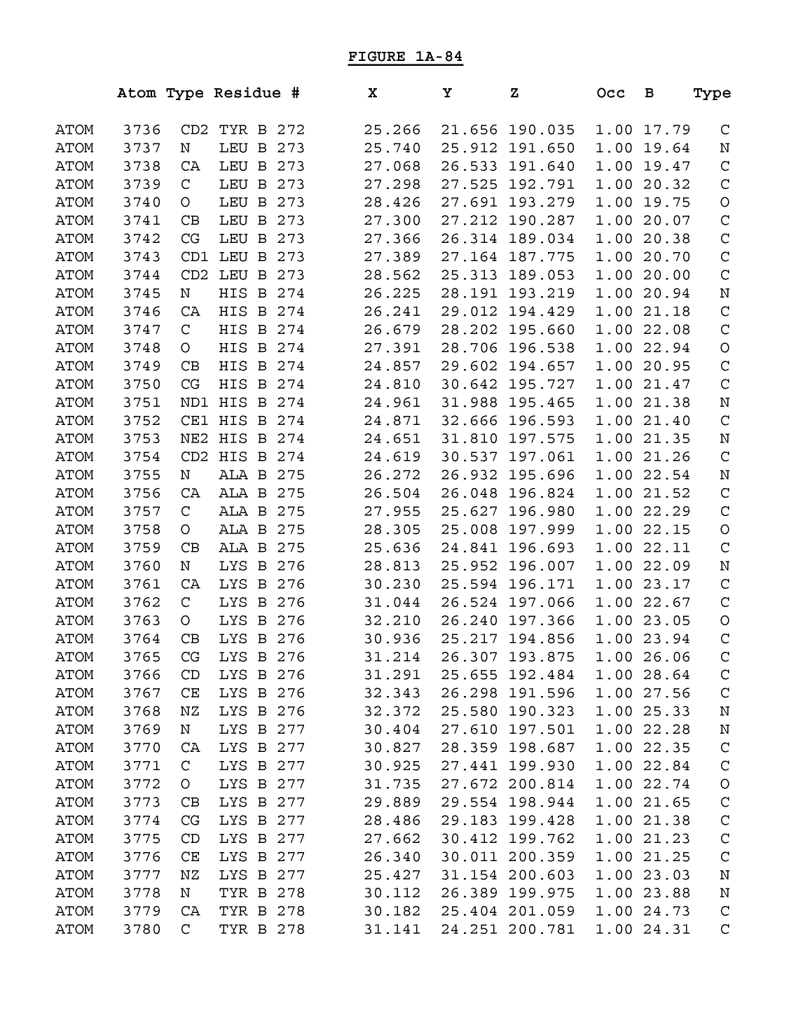

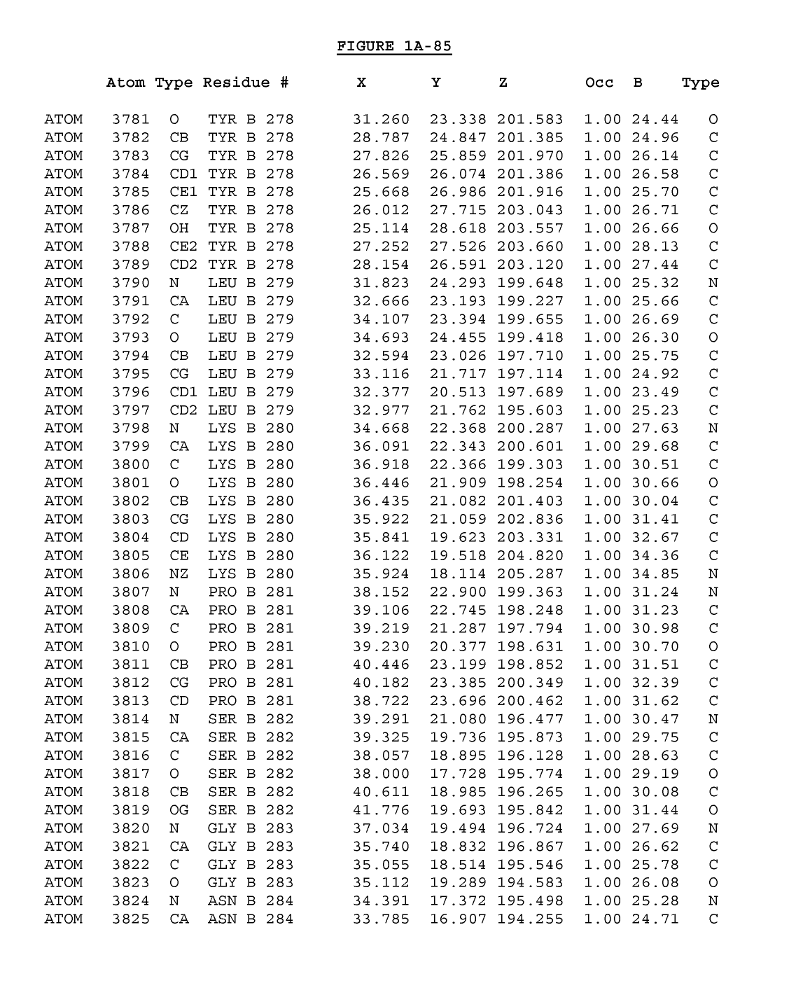

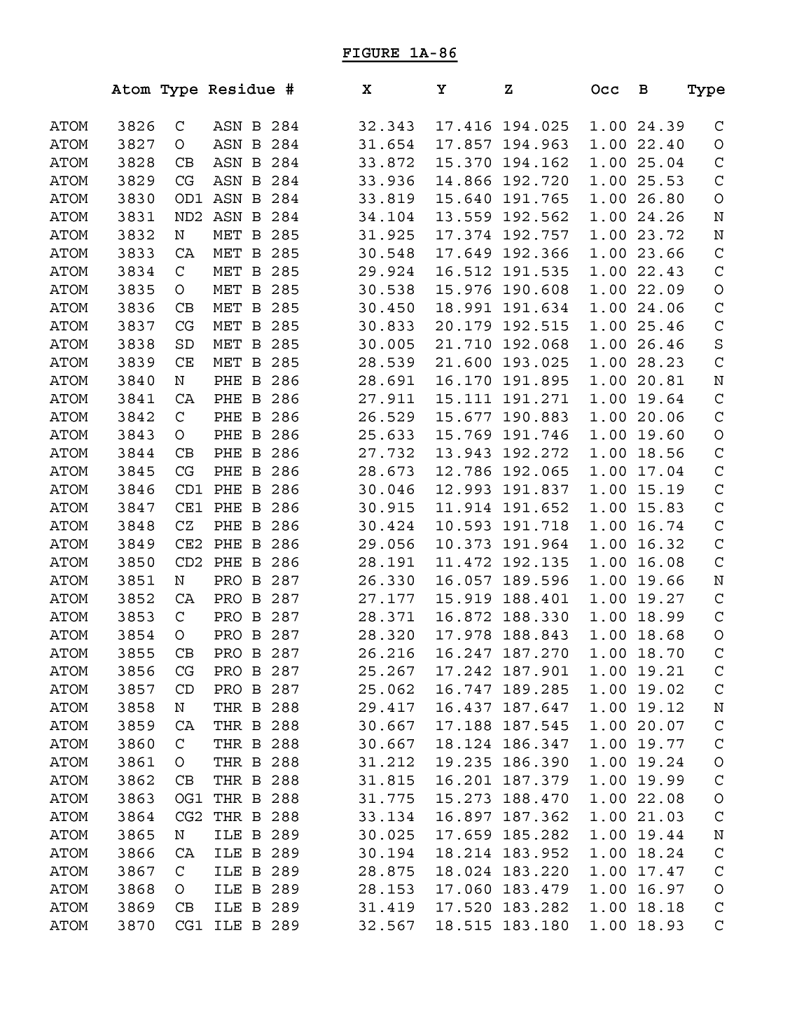

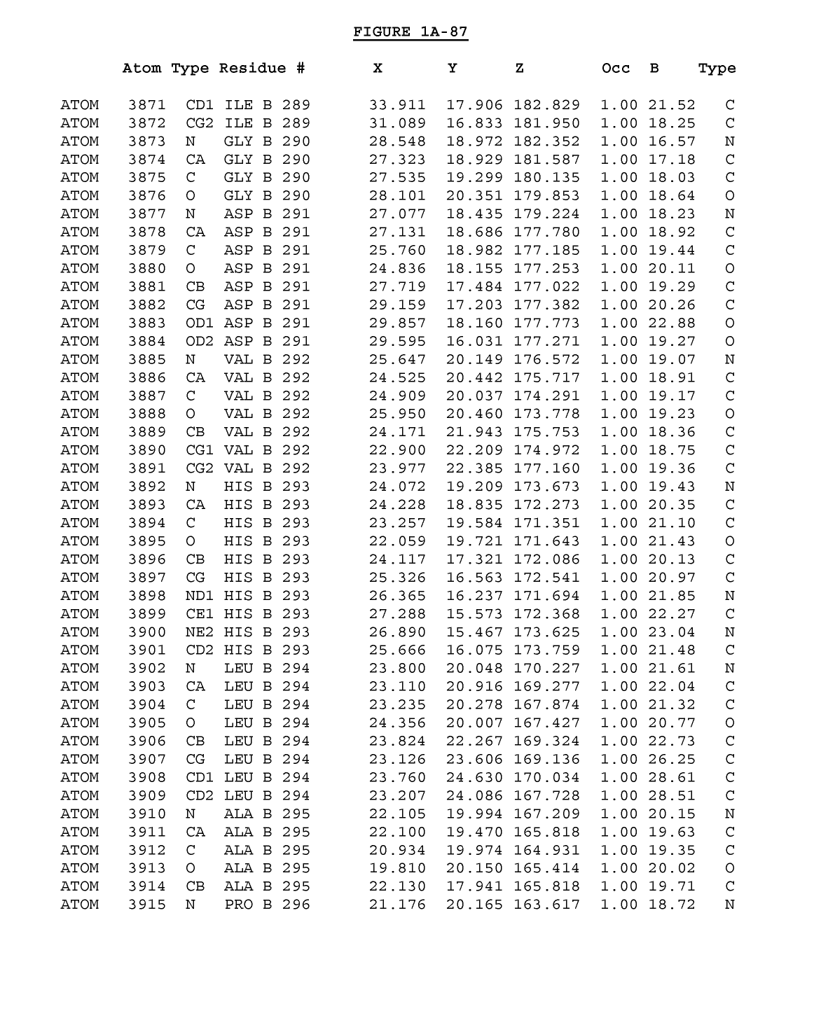

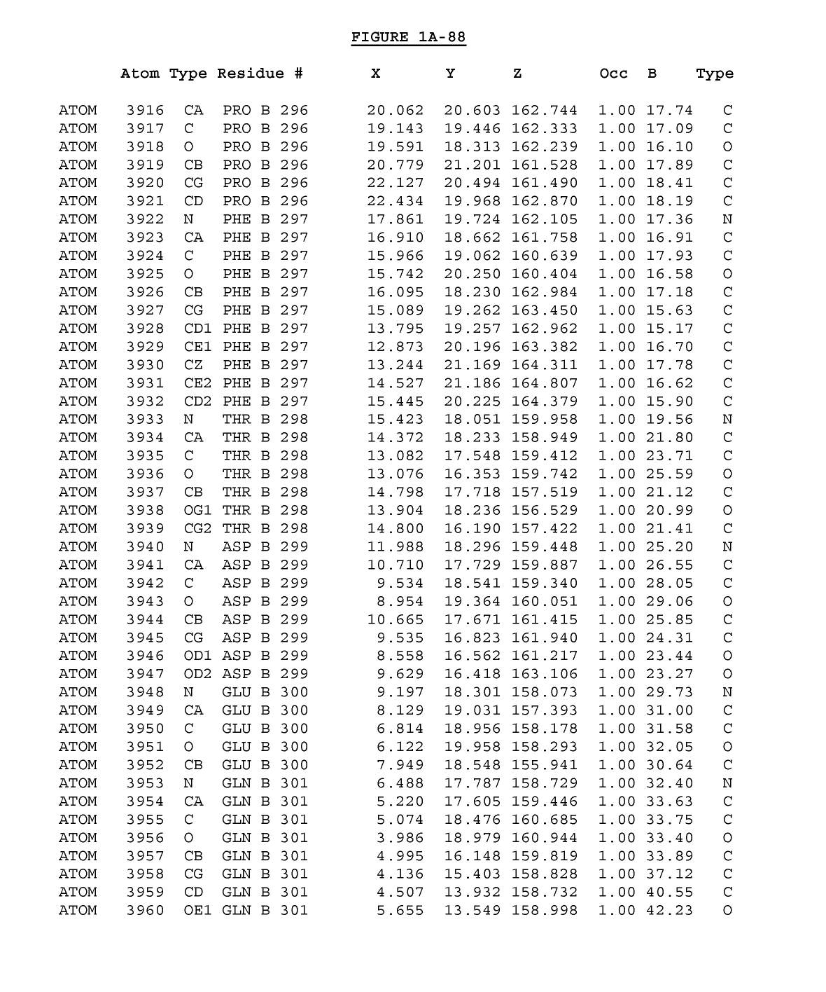

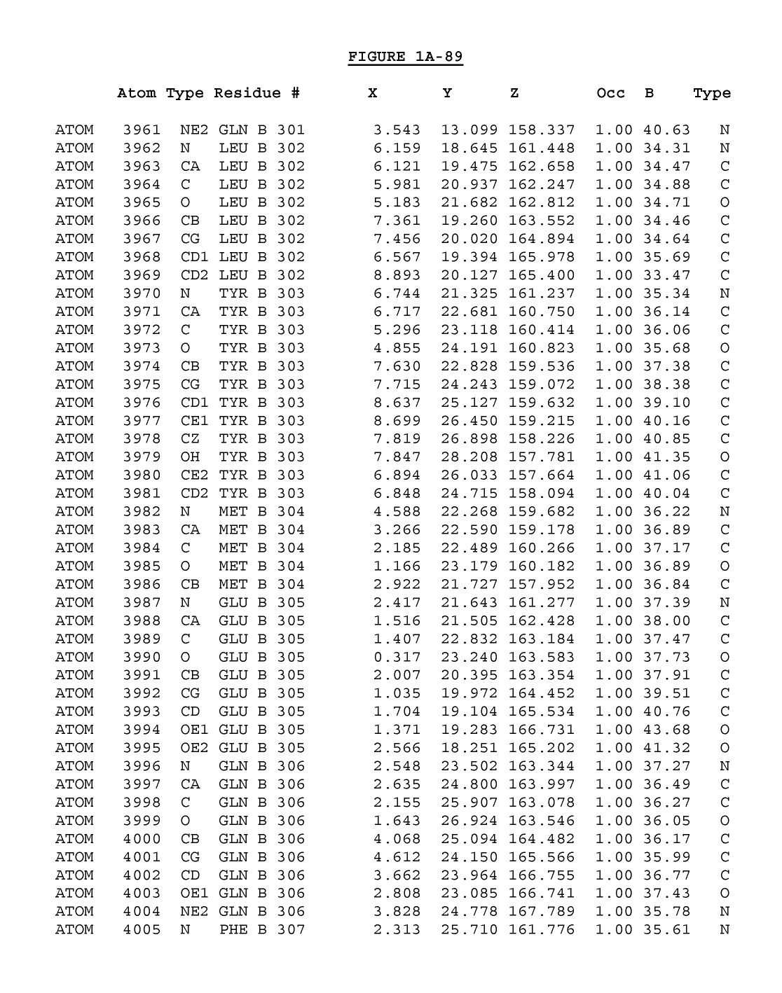

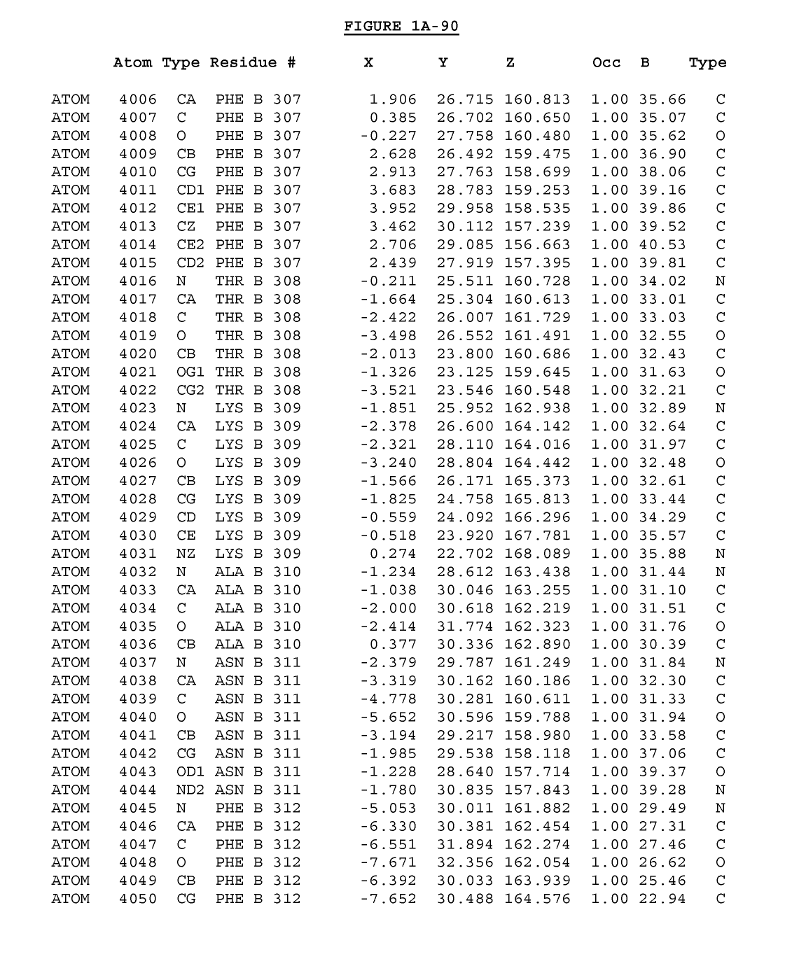

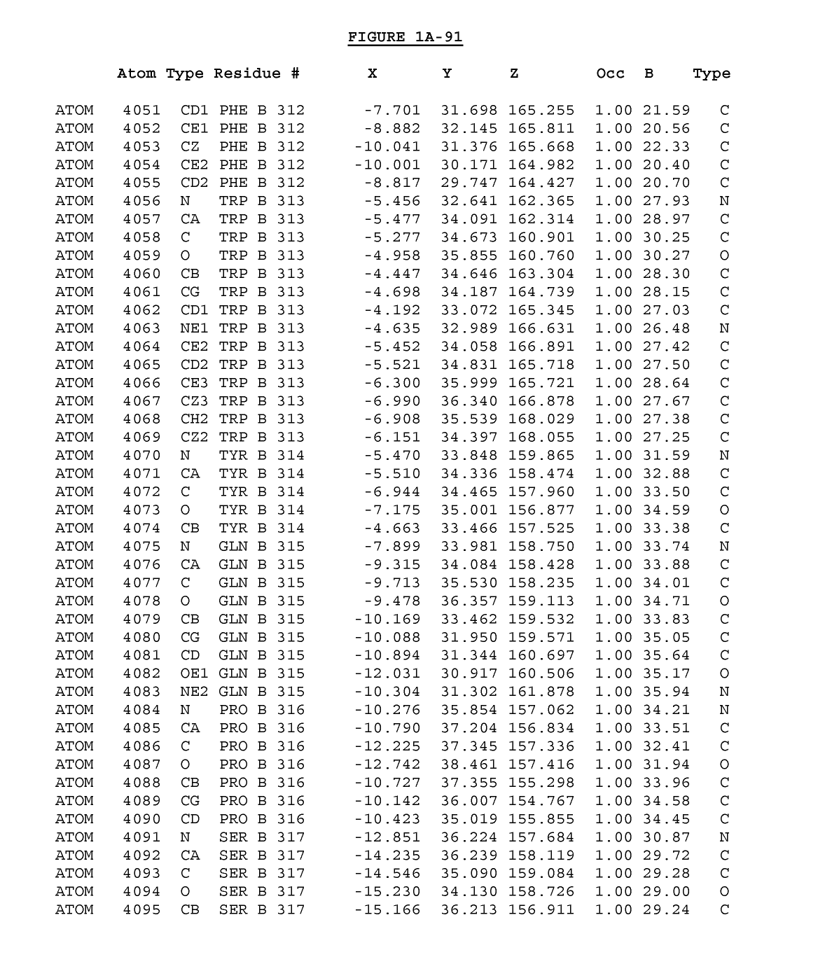

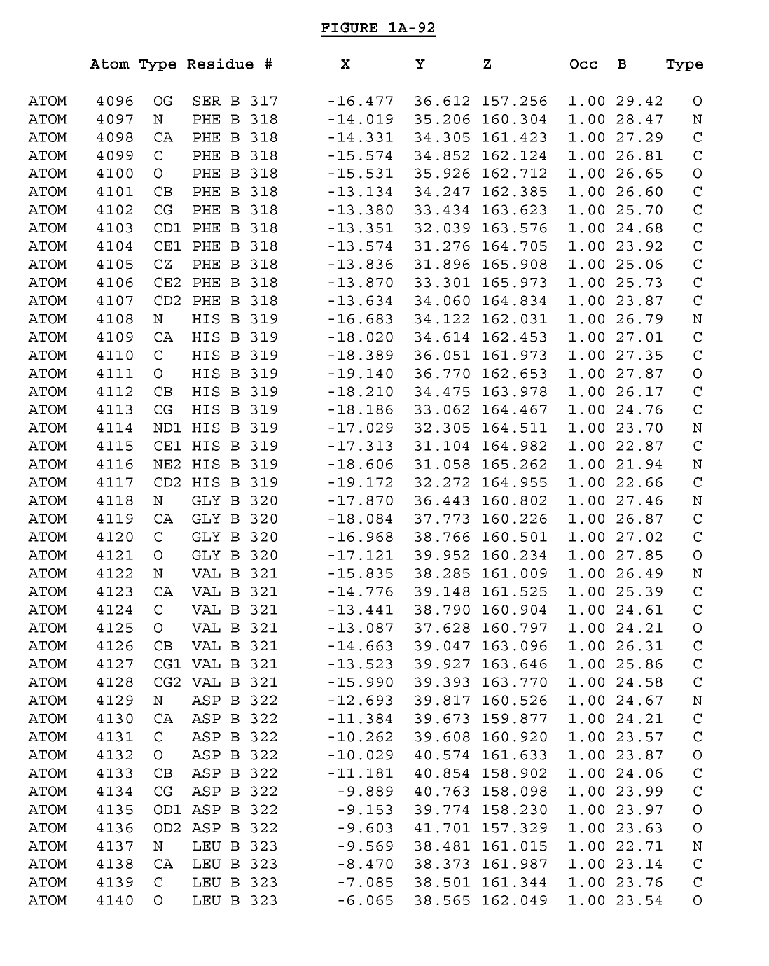

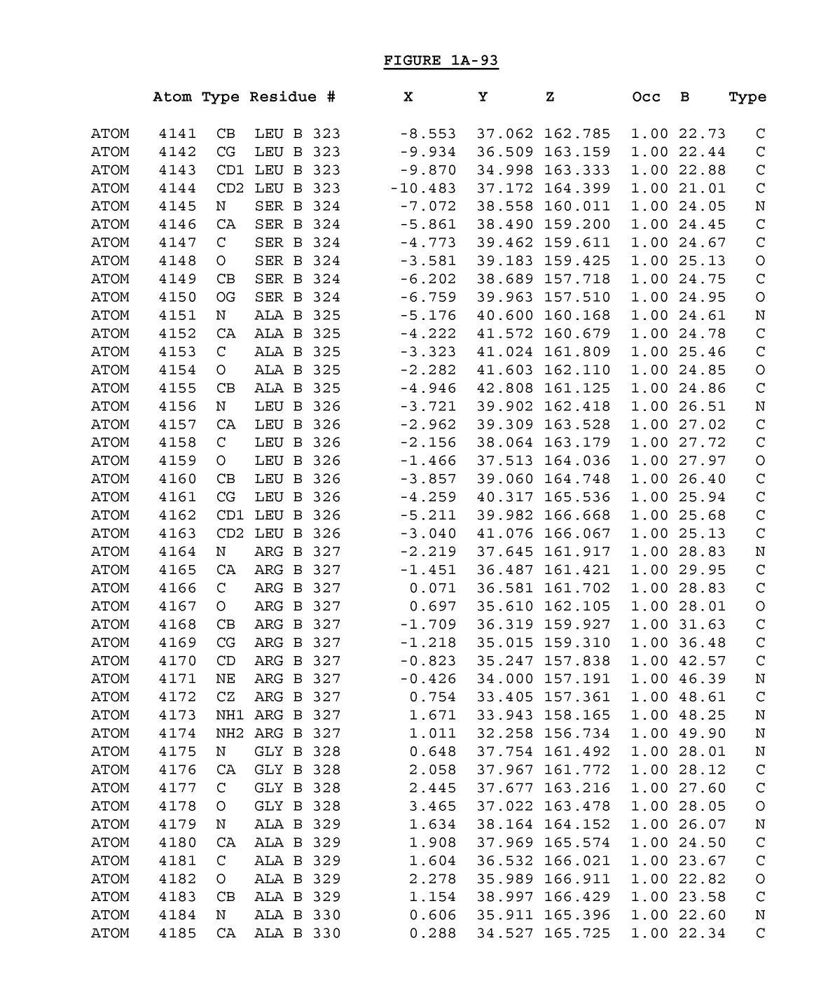

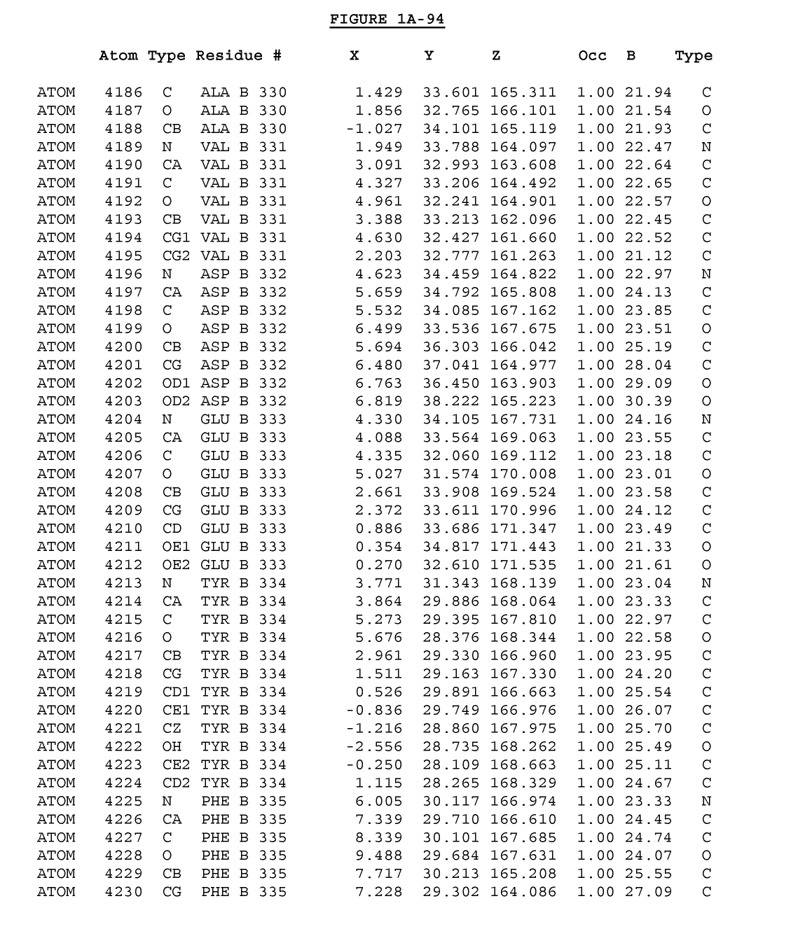

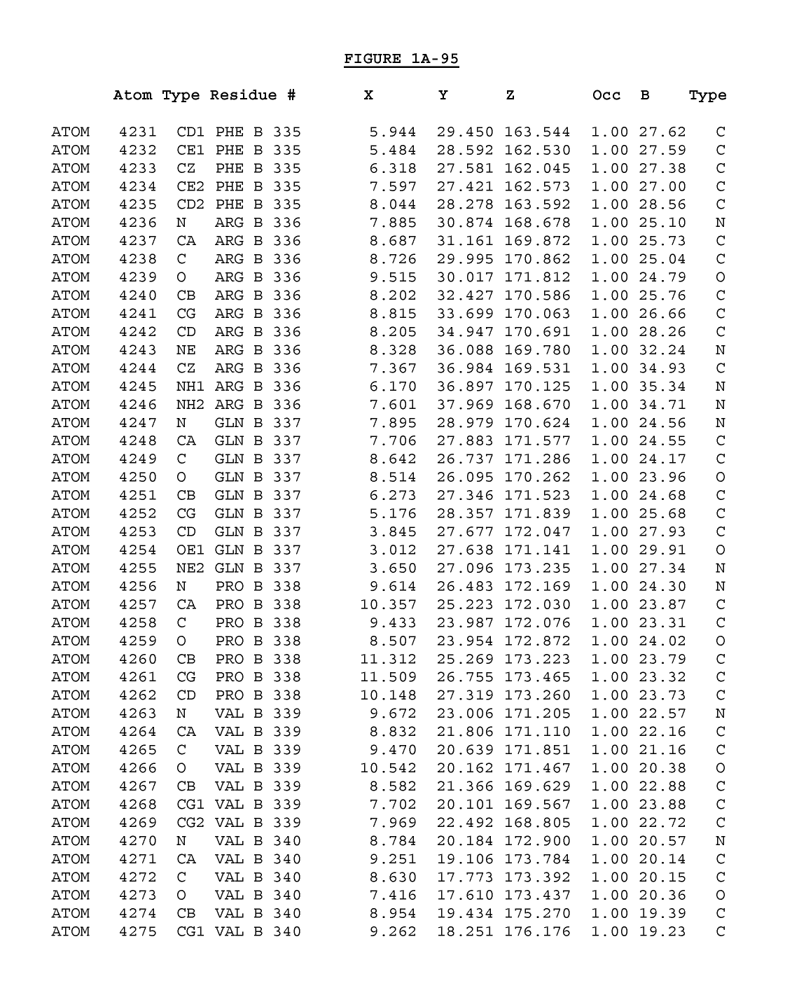

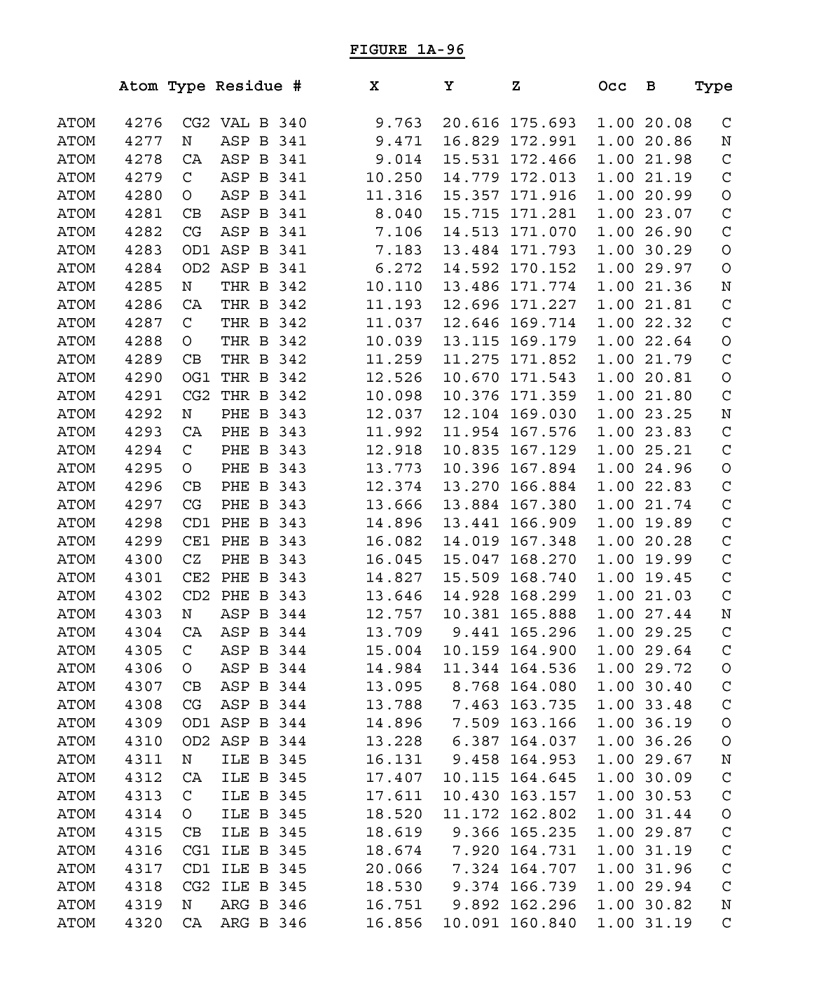

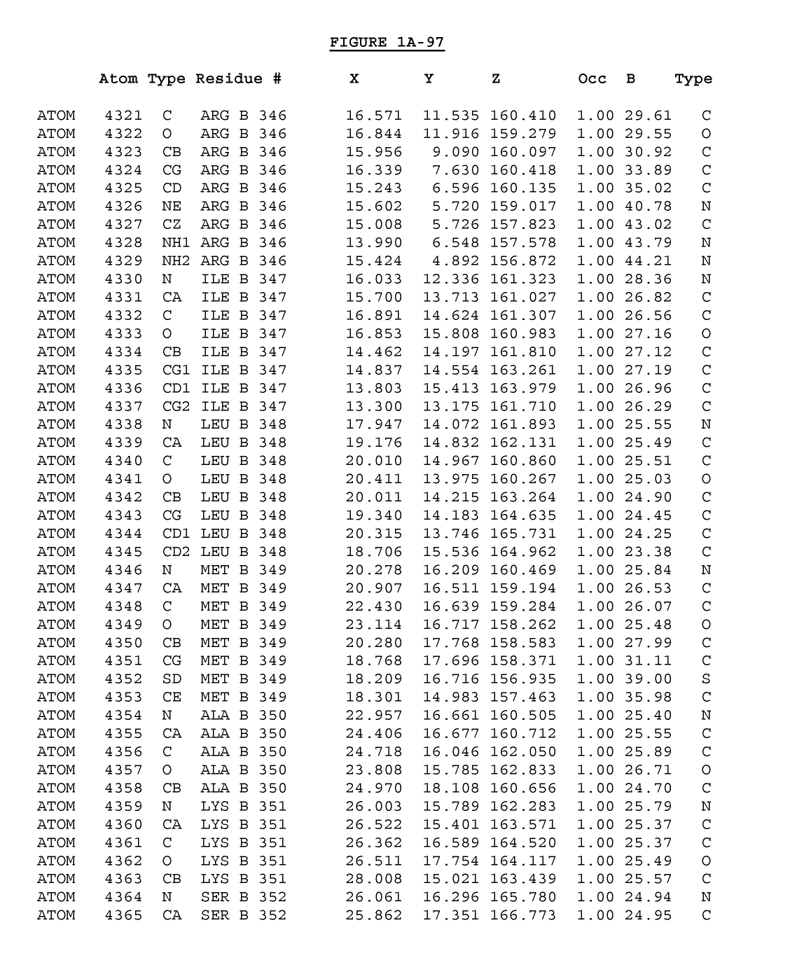

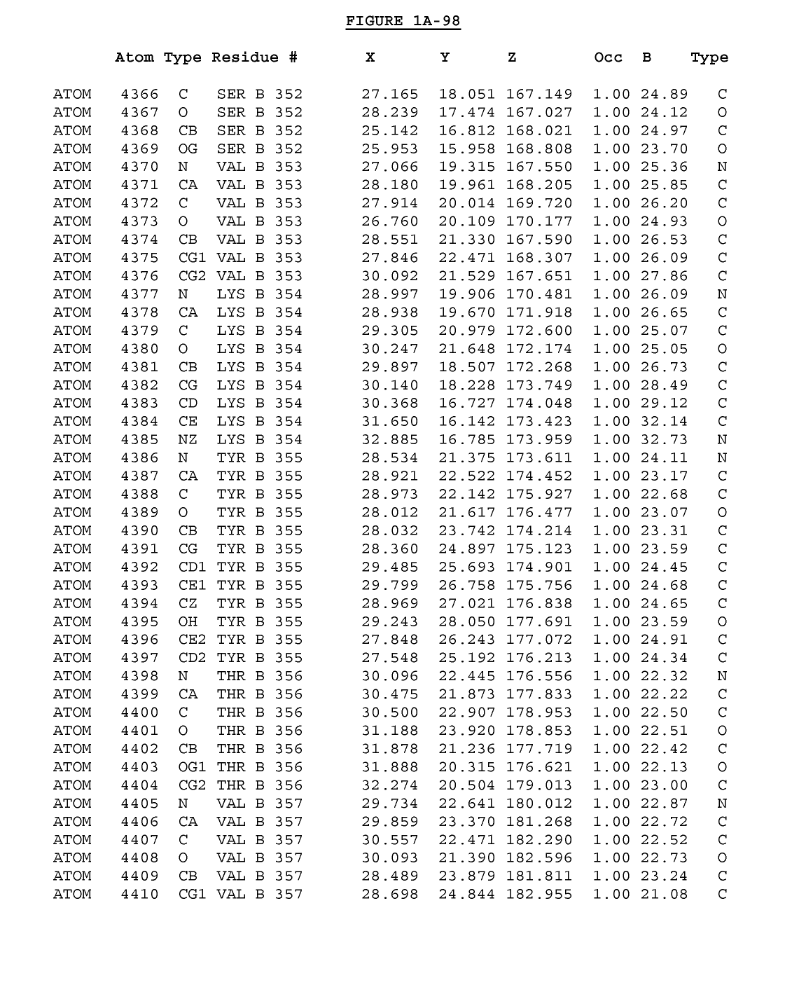

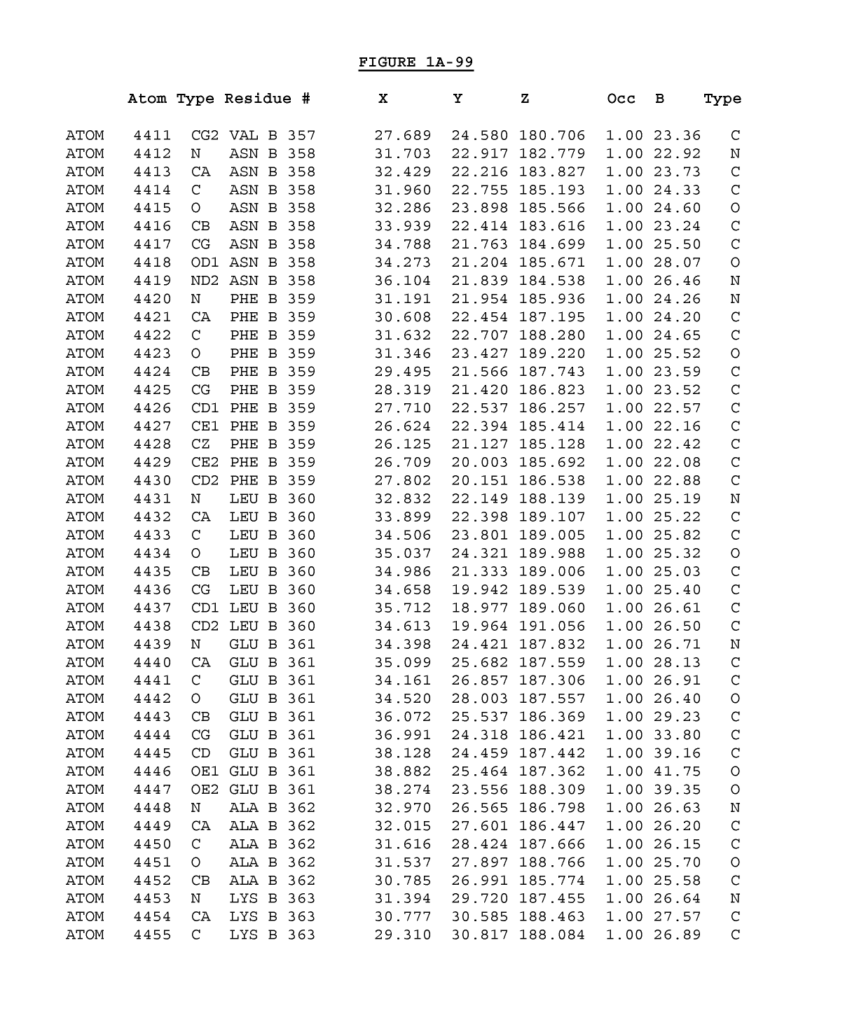

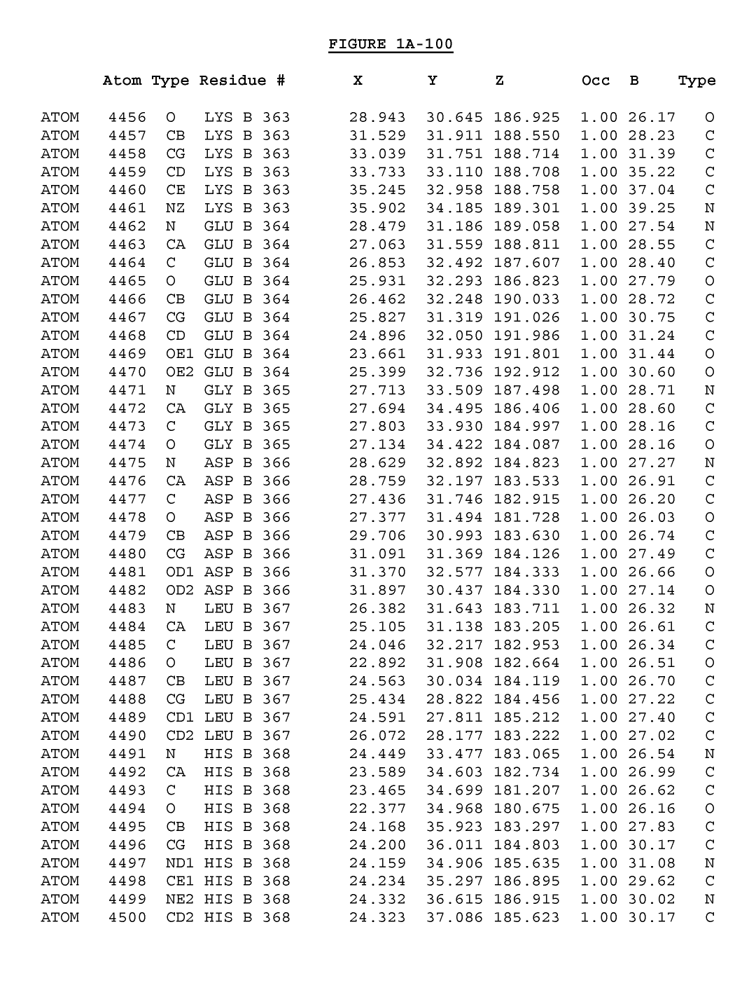

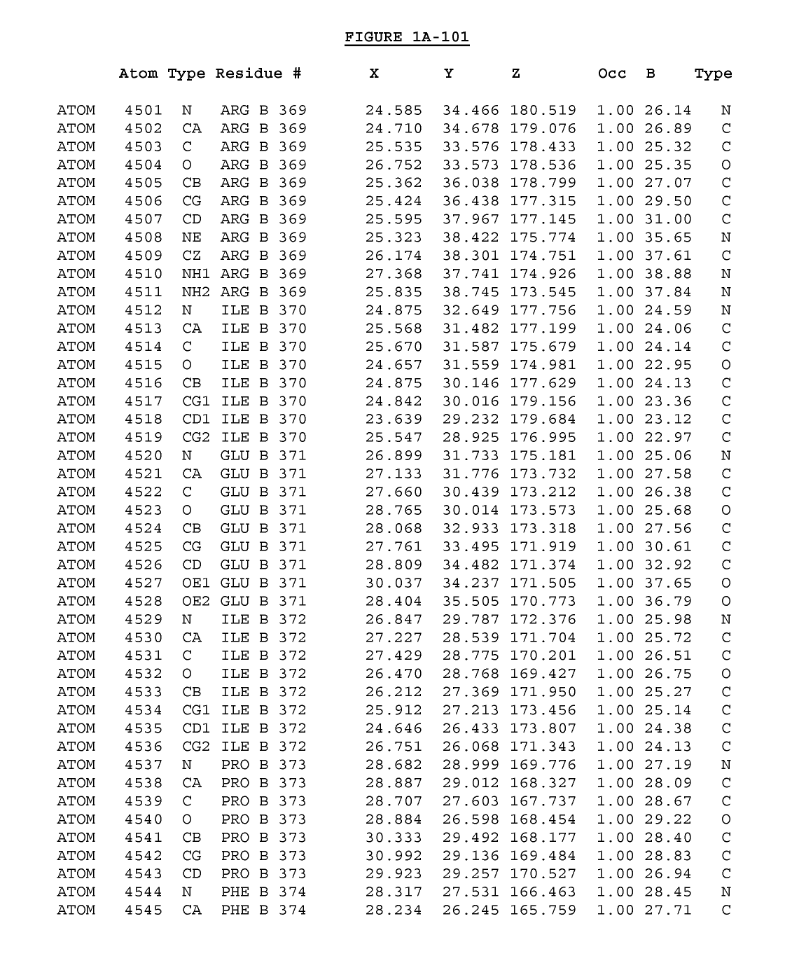

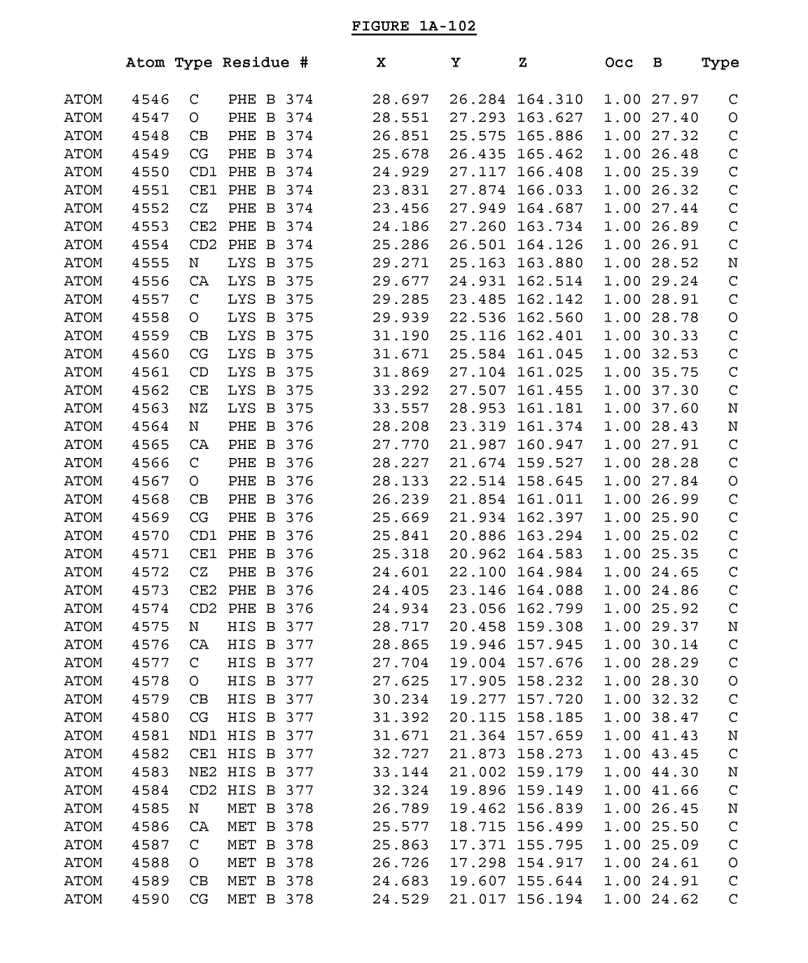

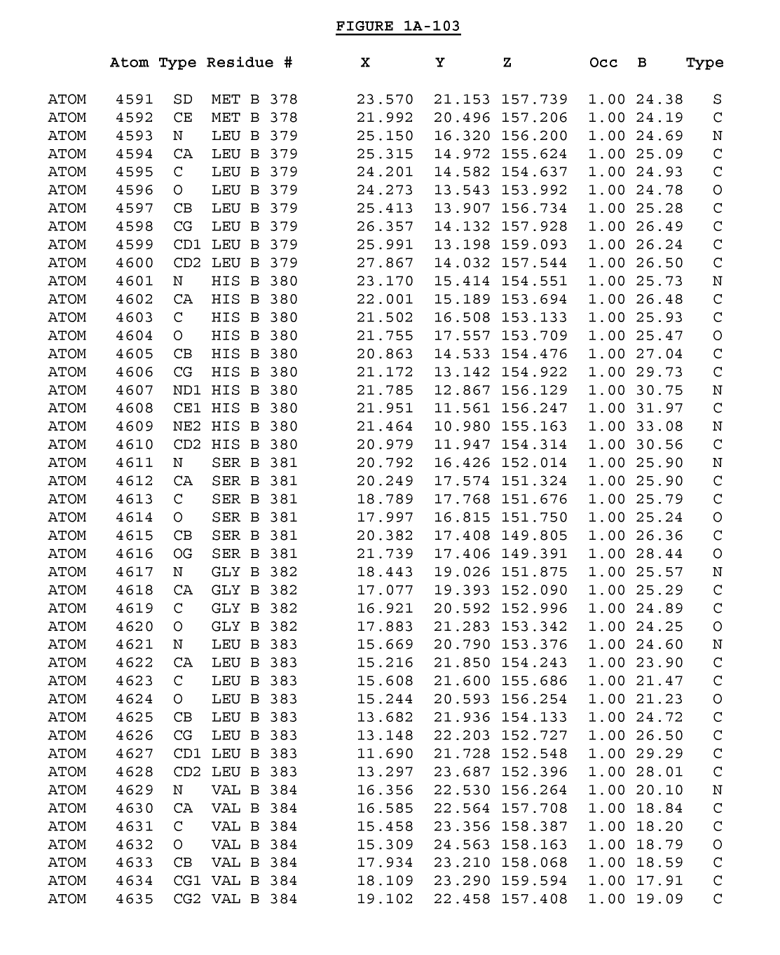

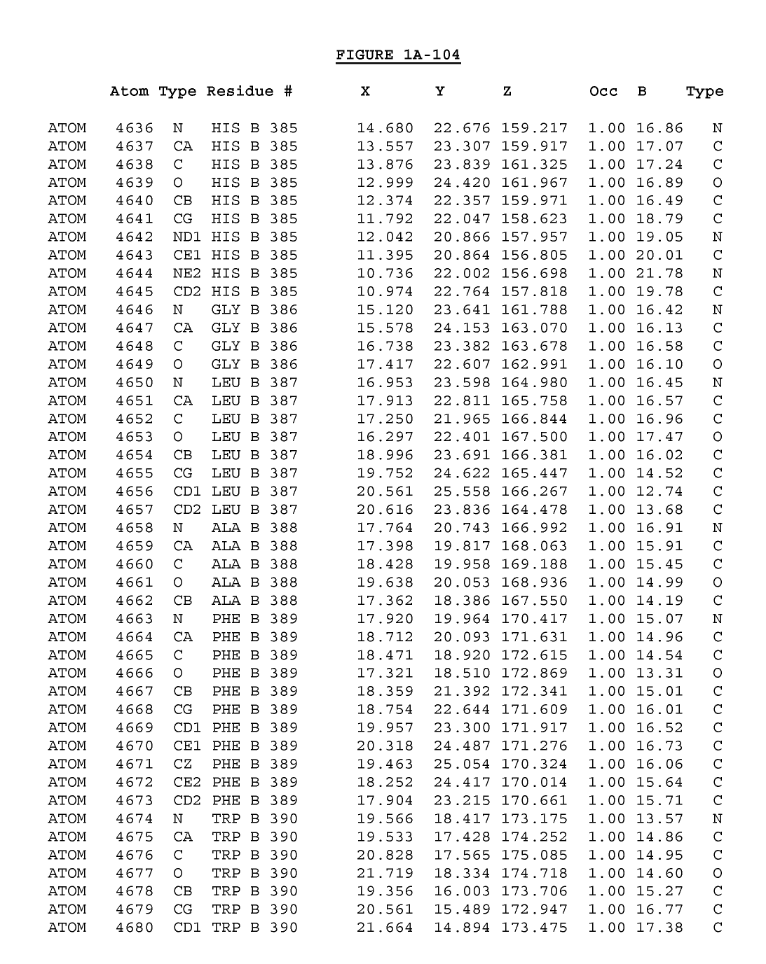

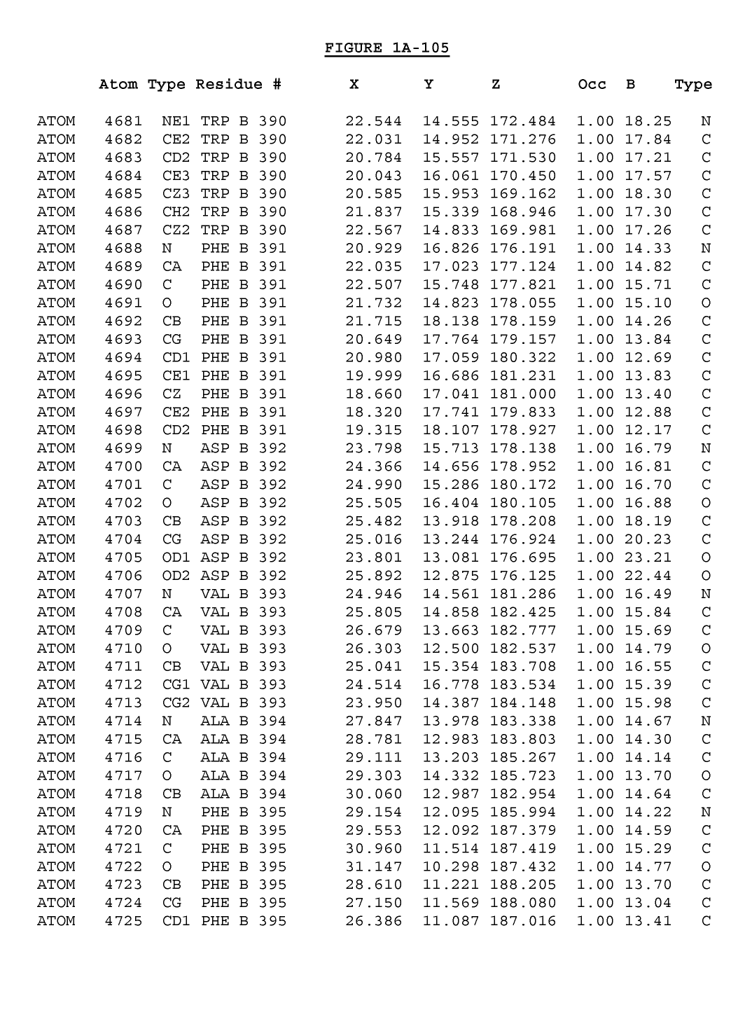

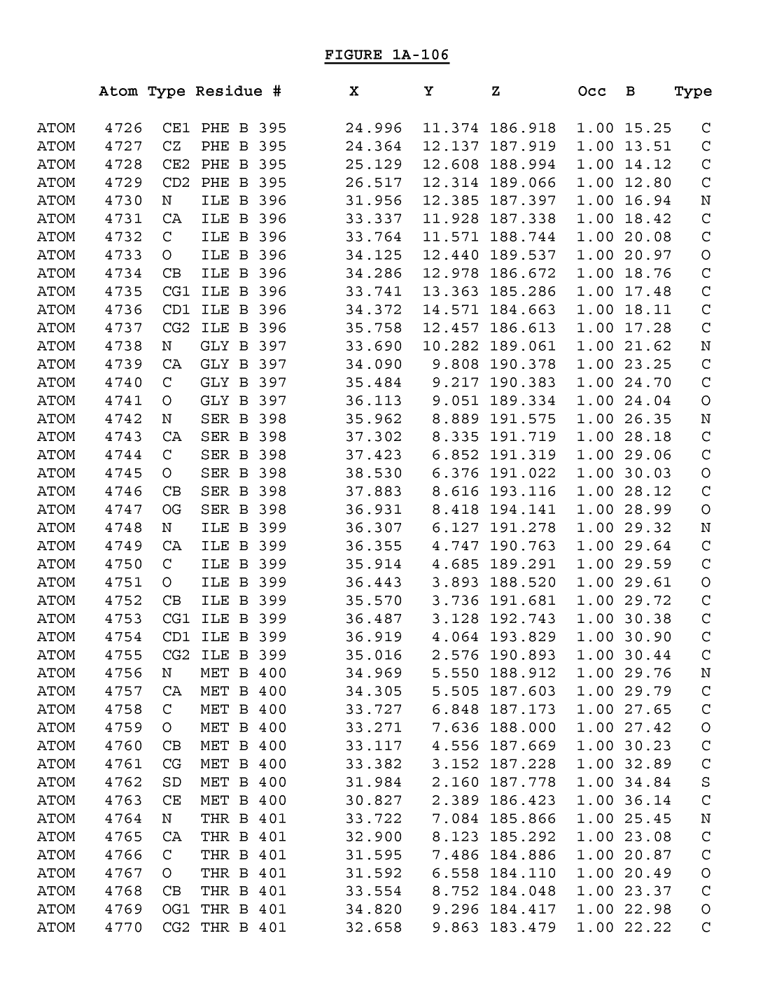

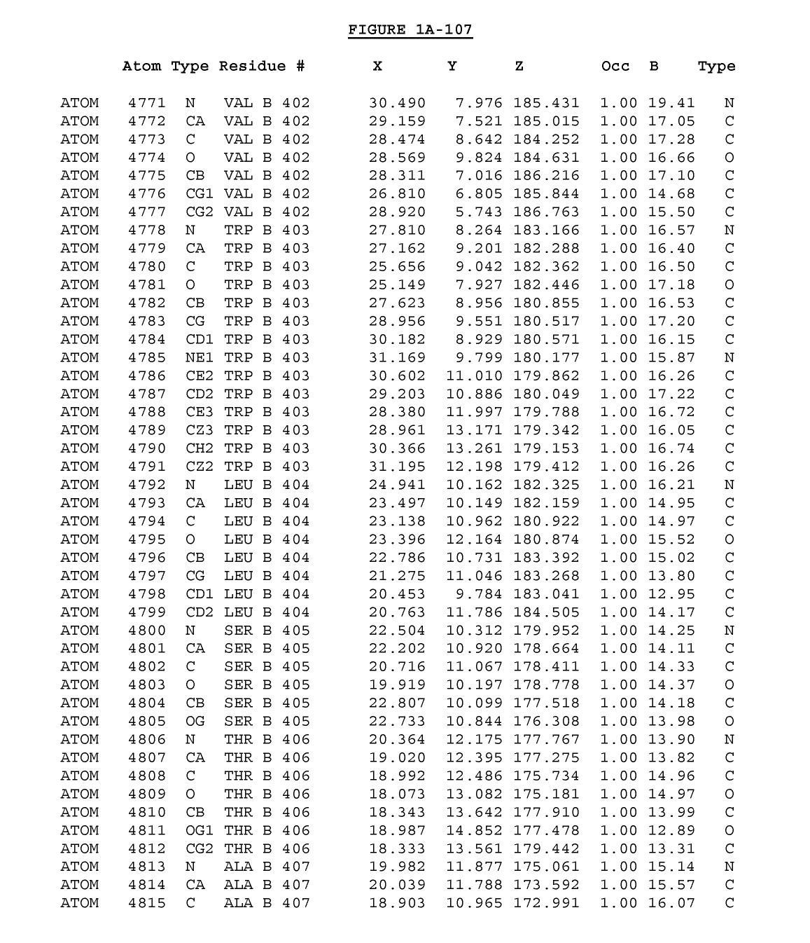

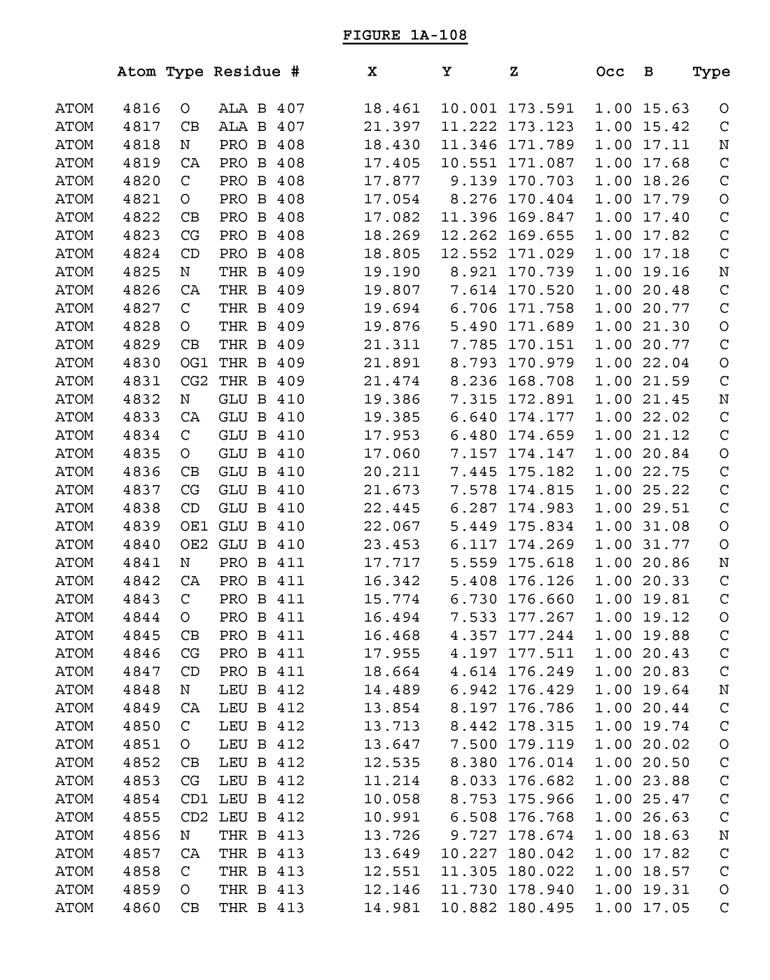

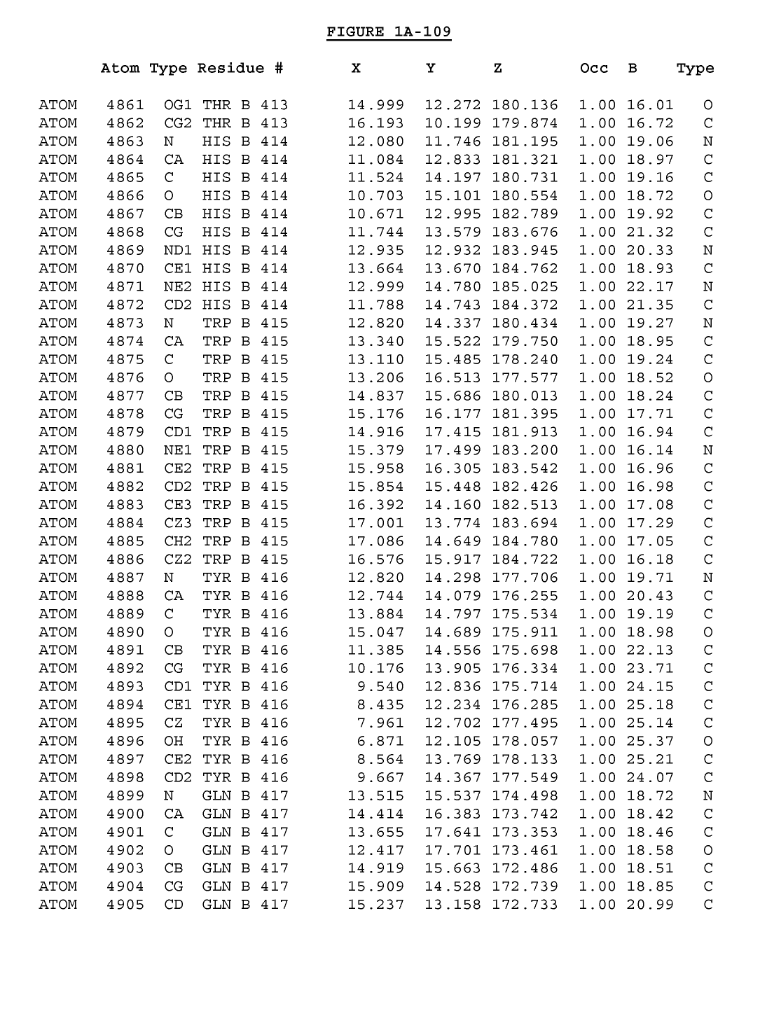

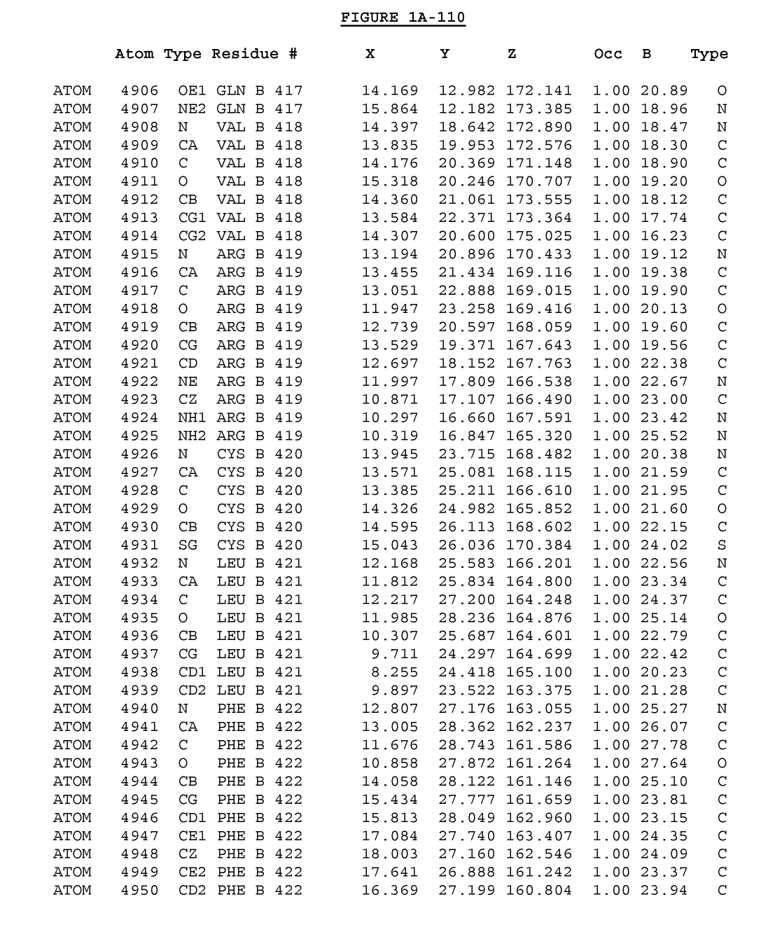

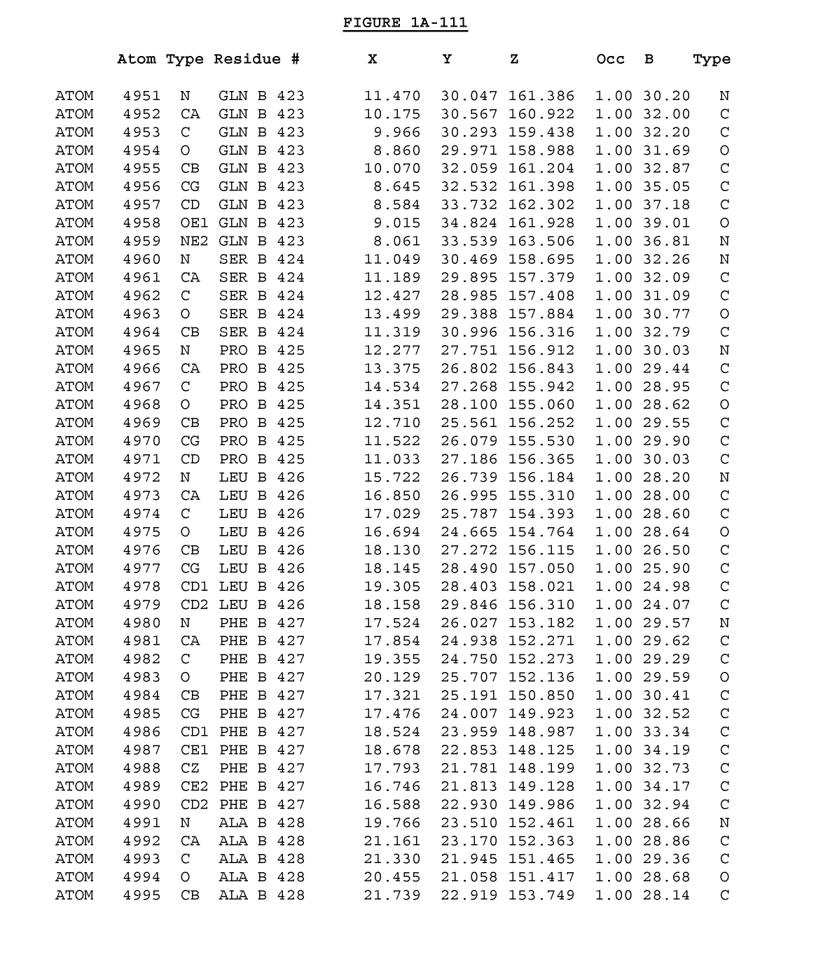

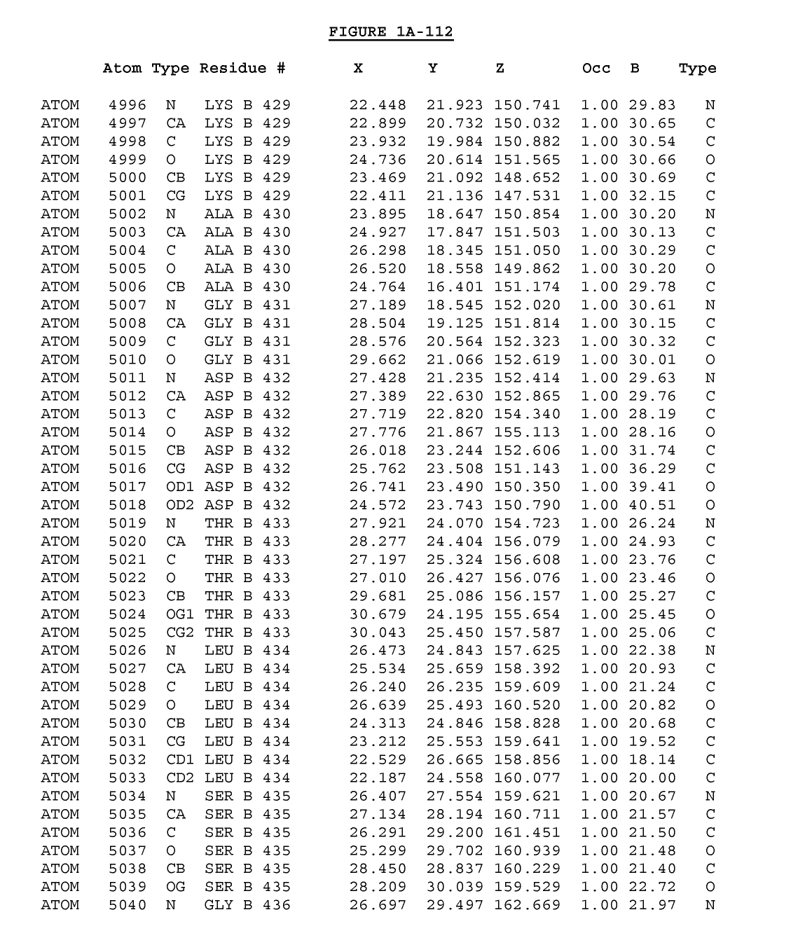

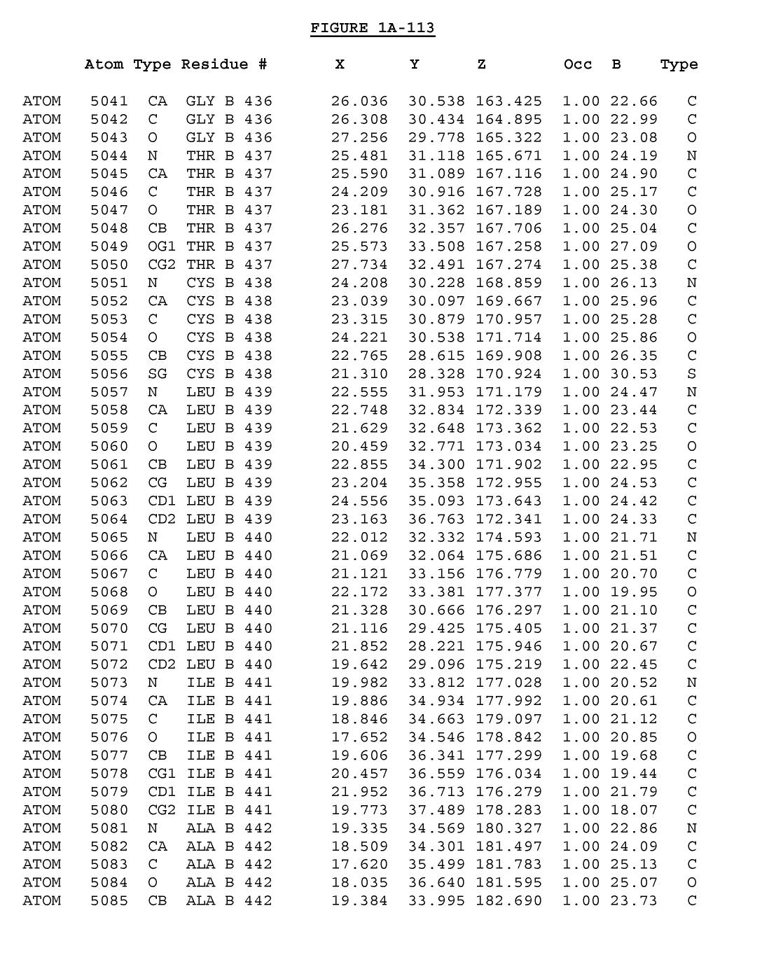

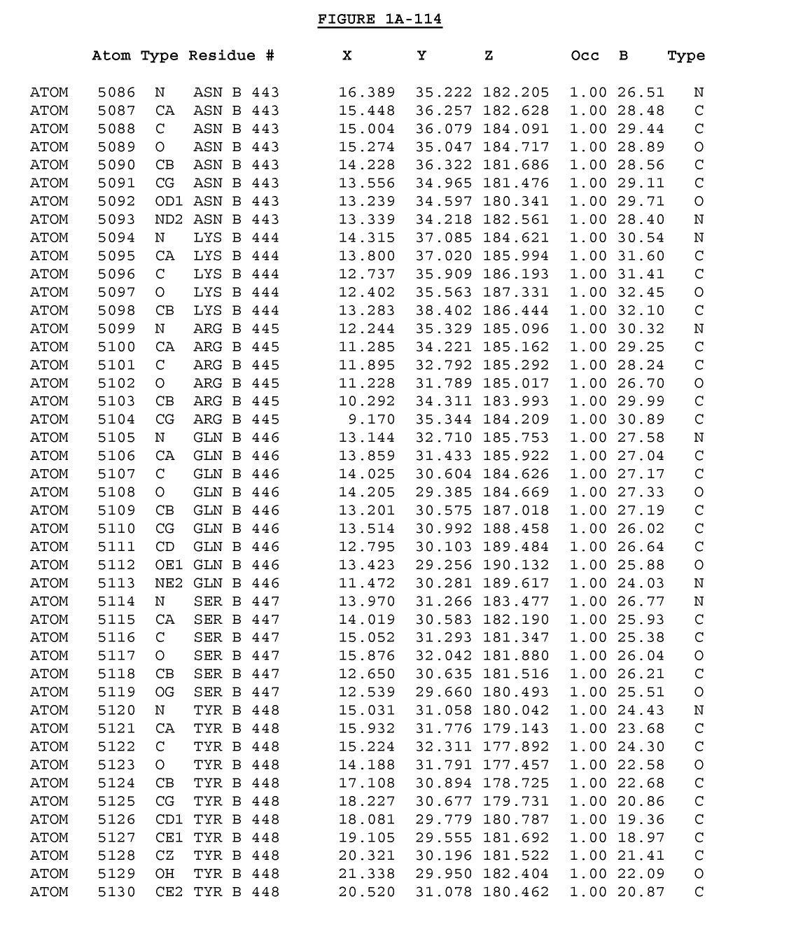

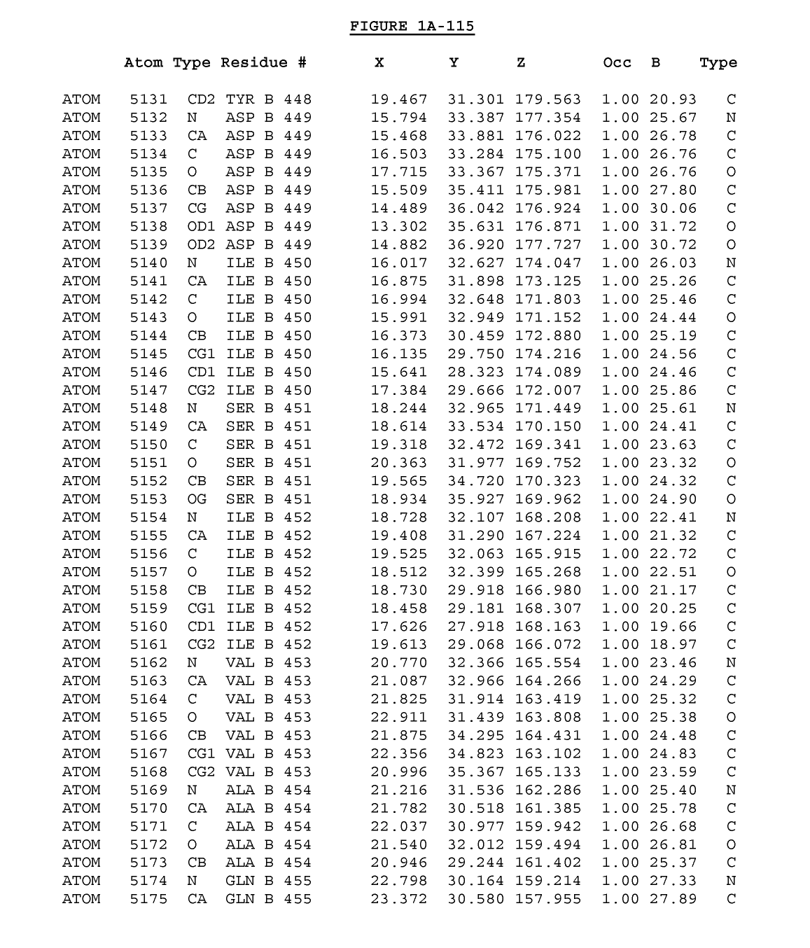

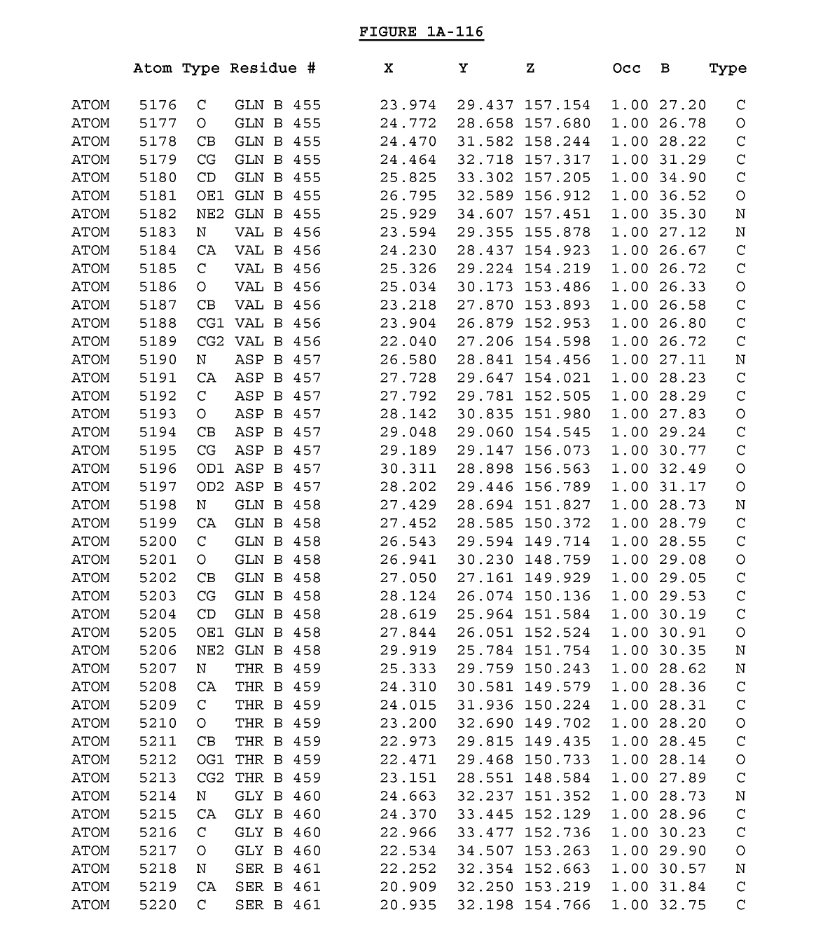

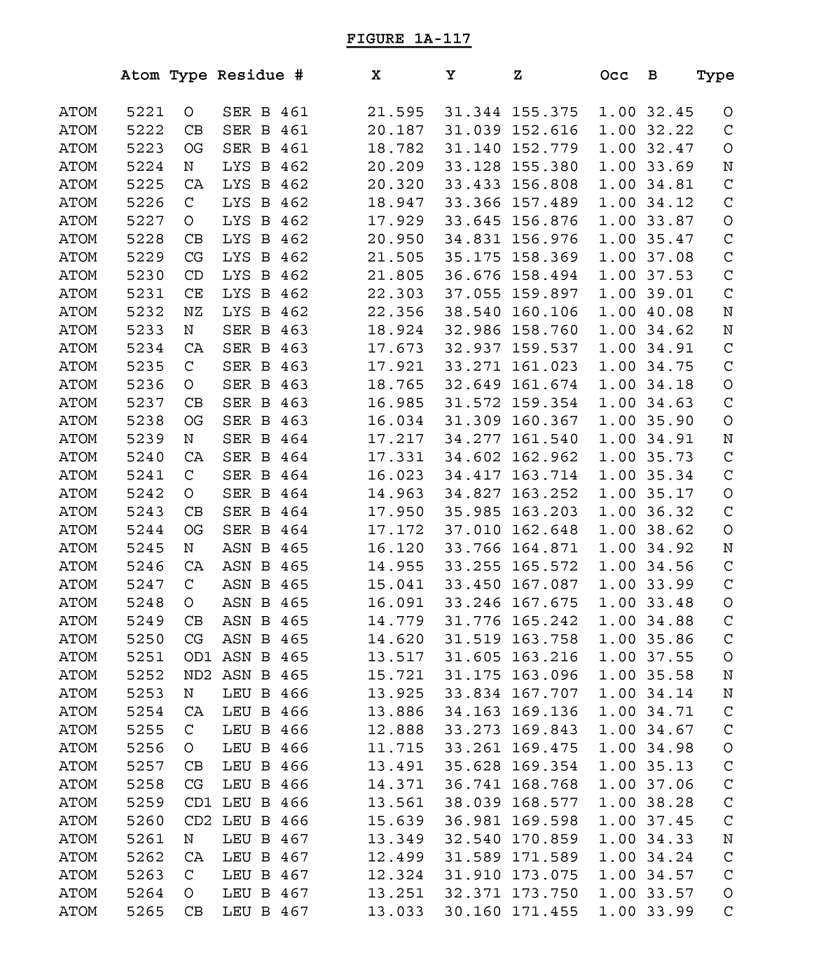

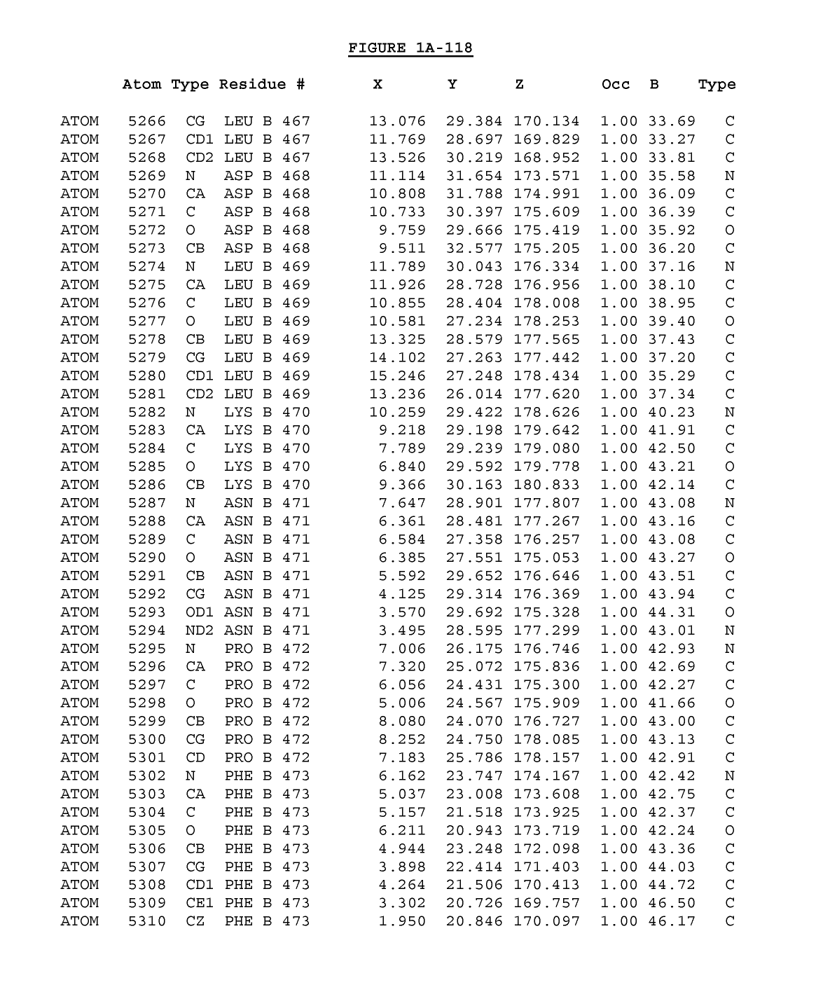

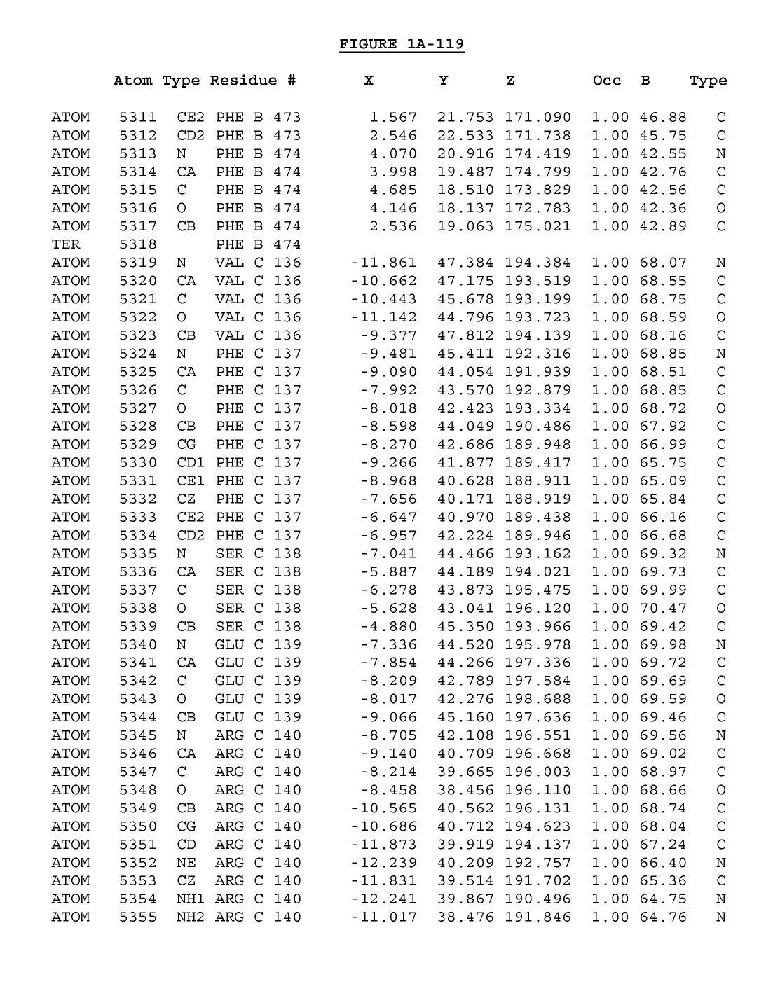

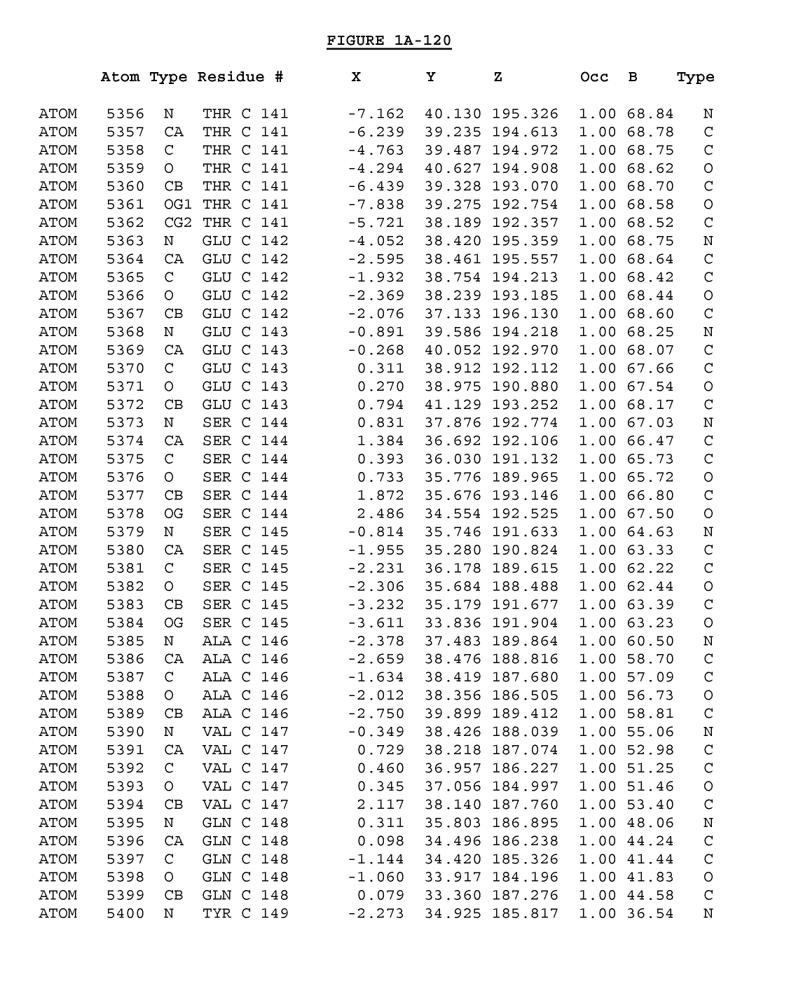

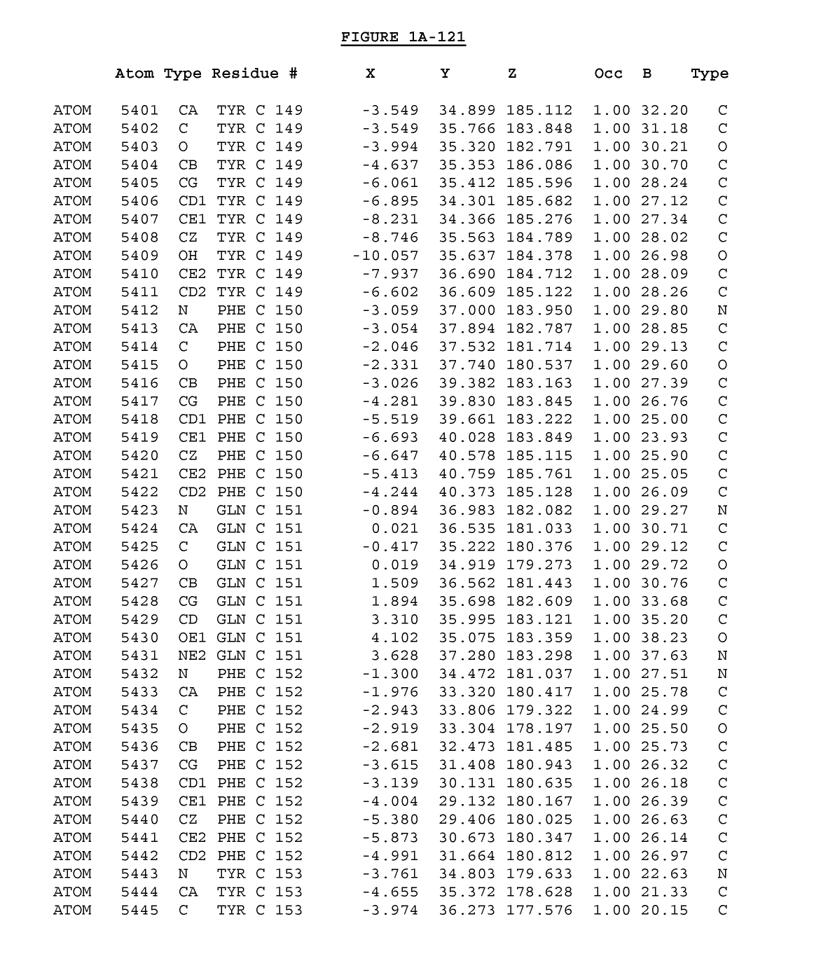

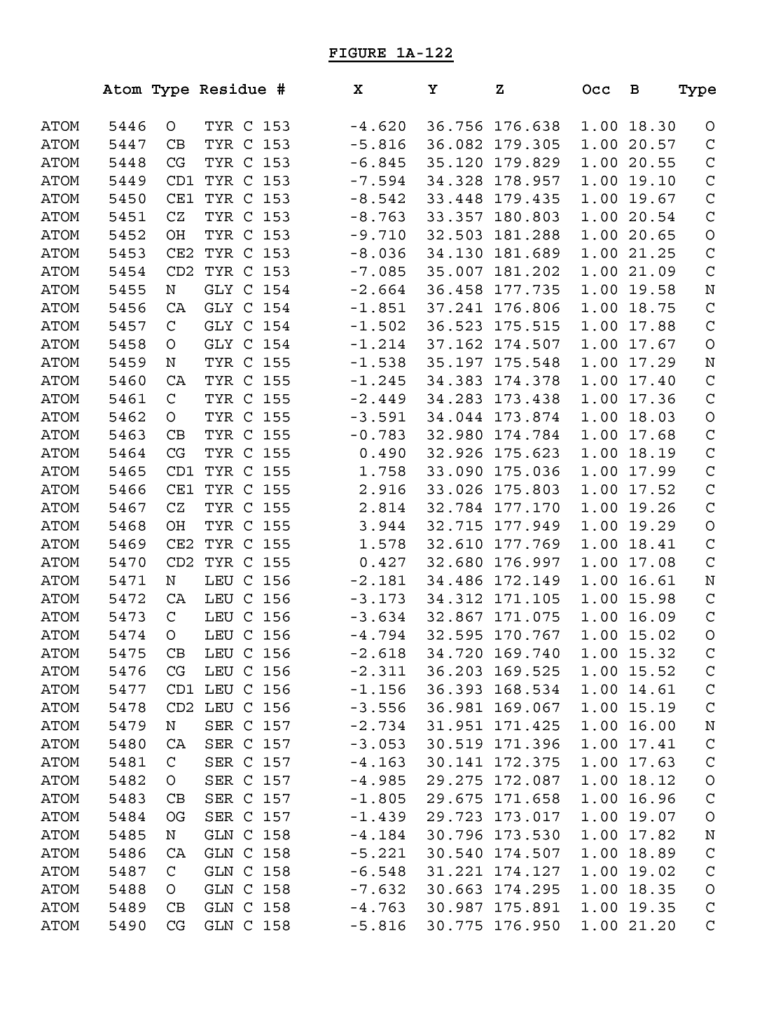

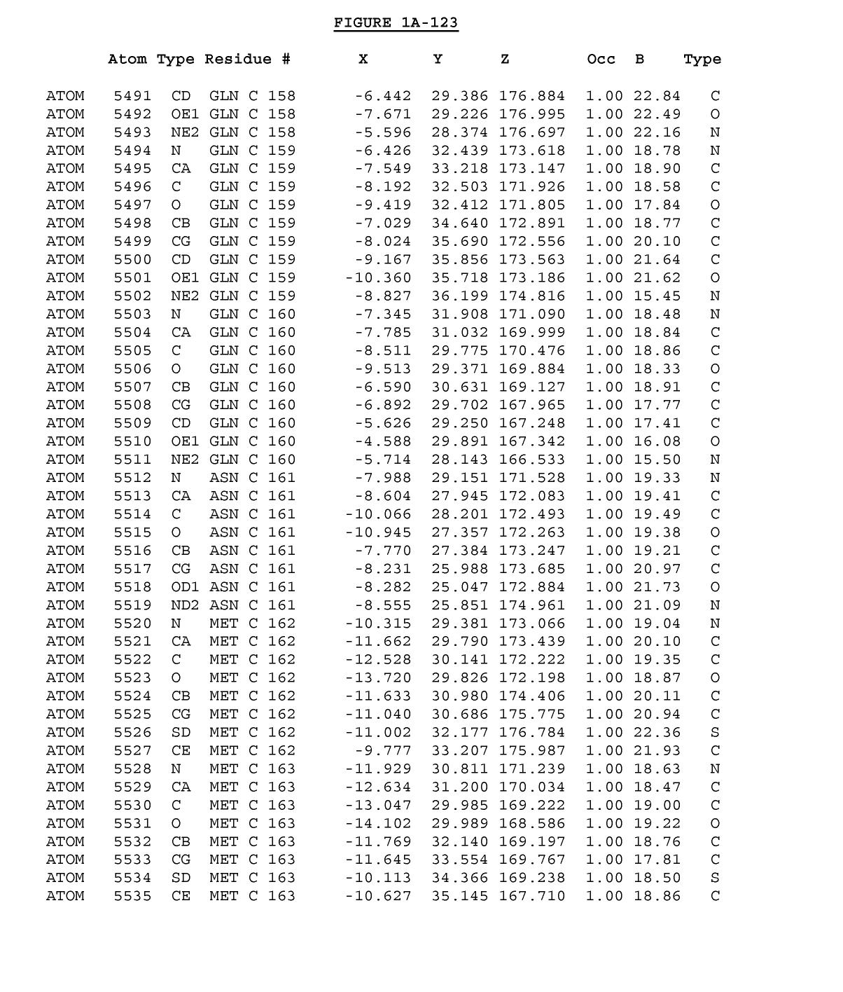

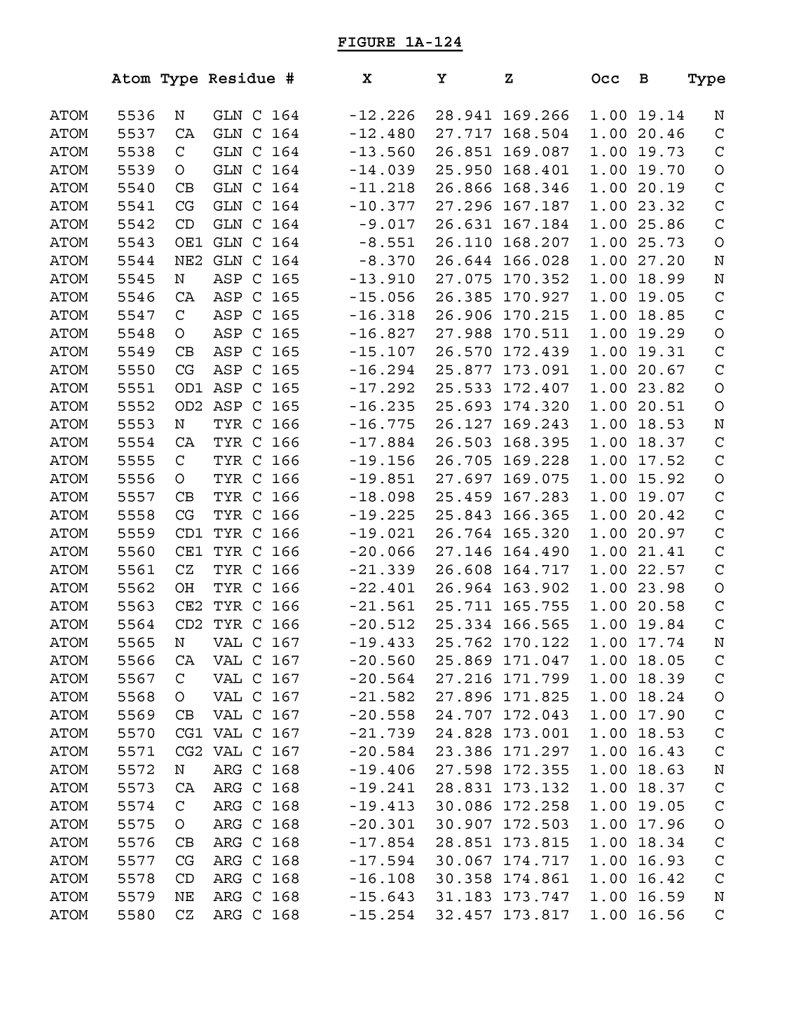

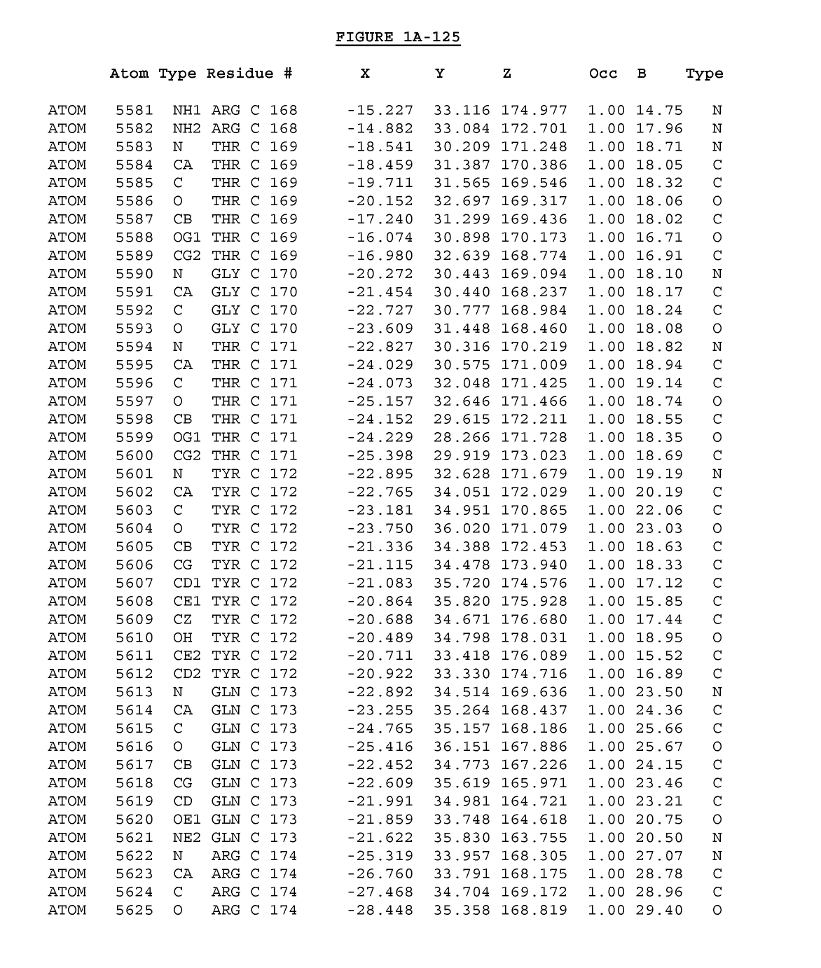

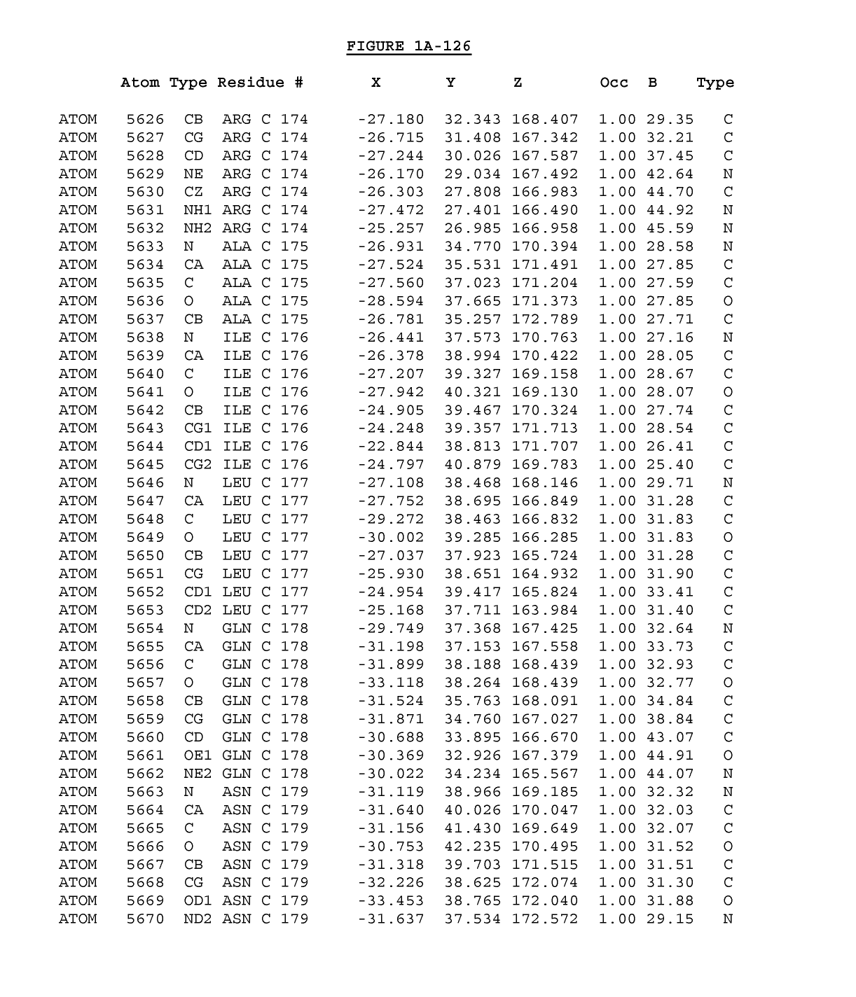

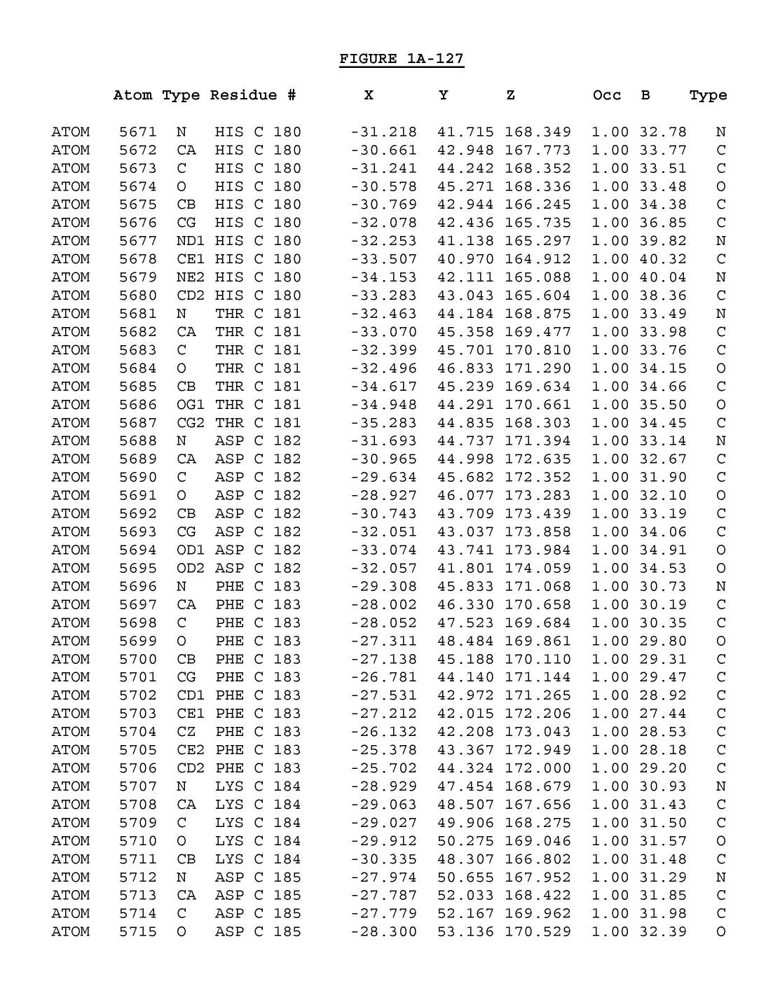

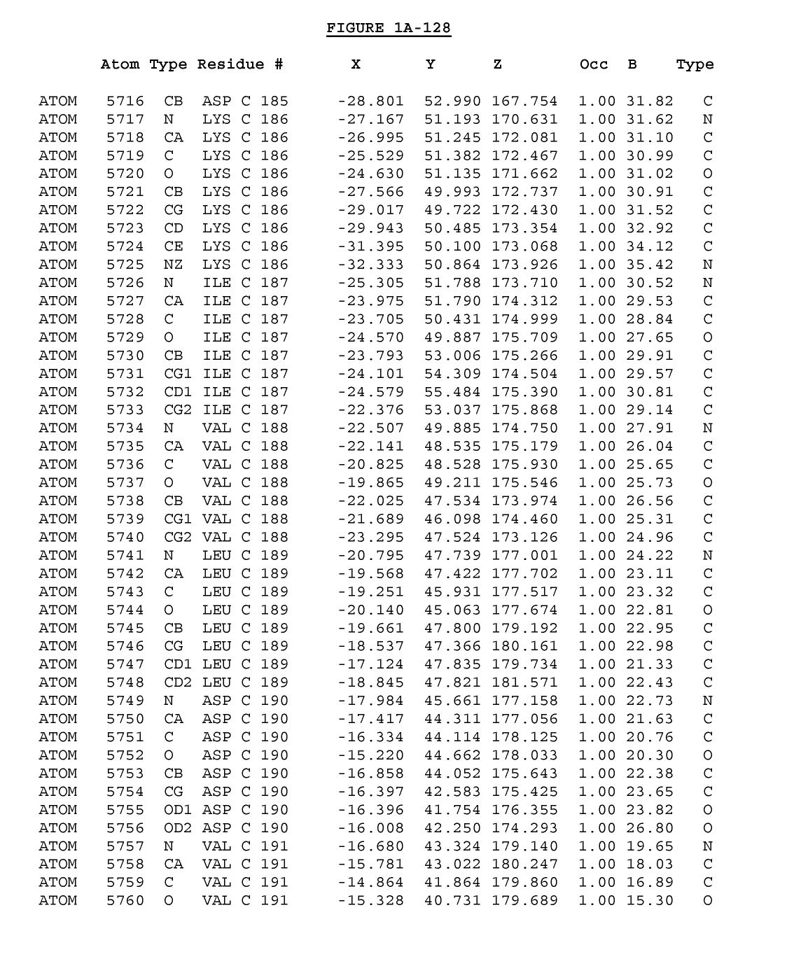

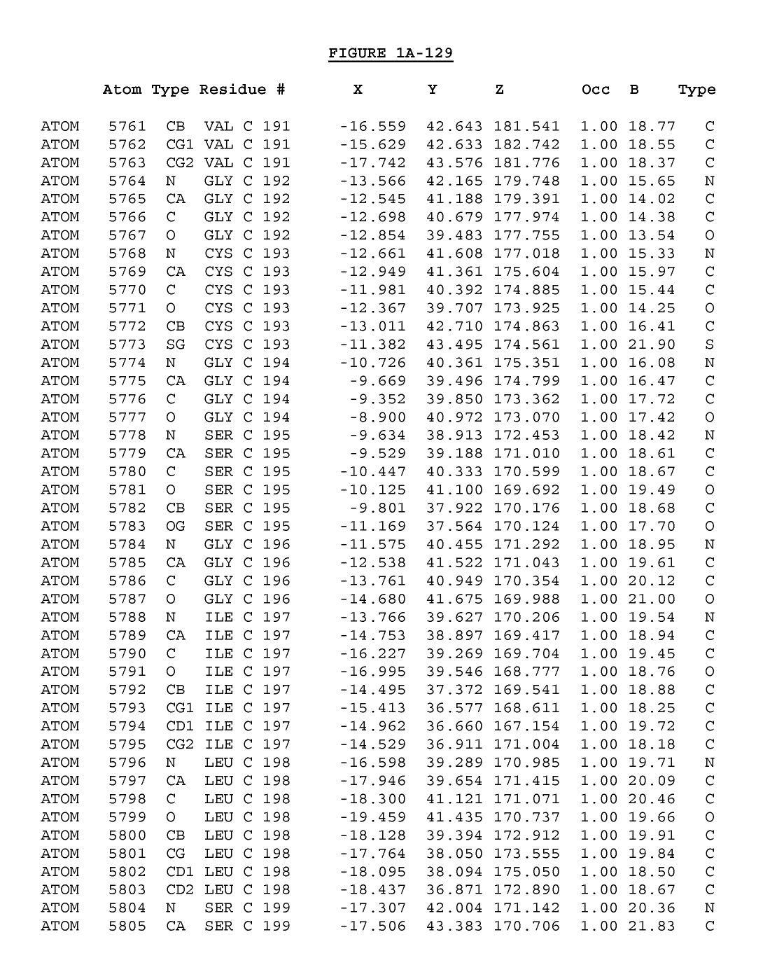

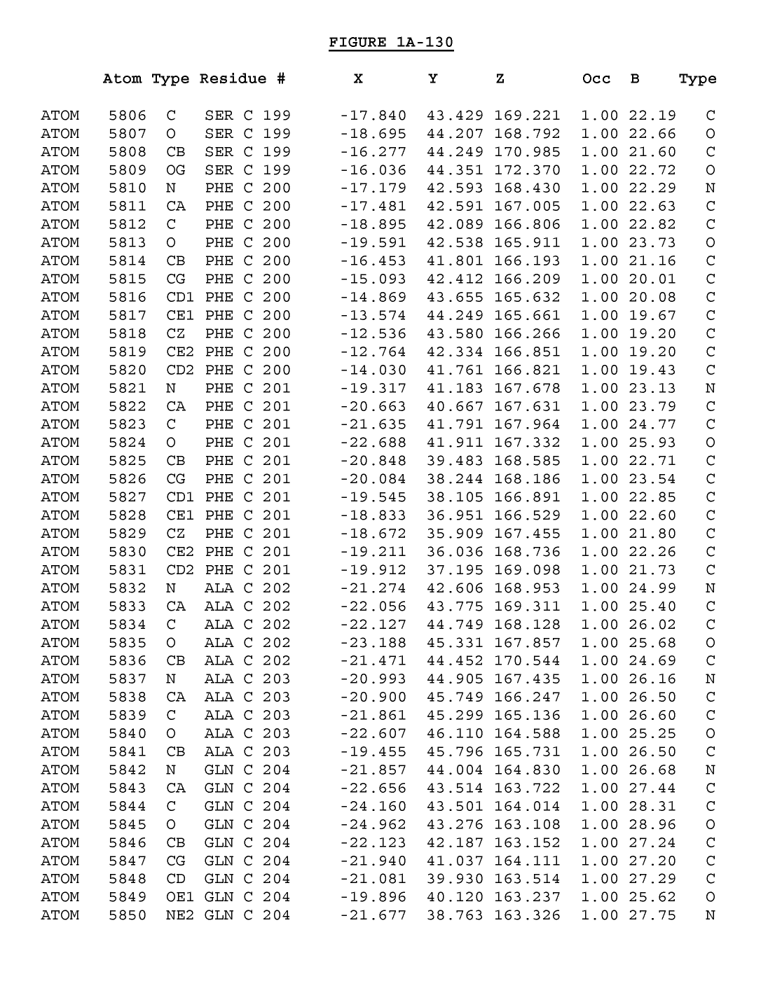

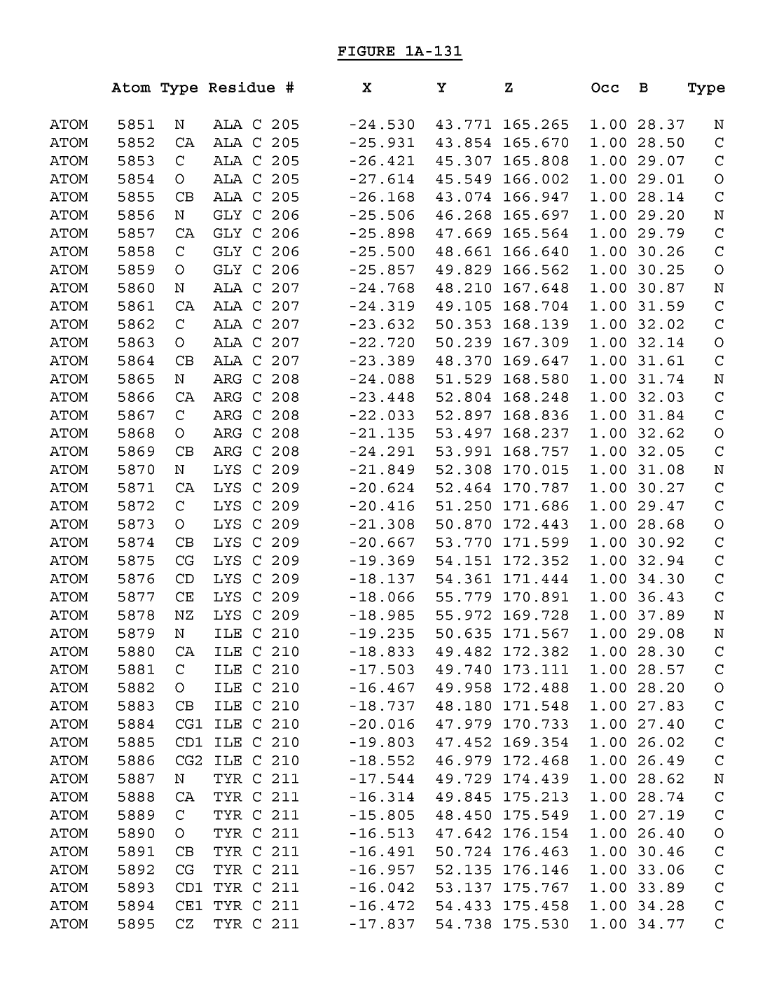

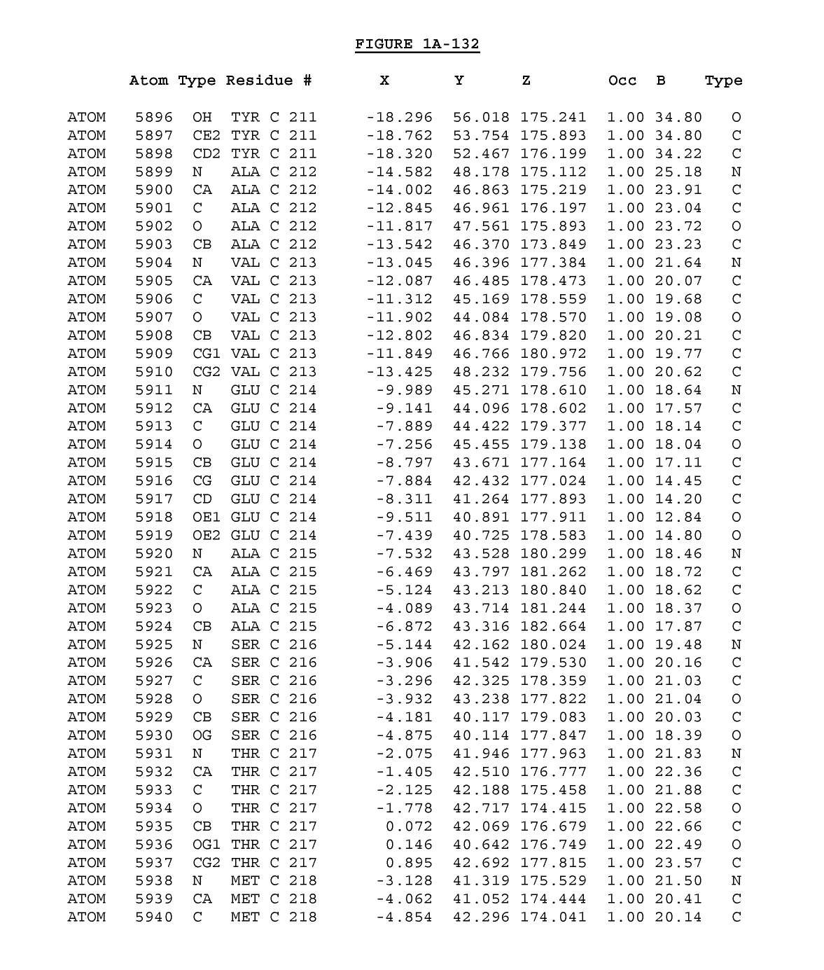

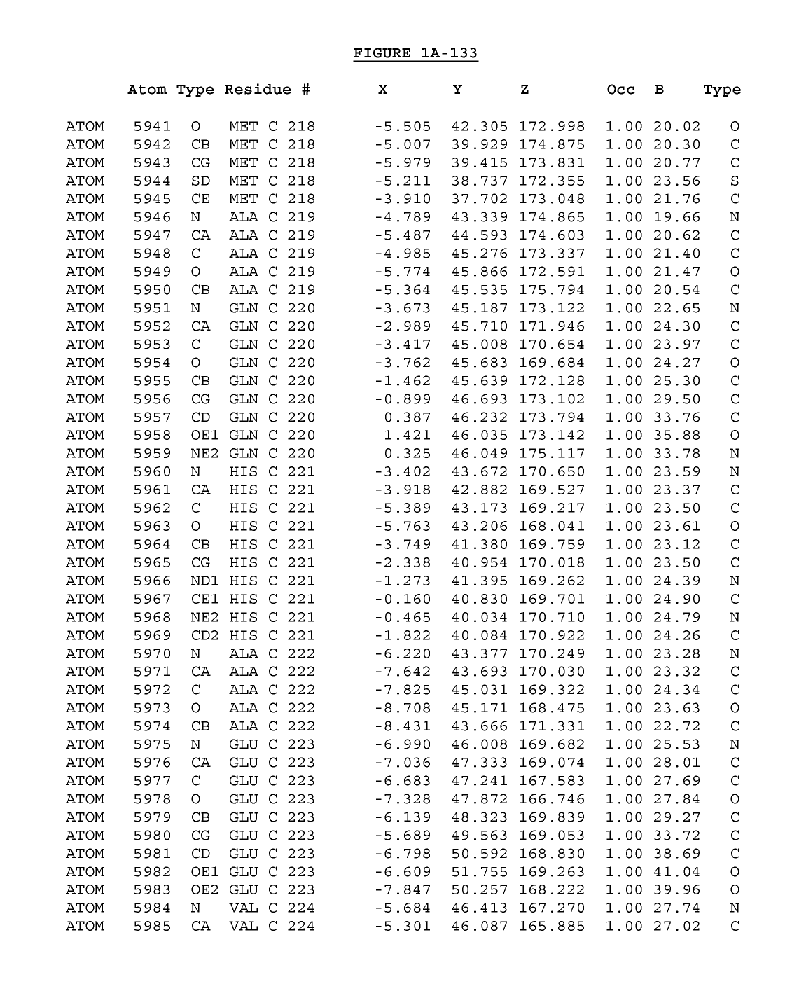

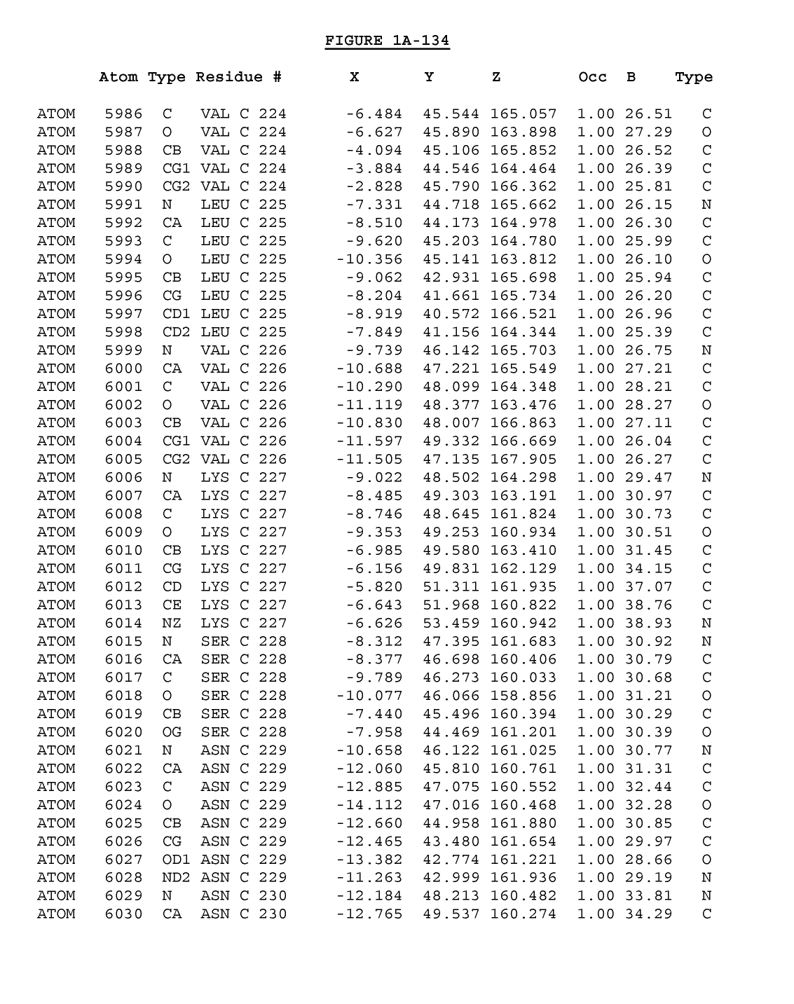

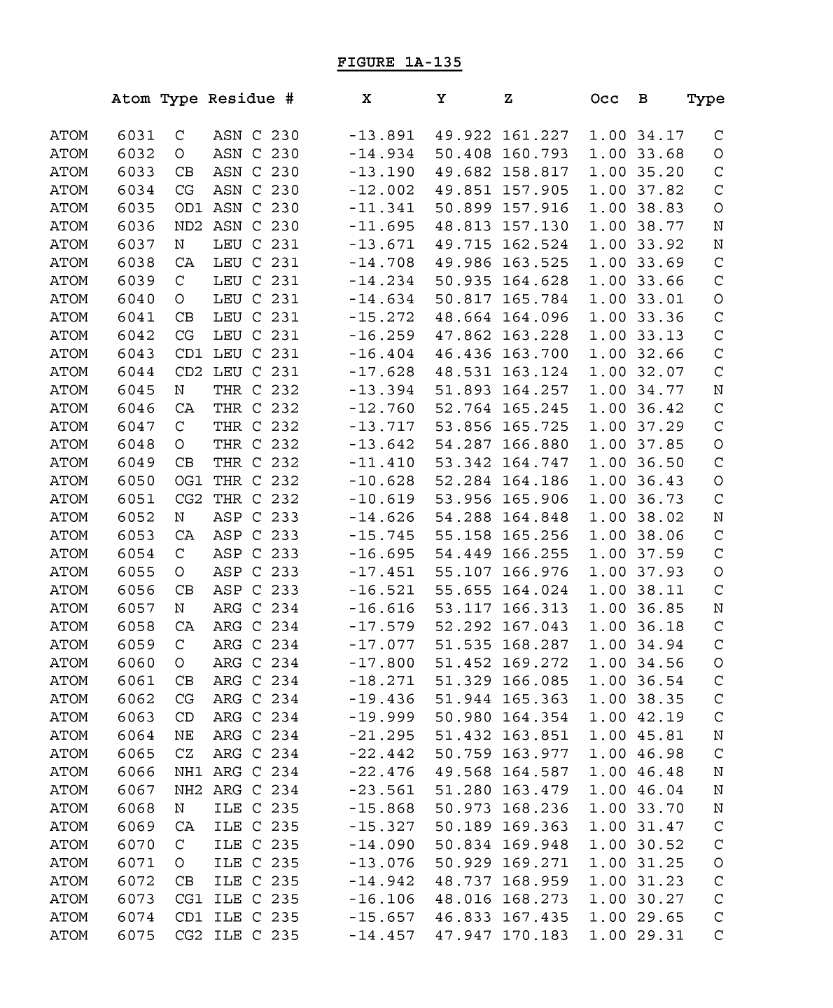

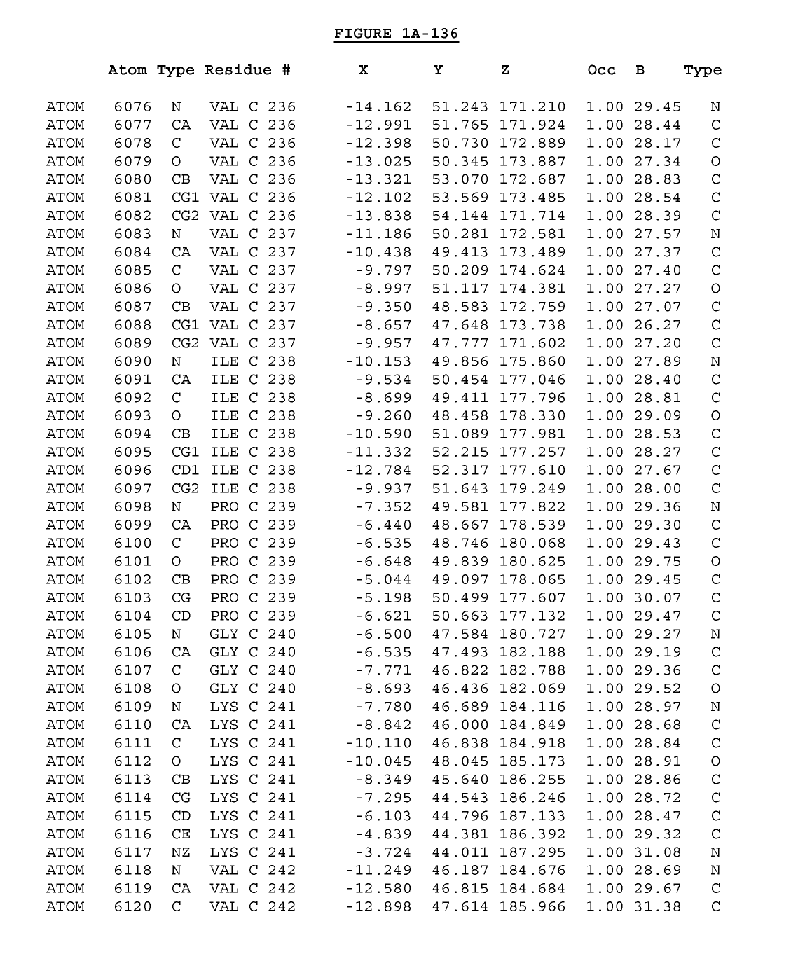

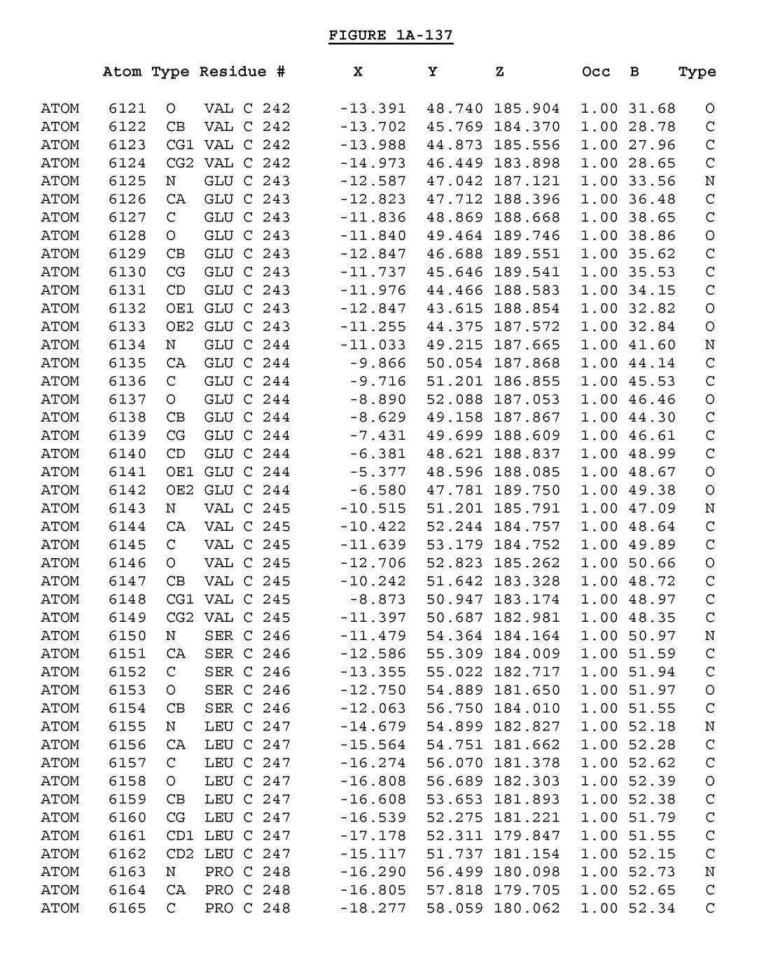

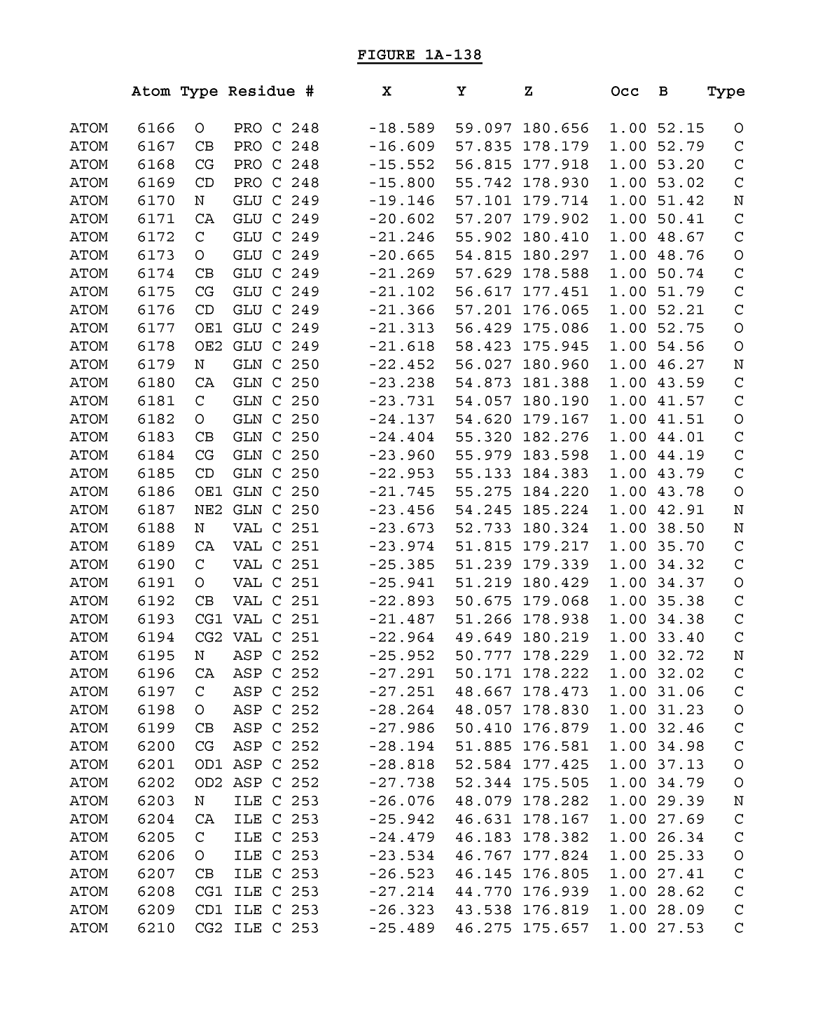

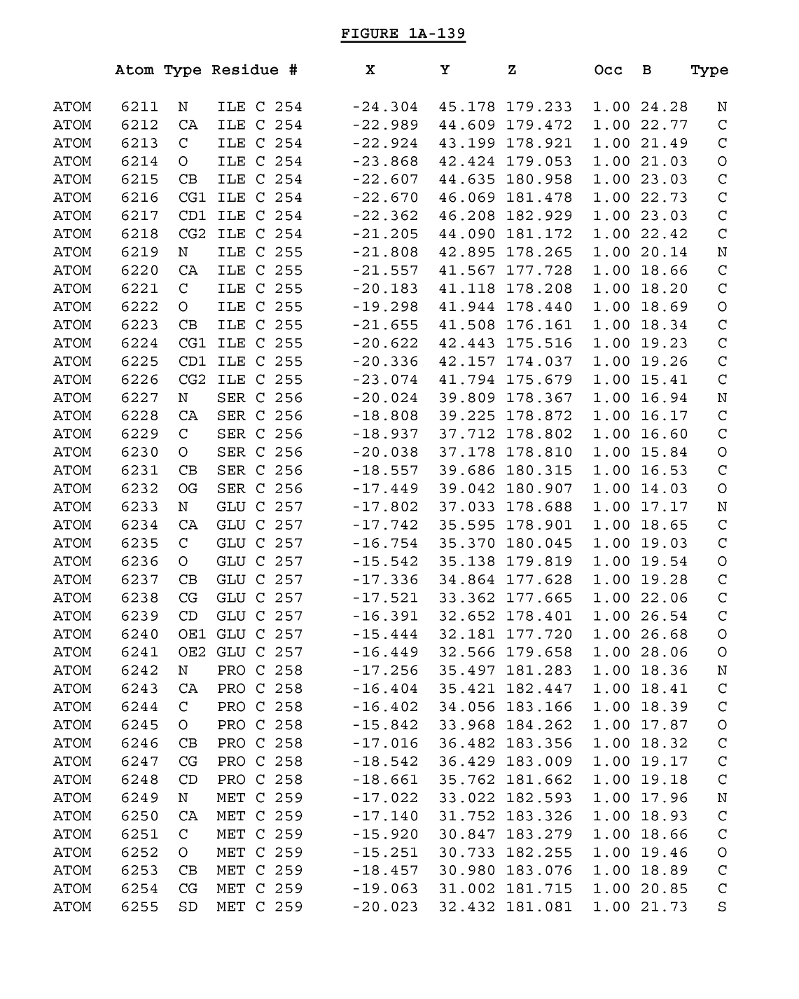

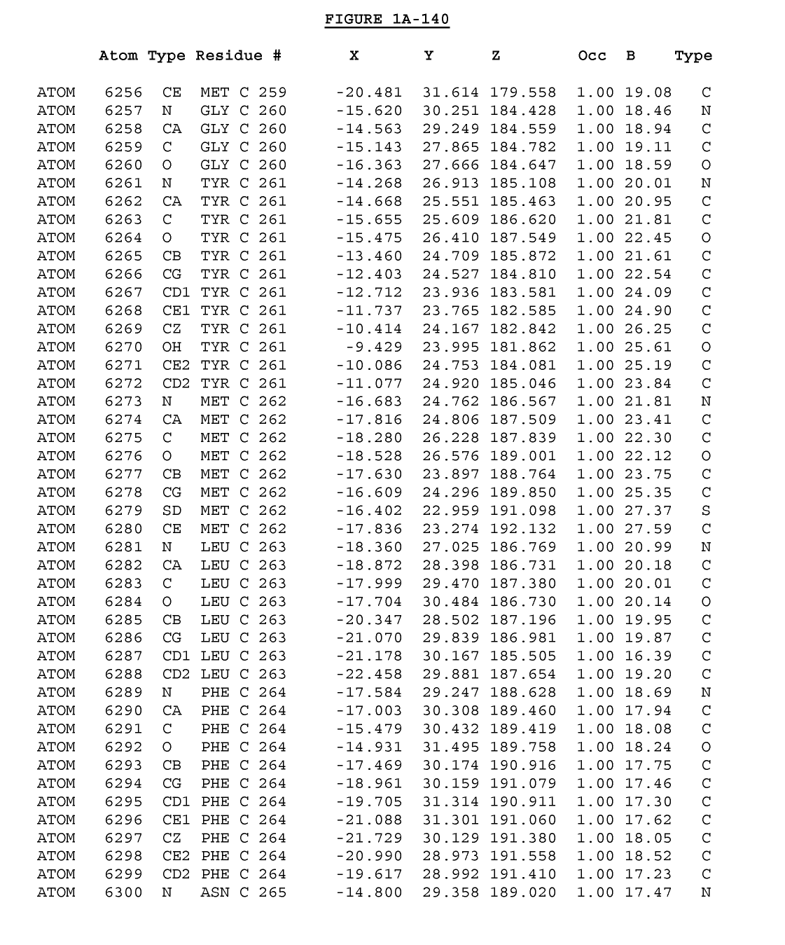

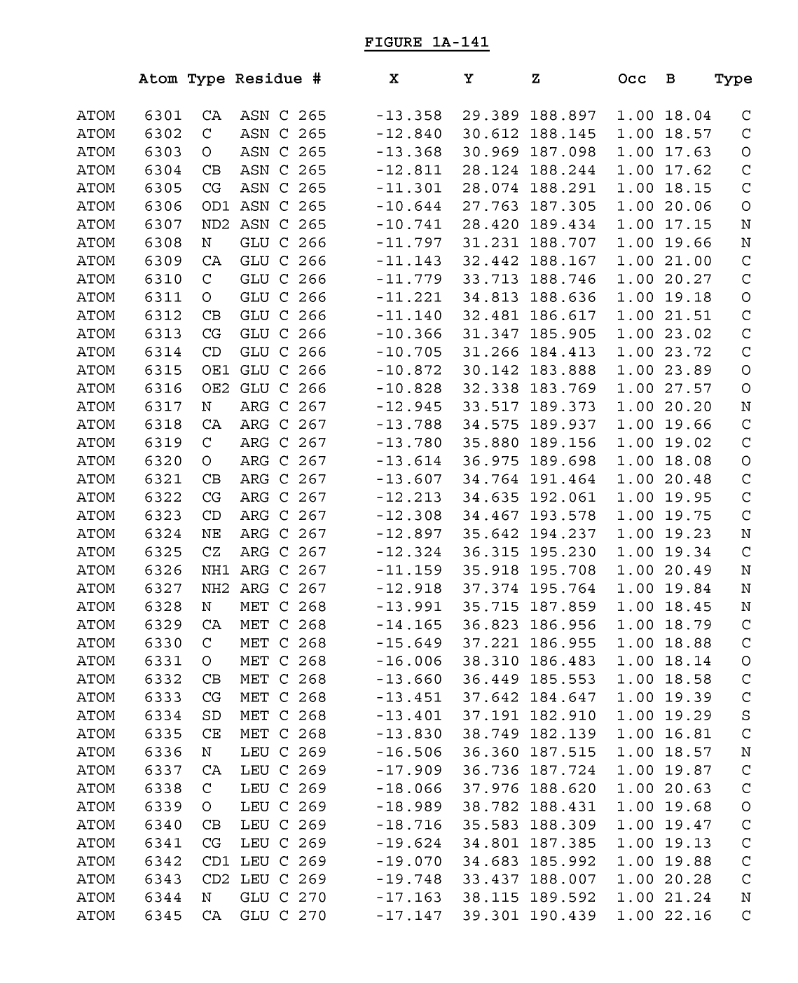

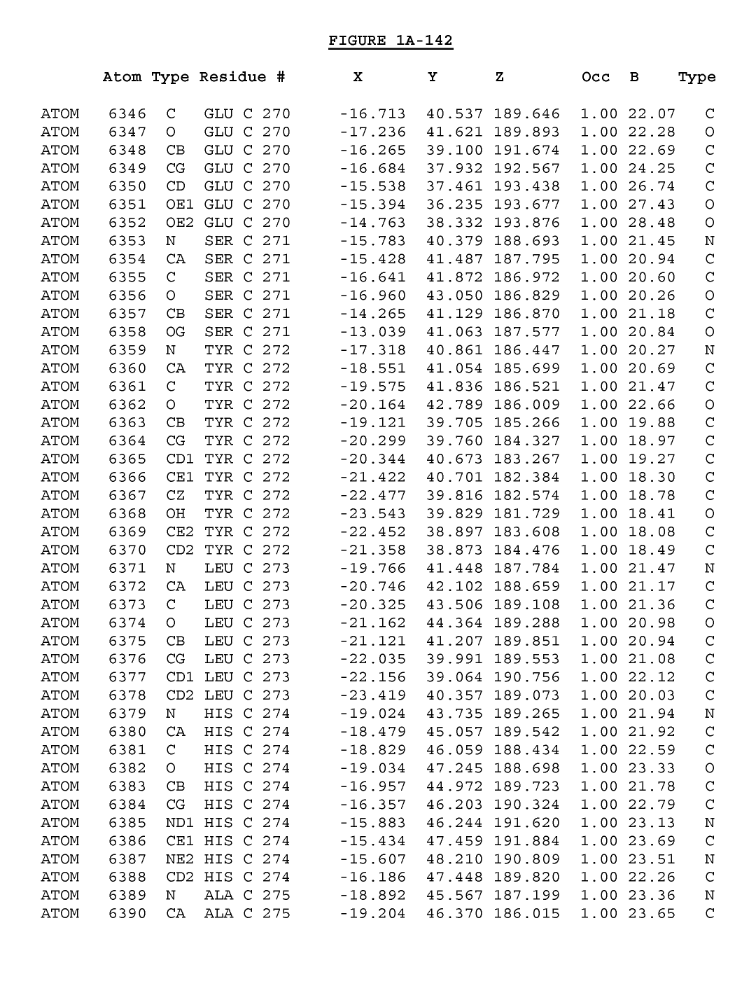

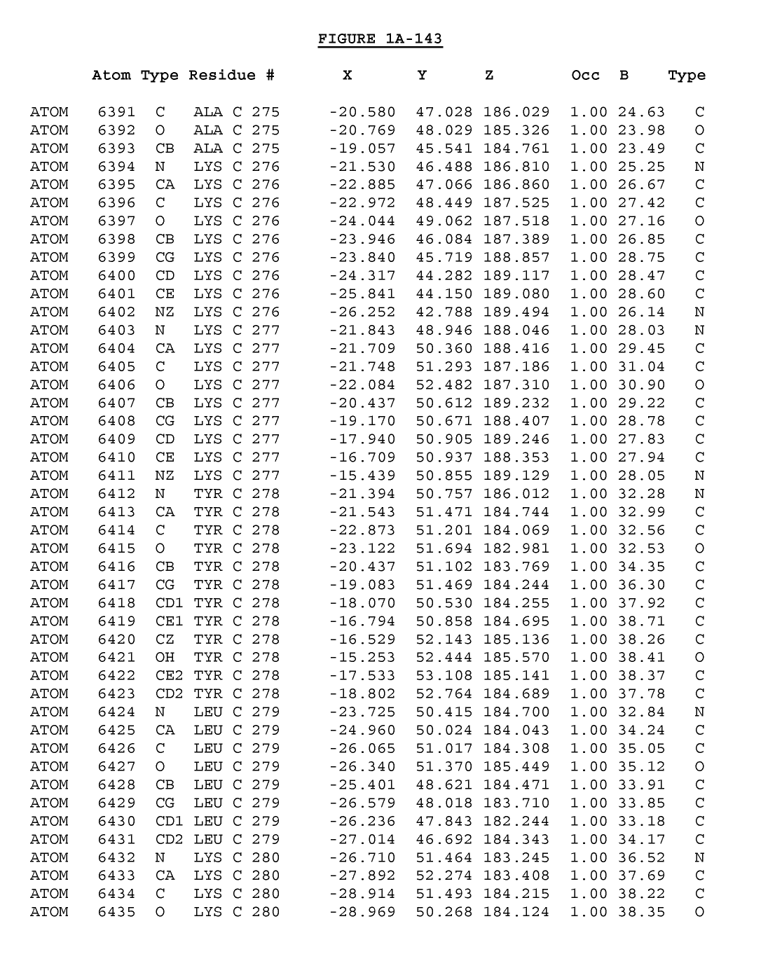

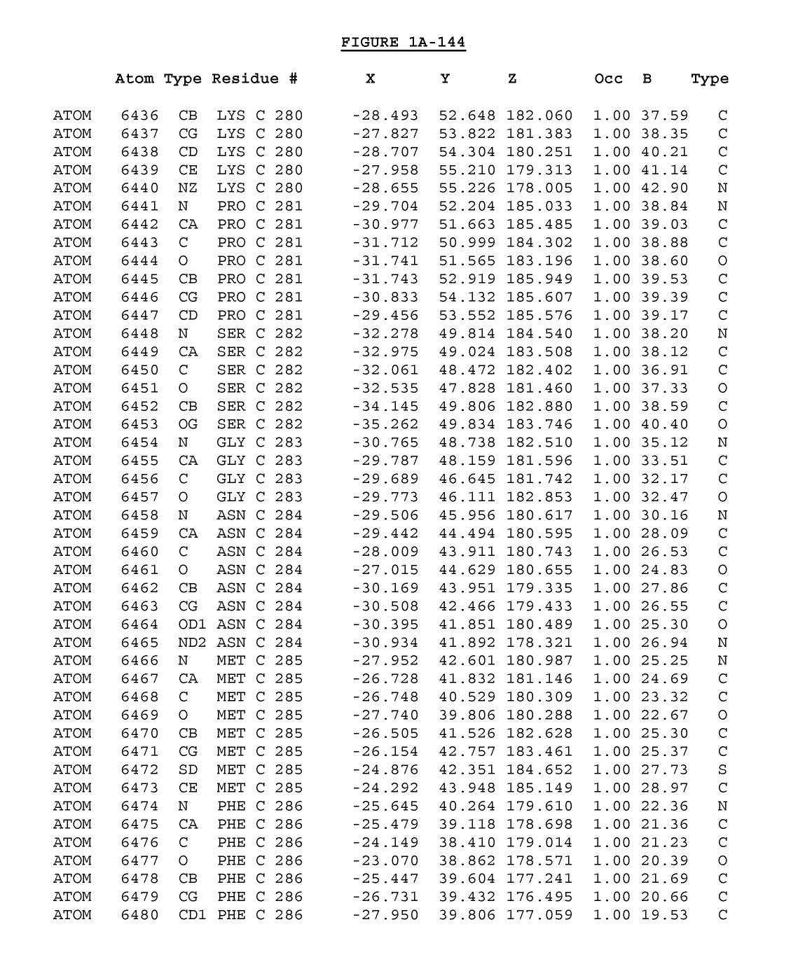

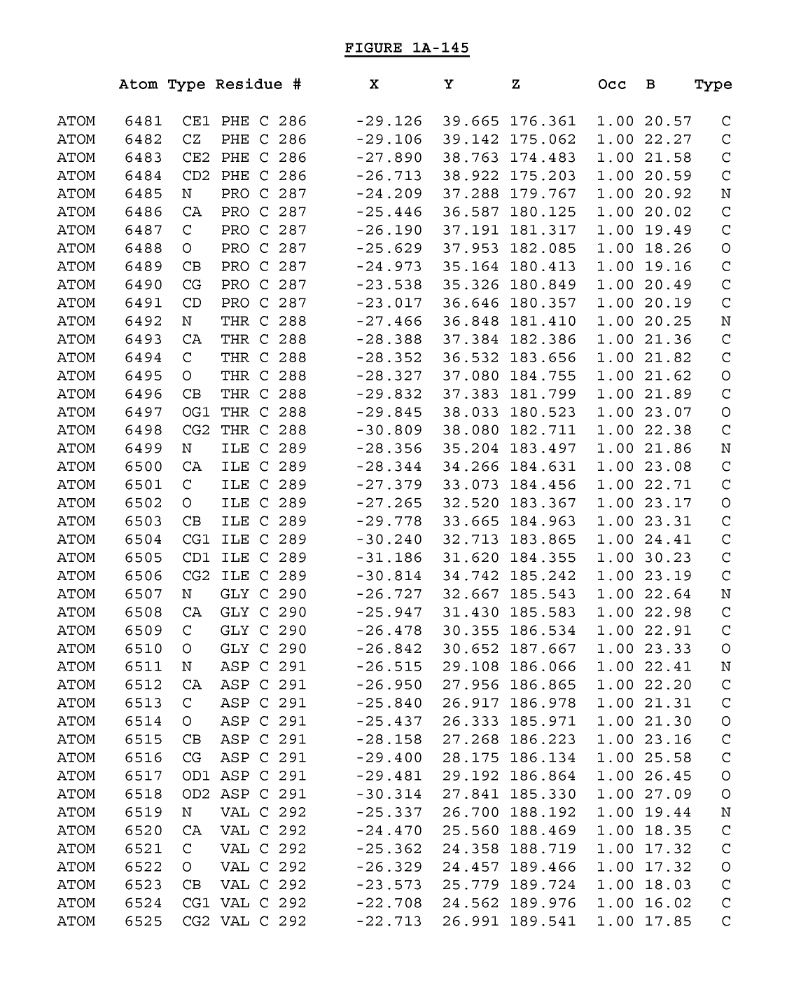

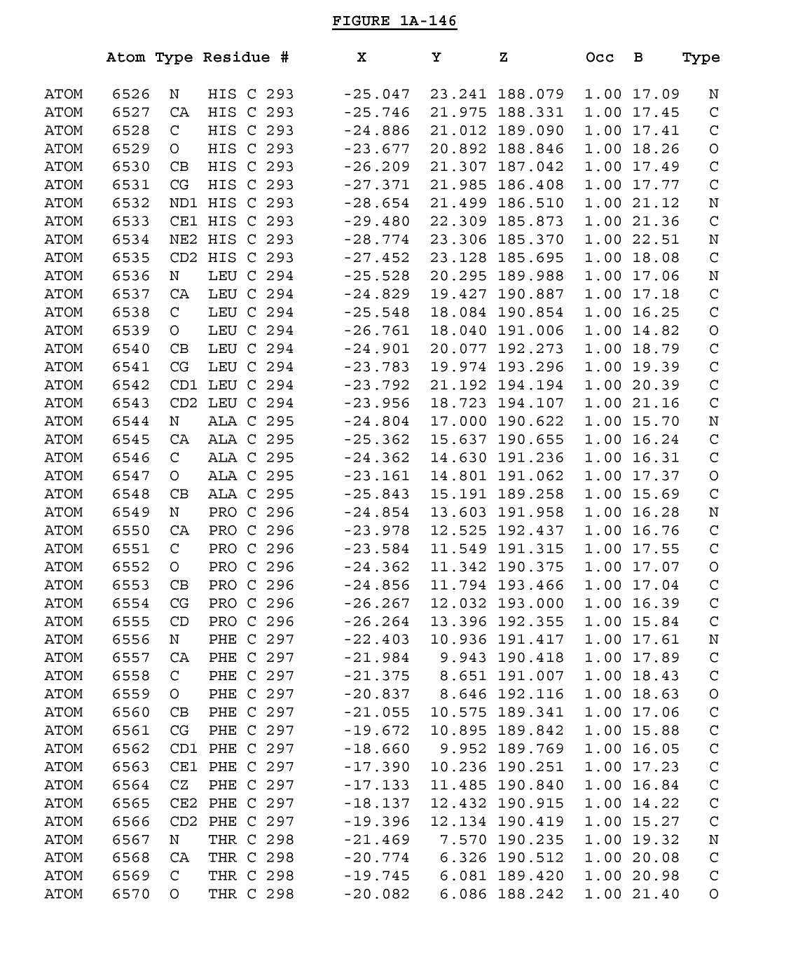

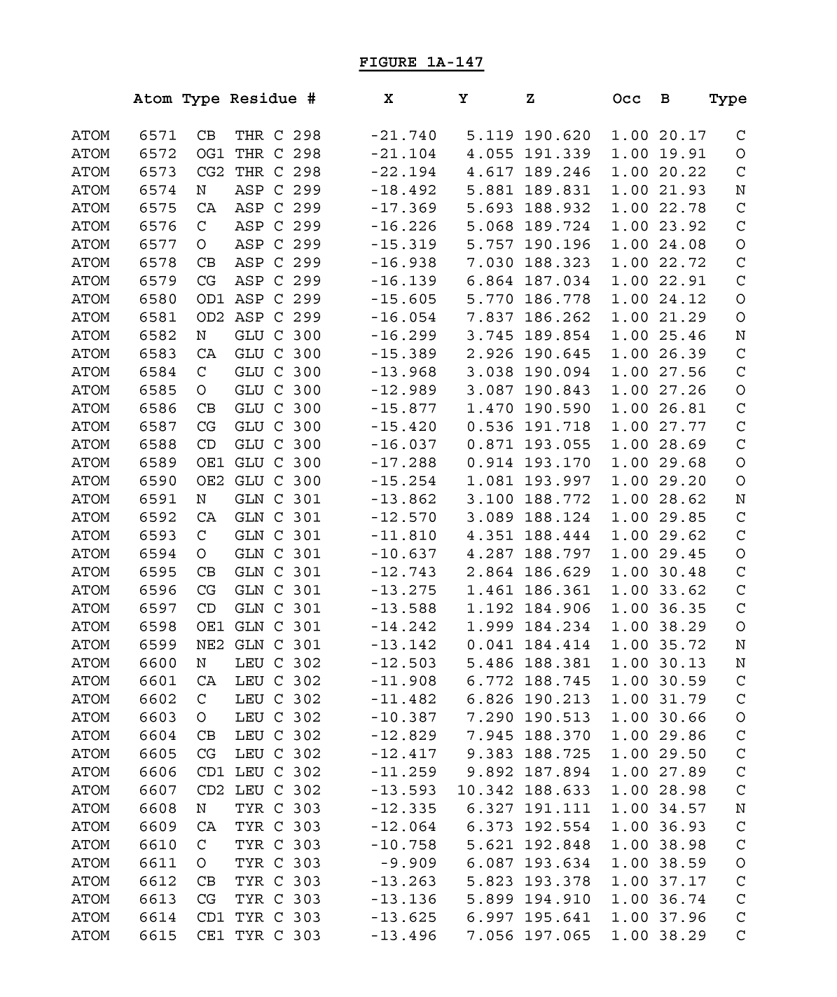

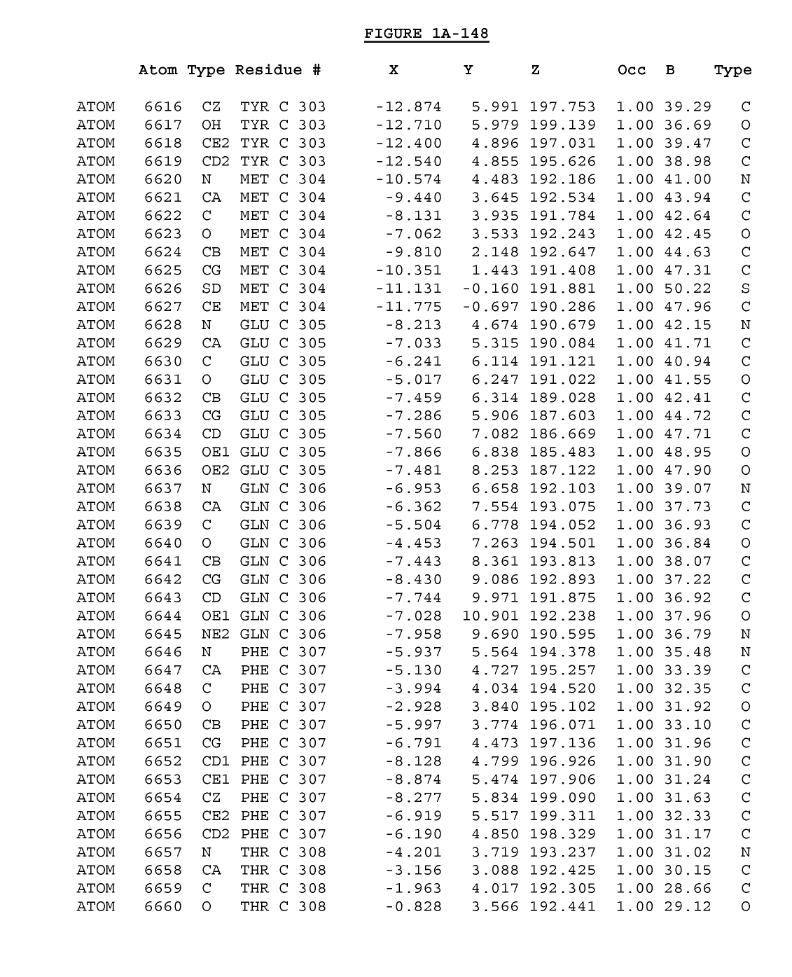

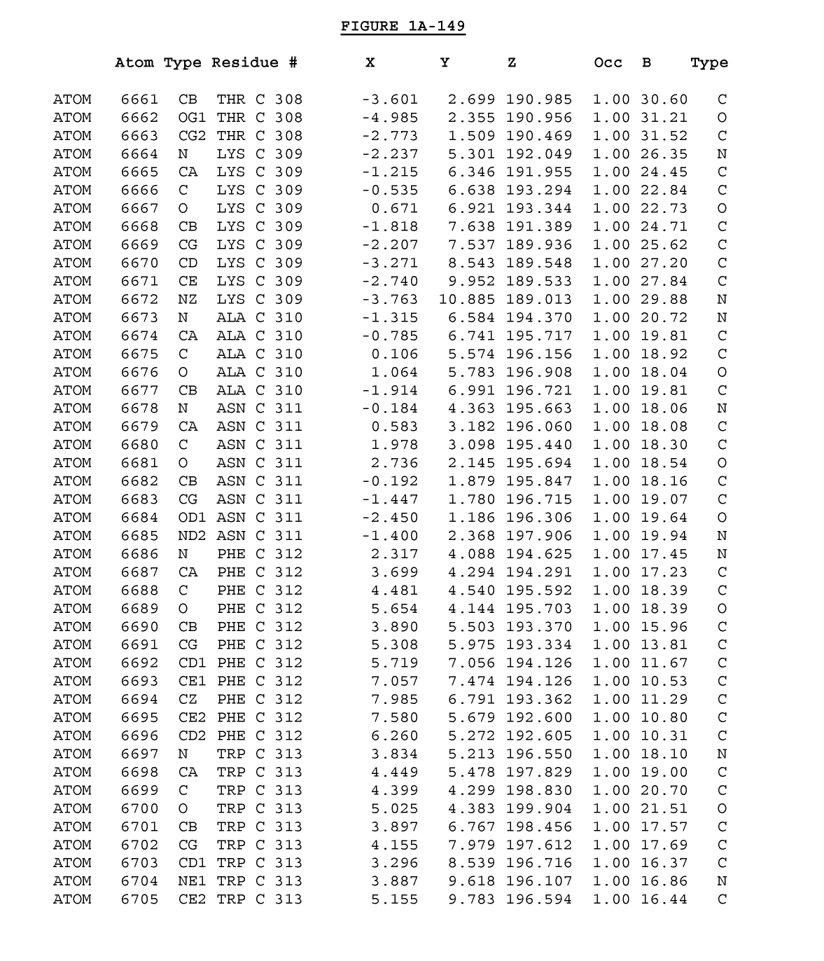

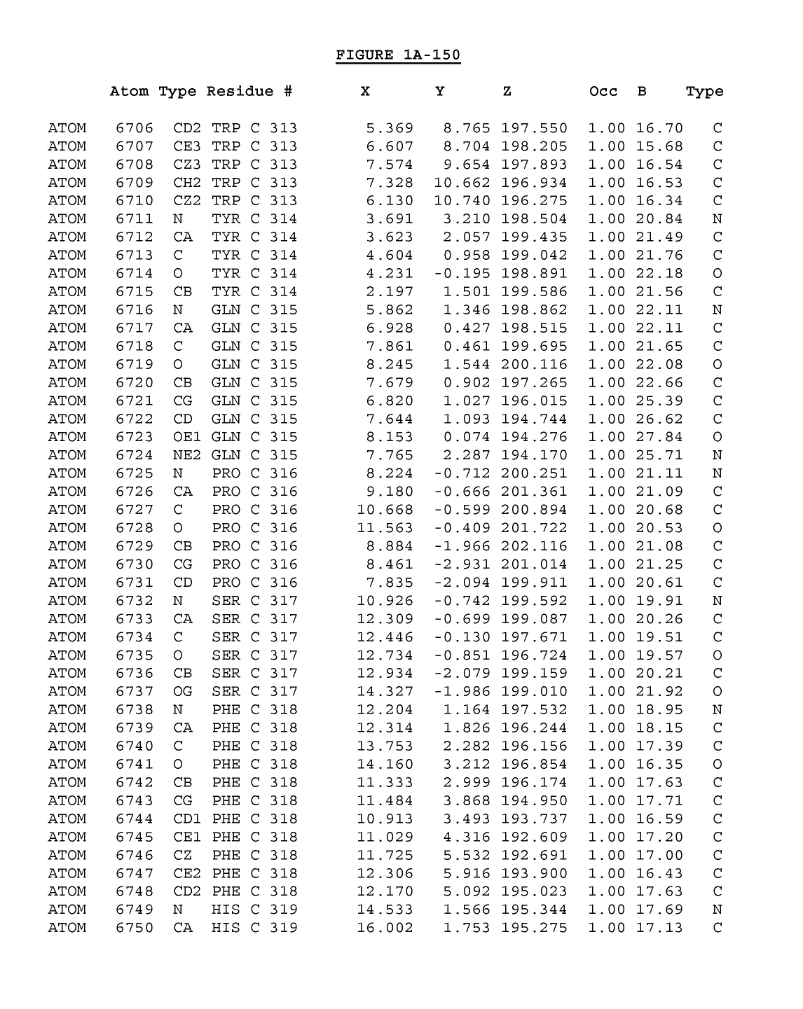

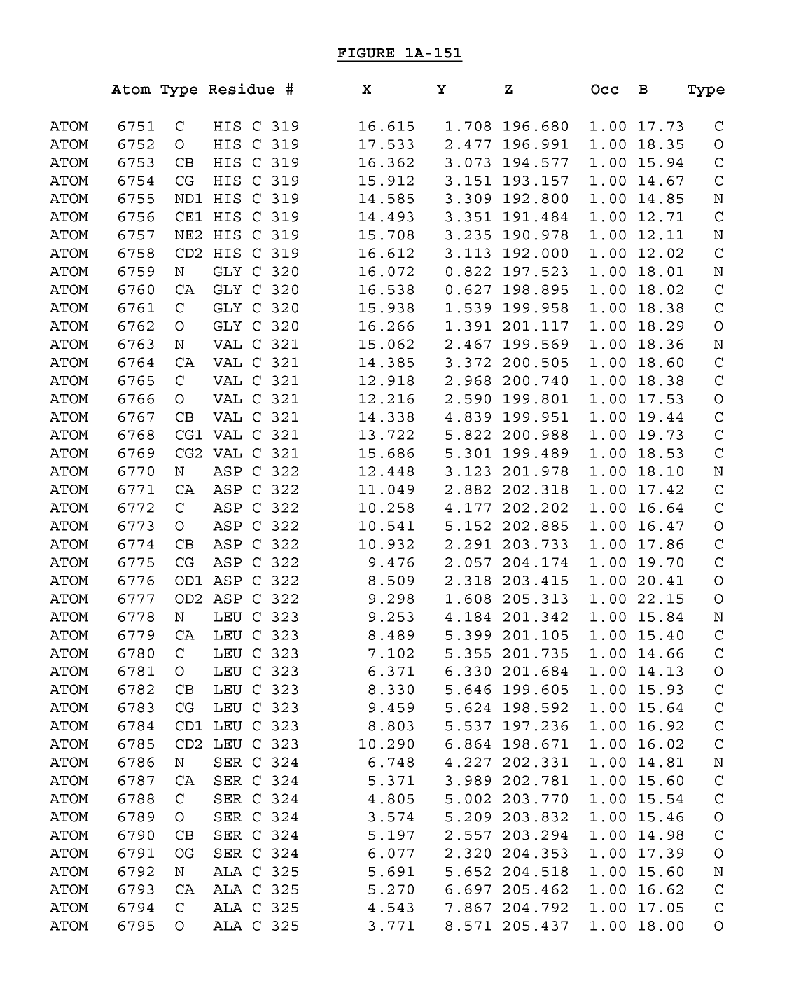

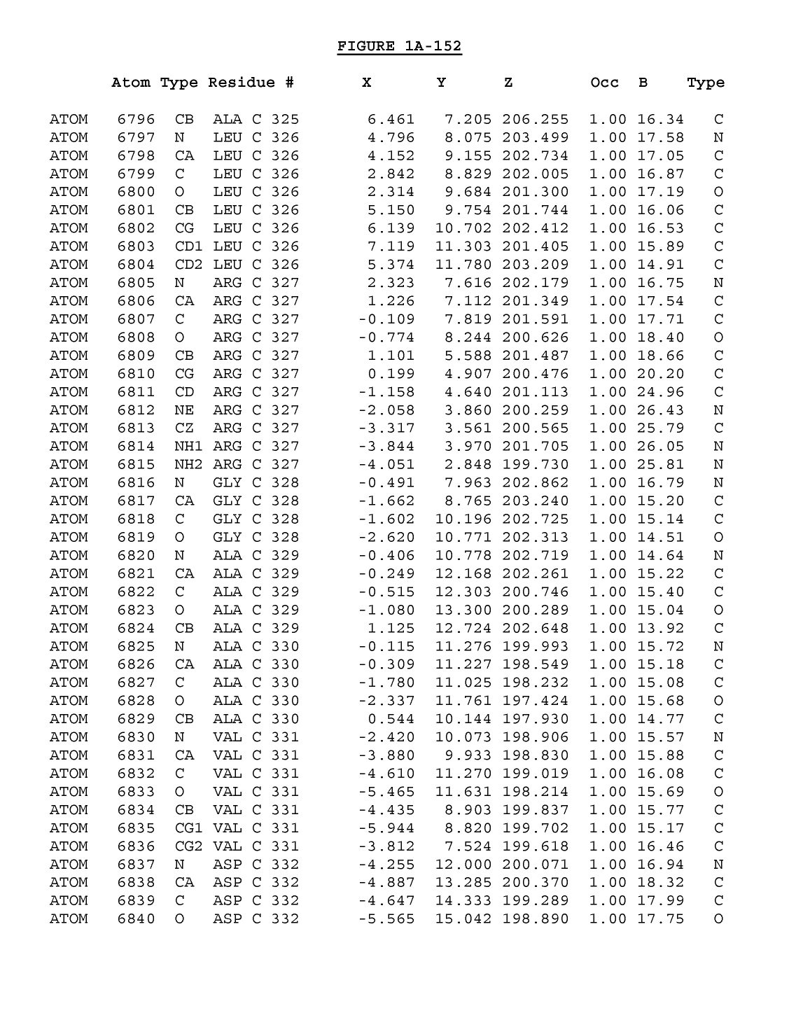

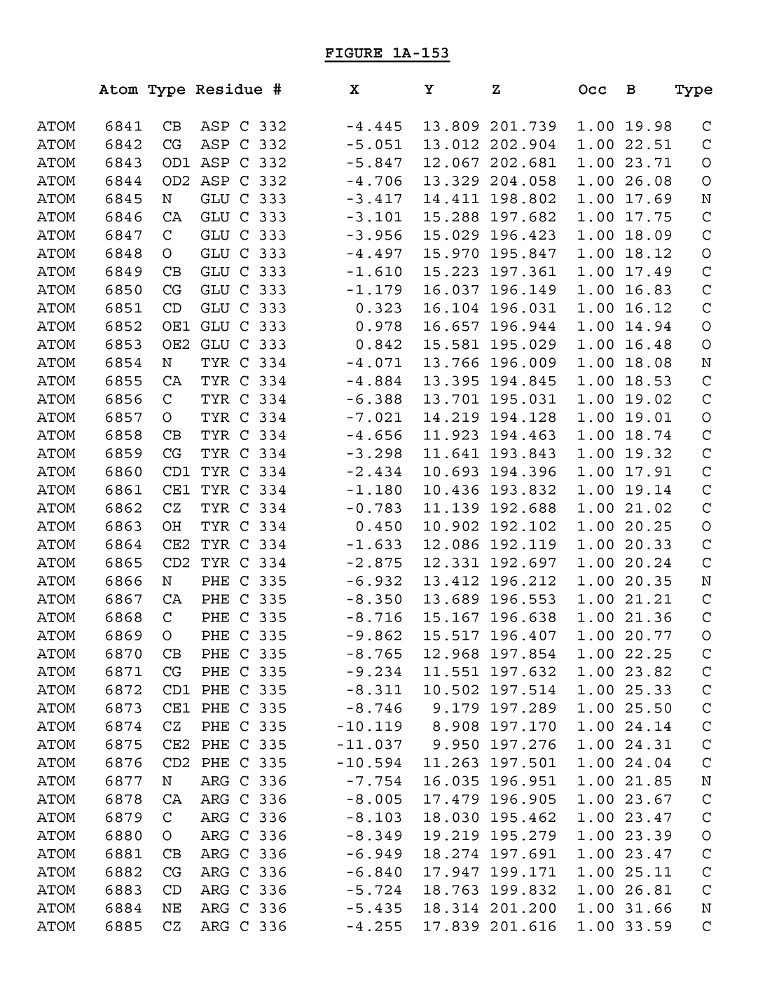

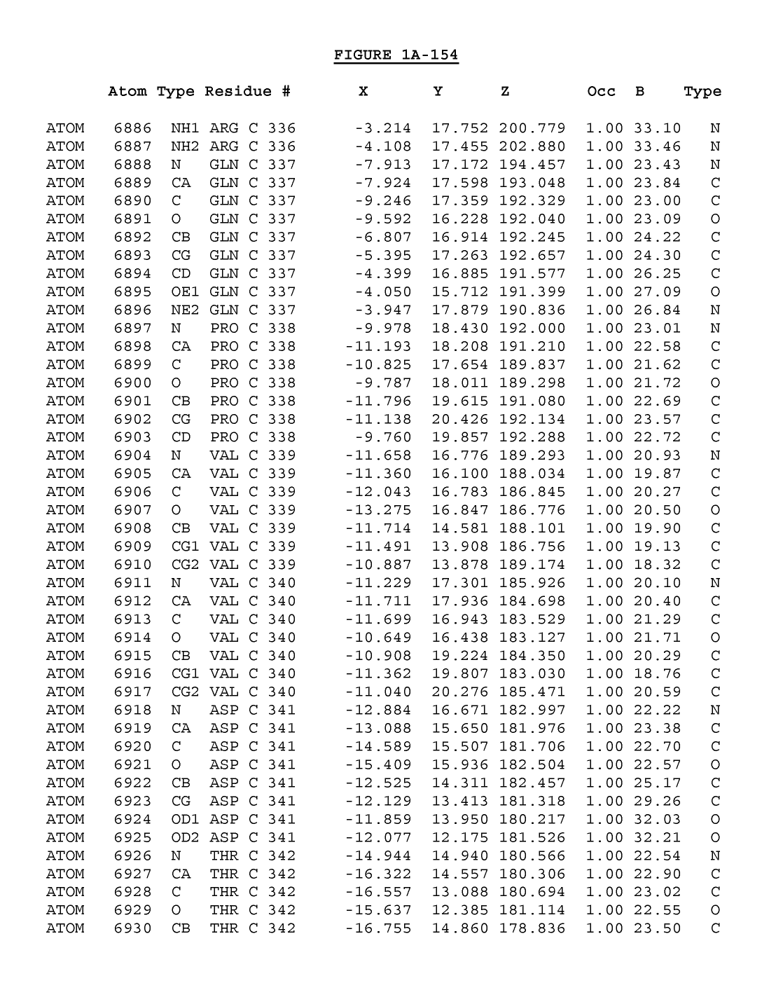

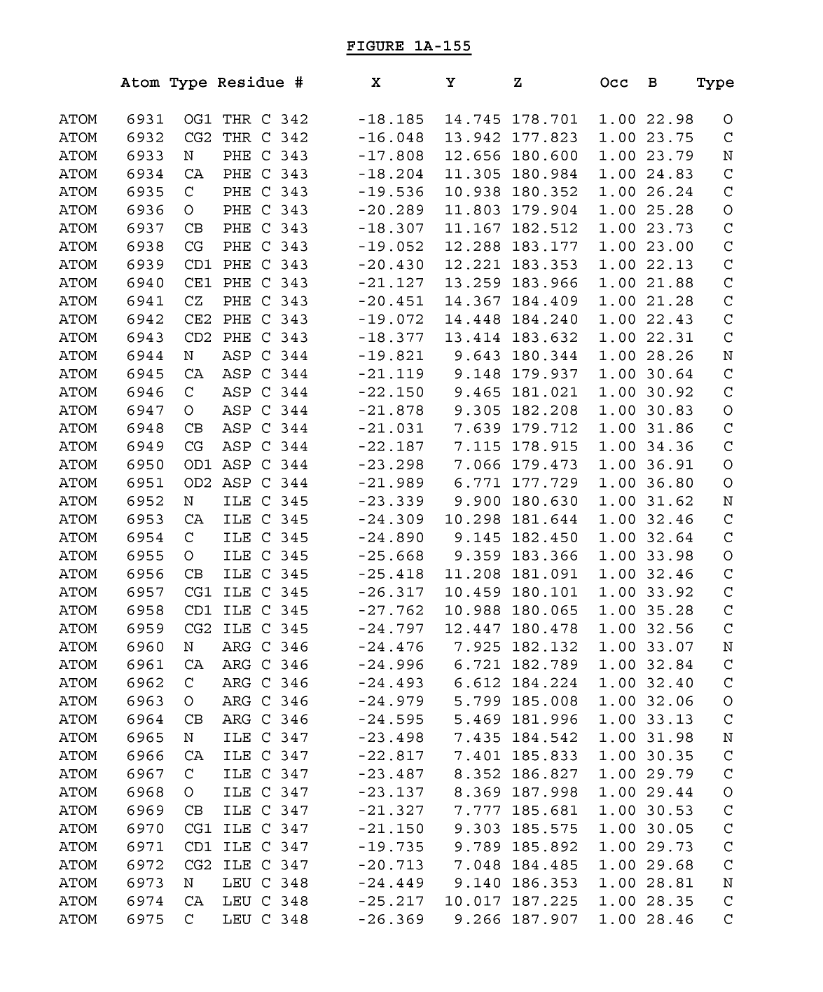

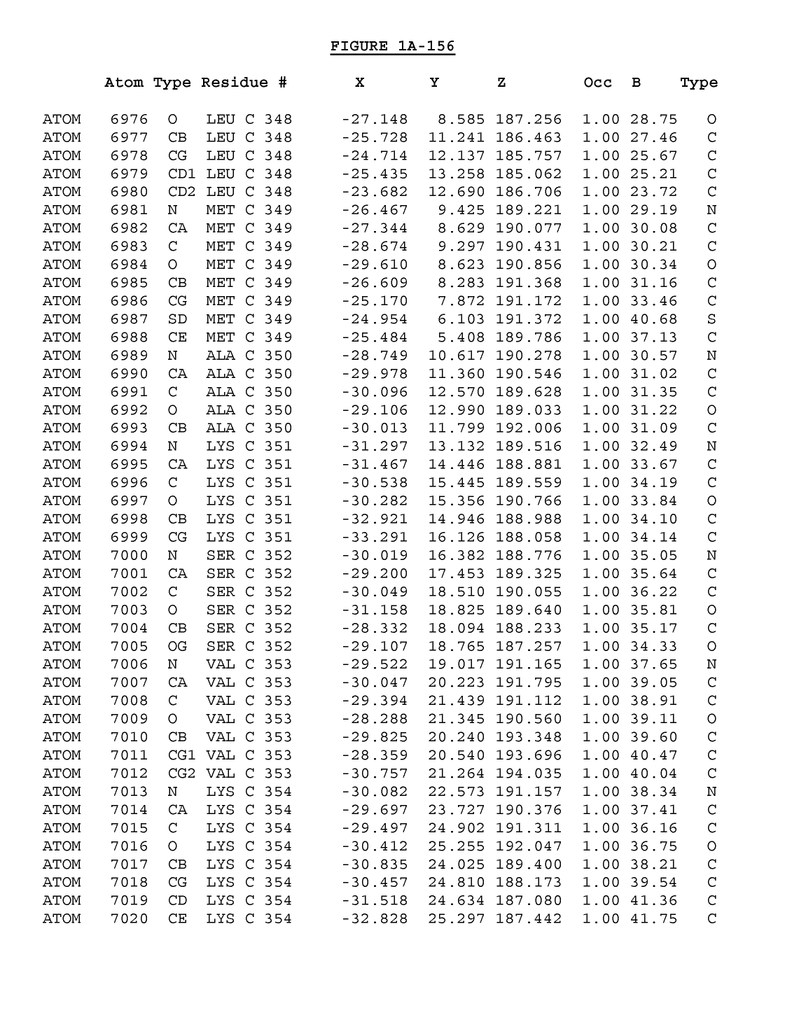

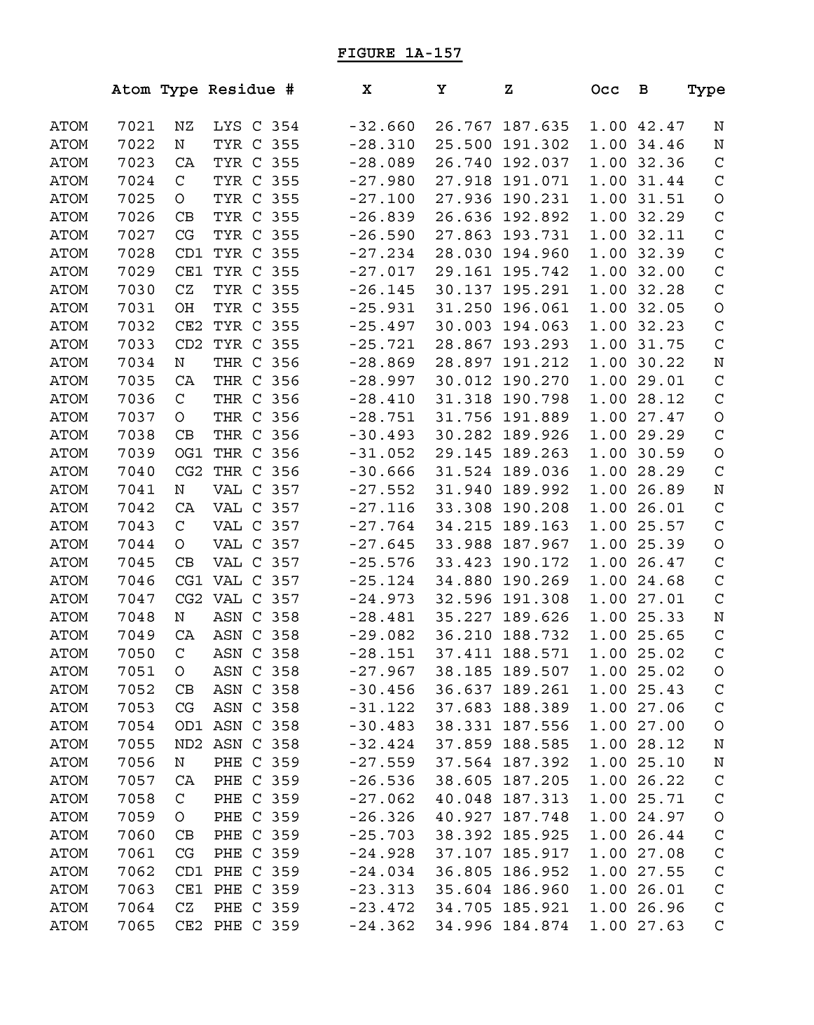

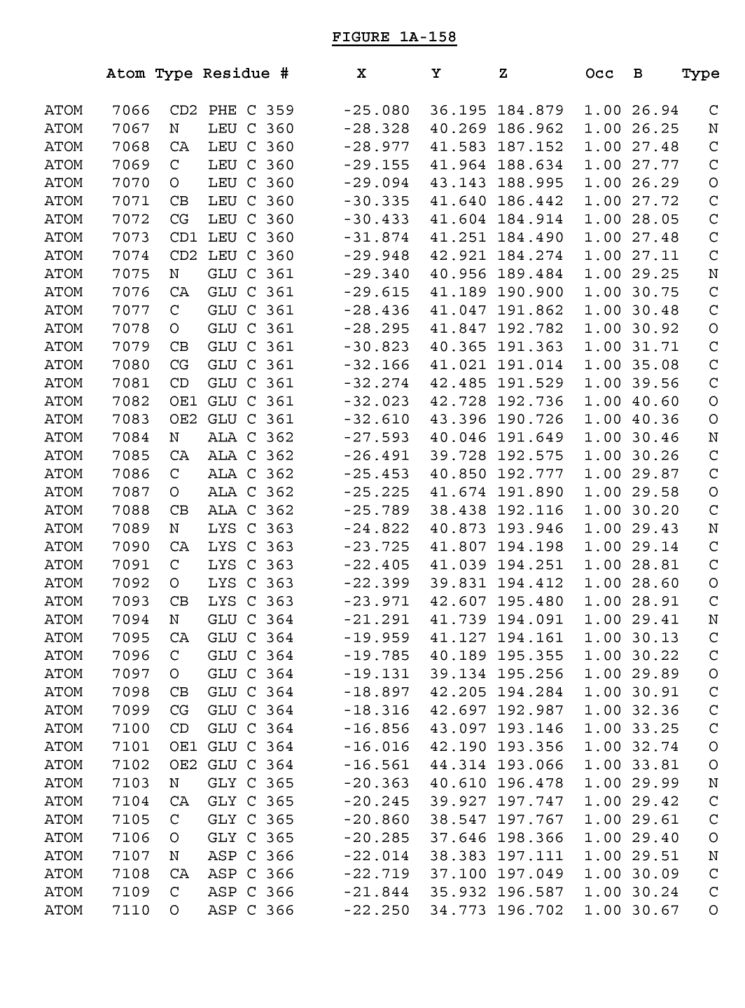

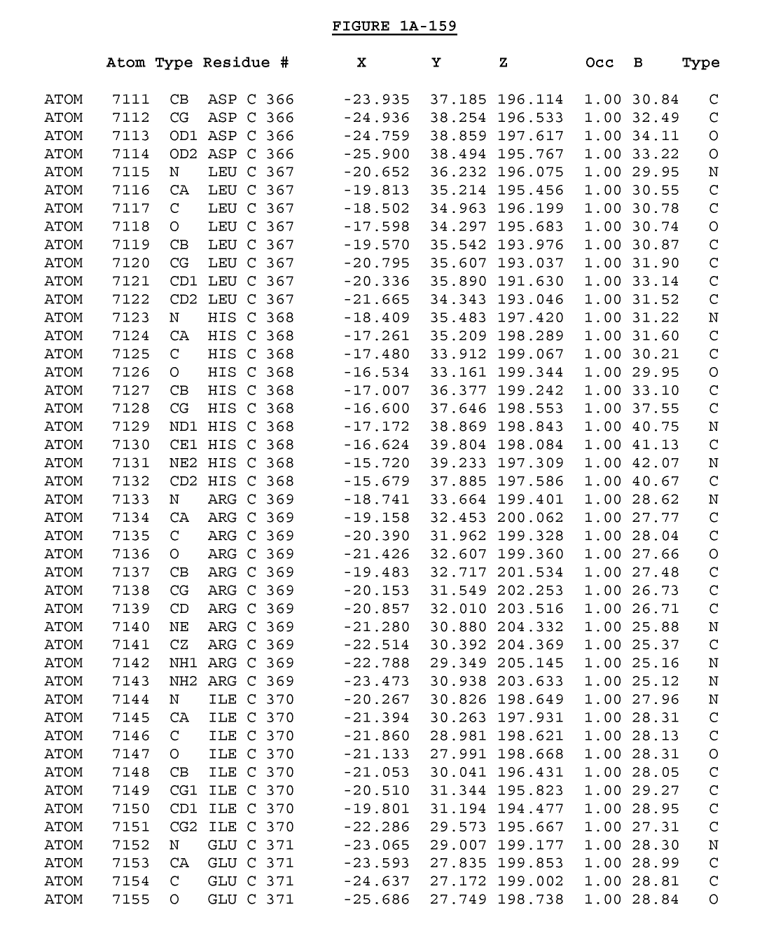

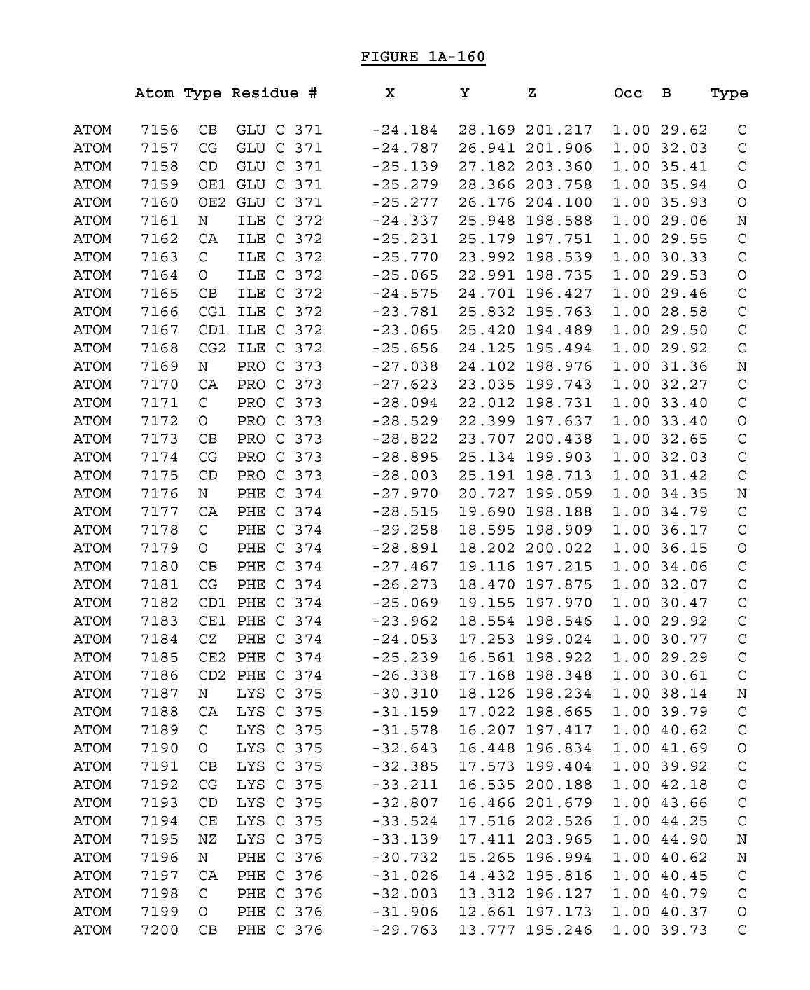

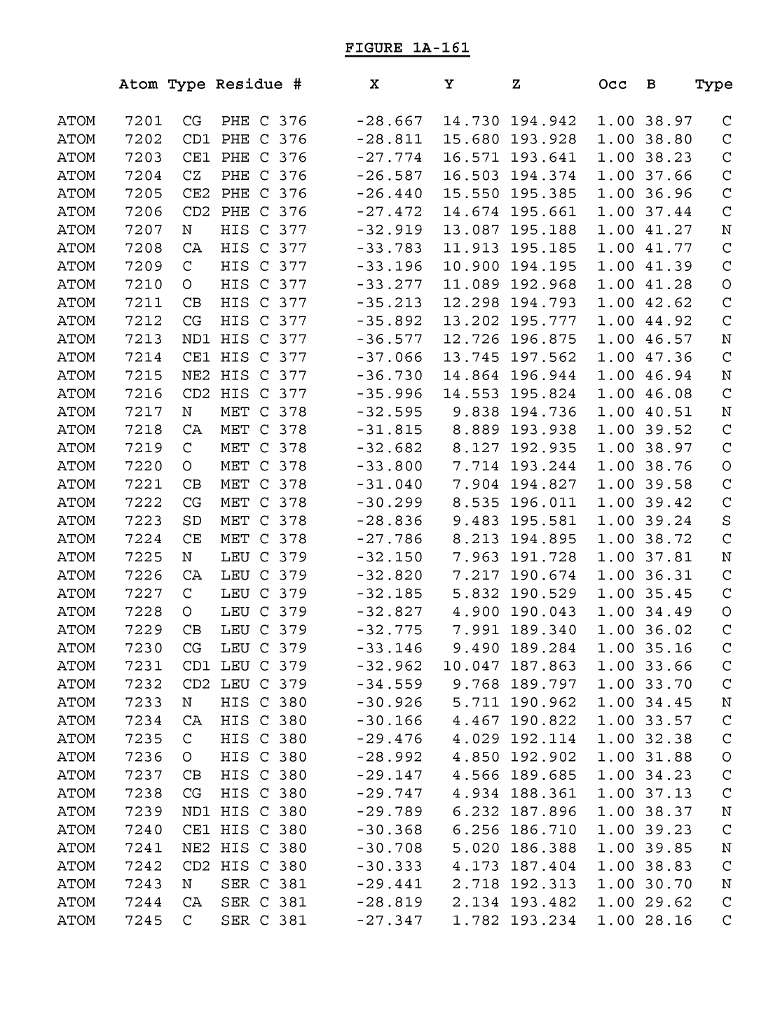

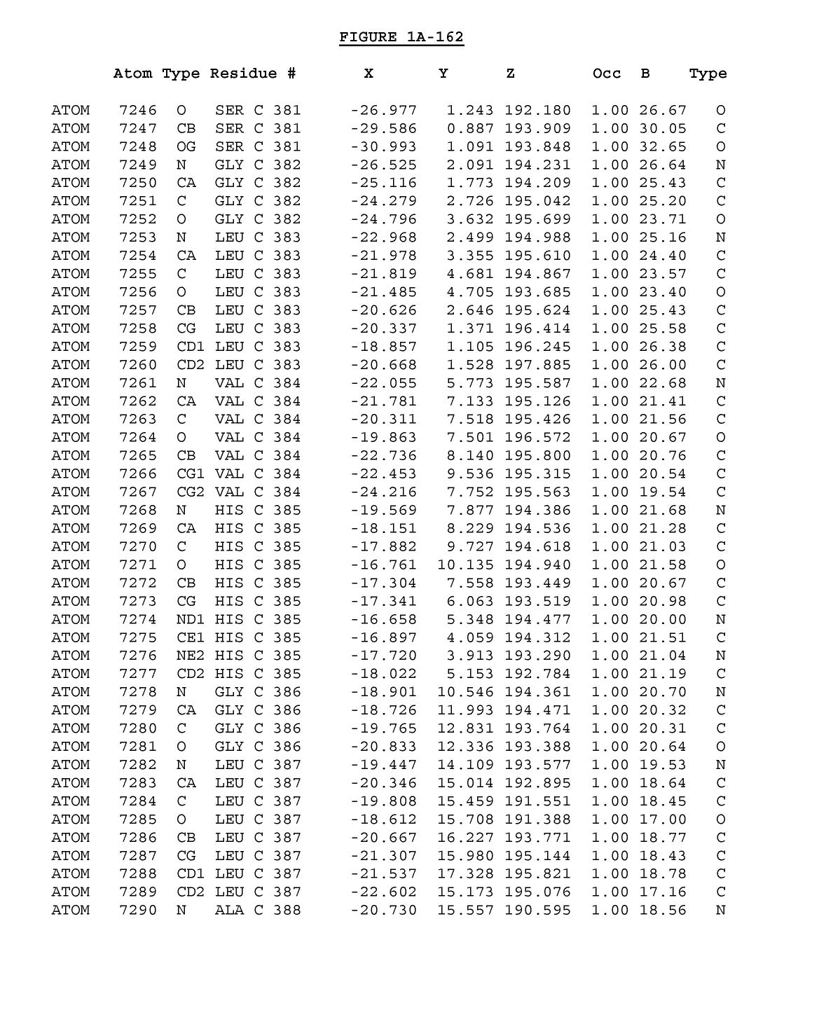

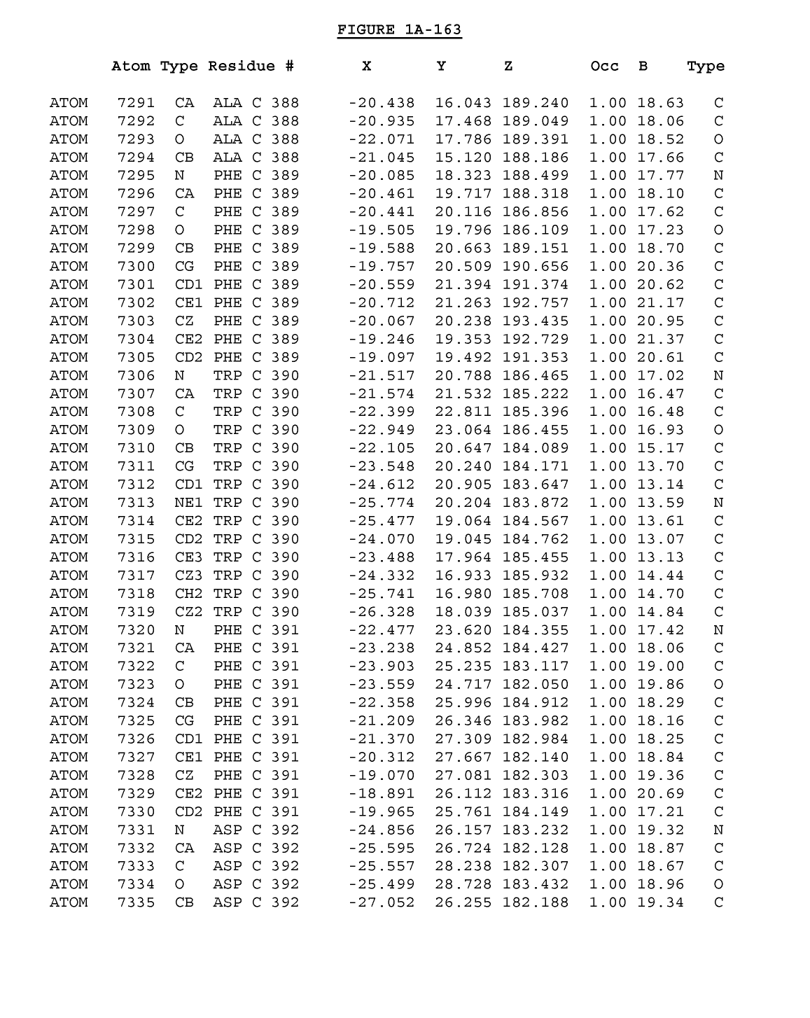

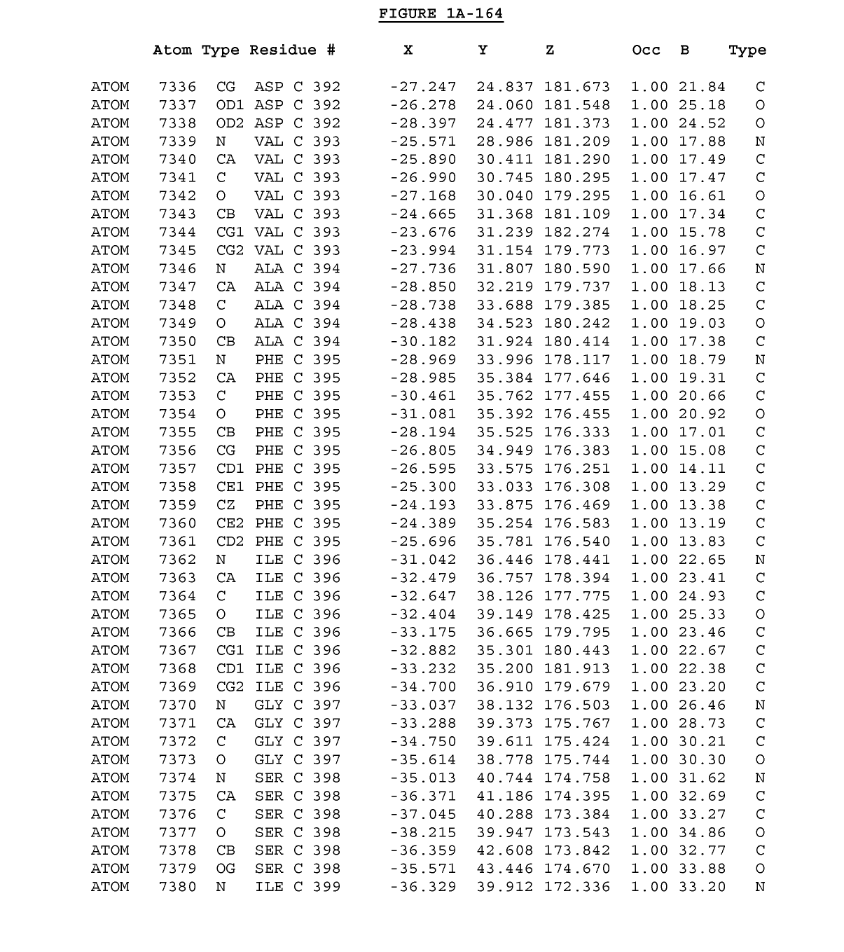

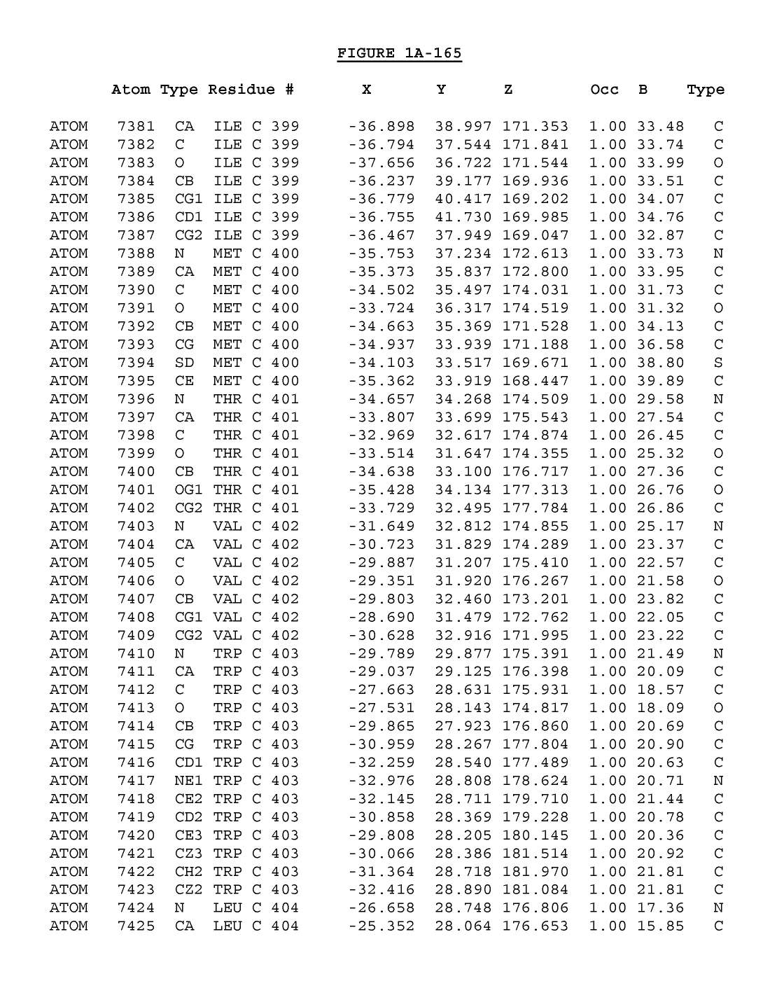

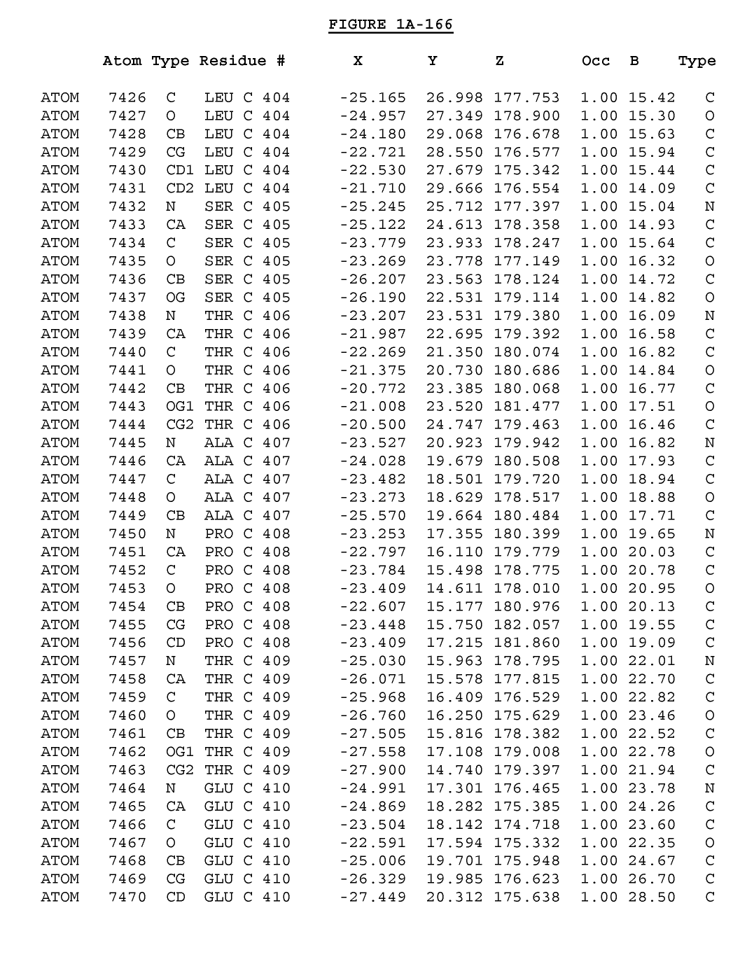

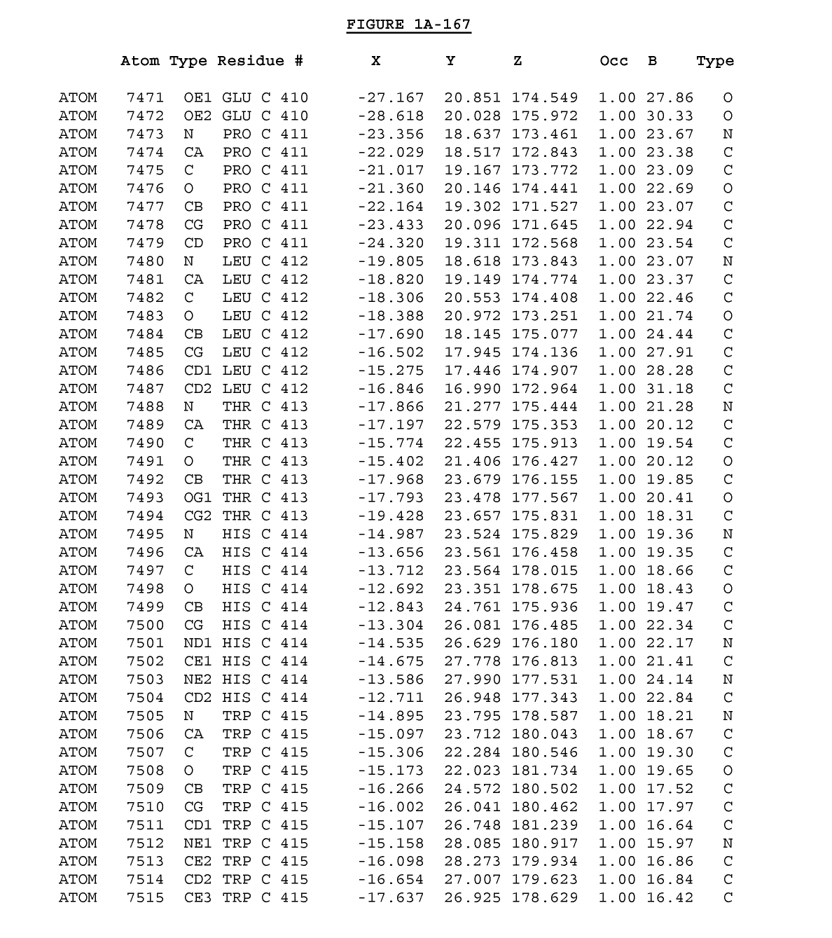

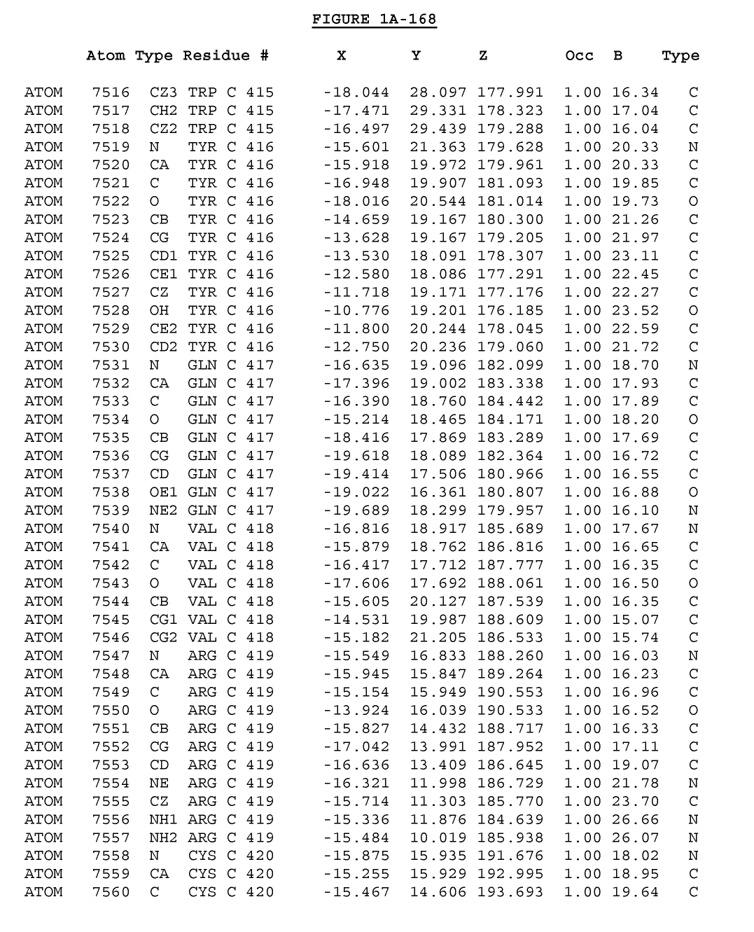

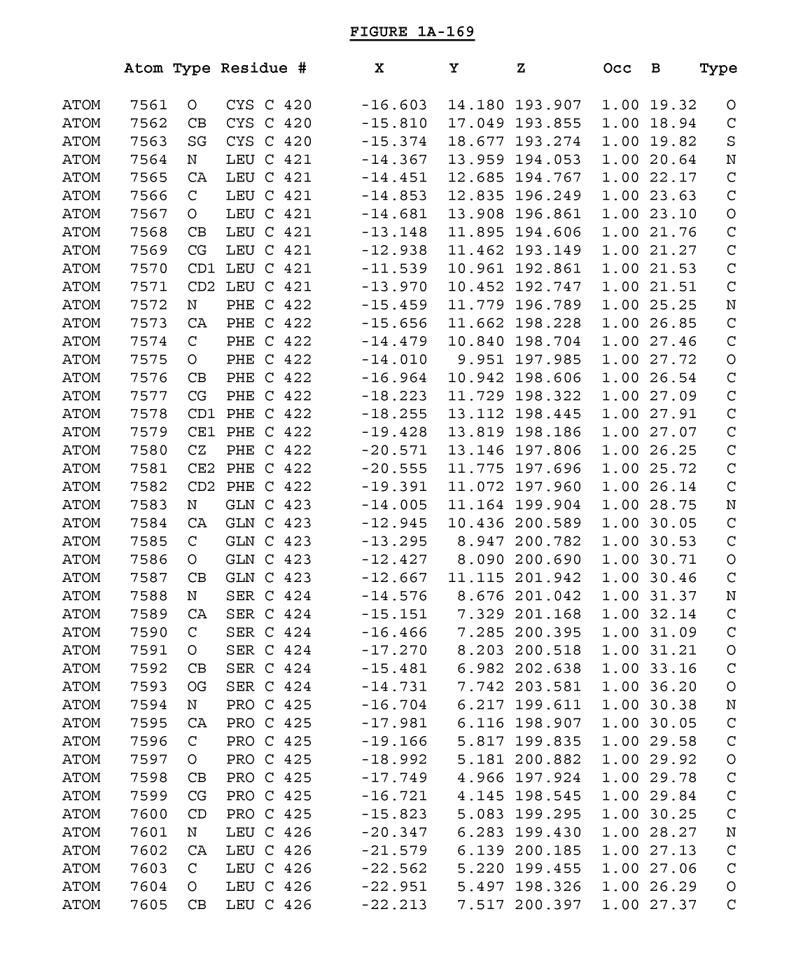

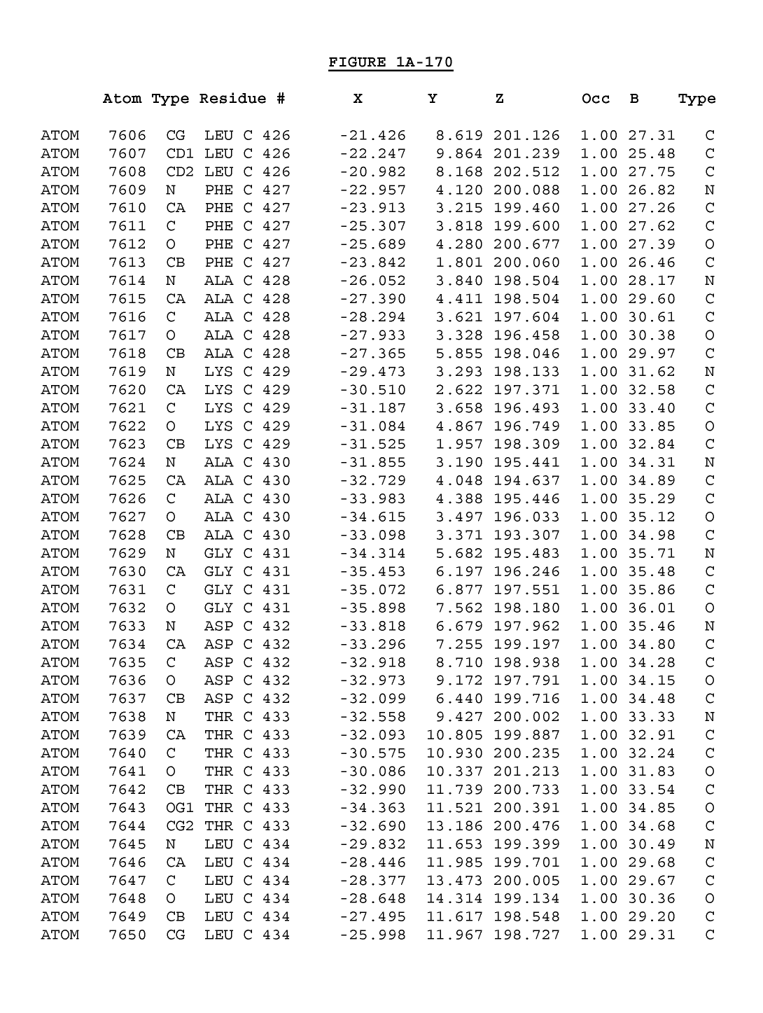

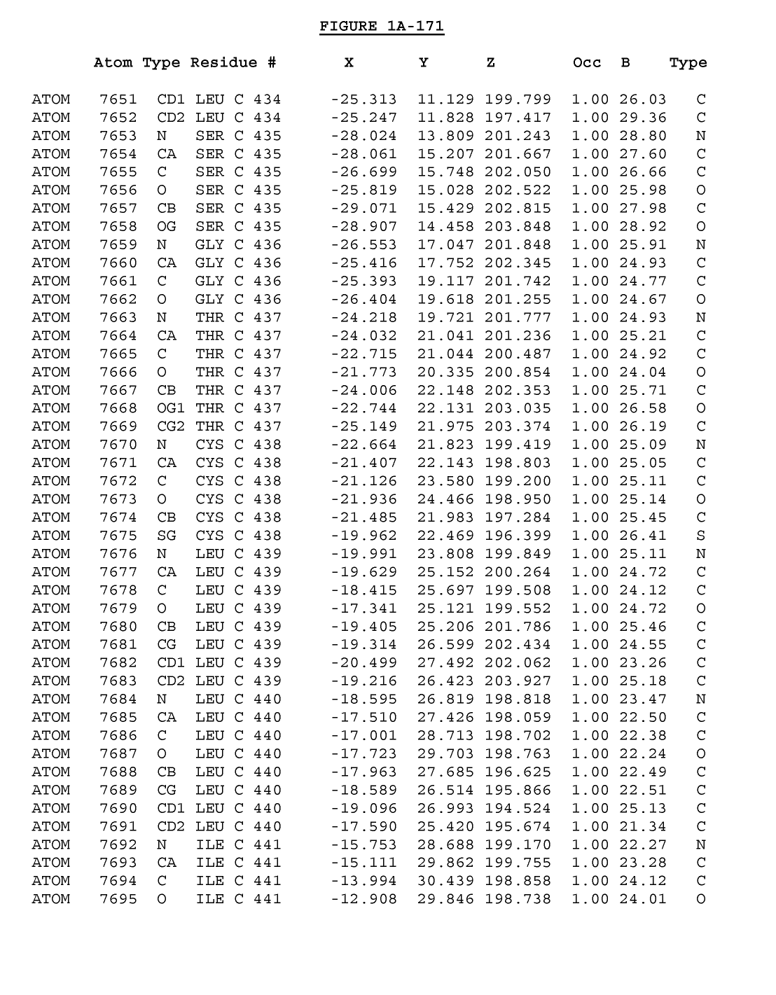

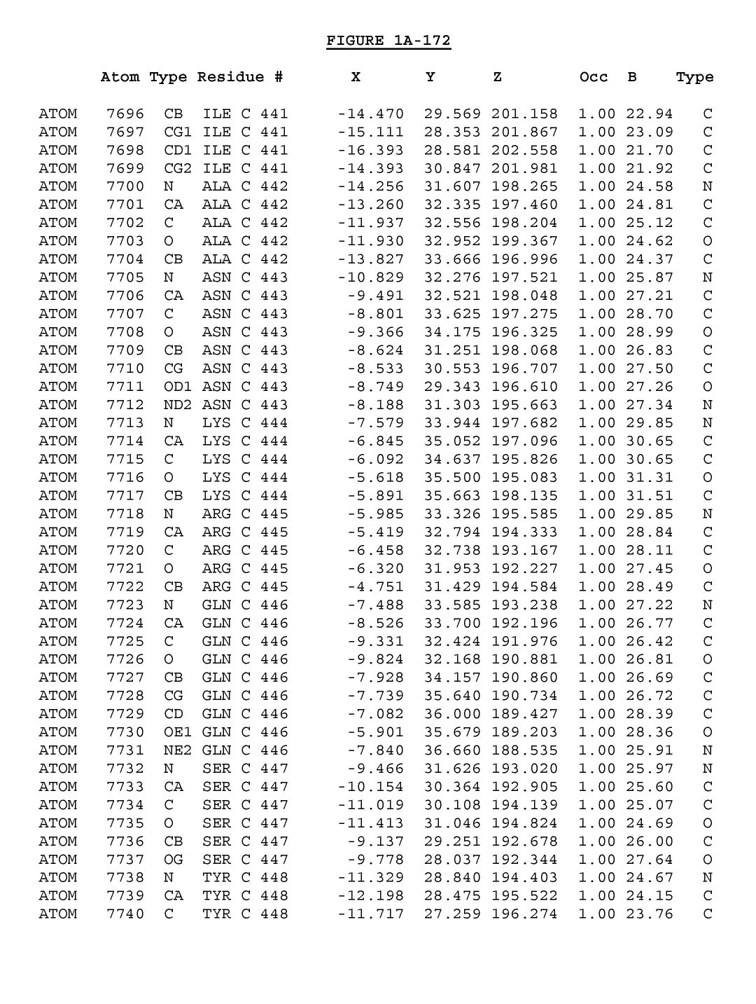

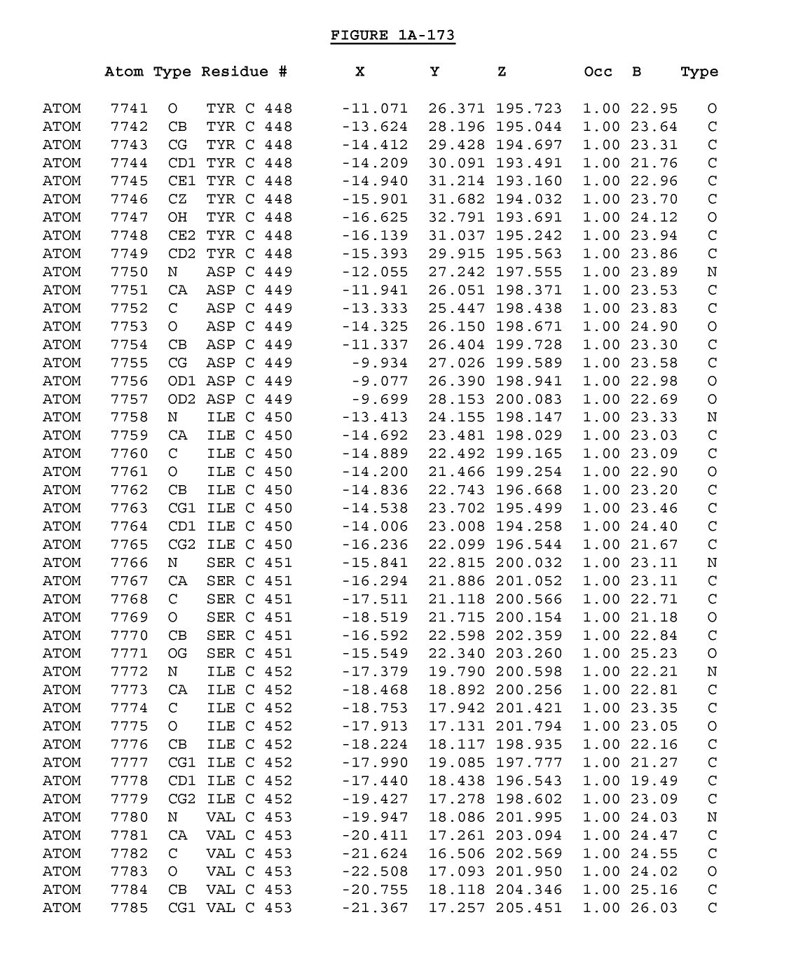

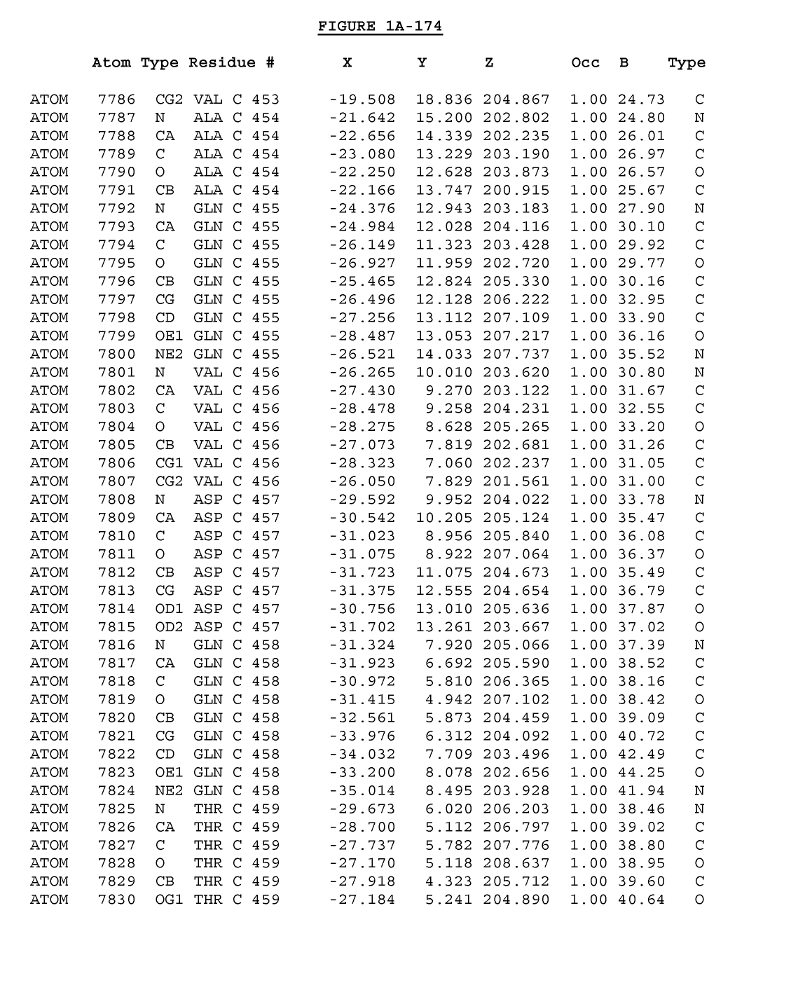

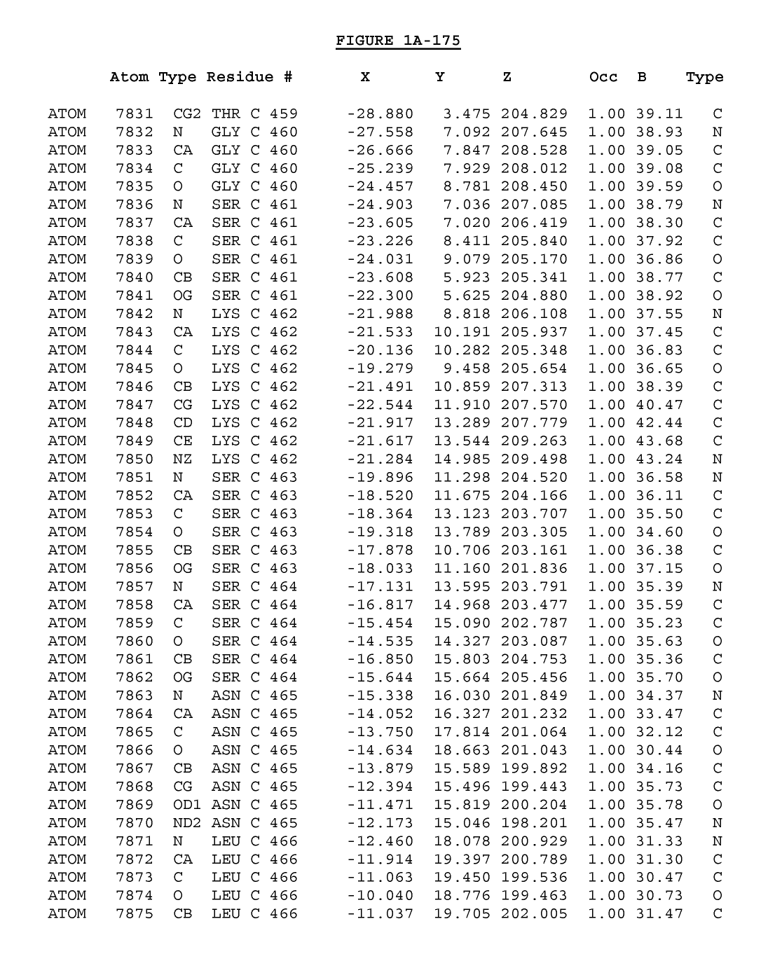

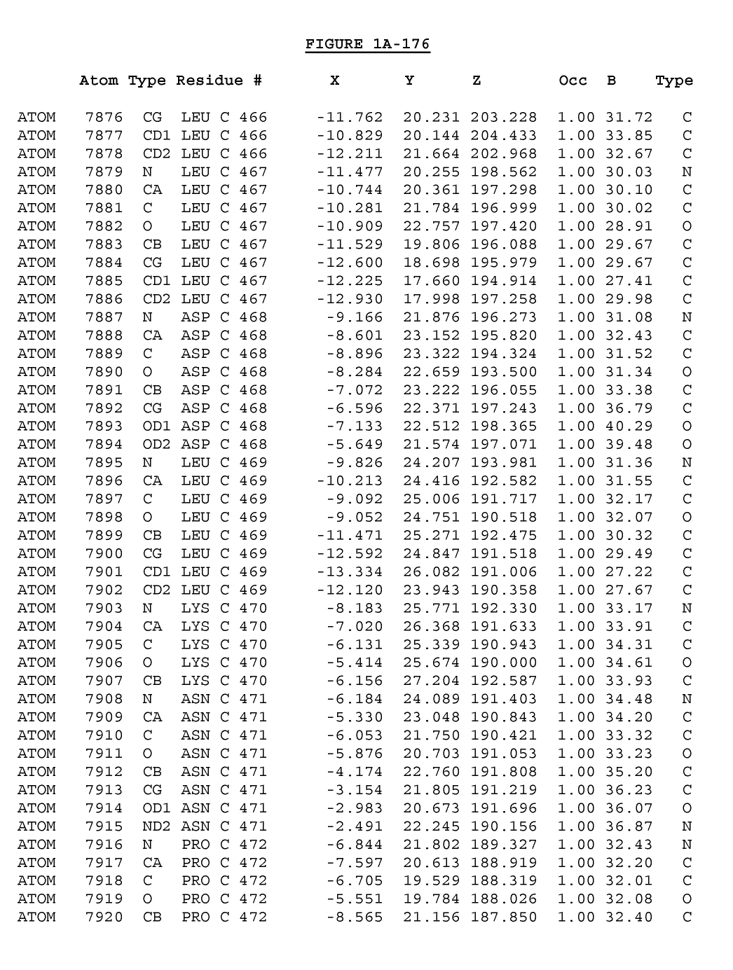

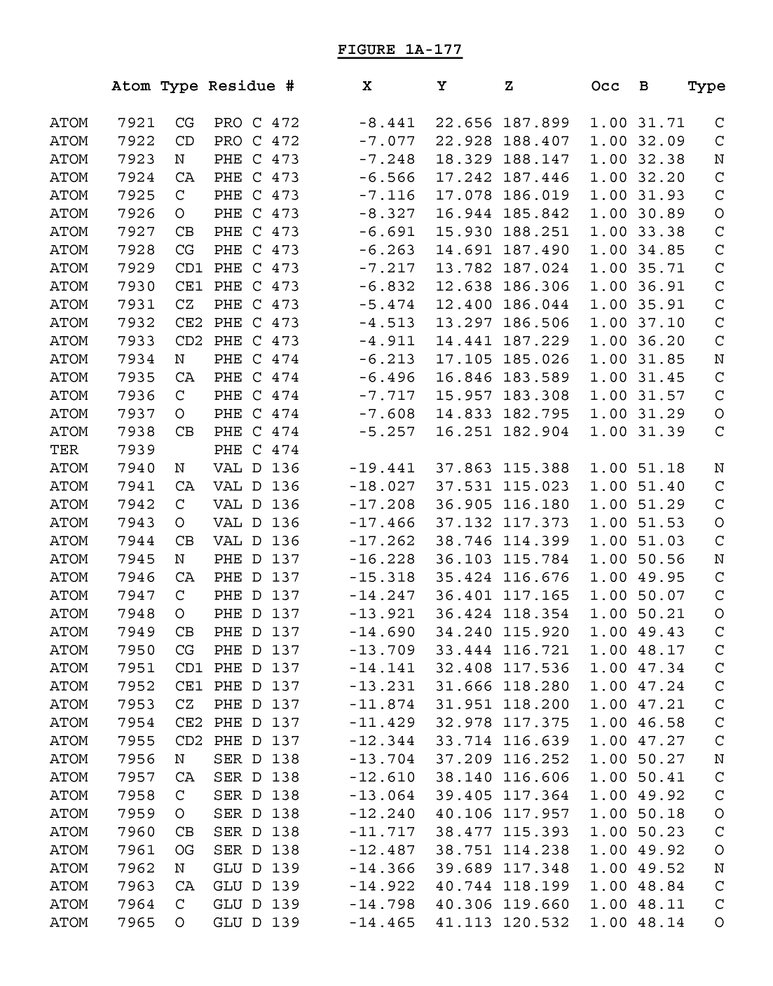

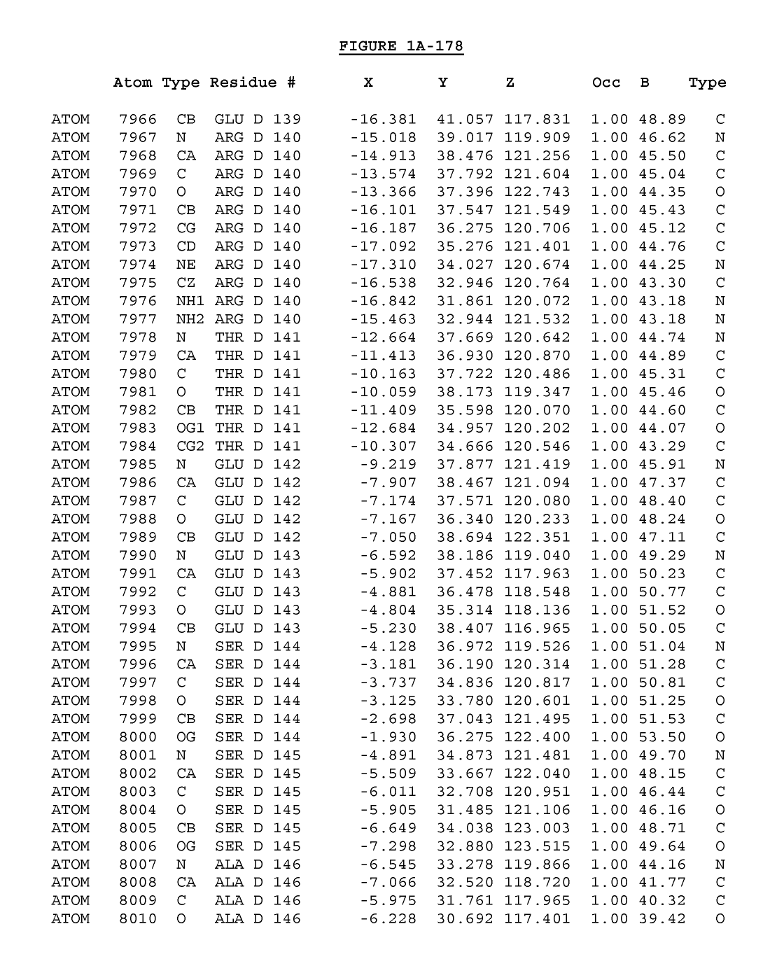

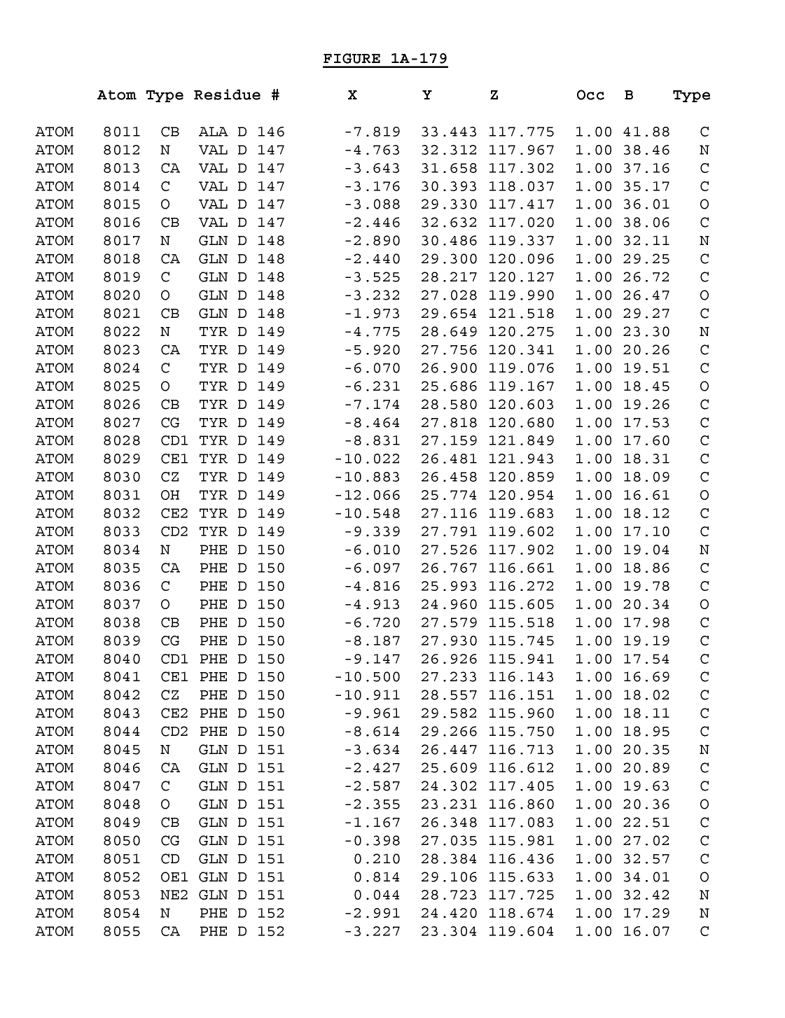

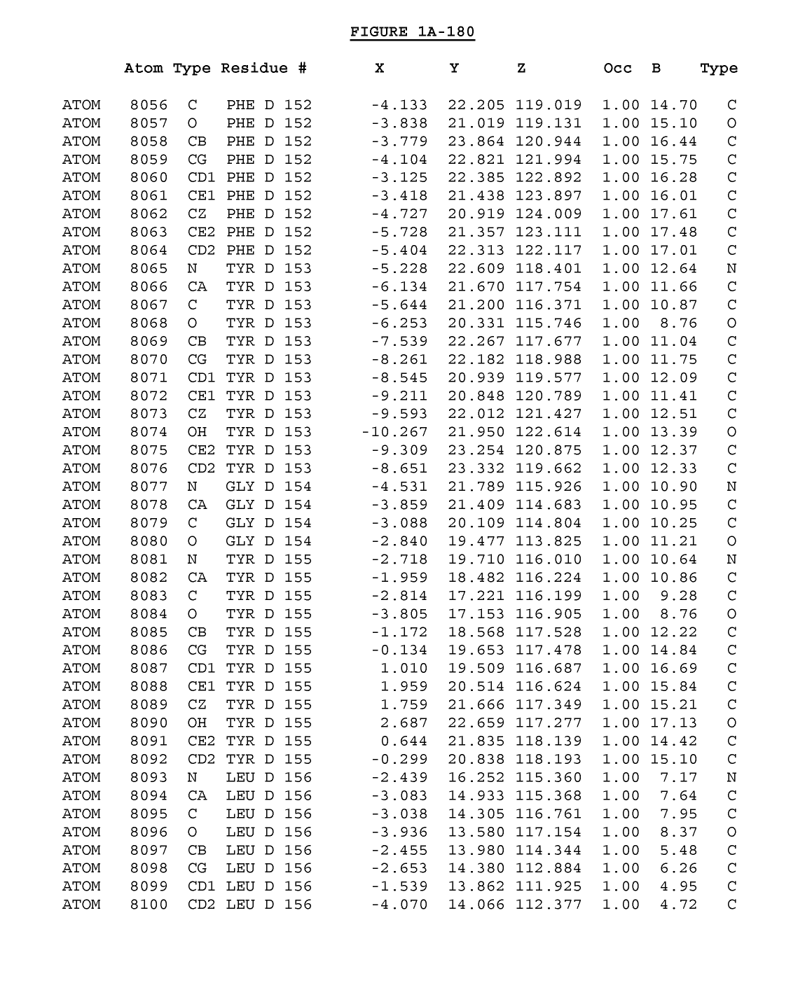

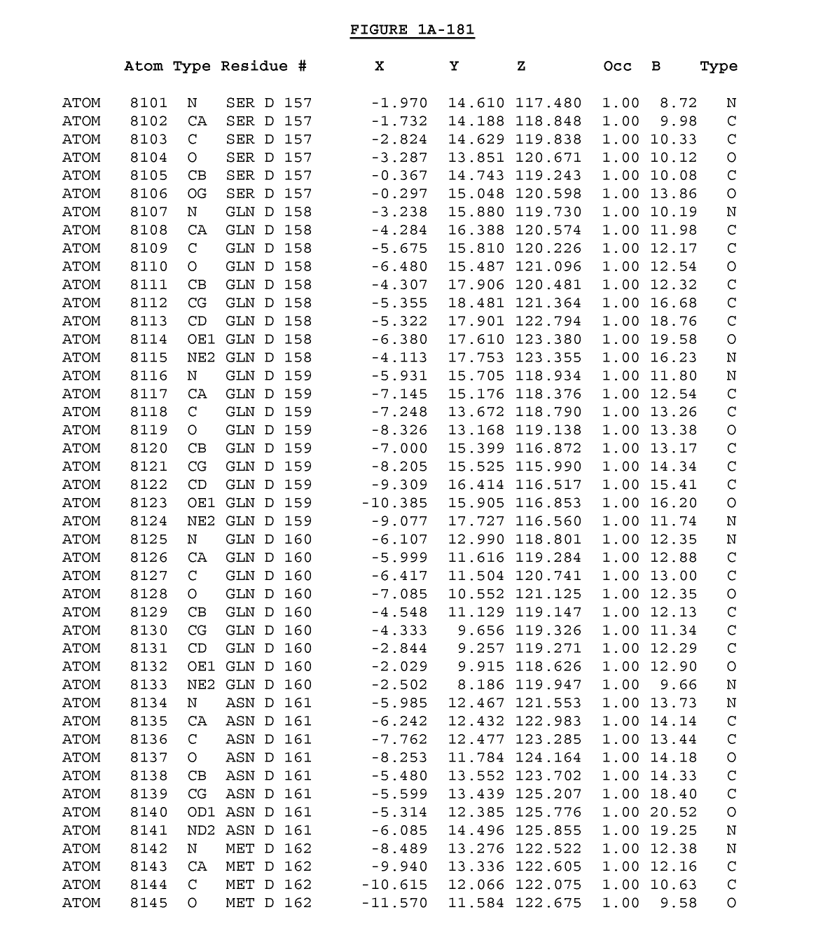

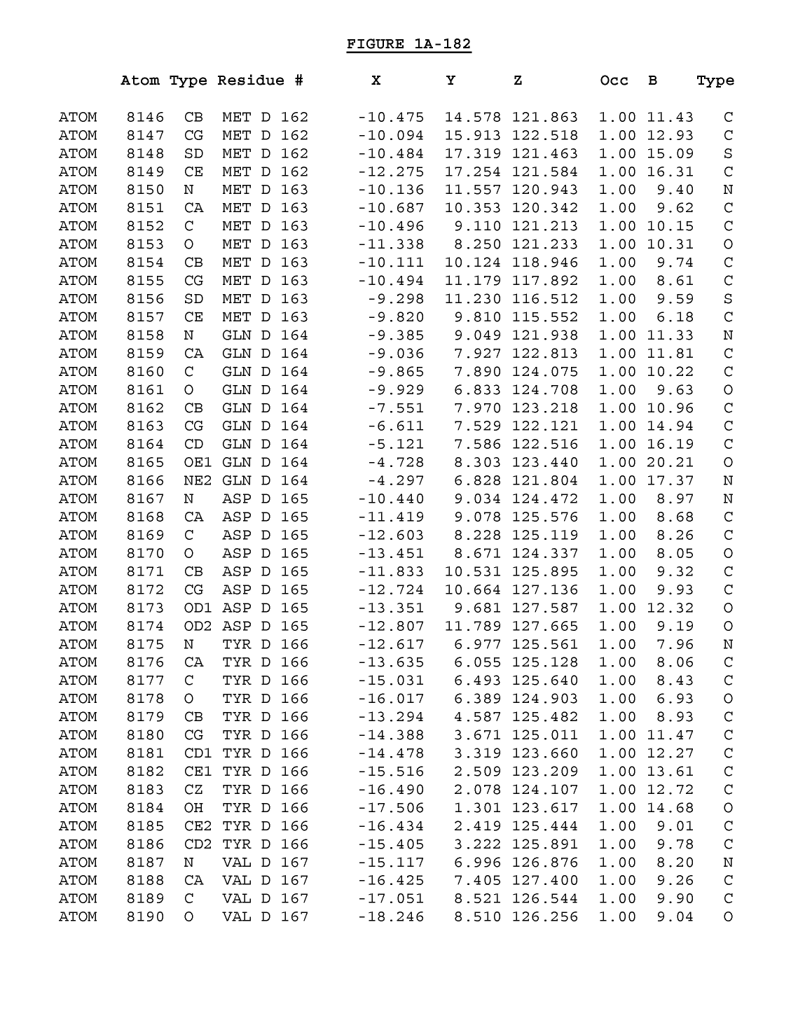

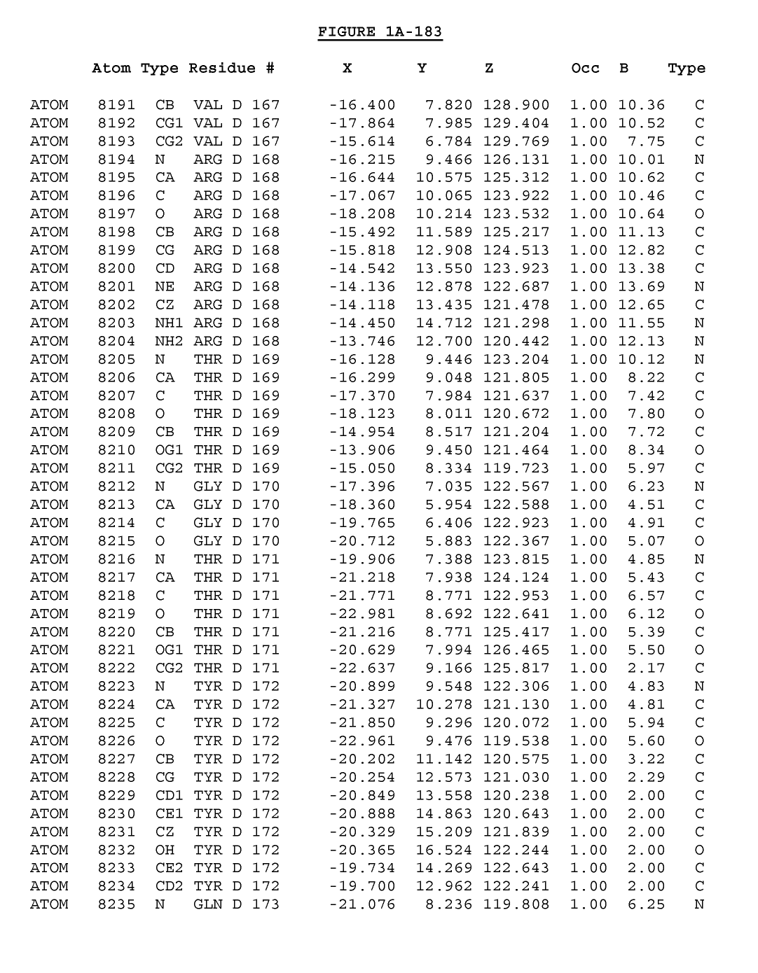

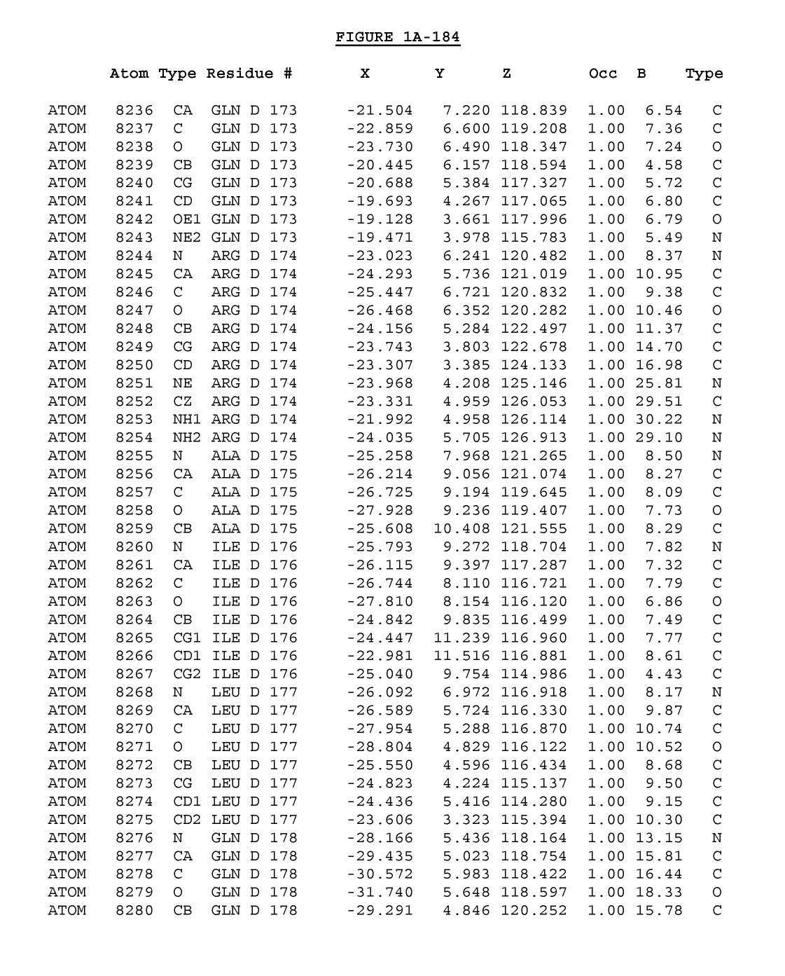

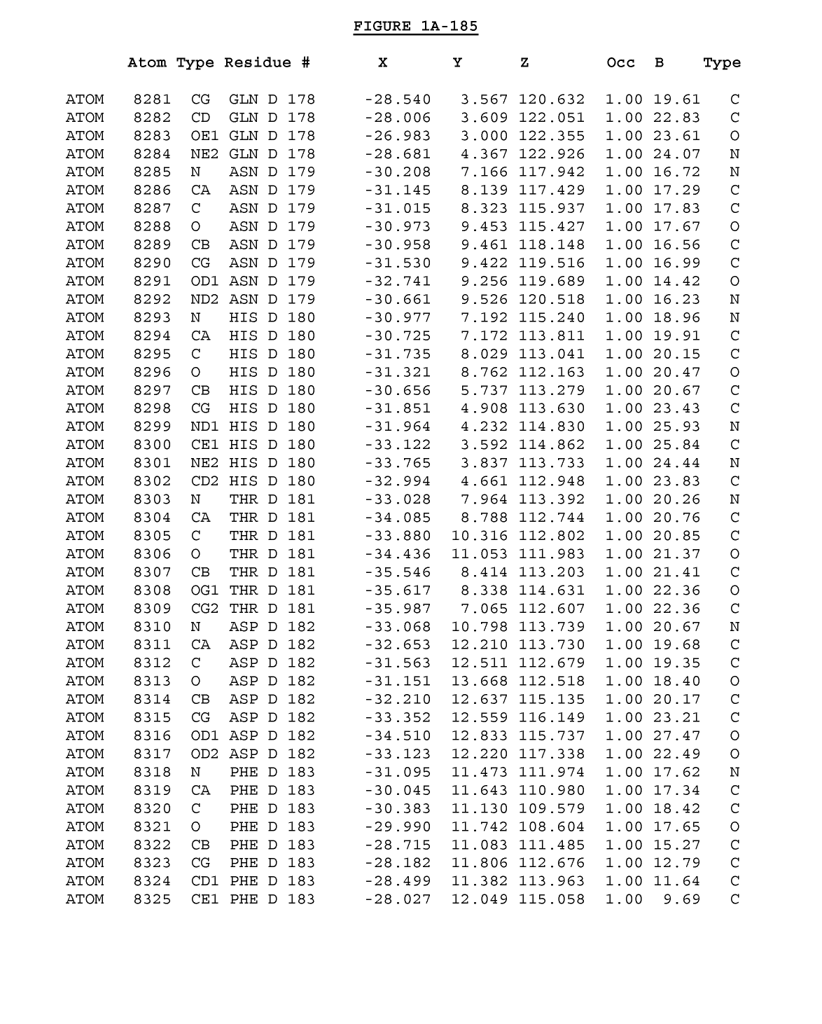

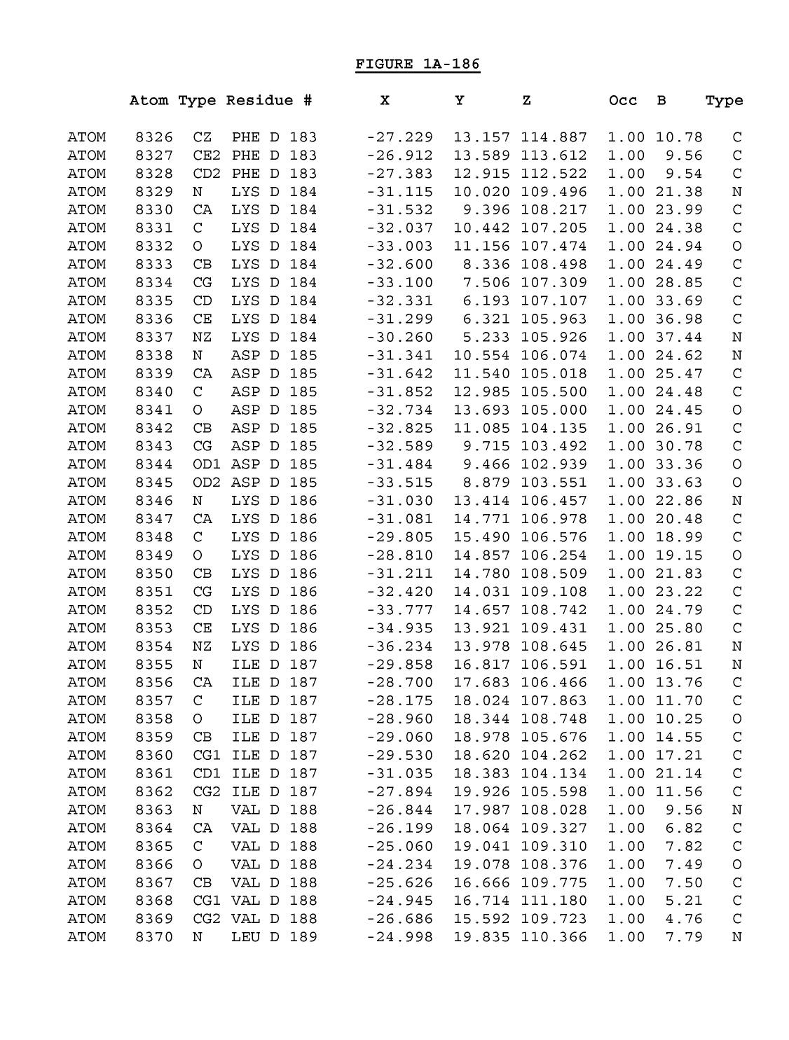

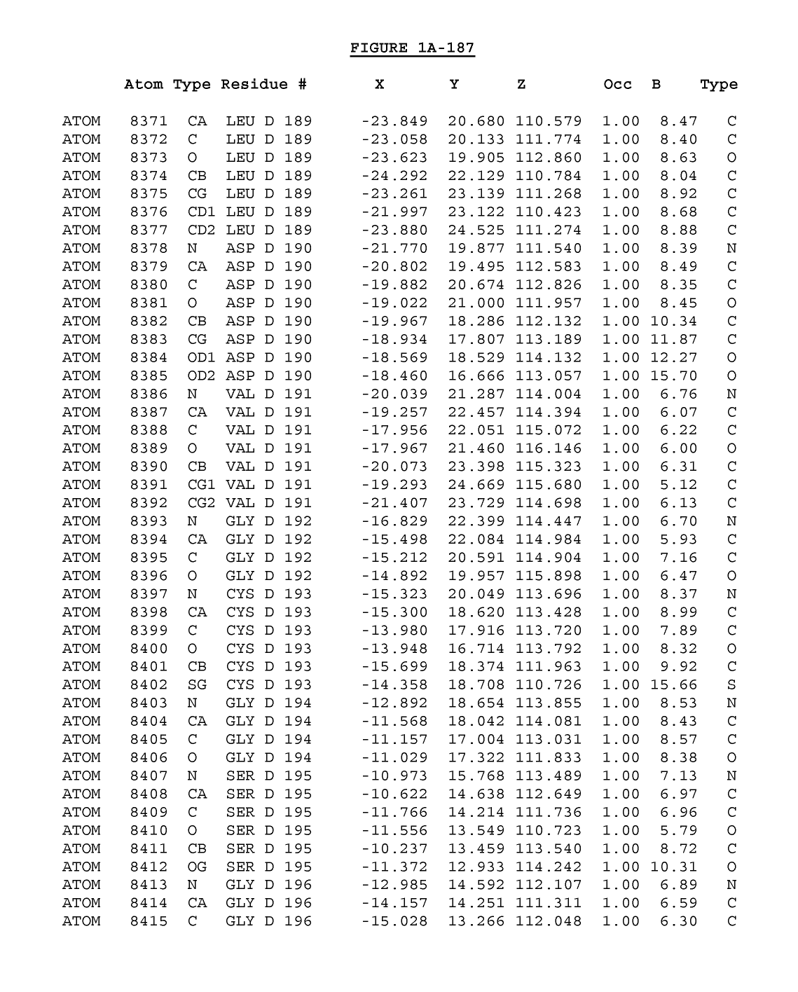

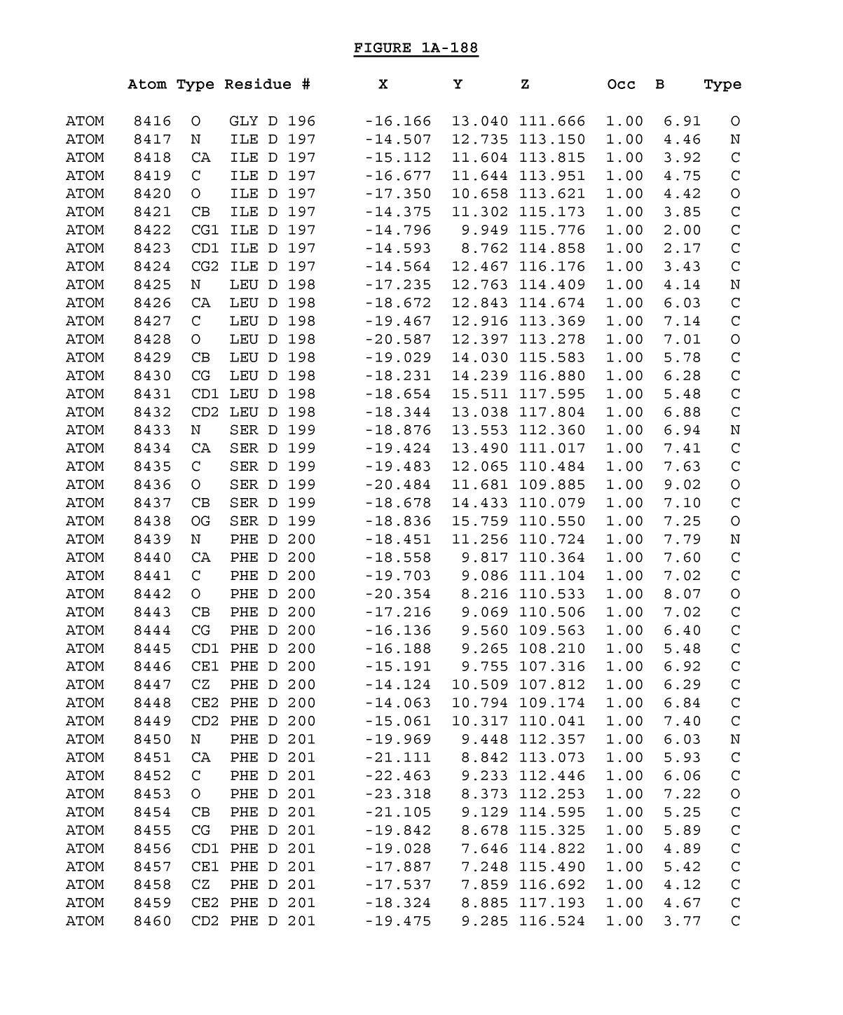

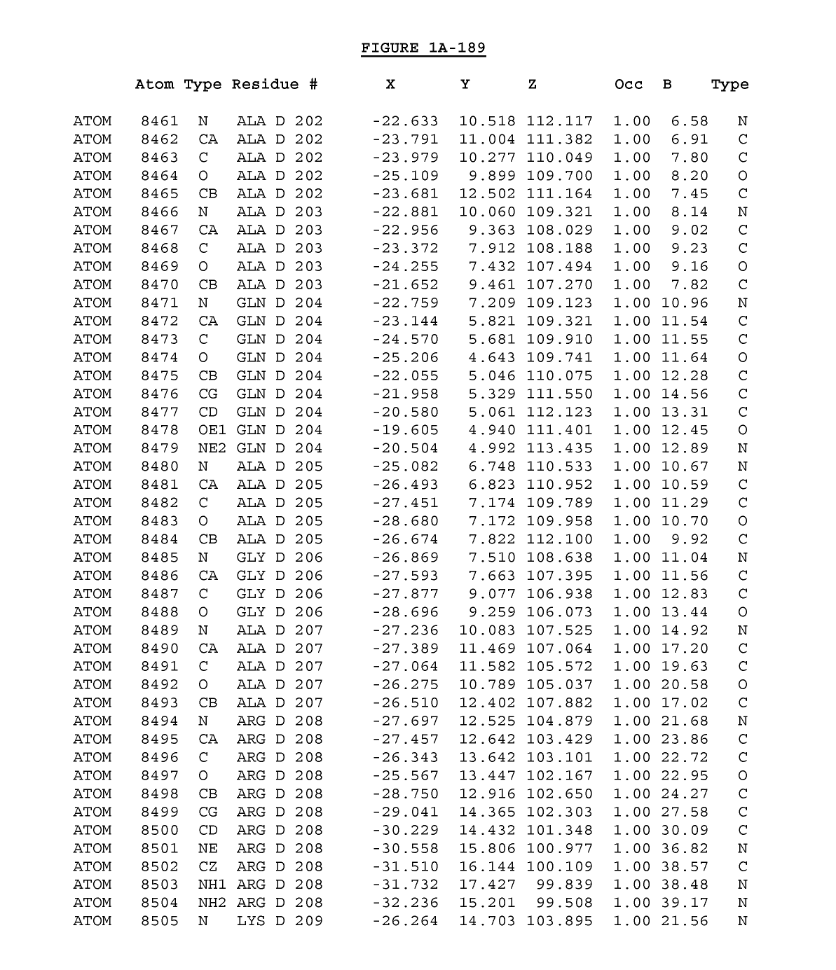

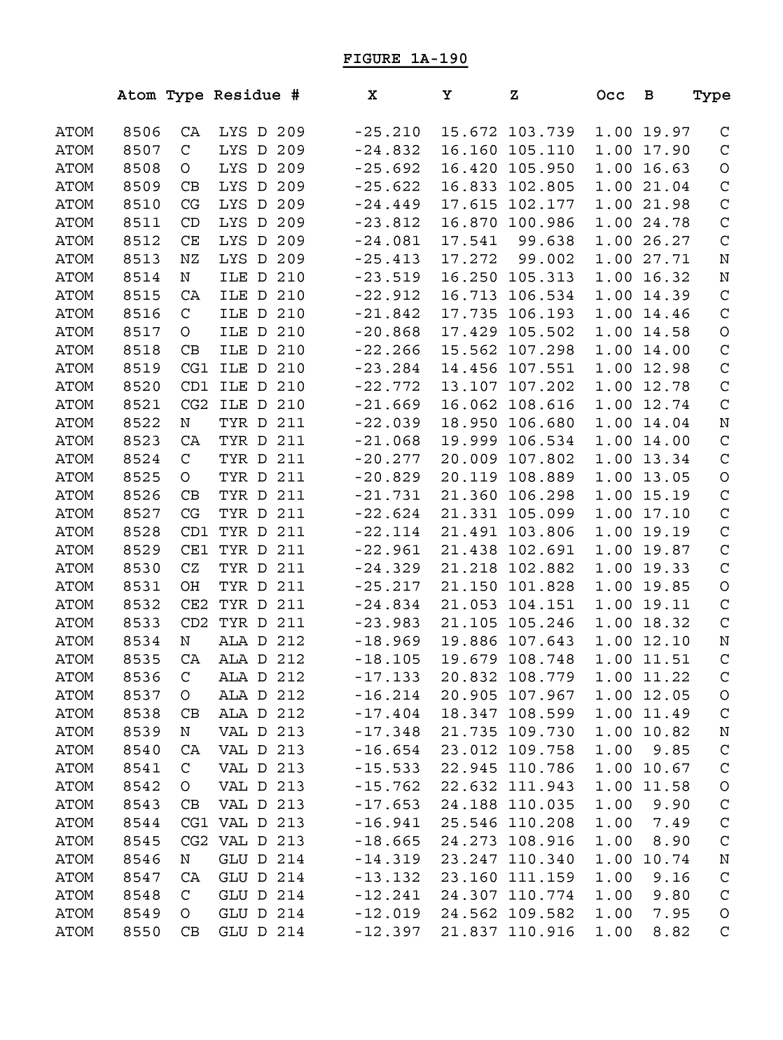

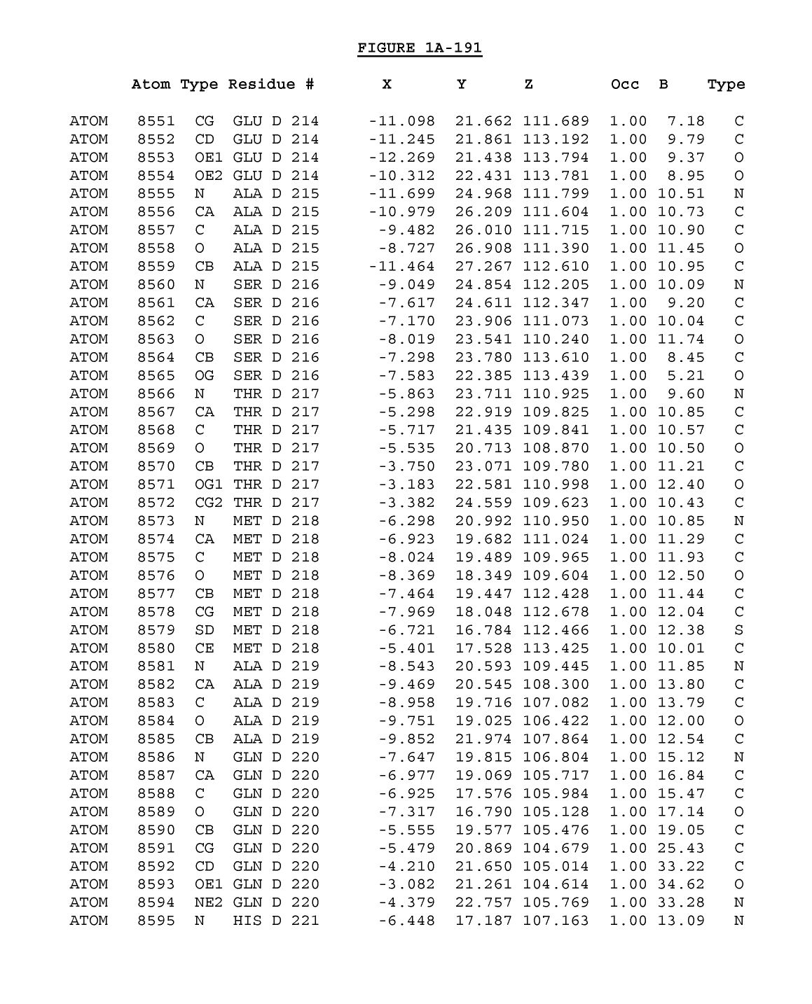

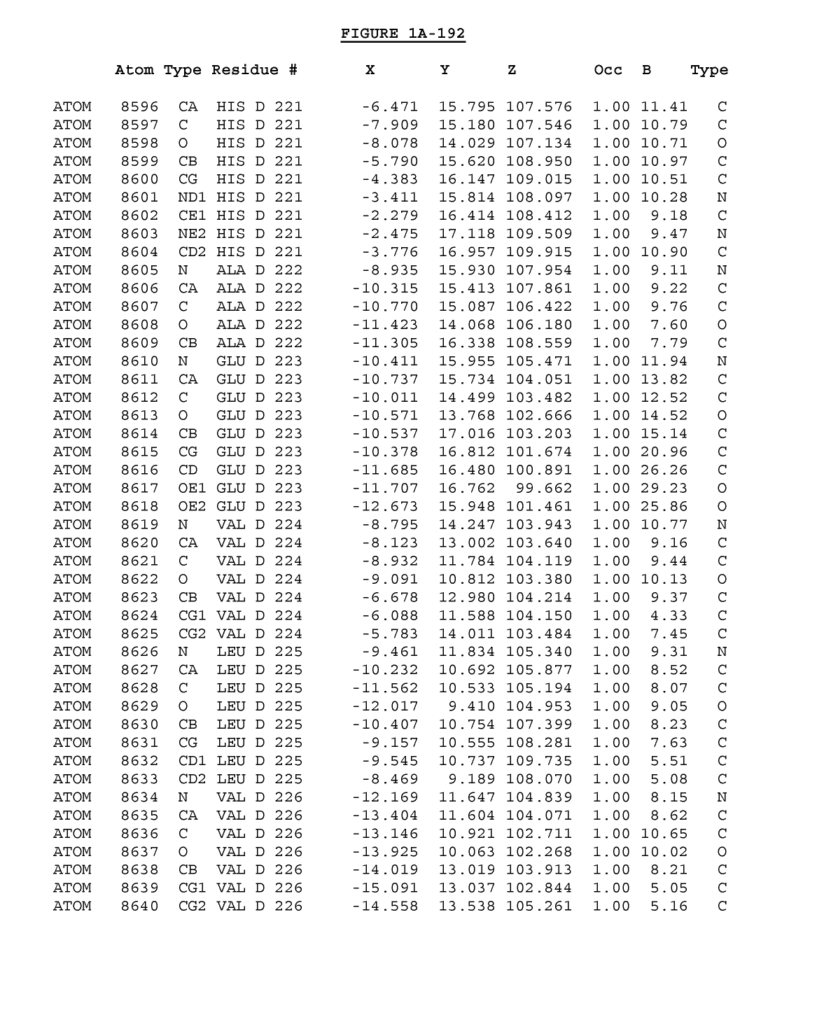

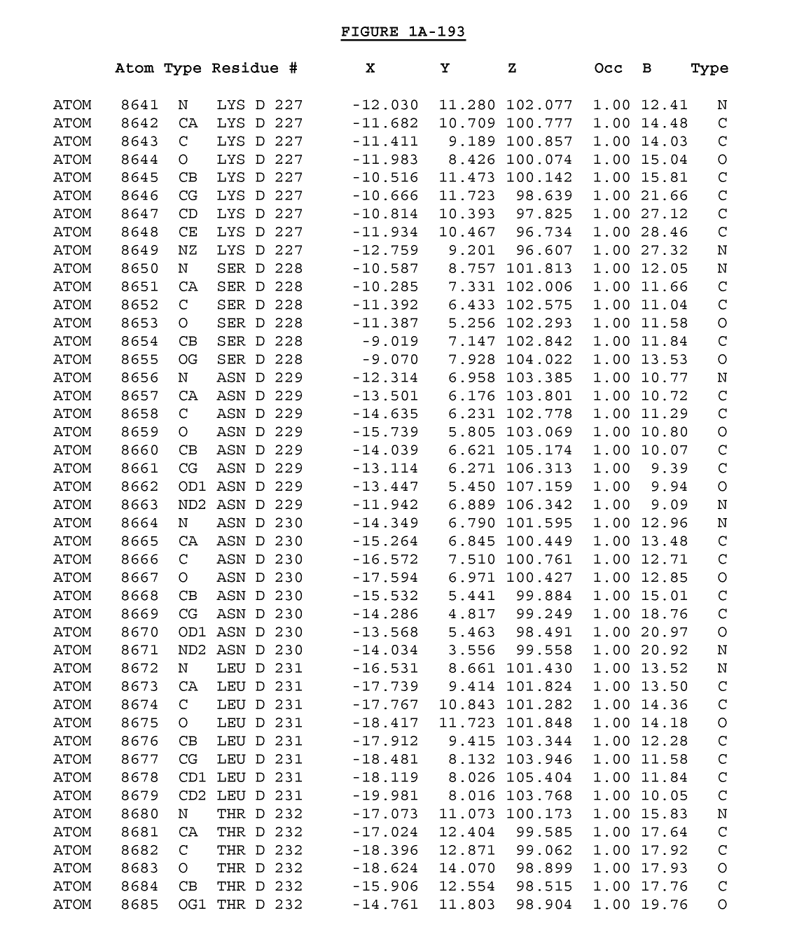

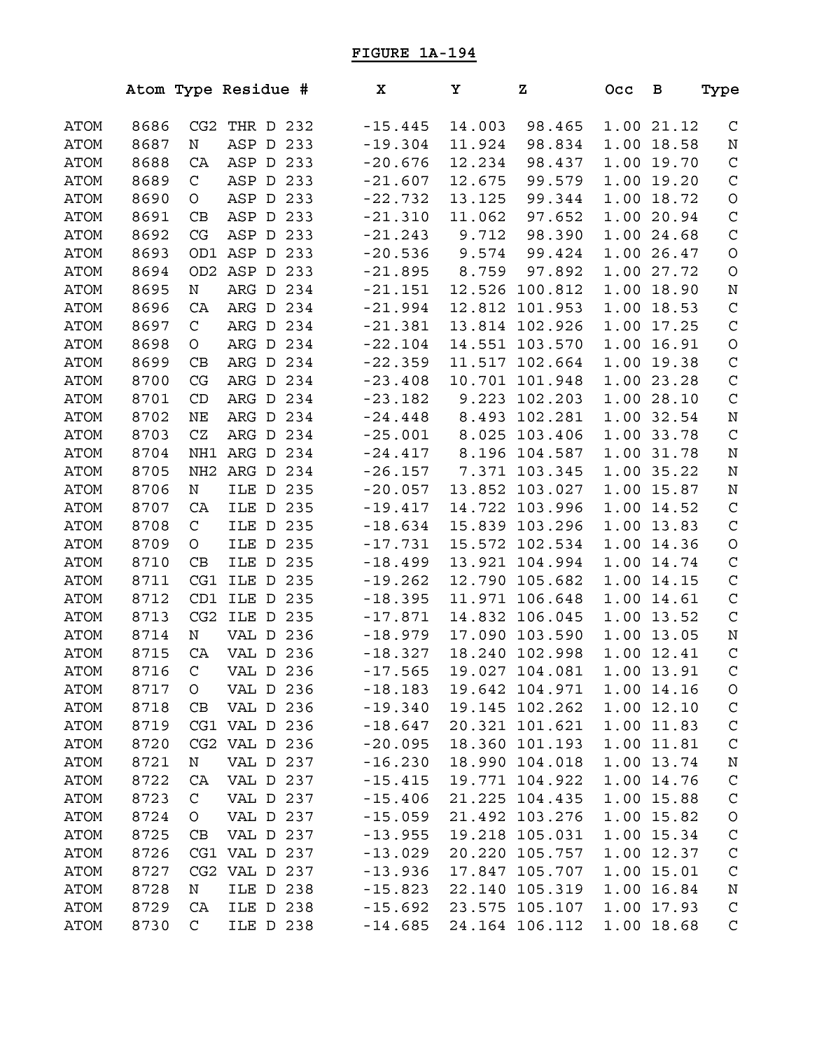

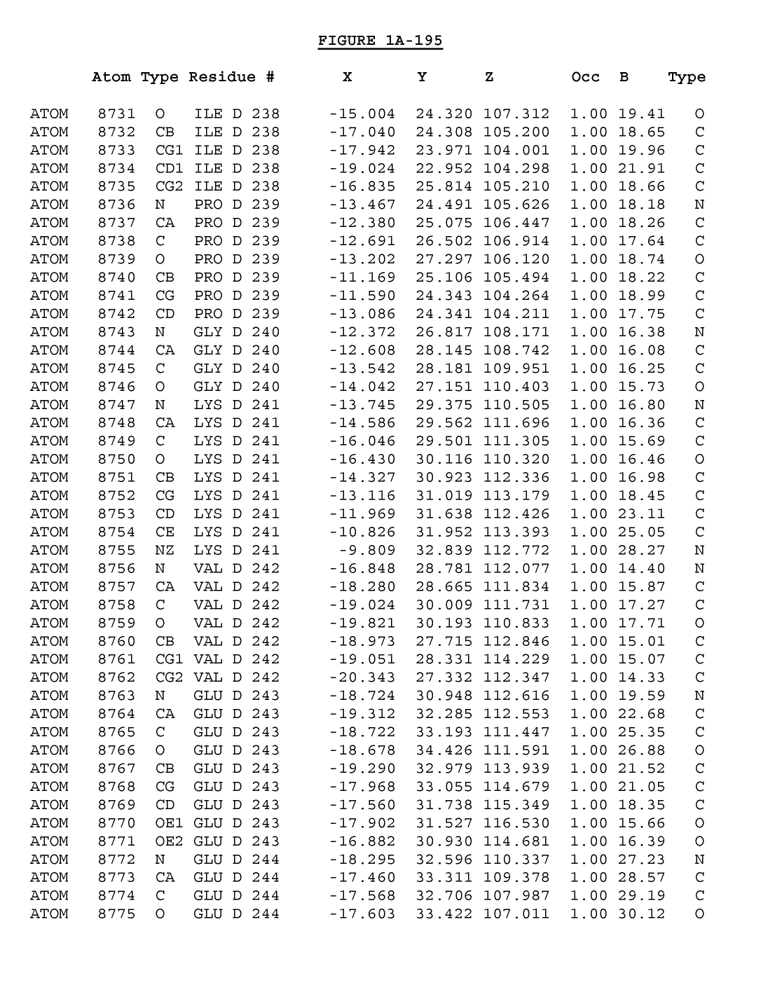

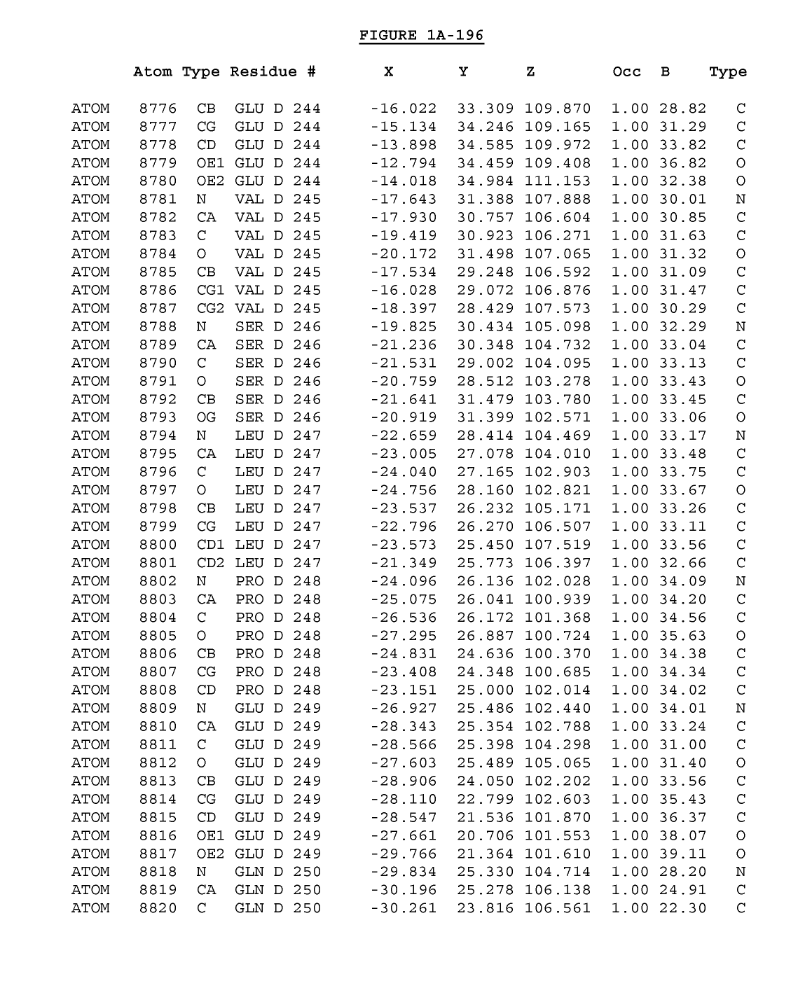

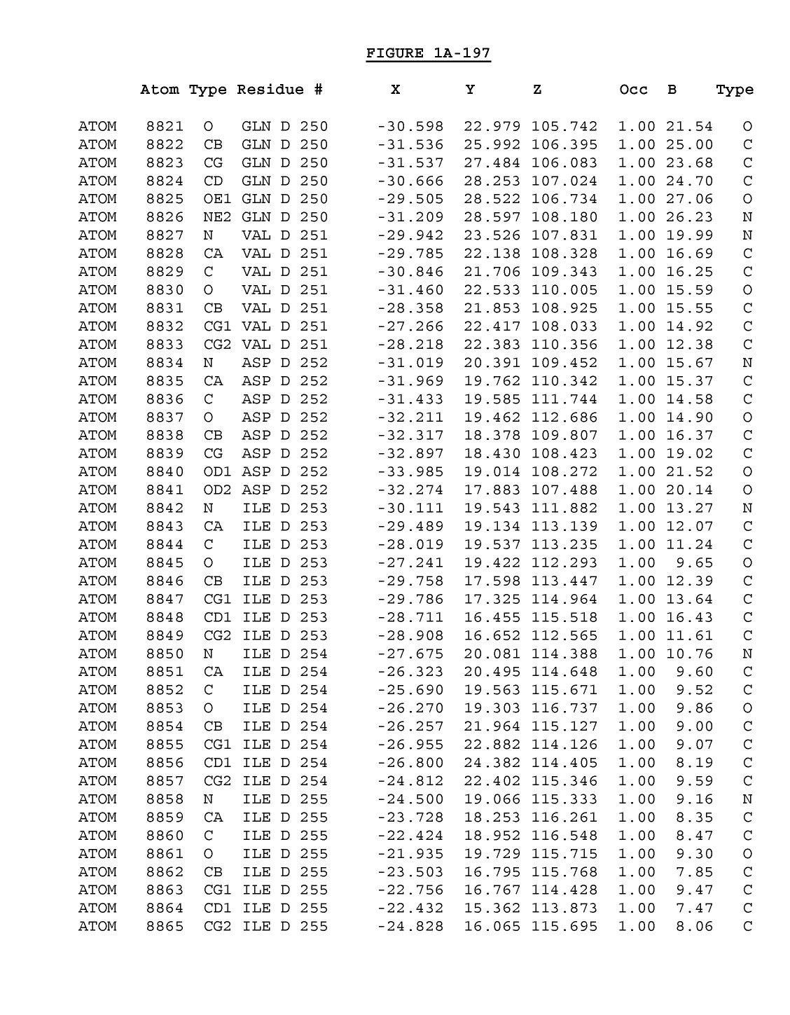

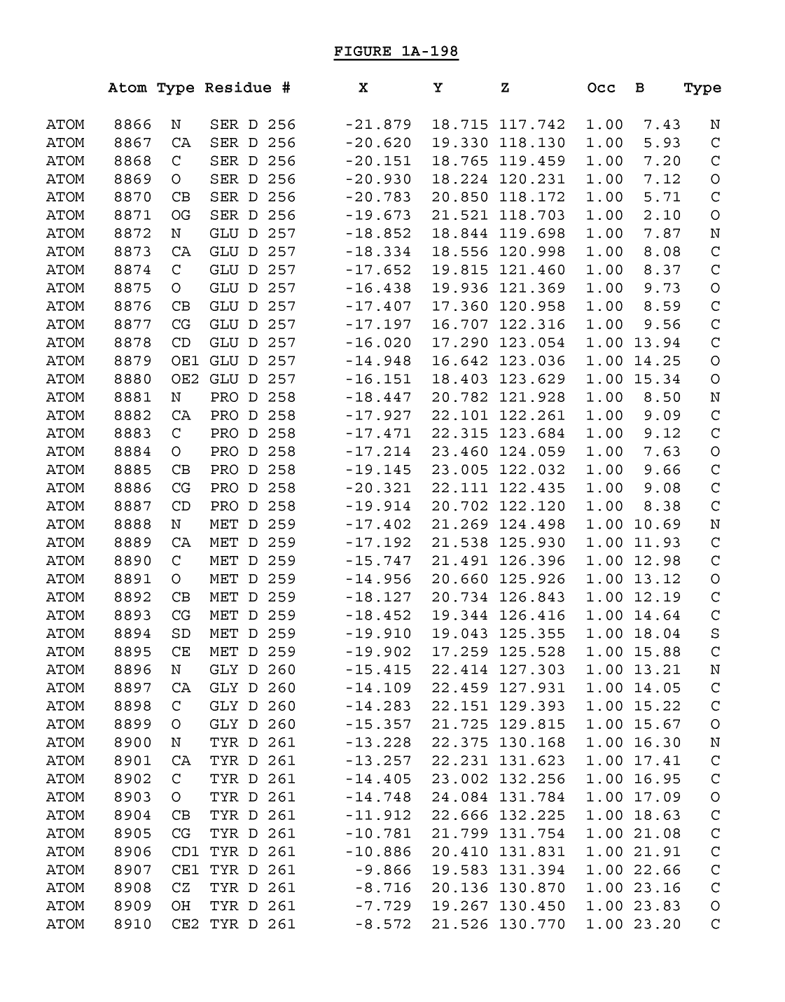

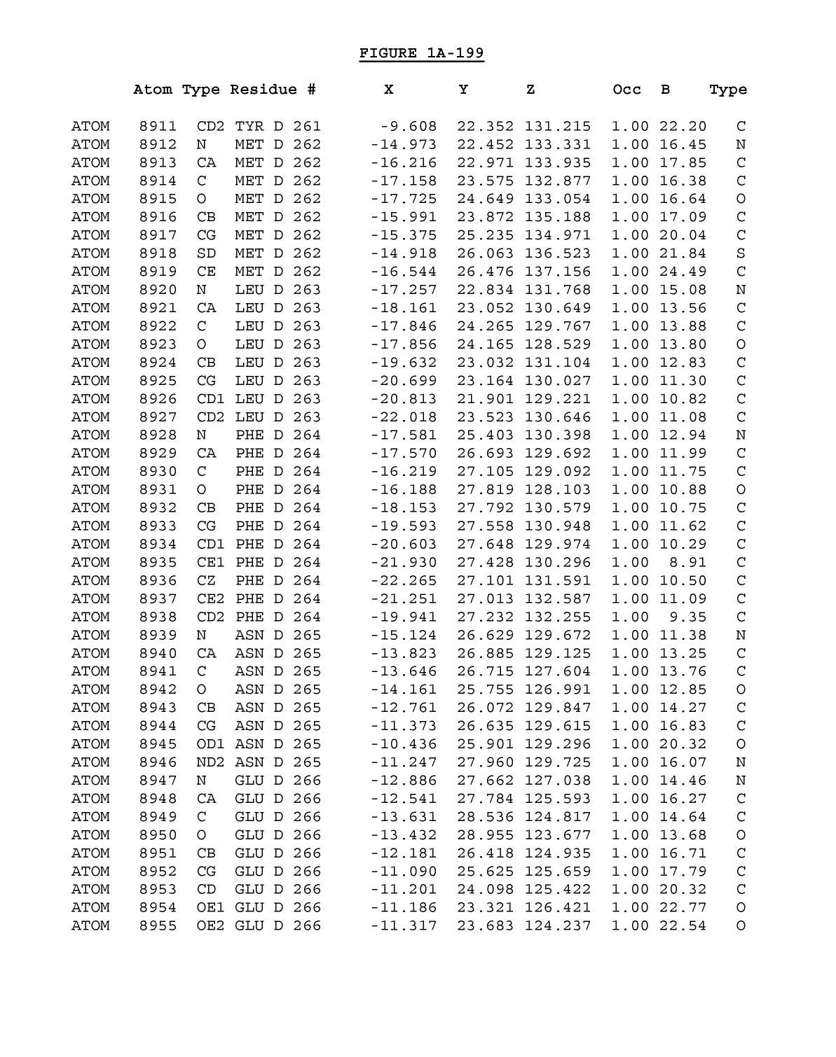

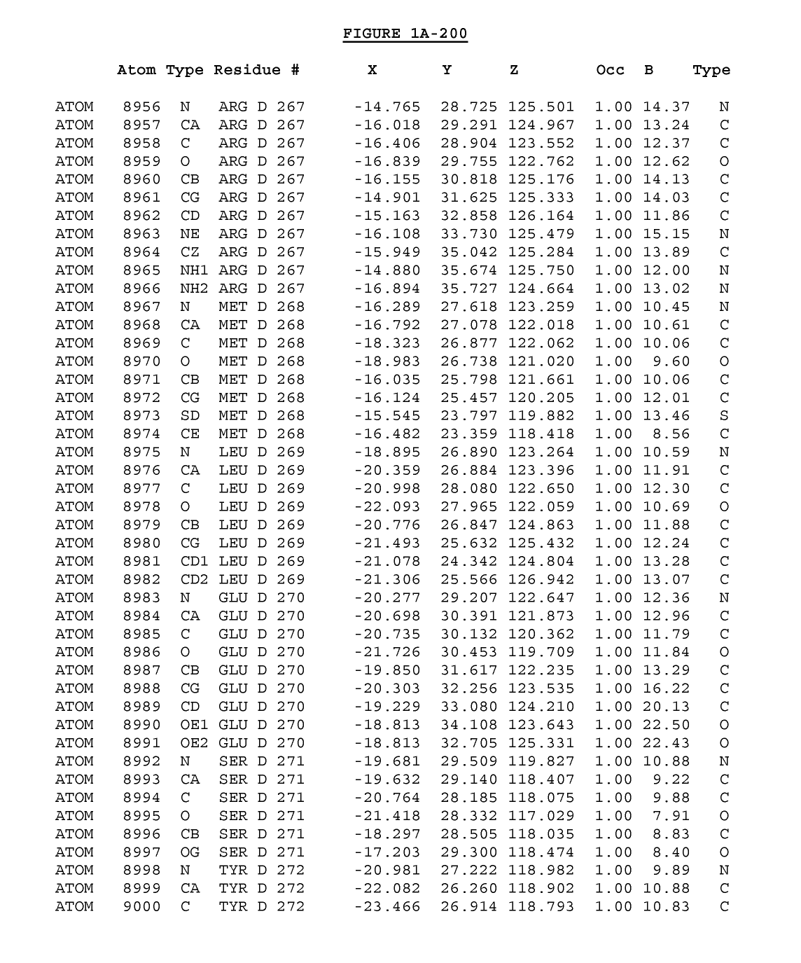

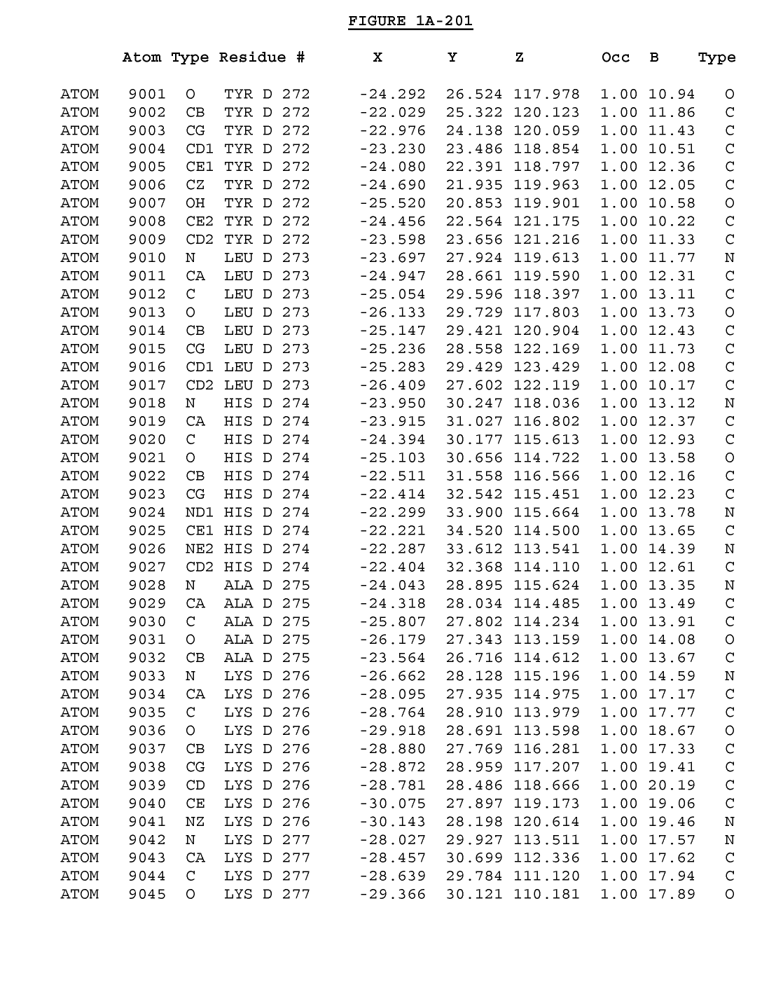

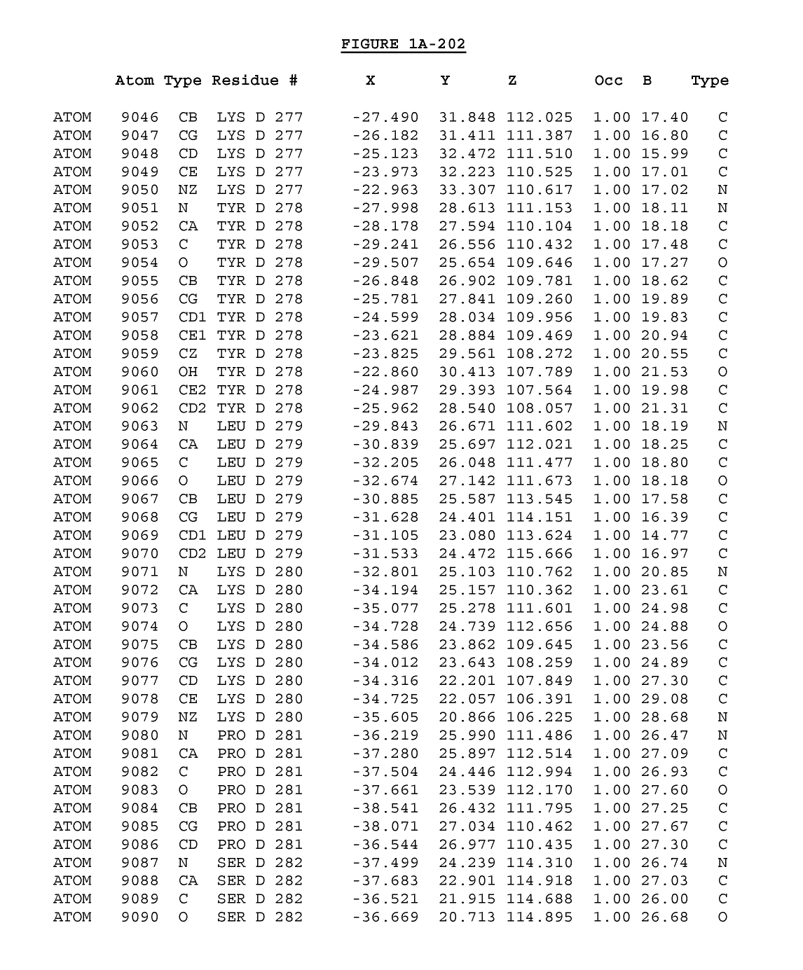

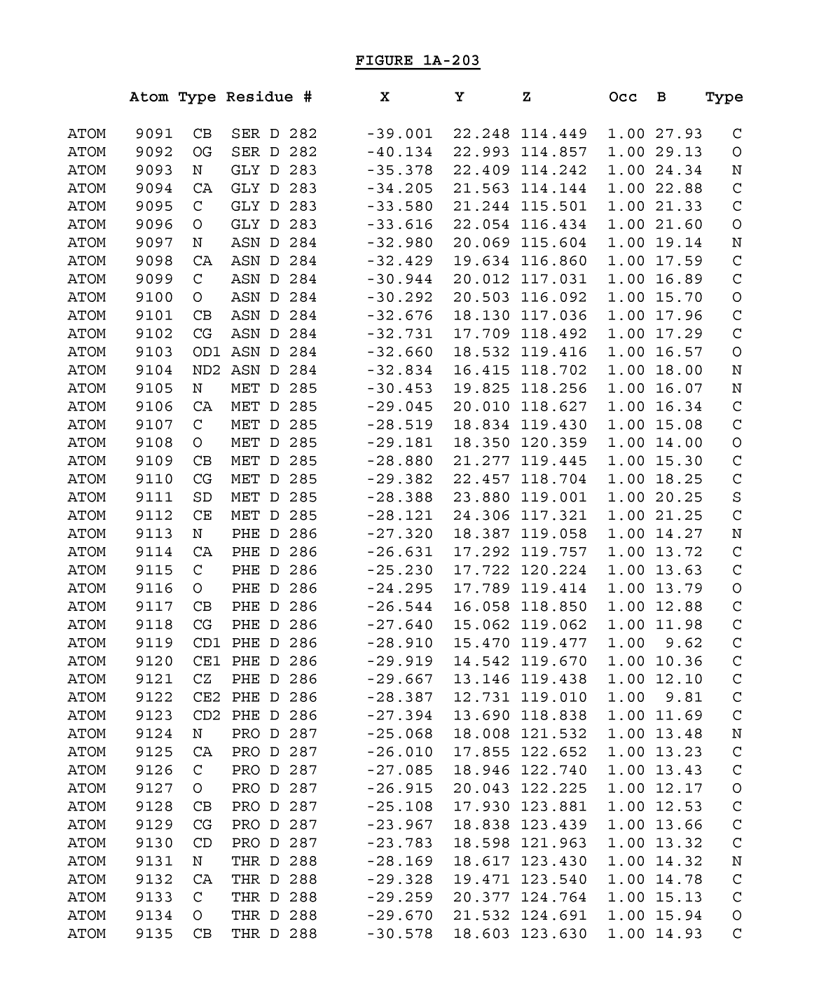

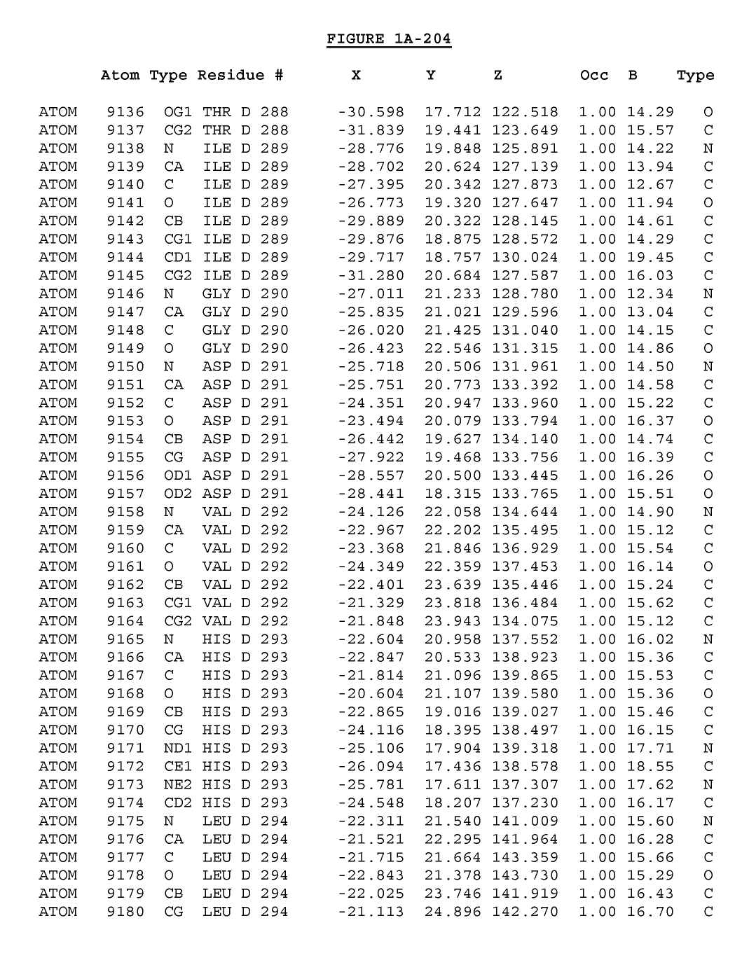

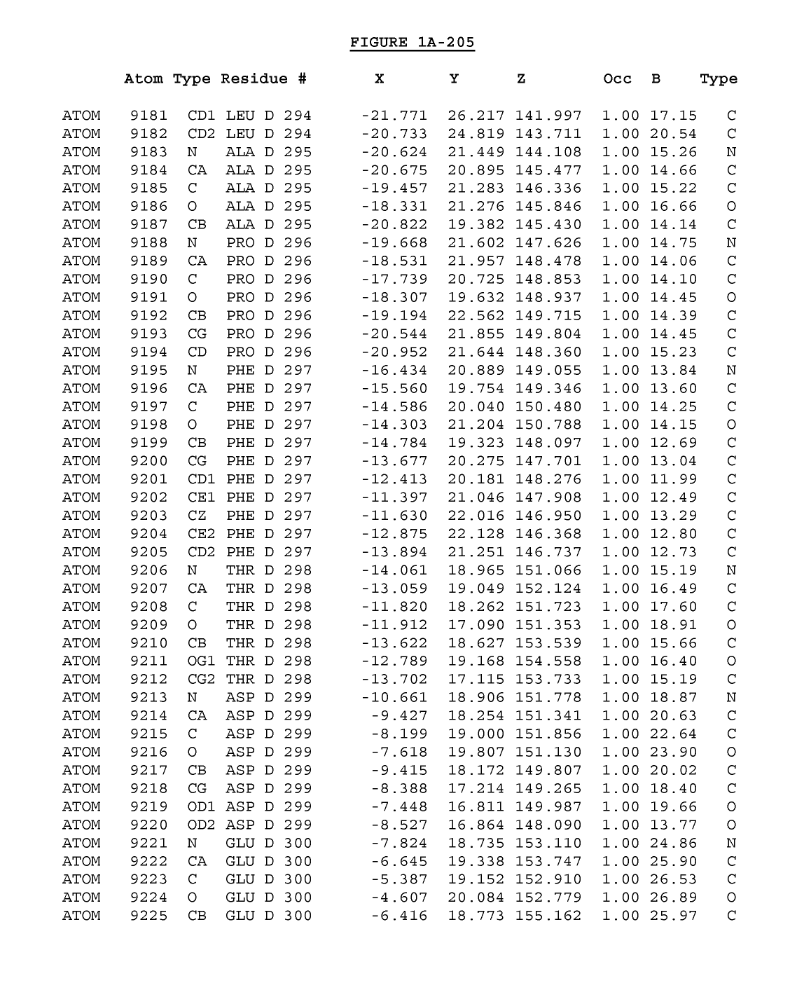

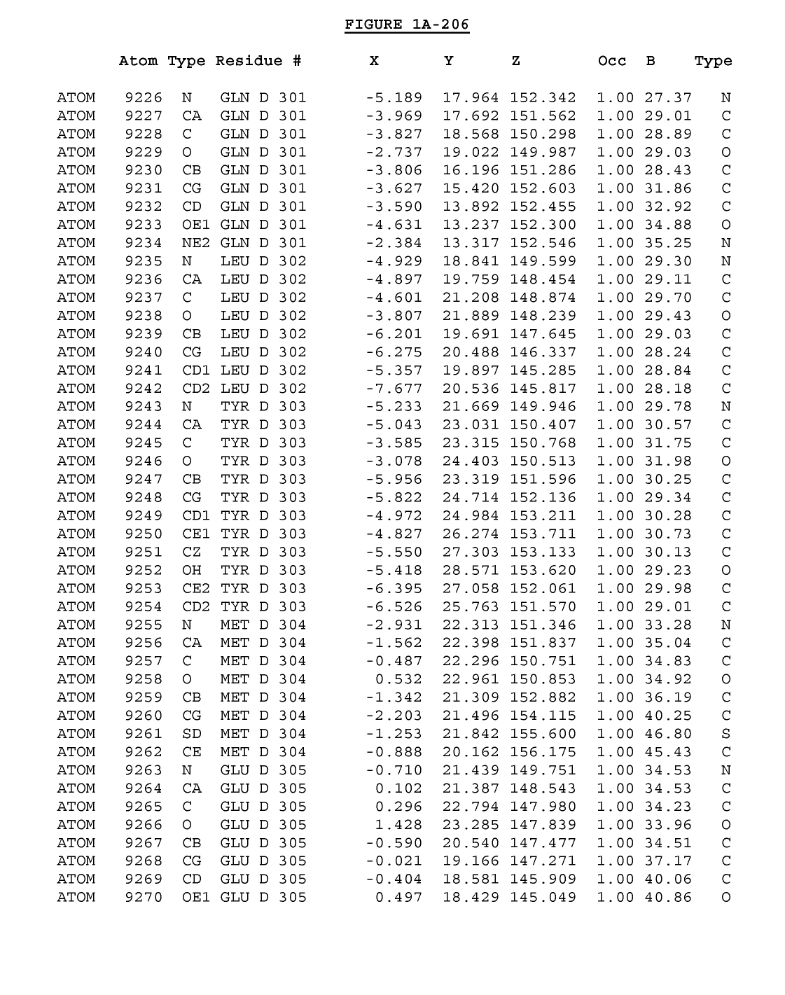

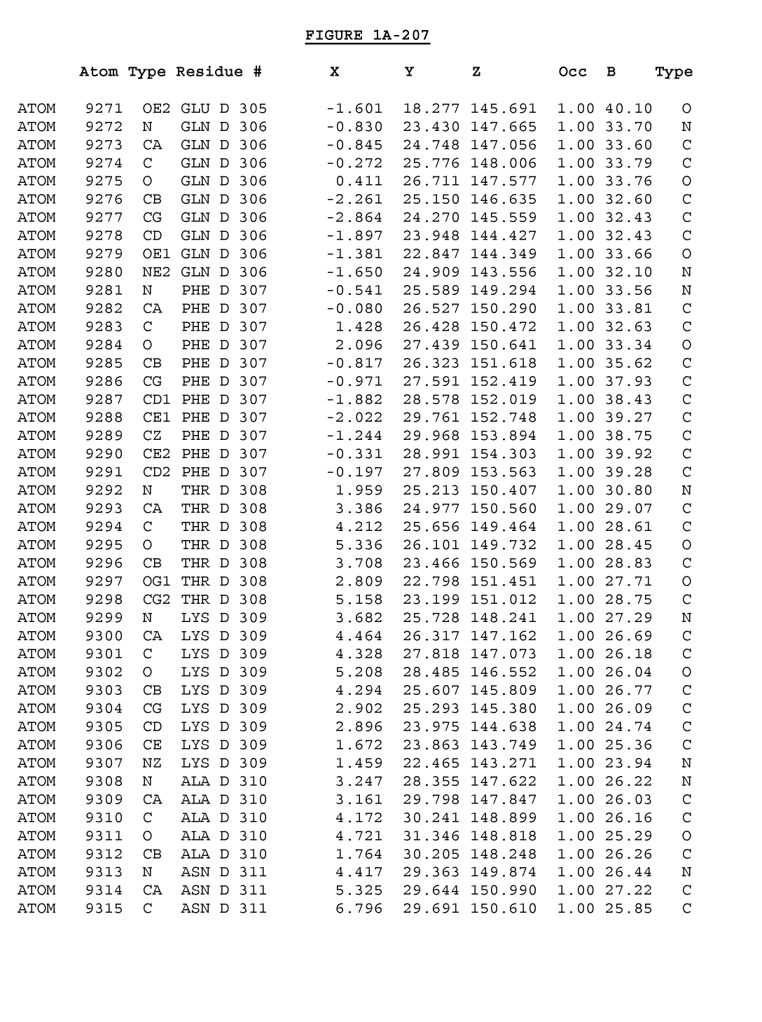

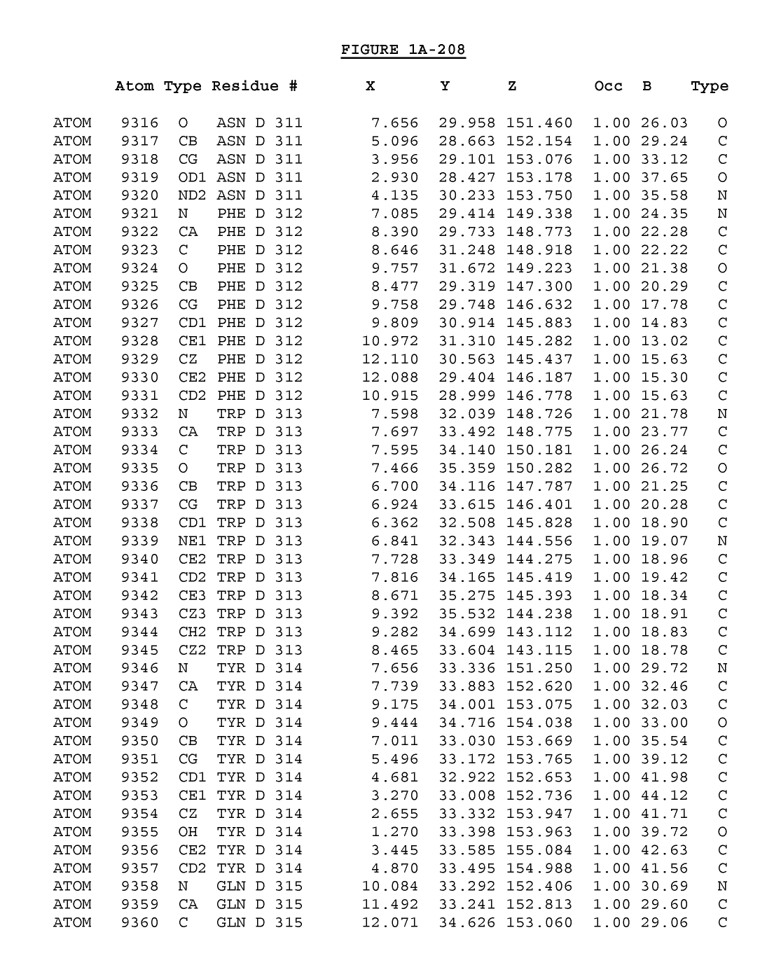

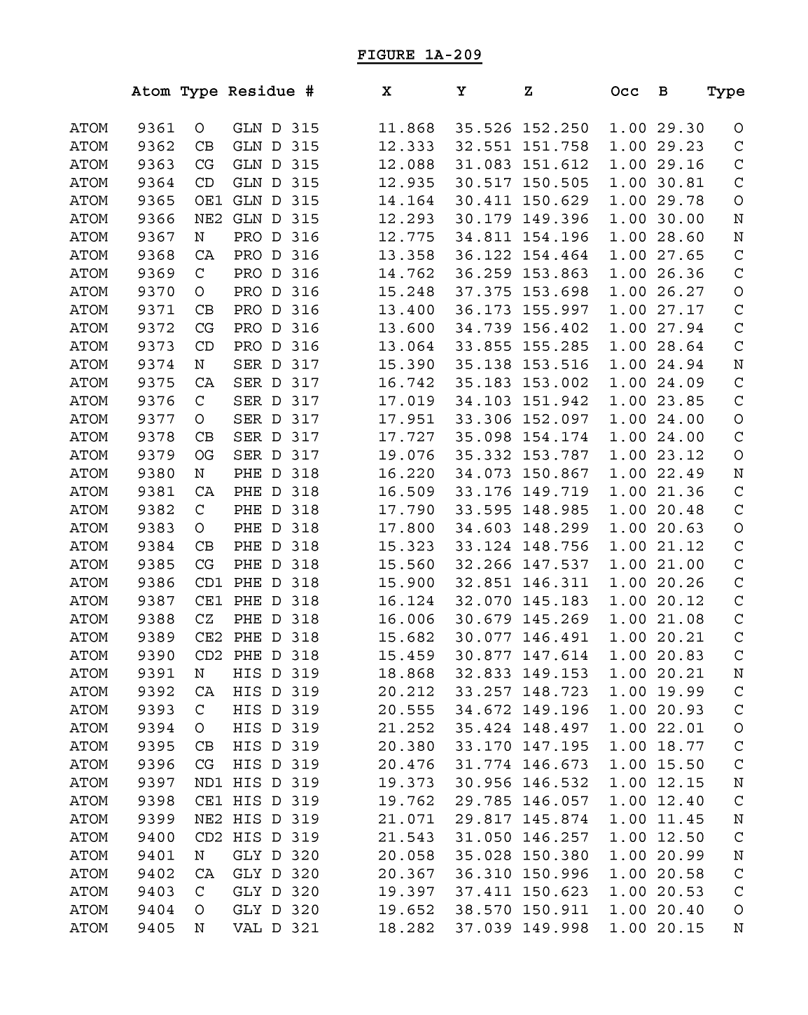

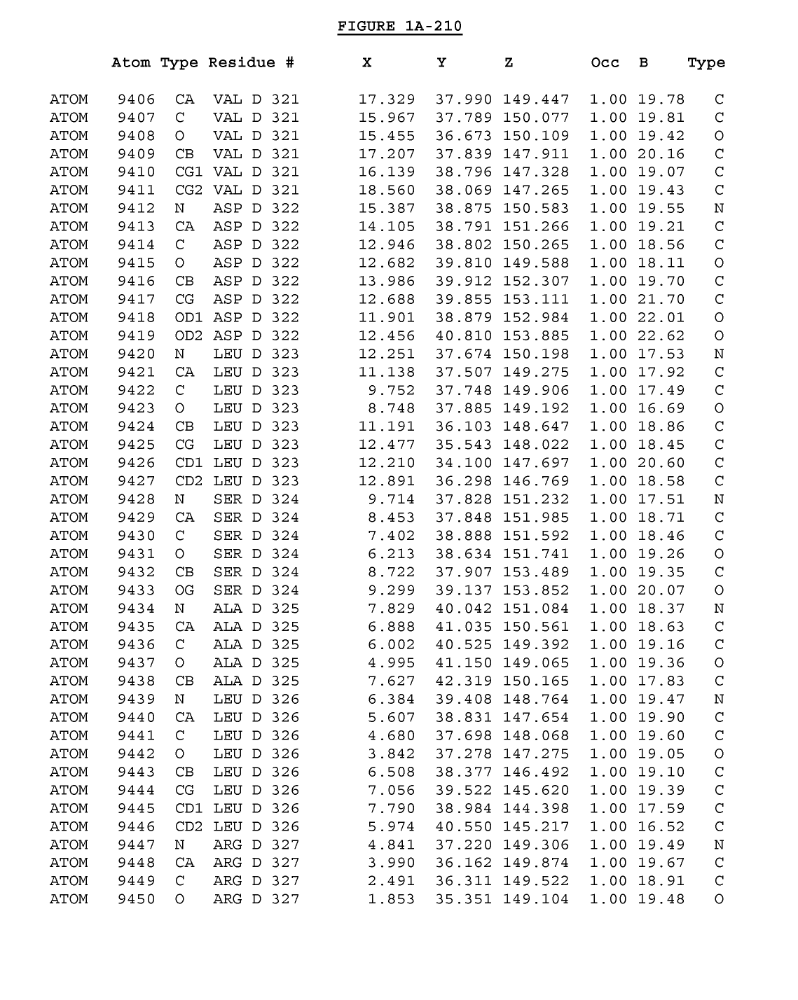

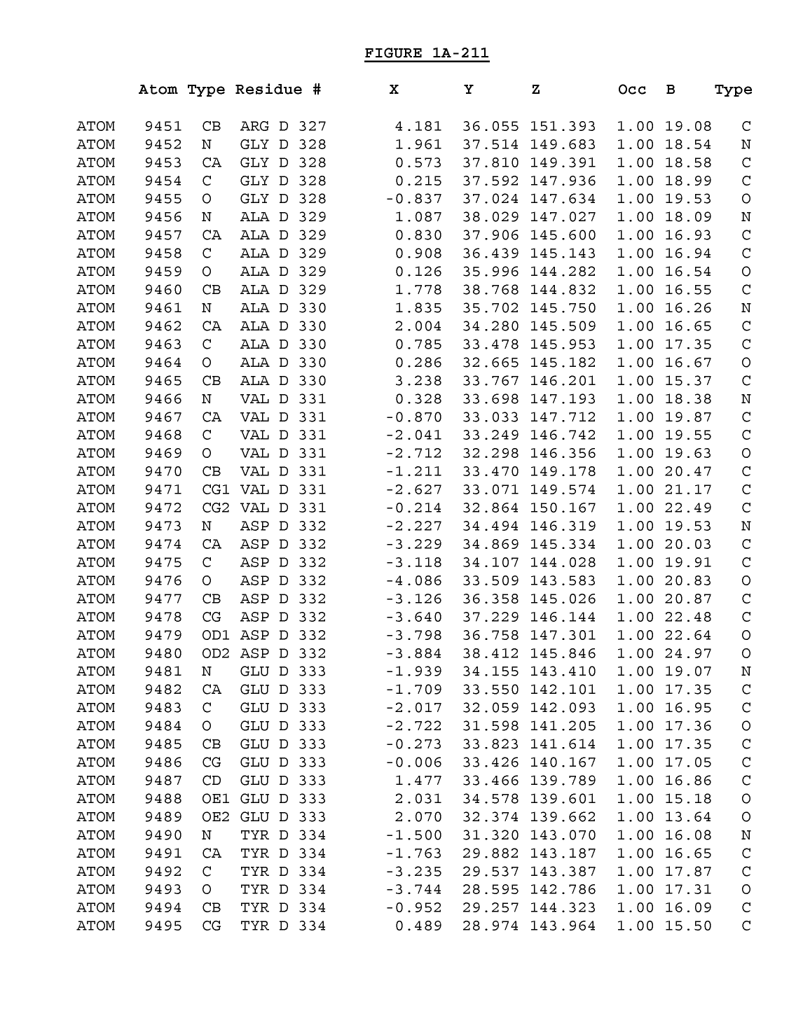

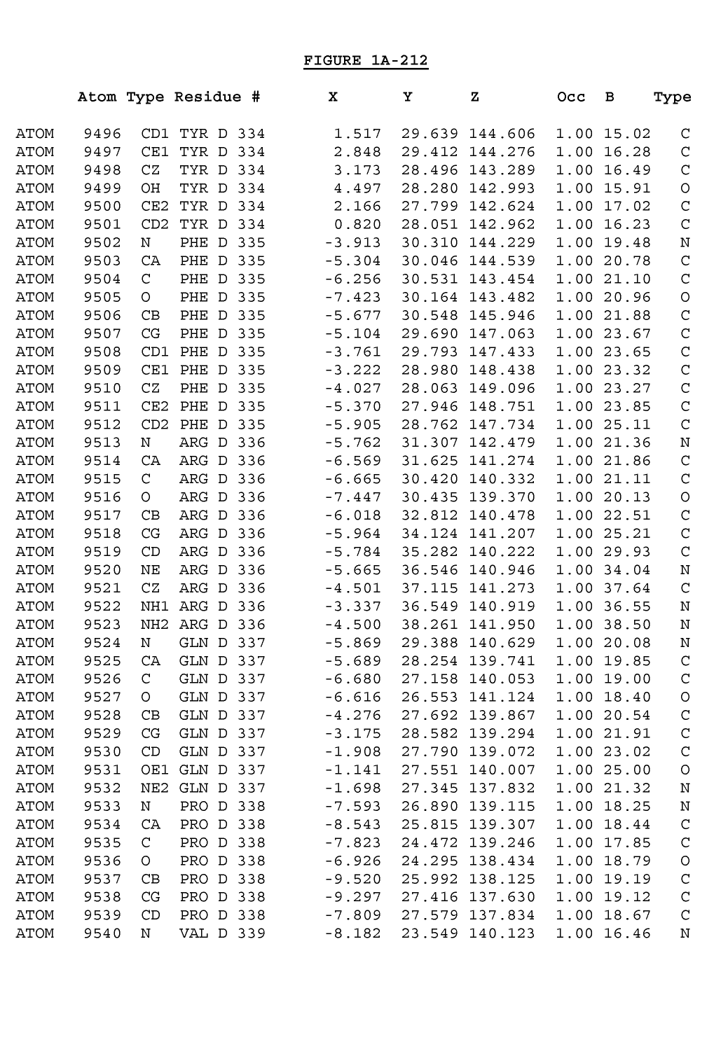

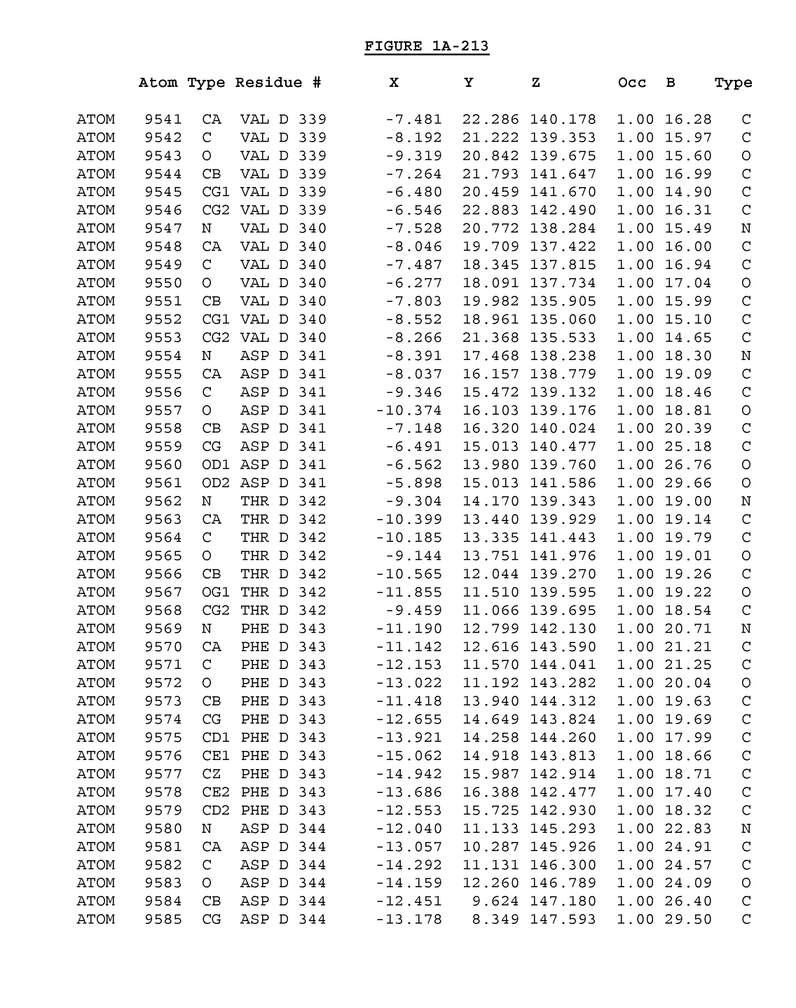

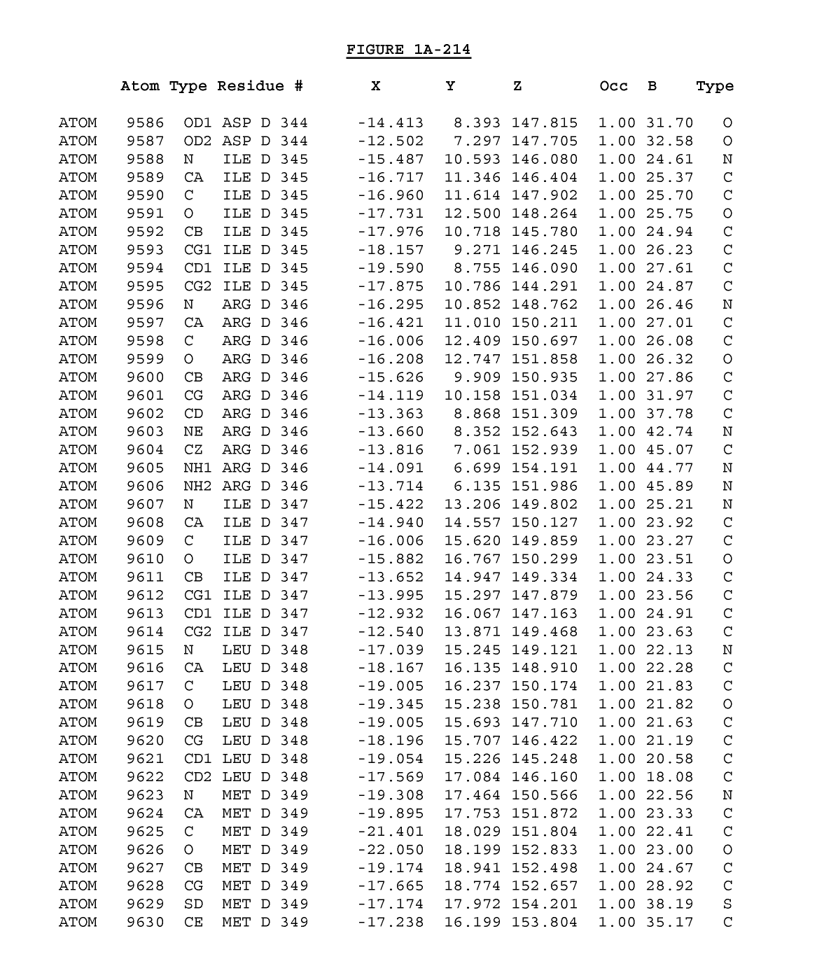

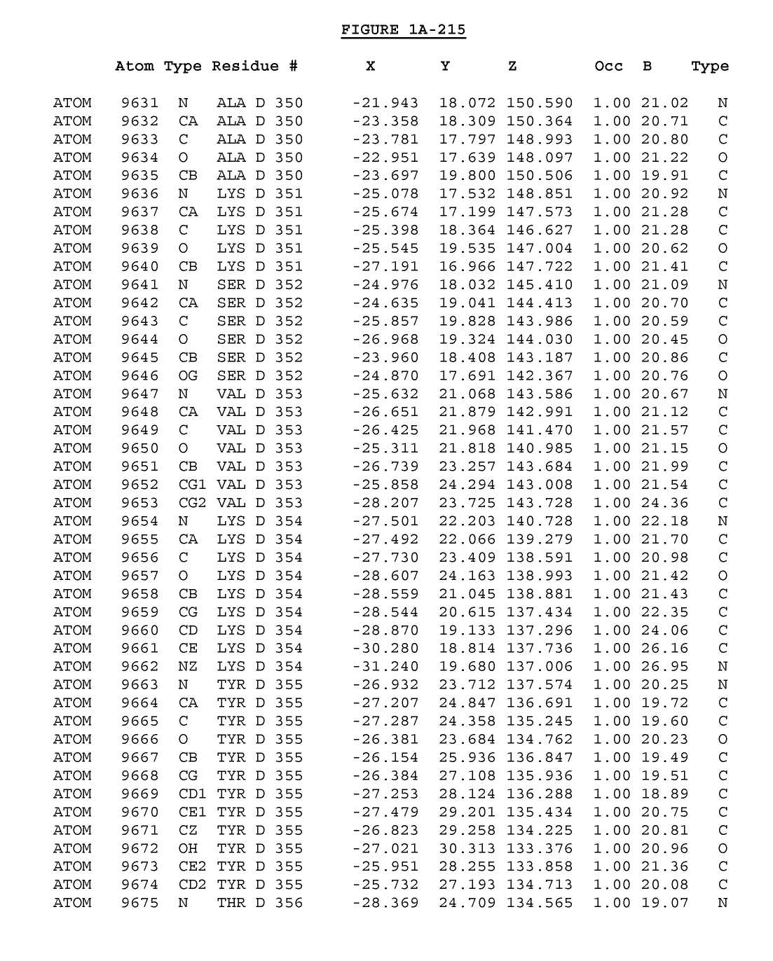

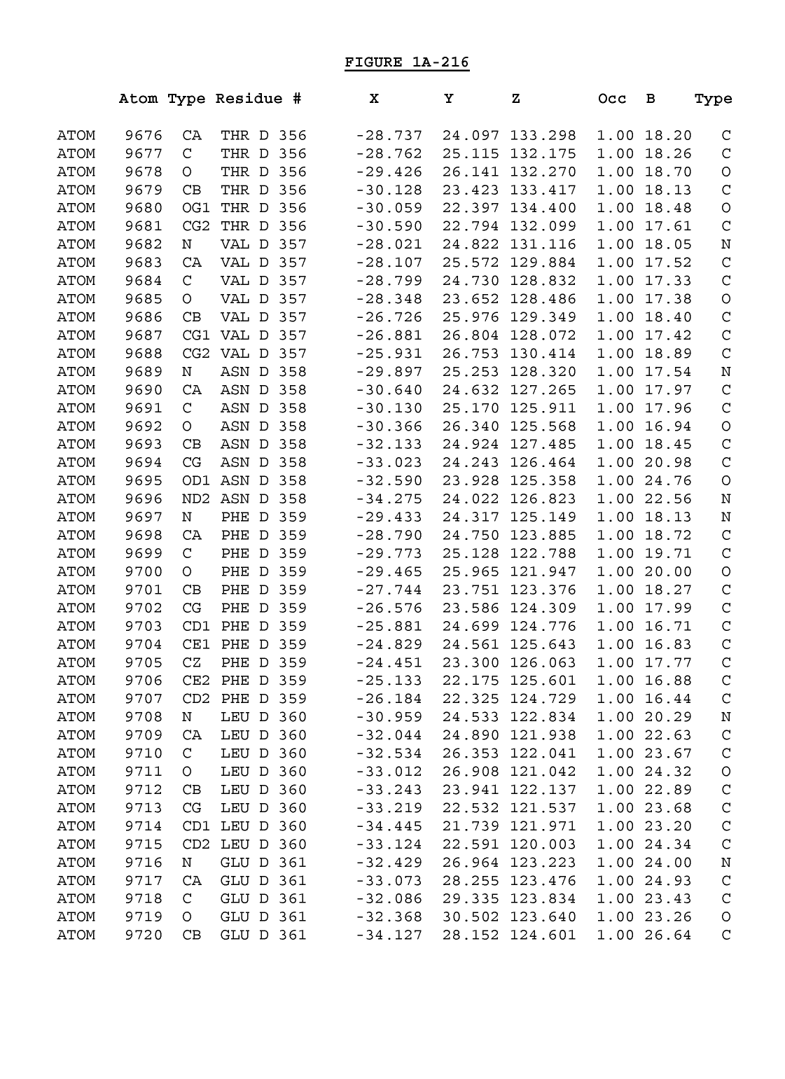

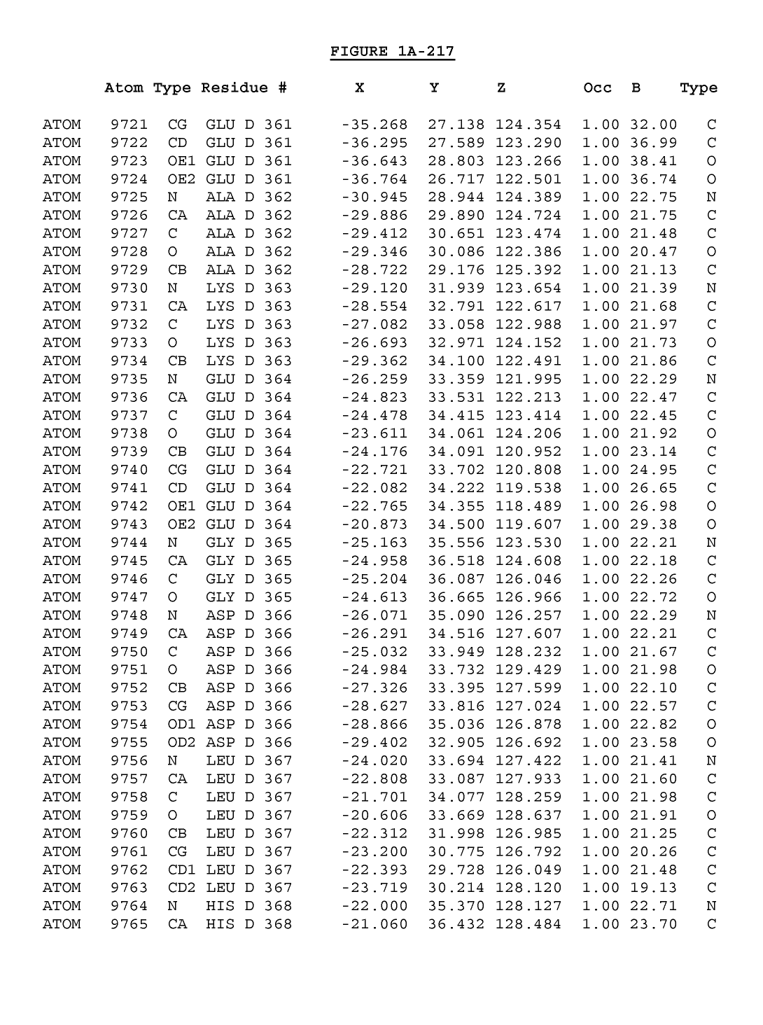

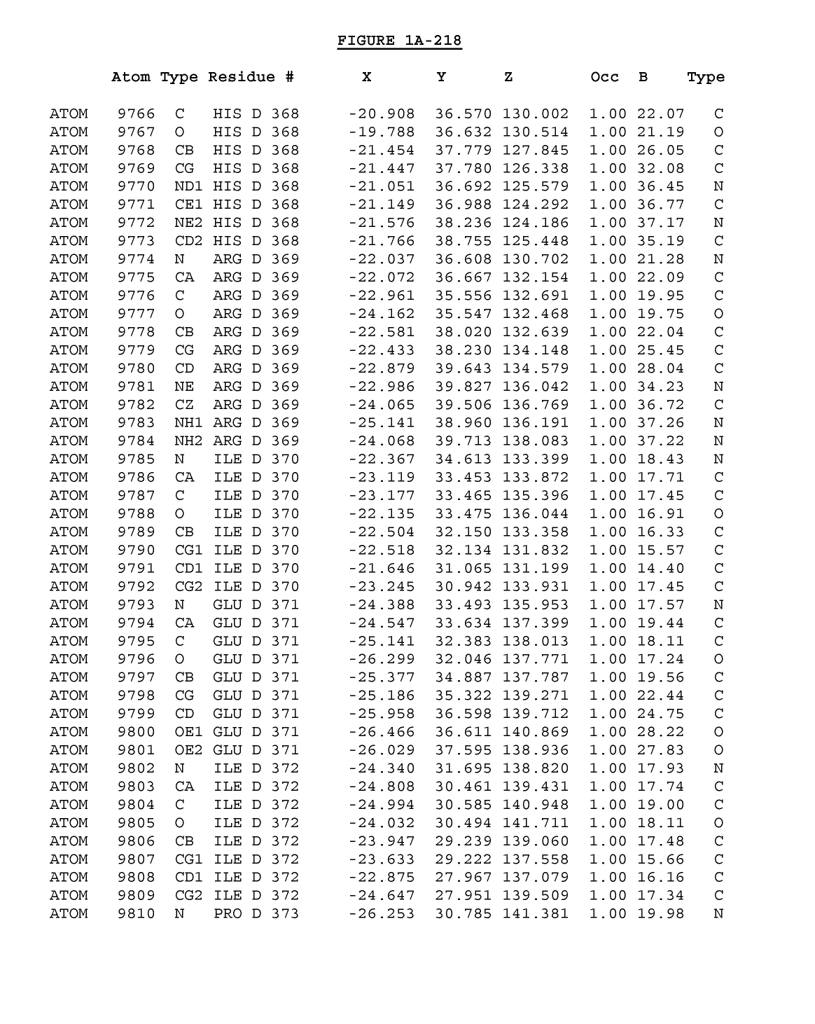

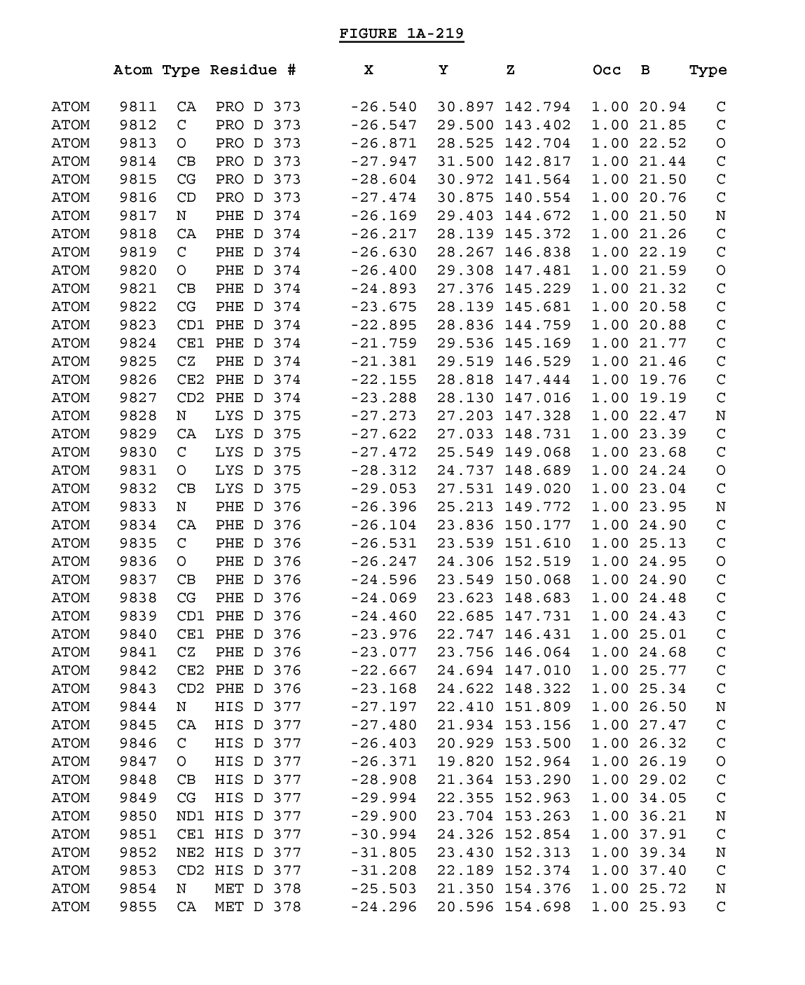

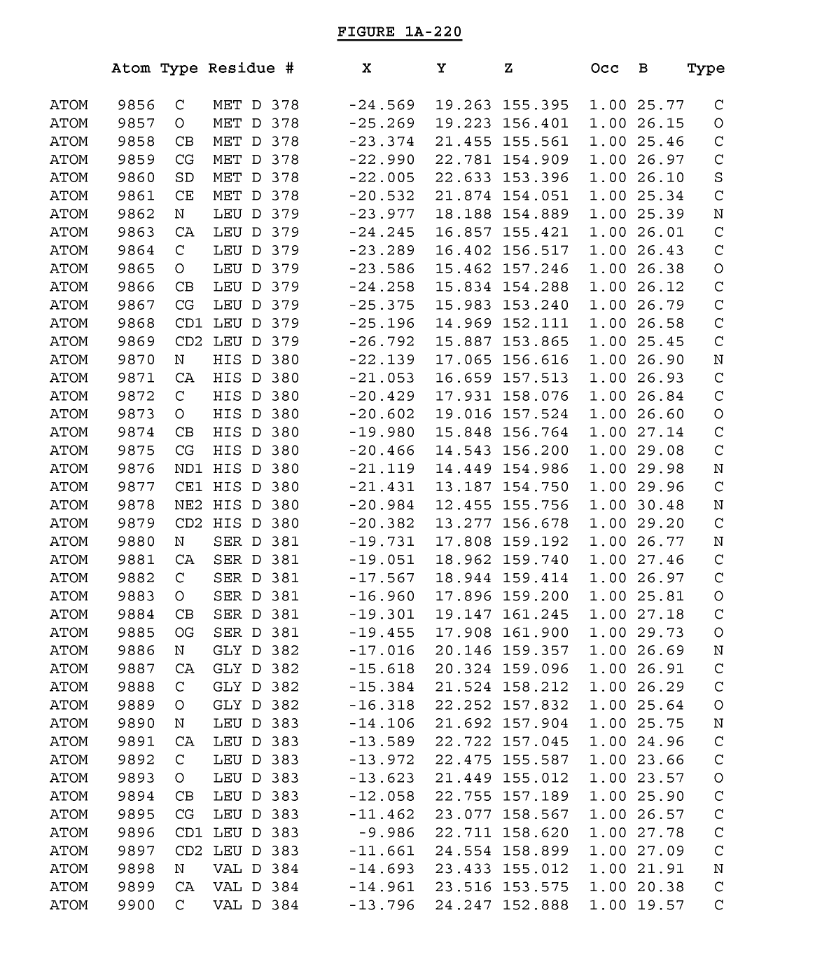

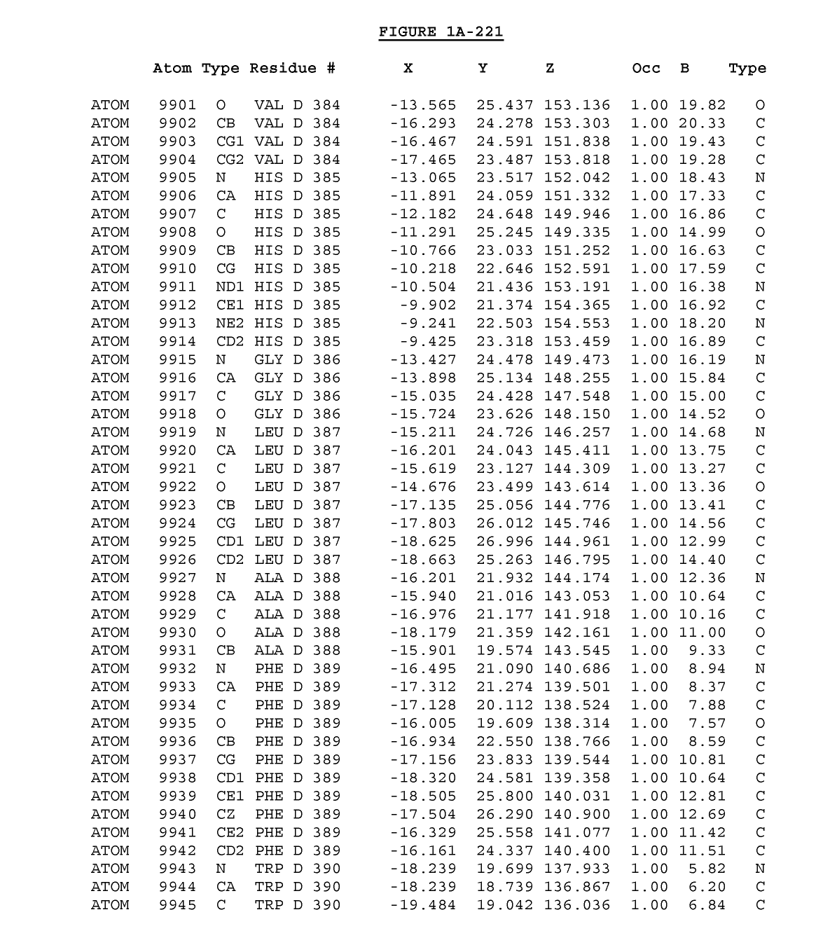

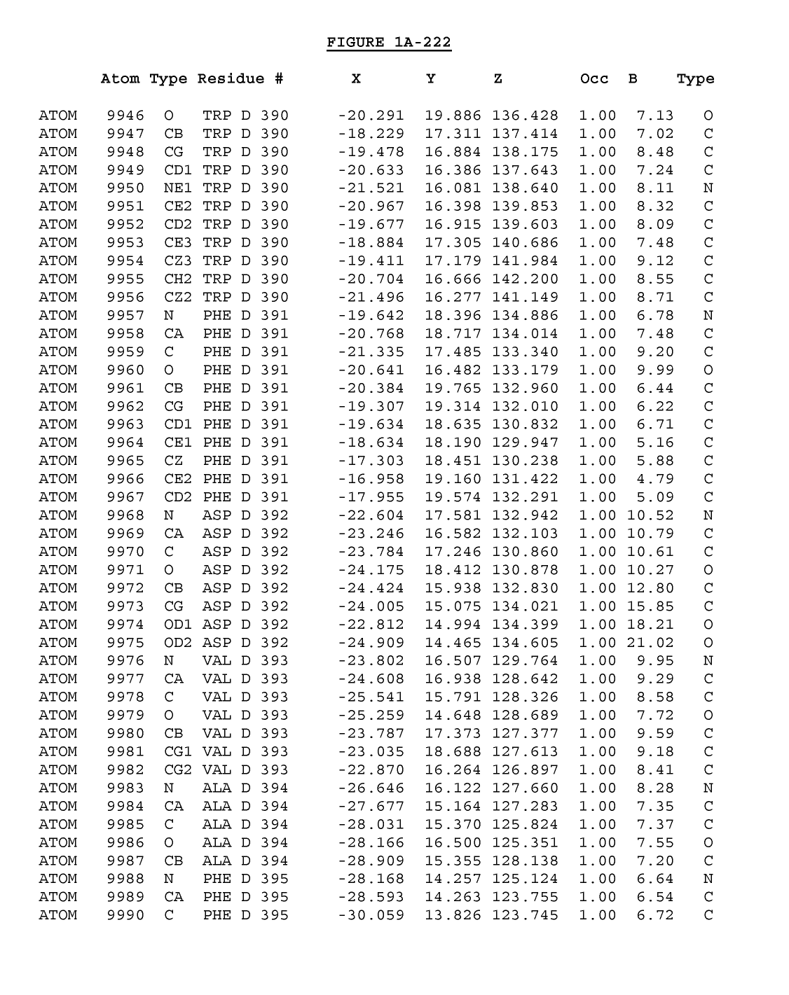

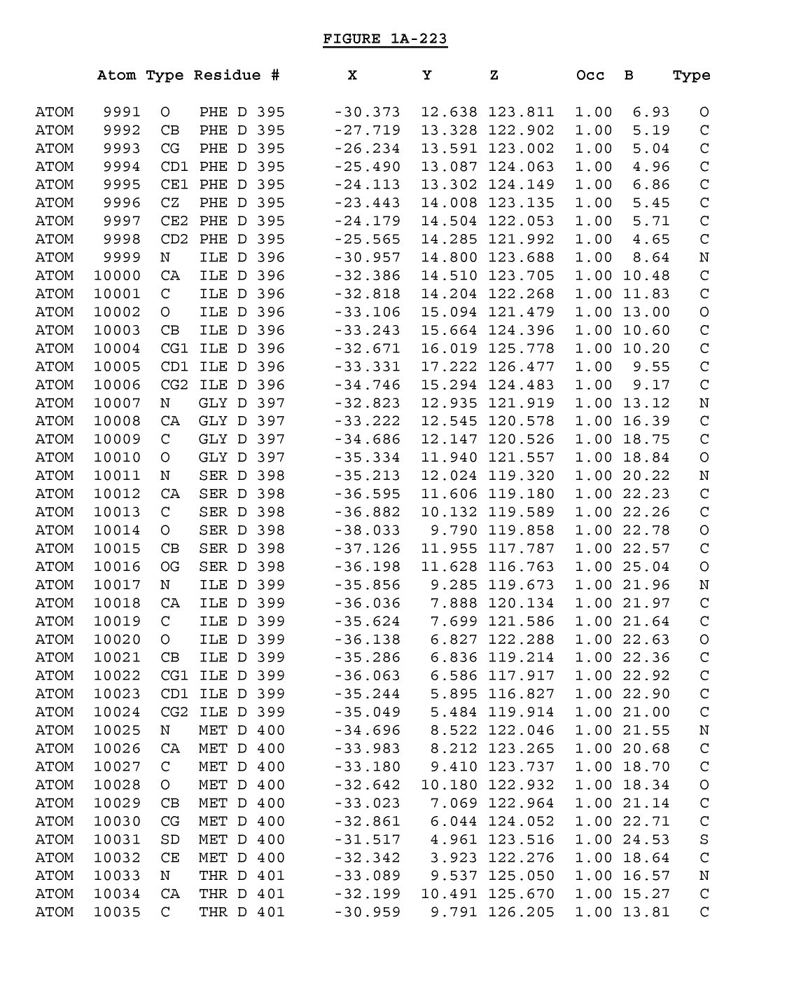

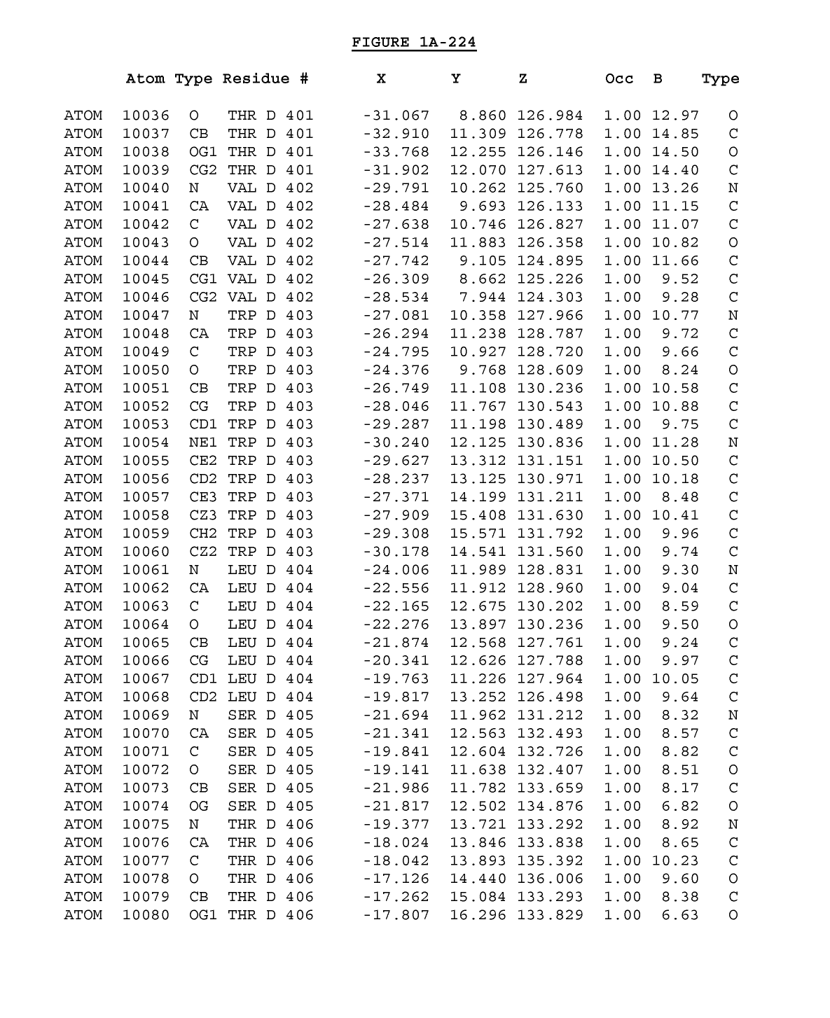

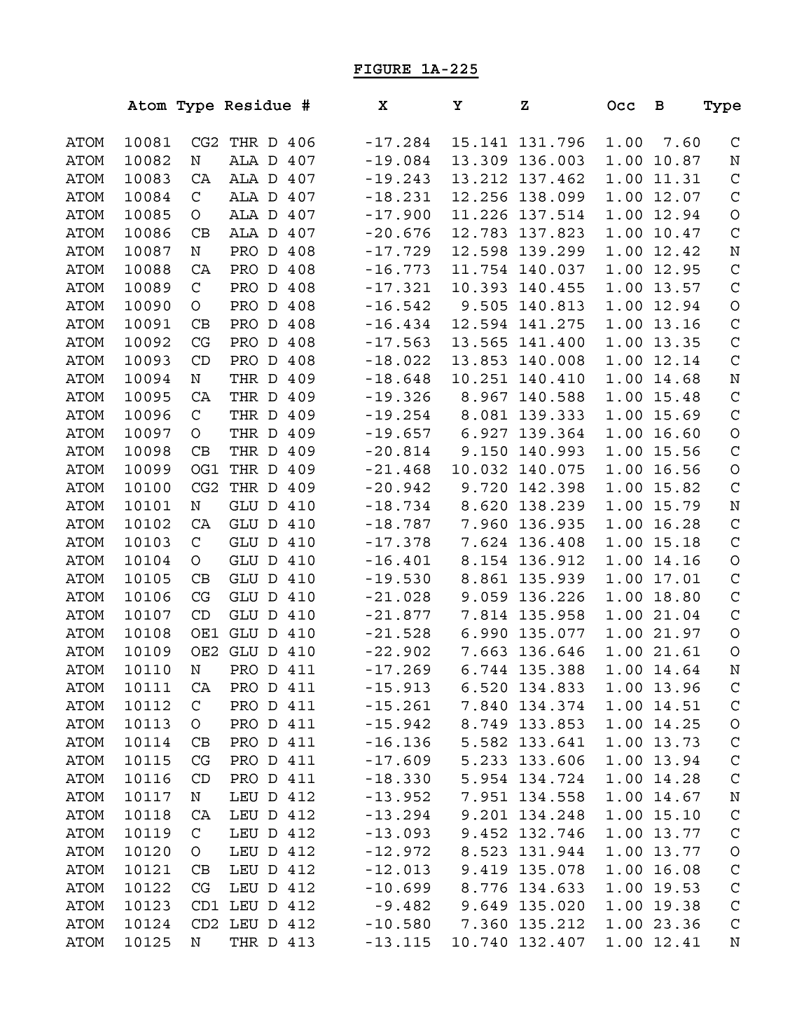

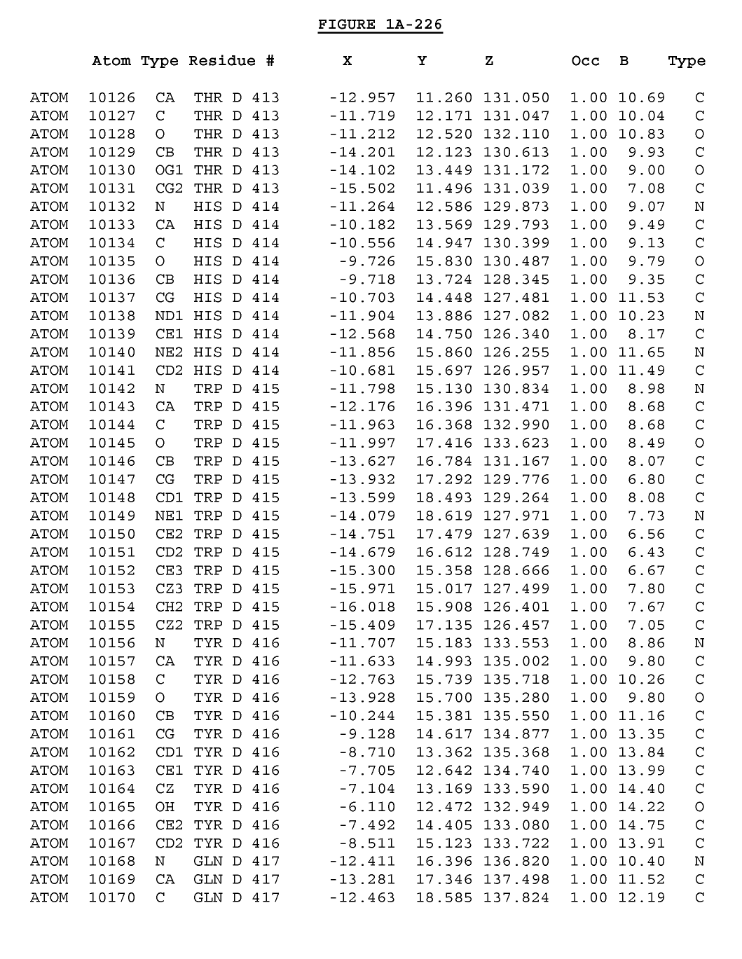

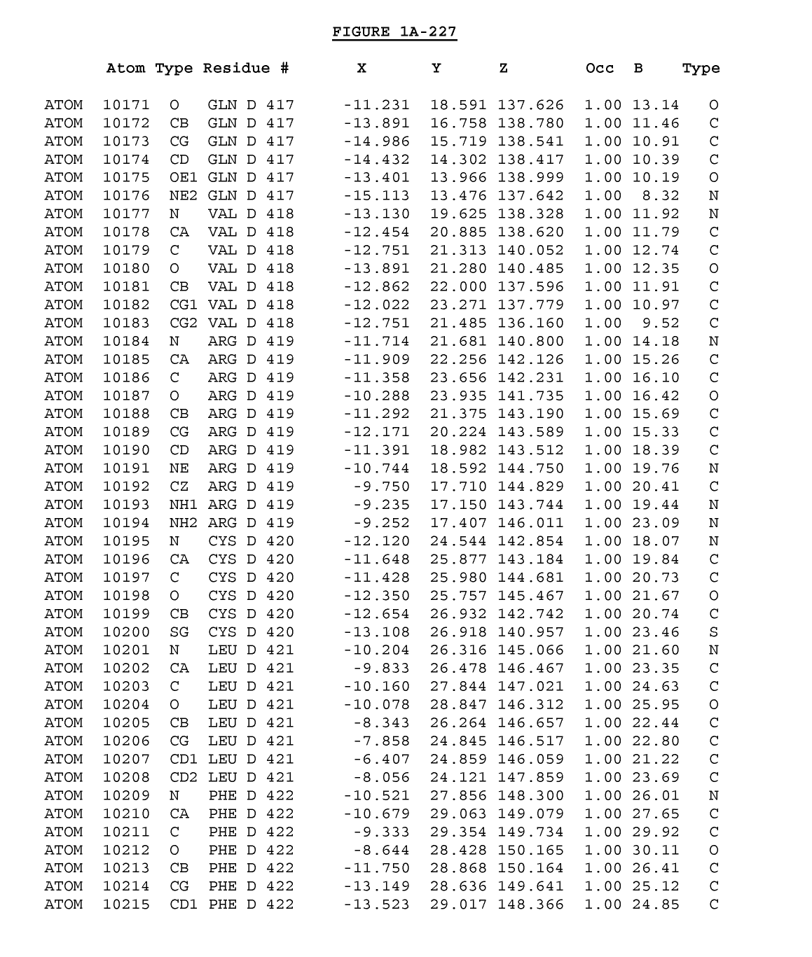

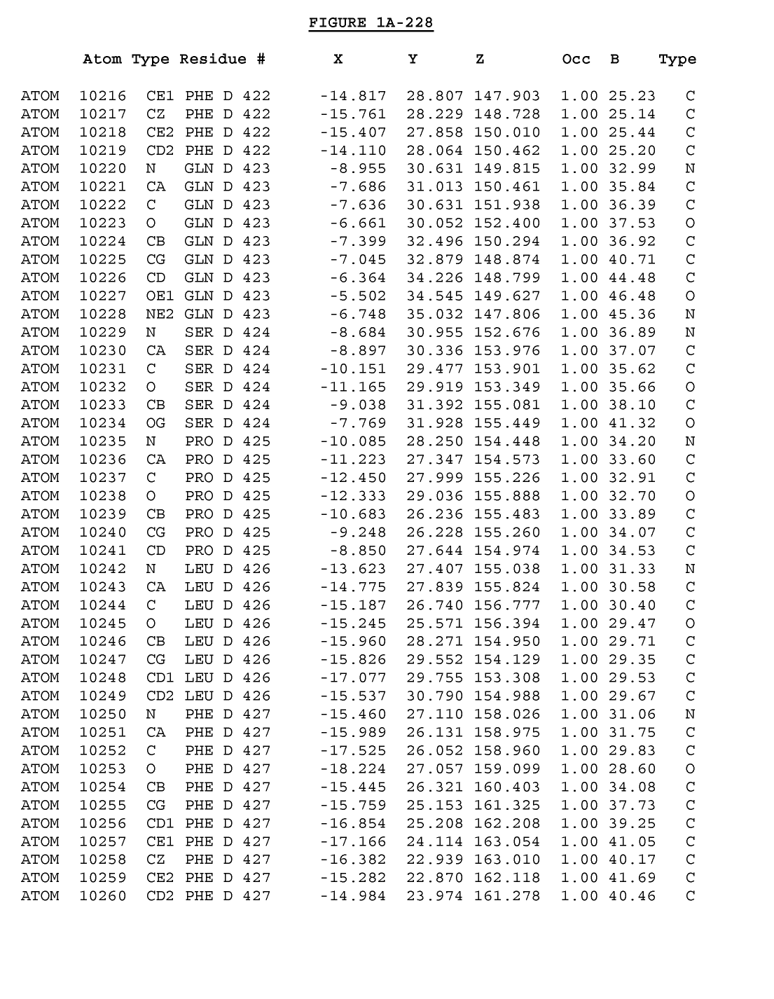

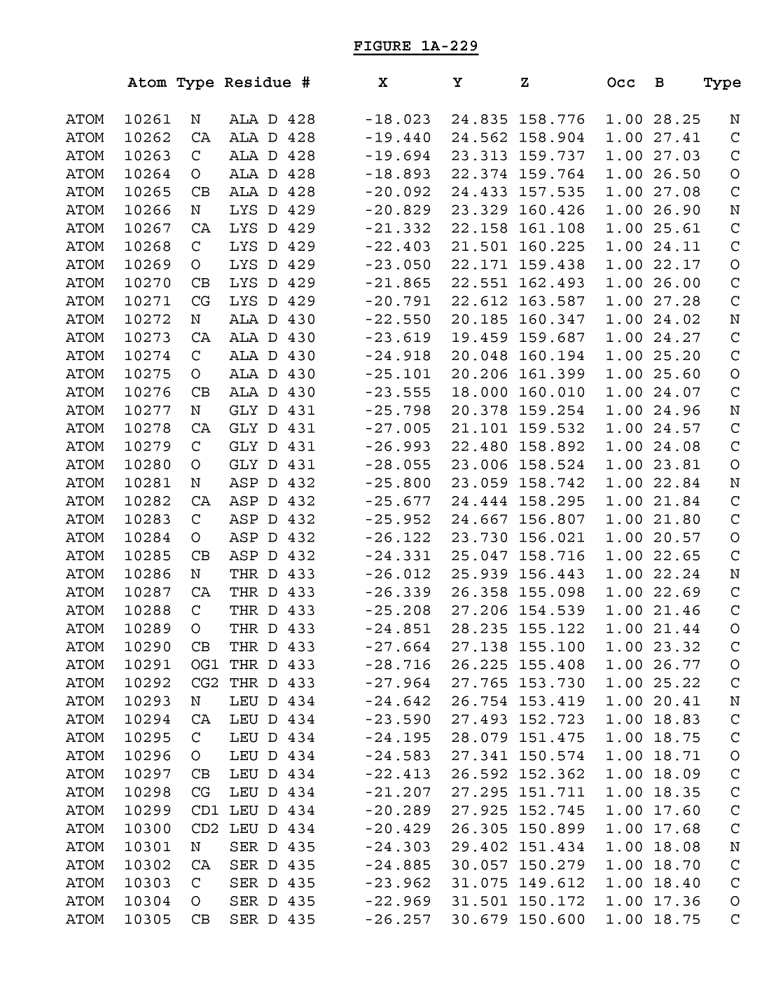

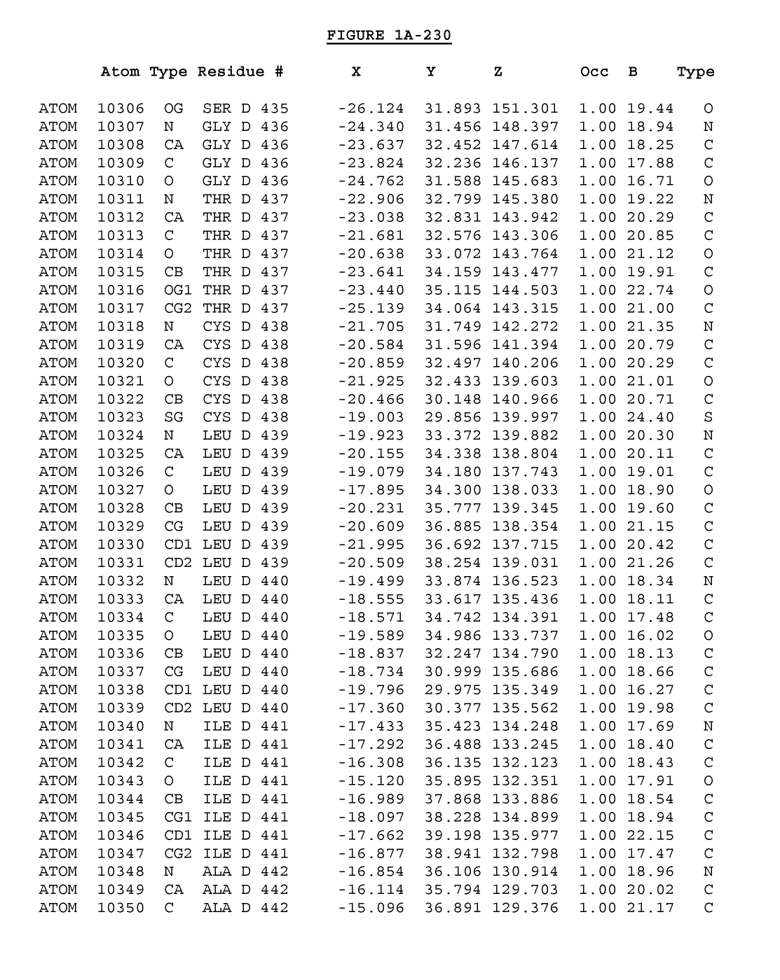

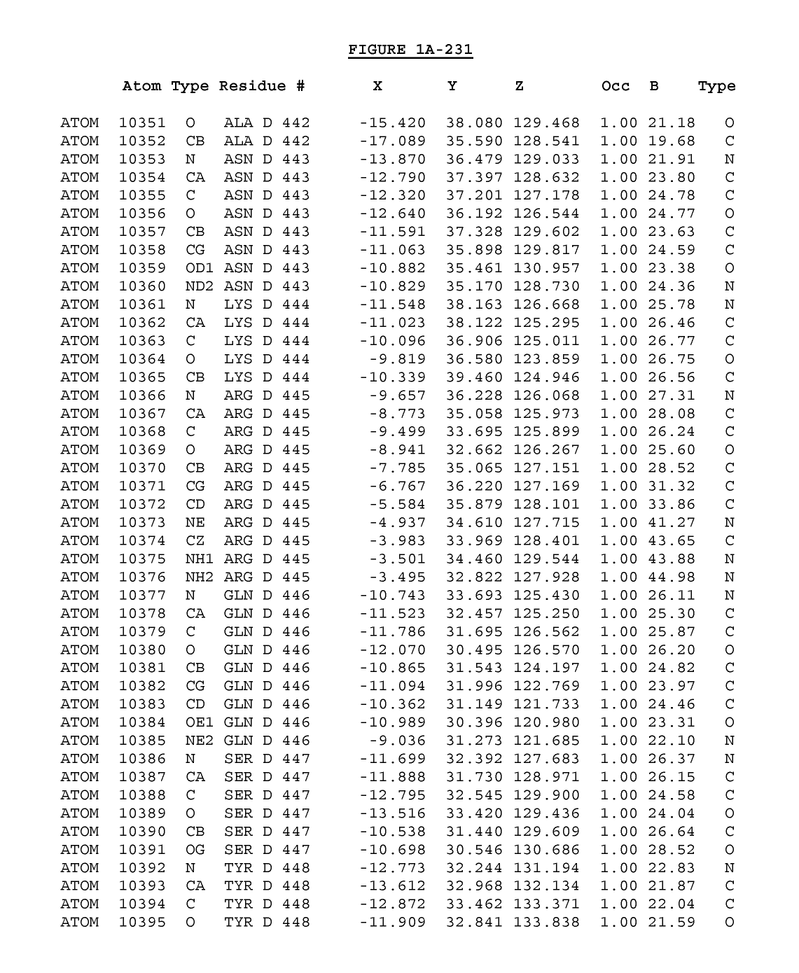

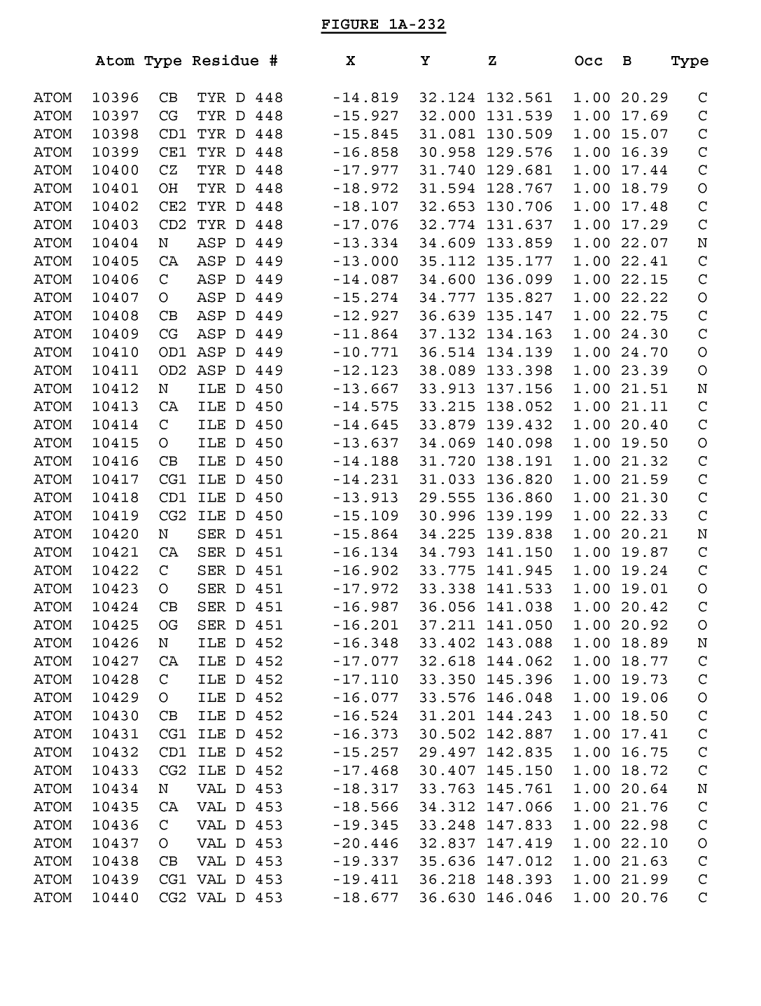

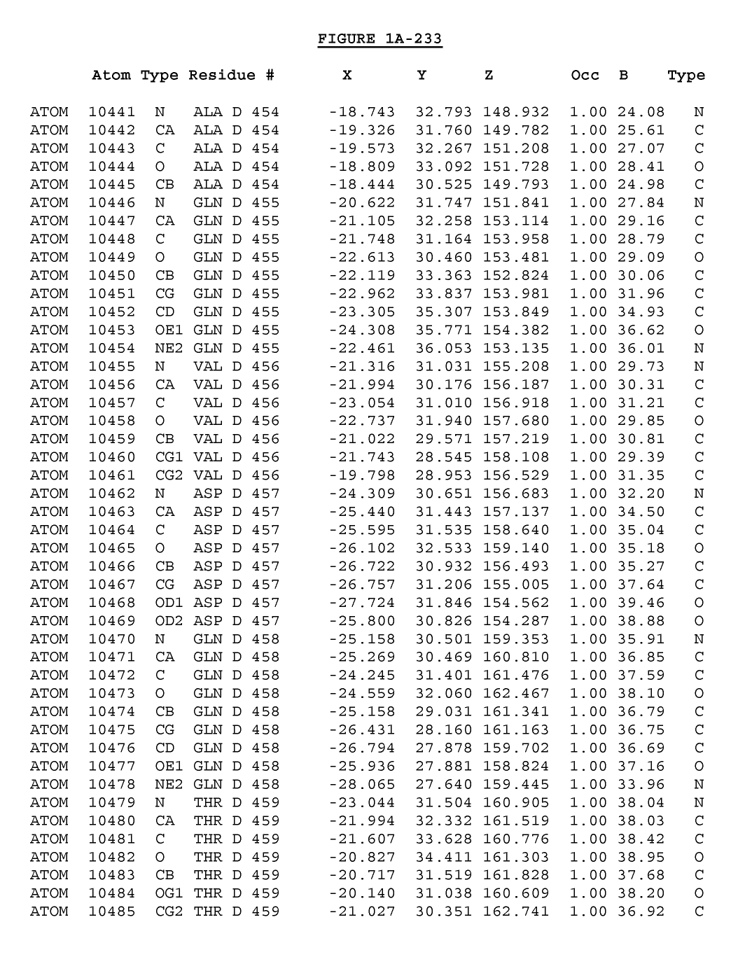

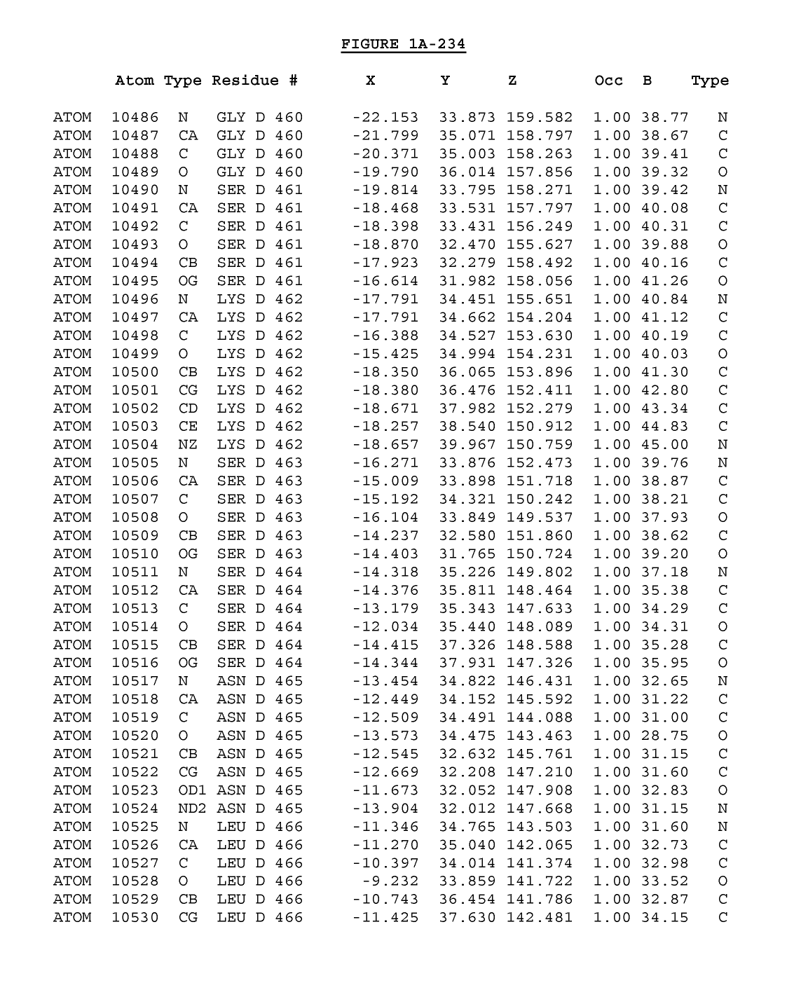

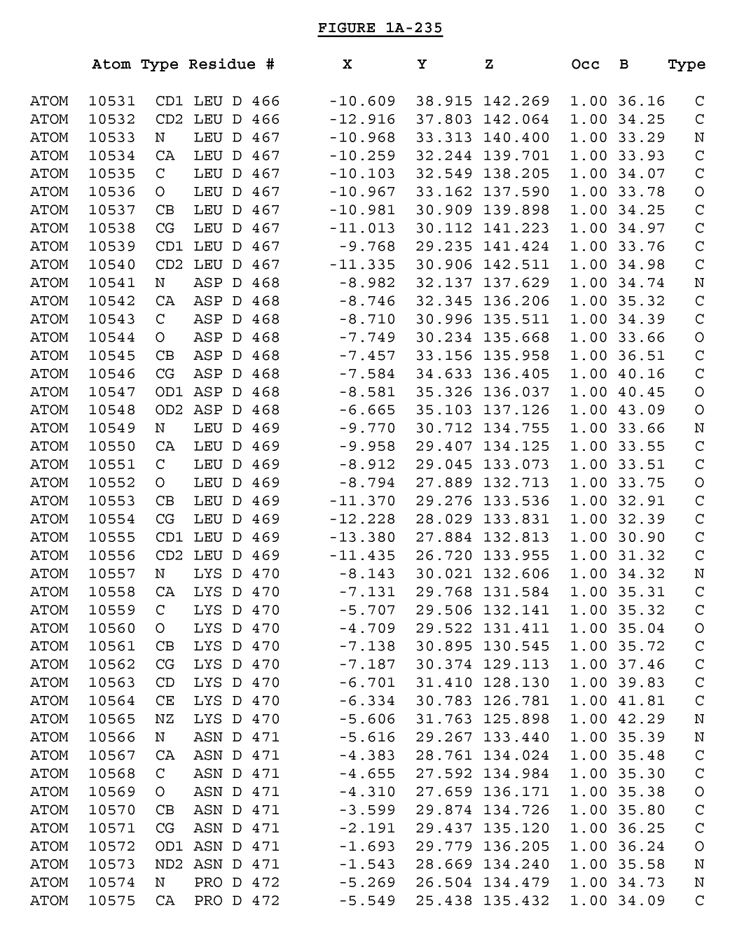

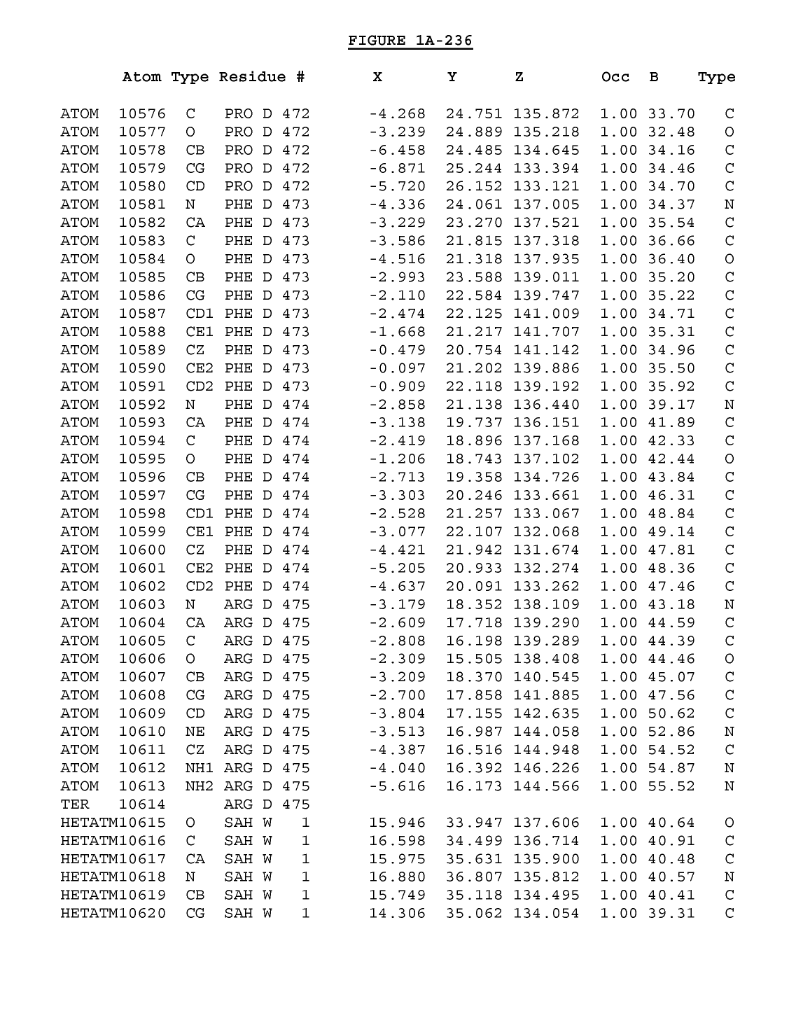

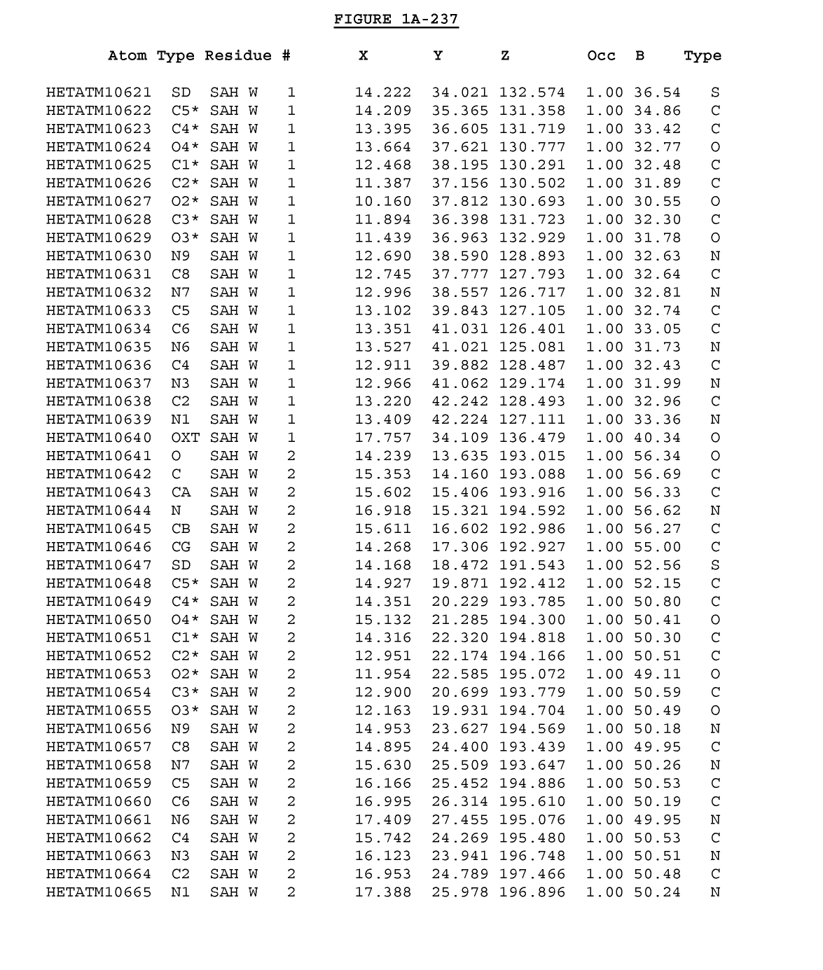

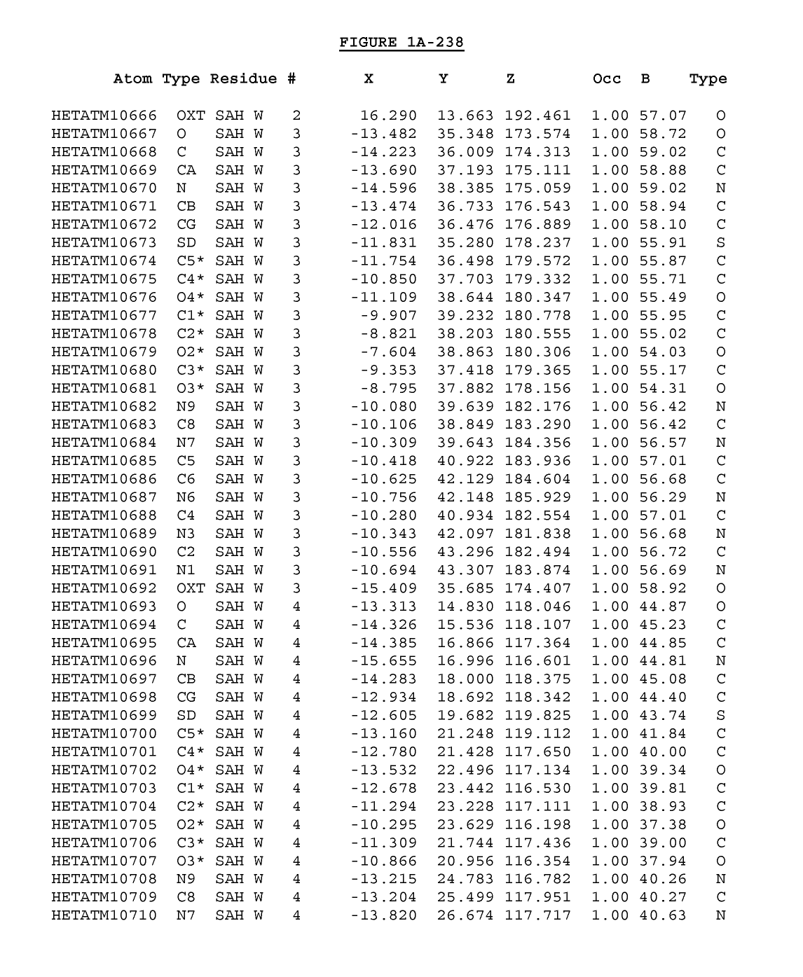

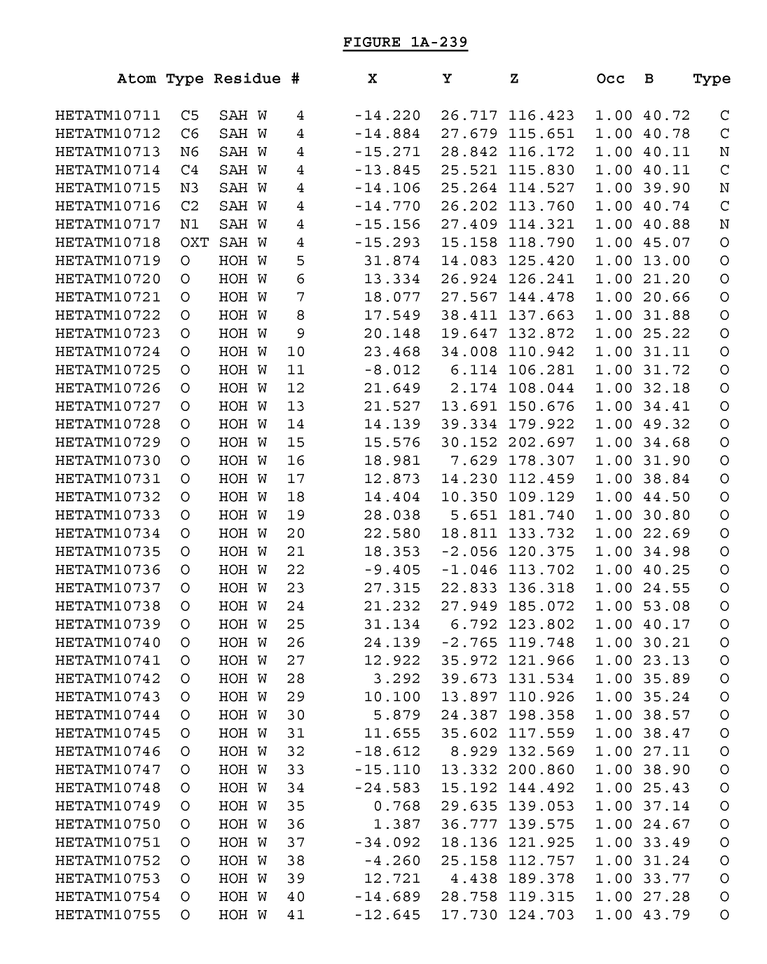

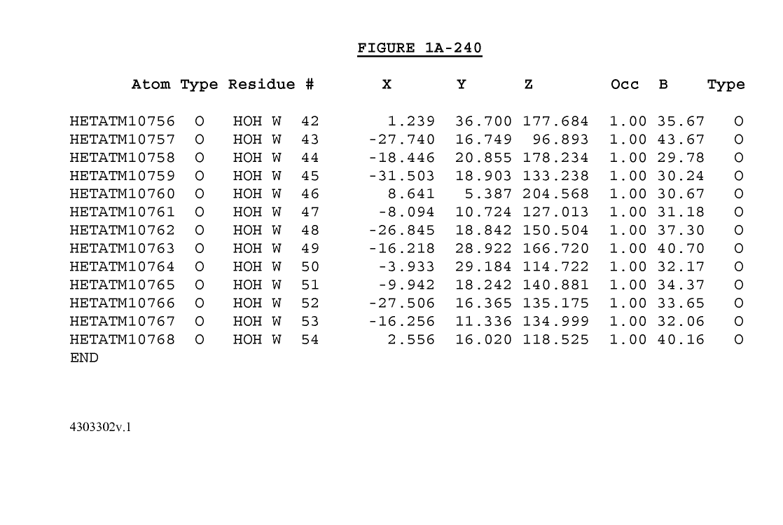

The term “contact score” refers to a measure of shape complementarity between the chemical entity and binding pocket, which is correlated with an RMSD value obtained from a least square superimposition between all or part of the atoms of the chemical entity and all or part of the atoms of the ligand bound (for example, SAM or some other binder) in the binding pocket according to FIG. 1. The docking process may be facilitated by the contact score or RMSD values. For example, if the chemical entity moves to an orientation with high RMSD, the system will resist the motion. A set of orientations of a chemical entity can be ranked by contact score. A lower RMSD value should give a higher contact score. See Meng et al. J. Comp. Chem., 4, 505-524 (1992).

The term “correspond to” or “corresponding amino acids”, when used in the context of the relationship between amino acid residues of any protein and CARM1 amino acid residues, refers to particular amino acids or analogues thereof that align to amino acids in the human CARM1 protein. Each of these amino acids may be an identical, mutated, chemically modified, conserved, conservatively substituted, functionally equivalent or homologous amino acid, when compared to the CARM1 amino acid to which it could be aligned by those skilled in the art. For example, the following are examples of CARM1 amino acid residues that correspond to PRMT7 amino acid residues: F200:M80 and H221:A102 (the identity of the CARM1 residue is listed first; its position is indicated using CARM1 sequence numbering; and the identity of the PRMT7 residue is given at the end).

Methods for identifying a corresponding amino acid are known in the art and are based upon sequence, structural alignment, its functional position or a combination thereof, as compared to the CARM1 protein. For example, corresponding amino acids may be identified by superimposing the backbone atoms of the amino acids in CARM1 and another protein using well known software applications, such as QUANTA (Molecular Simulations, Inc., San Diego, Calif. ©1998, 2000; Accelrys ©2001, 2002). The corresponding amino acids may also be identified using sequence alignment programs such as the “bestfit” program or CLUSTAL W Alignment Tool (Higgins D. G., et al., Methods Enzymol., 266: 383-402 (1996)).

The term “crystallization solution” refers to a solution which promotes crystallization comprising at least one agent, including a buffer, one or more salts, a precipitating agent, one or more detergents, sugars or organic compounds, lanthanide ions, a poly-ionic compound, and/or stabilizer.

The term “docking” refers to orienting, rotating, or translating a chemical entity in the binding pocket, domain, molecule or molecular complex or portion thereof based on distance geometry or energy. Docking may be performed by distance geometry methods that find sets of atoms of a chemical entity that match sets of sphere centers of the binding pocket, domain, molecule or molecular complex or portion thereof. See Meng et al. J. Comp. Chem., 4, 505-524 (1992). Sphere centers are generated by providing an extra radius of given length from the atoms (excluding hydrogen atoms) in the binding pocket, domain, molecule or molecular complex or portion thereof. Real-time interaction energy calculations, energy minimizations or rigid-body minimizations (Gschwend, et al., J. Mol. Recognition, 9:175-186 (1996)) can be performed during or after orientation of the chemical entity to facilitate docking. For example, interactive docking experiments can be designed to follow the path of least resistance. If the user in an interactive docking experiment makes a move to increase the energy, the system will resist that move. However, if that user makes a move to decrease energy, the system will favor that move by increased responsiveness. (Cohen, et al., J. Med. Chem. 33:889-894 (1990)). Docking can also be performed by combining a Monte Carlo search technique with rapid energy evaluation using molecular affinity potentials. See Goodsell and Olson, Proteins: Structure, Function and Genetics 8:195-202 (1990). Software programs that carry out docking functions include but are not limited to MATCHMOL (Cory et al., J Mol. Graphics, 2, 39 (1984); MOLFIT (Redington, Comput. Chem., 16, 217 (1992)) and DOCK (Meng et al., supra). Other software, such as GLIDE (Sherman et al., Chem. Biol. Drug Des., 67, 83-84 (2006)) allow for the dynamic docking of a ligand to an “induced fit” conformation of a protein derived from the starting coordinates of a protein target by stripping back certain side chains near the binding site of the provided protein, docking into the stripped-back site, reintroducing the side chains, and relaxing the complex.

The term “domain” refers to a structural unit of the CARM1 protein or homologue. The domain can comprise a binding pocket, a sequence or structural motif.

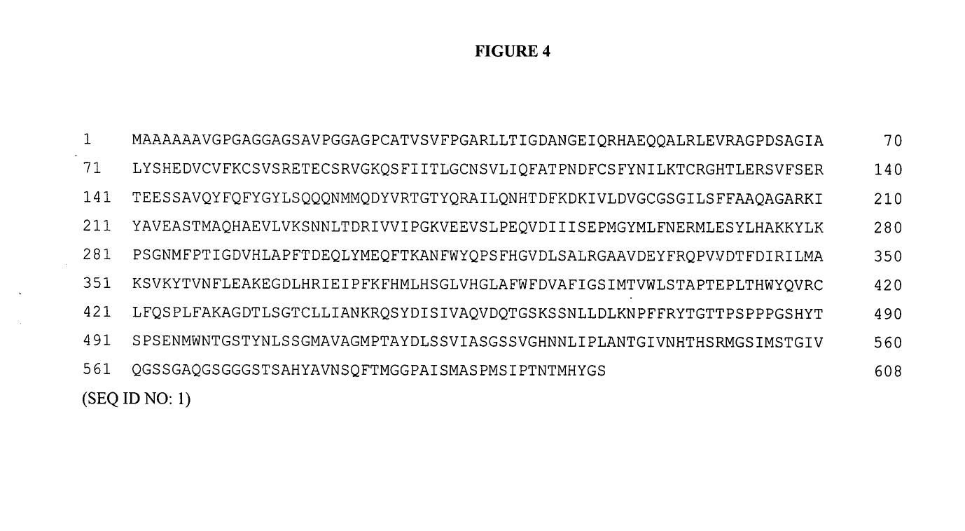

The term “full-length CARM1” refers to the complete human CARM1 (NCBI GeneID: 10498) protein, which includes the methtransferase domain and the putative transactivation domain (amino acid residues 1 to 608; GenBank accession no. NP_{—}954592; SEQ ID NO:1, FIG. 4).

The term “CARM1-like” refers to all or a portion of a molecule or molecular complex that has a commonality of shape with all or a portion of the CARM1 protein. For example, in the CARM1-like SAM binding pocket, the commonality of shape is defined by a root mean square deviation of the structure coordinates of the backbone atoms between the amino acids in the CARM1-like SAM binding pocket and the CARM1 amino acids in the CARM1 SAM binding pocket (as set forth in FIG. 1A). Compared to the amino acids of the CARM1 binding pocket, the corresponding amino acid residues in the CARM1-like binding pocket may or may not be identical. Depending on the set of CARM1 amino acid residues that define the CARM1 SAM binding pocket, one skilled in the art would be able to locate the corresponding amino acids that define a CARM1-like binding pocket in a protein based on sequence or structural homology.

The term “CARM1 protein complex” or “CARM1 homologue complex” refers to a molecular complex formed by associating the CARM1 protein or CARM1 homologue with a chemical entity, for example, a ligand, a substrate, nucleotide amino acid, non-natural nucleotide amino acid, amino acid, an agonist or antagonist, binder, antibody, drug or compound.

The term “generating a three-dimensional structure” or “generating a three-dimensional representation” refers to converting the lists of structure coordinates into structural models or graphical representations in three-dimensional space. This can be achieved through commercially or publicly available software. A model of a three-dimensional structure of a molecule or molecular complex can thus be constructed on a computer screen by a computer that is given the structure coordinates and that comprises the correct software. The three-dimensional structure may be displayed or used to perform computer modeling or fitting operations. In addition, the structure coordinates themselves, without the displayed model, may be used to perform computer-based modeling and fitting operations.

The term “homologue of CARM1 domain” or “CARM1 domain homologue” refers to the domain of a protein that is at least 70%, 80%, 90%, 95%, 96%, 97%, 98%, 99% or greater than 99% identical in sequence to the corresponding domain of human CARM1 protein and retains CARM1 methyltransferase activity. In one embodiment, the homologue is at least 95%, 96%, 97%, 98% or 99% identical in sequence to the corresponding human CARM1 domain, and has conservative mutations as compared to human CARM1 domain. The homologue can be a CARM1 domain from another species, or the foregoing human CARM1 domain with mutations, conservative substitutions, additions, deletions or a combination thereof. Such animal species include, but are not limited to, mouse, rat, a primate such as monkey or other primates.

The term “homology model” refers to a structural model derived from known three-dimensional structure(s). Generation of the homology model, termed “homology modeling”, can include sequence alignment, residue replacement, residue conformation adjustment through energy minimization, or a combination thereof.

The term “interaction energy” refers to the energy determined for the interaction of a chemical entity and a binding pocket, domain, molecule or molecular complex or portion thereof. Interactions include but are not limited to one or more of covalent interactions, non-covalent interactions such as hydrogen bond, electrostatic, hydrophobic, aromatic, van der Waals interactions, and non-complementary electrostatic interactions such as repulsive charge-charge, dipole-dipole and charge-dipole interactions. As interaction energies are measured in negative values, the lower the value the more favorable the interaction.

The term “motif” refers to a group of amino acid residues in the CARM1 protein or homologue that defines a structural compartment or carries out a function in the protein or homologue, for example, catalysis or structural stabilization, or methylation. The motif may be conserved in sequence, structure and function. The motif can be contiguous in primary sequence or three-dimensional space. An example of a motif includes but is not limited to the residues lining the SAM-binding site.

The term “part of a binding pocket” refers to less than all of the amino acid residues that define the binding pocket. The structure coordinates of amino acid residues that constitute part of a binding pocket may be specific for defining the chemical environment of the binding pocket, or useful in designing fragments of a binder that may interact with those residues. For example, the portion of amino acid residues may be key residues that play a role in ligand binding, or may be residues that are spatially related and define a three-dimensional compartment of the binding pocket. The amino acid residues may be contiguous or non-contiguous in primary sequence. In one embodiment, part of the binding pocket has at least two amino acid residues, preferably at least three, eight, fourteen or fifteen amino acid residues.

The term “part of a CARM1 protein” or “part of a CARM1 homologue” refers to less than all of the amino acid residues of a CARM1 protein or homologue. In one embodiment, part of the CARM1 protein or homologue defines the binding pockets, domains, sub-domains, and motifs of the protein or homologue. The structure coordinates of amino acid residues that constitute part of a CARM1 protein or homologue may be specific for defining the chemical environment of the protein, or useful in designing fragments of a binder that interact with those residues. The portion of amino acid residues may also be spatially related residues that define a three-dimensional compartment of the binding pocket, motif, or domain. The amino acid residues may be contiguous or non-contiguous in primary sequence. For example, the portion of amino acid residues may be key residues that play a role in ligand or substrate binding, peptide binding, antibody binding, catalysis, structural stabilization or degradation.

The term “quantified association” refers to calculations of distance geometry and energy. Energy can include but is not limited to interaction energy, free energy and deformation energy. See Cohen, supra.

The term “root mean square deviation” or “RMSD” refers to the square root of the arithmetic mean of the squares of the deviations from the mean. It is a way to express the deviation or variation from a trend or object. For purposes of this invention, the “root mean square deviation” defines the variation in the backbone of a protein from the backbone of CARM1, a binding pocket, a motif, a domain, or portion thereof, as defined by the structure coordinates of CARM1 described herein. It would be readily apparent to those skilled in the art that the calculation of RMSD involves standard error of ±0.1 Å.

The term “soaked” refers to a process in which a crystal is transferred to a solution containing a compound of interest.

The term “structure coordinates” refers to Cartesian coordinates derived from mathematical equations related to the patterns obtained on diffraction of a monochromatic beam of X-rays by the atoms (scattering centers) of a protein or protein complex in crystal form. The diffraction data are used to calculate an electron density map of the repeating unit of the crystal. The electron density maps are then used to establish the positions of the individual atoms of the molecule or molecular complex.

The term “sub-domain” refers to a portion of a domain.

The term “substantially all of a CARM1 binding pocket” or “substantially all of a CARM1 protein” refers to all or almost all of the amino acids in the CARM1 binding pocket or protein. For example, substantially all of a CARM1 binding pocket can be 100%, 95%, 90%, 80%, or 70% of the residues defining the CARM1 binding pocket or protein.

The term “substrate binding pocket” refers to the binding pocket for a substrate of CARM1 or homologue thereof. A substrate is generally defined as the molecule upon which an enzyme performs catalysis. Natural substrates, synthetic substrates or peptides, or mimics of natural substrates of CARM1 or homologue thereof may associate with the substrate binding pocket

The term “sufficiently homologous to CARM1” refers to a protein that has a sequence identity of at least 25% compared to CARM1 protein. In other embodiments, the sequence identity is at least 40%. In other embodiments, the sequence identity is at least 50%, 60%, 70%, 80%, 90%, 95%, 96%, 97%, 98% or 99%.

The term “three-dimensional structural information” refers to information obtained from the structure coordinates. Structural information generated can include the three-dimensional structure or graphical representation of the structure. Structural information can also be generated when subtracting distances between atoms in the structure coordinates, calculating chemical energies for a CARM1 molecule or molecular complex or homologues thereof, calculating or minimizing energies for an association of a CARM1 molecule or molecular complex, or homologues thereof to a chemical entity.

Crystallizable Compositions and Crystals of a CARM1 Domain and Complexes Thereof

In one embodiment, the invention provides a crystallizable composition comprising a CARM1 domain or its homologue. In another embodiment, the crystallizable composition further comprises a buffer that maintains pH between about 8.0 and 12.0 and 0.1-5 M ammonium sulfate. In certain embodiments, the crystallizable composition comprises equal volumes of a solution of a CARM1 domain or a homologue thereof (11 mg/ml) in the presence of 0.5 mM S-Adenosyl-L-Homocysteine, 2.2 mM ammonium sulfate, and 100 mM Hepes pH 8.5. In other embodiments, the crystallizable composition comprises equal volumes of a solution of a CARM1 domain or a homologue thereof (11 mg/ml) in the presence of 0.5 mM S-Adenosyl-L-Homocysteine, 2.2 mM ammonium sulfate, and 100 mM Tris HCl pH 8.5.

According to another embodiment, the invention provides a crystal comprising a CARM1 domain or its homologue. Preferably, the native crystal has a unit cell dimension of a=74.852, b=98.629 Å, c=207.316 Å and belongs to space group P_{21 21 2}. It will be readily apparent to those skilled in the art that the unit cells of such a crystal composition may deviate ±2% from the above cell dimensions depending on the deviation in the unit cell calculations.

As used herein, the CARM1 domain in the crystallizable compositions or crystals can be amino acids X-Y of SEQ ID NO:1 (FIG. 4), where X is one of 27, 60, 93, 128, 133, or 140, and Y is one of 472, 480, 521, or 608 of SEQ ID NO:1. The homologue thereof can be any of the aforementioned amino acids with conservative substitutions, deletions or additions, to the extent that any substitutions, deletions or additions maintains a CARM1 methyltransferase activity in the homologue; preferably the homologue with substitutions, deletions or additions is at least 70%, 80%, 90%, 95%, 96%, 97%, 98%, or 99% identical to one of the aforementioned. Preferably, the homologue with substitutions, deletions or additions is at least 80%, 90%, 95%, 96%, 97%, 98%, or 99% identical to one of the aforementioned.

The CARM1 protein or its homologue may be produced by any well-known method, including synthetic methods, such as solid phase, liquid phase and combination solid phase/liquid phase syntheses; recombinant DNA methods, including cDNA cloning, optionally combined with site directed mutagenesis; and/or purification of the natural products.

Methods of Obtaining Crystals of a CARM1 Domain or its Homologues

The invention also relates to a method of obtaining a crystal of a CARM1 domain or homologue thereof, comprising the steps of:

a) optionally producing and purifying a CARM1 domain or homologue thereof;

b) combining a crystallization solution with said CARM1 domain or homologue thereof to produce a crystallizable composition; and

c) subjecting the composition to conditions which promote crystallization and obtaining said crystal.

In another embodiment, the invention provides methods of obtaining crystals of a CARM1 domain protein, a homologue thereof, or complexes thereof using the steps set forth above. In one embodiment, step (b) is performed with a CARM1 domain or homologue thereof bound to a chemical entity. In another embodiment, the above method further comprises the step of soaking said crystal in a solution comprising a chemical entity that binds to the CARM1 domain or homologue thereof.

In one embodiment the above method of obtaining a crystal of a CARM1 domain or homologue thereof, the step of optionally producing and purifying a CARM1 domain or homologue thereof comprises one or more of the steps of: (i) generating TOPO adapted plasmids encoding the target sequence, that optionally encode one or more polypeptide extensions of the N- or C-termini of the CARM1-like methyltransferase sequence [e.g. a His tag] that is known to be useful by those of skill in the art of protein production and purification; (ii) transfecting into an expression system, such as, for example, E. Coli or baculovirus; (iii) inducing expression of the CARM1-like methyltransferase protein product; (iv) screening for over-expression of particular constructs; and (v) purifying the over-expressed proteins.

In certain embodiments, the method of making crystals of a CARM1 domain, a homologue, or a CARM1 domain protein or homologue complex includes the use of a device for promoting crystallizations. Devices for promoting crystallization can include but are not limited to the hanging-drop, sitting-drop, sandwich-drop, dialysis, microbatch or microtube batch devices (U.S. Pat. Nos. 4,886,646, 5,096,676, 5,130,105, 5,221,410 and 5,400,741; Pav, S., et al., Proteins Struct. Funct. Genet, 20: 98-102 (1994); Chayen, Acta. Cryst., D54: 8-15 (1998), Chayen, Structure, 5: 1269-1274 (1997), D'Arcy et al., J. Cryst. Growth, 168: 175-180 (1996) and Chayen, J. Appl. Cryst., 30: 198-202 (1997), incorporated herein by reference). The hanging-drop, sitting-drop and some adaptations of the microbatch methods (D'Arcy et al., J. Cryst. Growth, 168: 175-180 (1996) and Chayen, J. Appl. Cryst., 30: 198-202 (1997)) produce crystals by vapor diffusion. The hanging drop and sitting drop containing the crystallizable composition is equilibrated against a reservoir containing a higher or lower concentration of precipitant. As the drop approaches equilibrium with the reservoir, the saturation of protein in the solution leads to the formation of crystals.

Microseeding may be used to increase the size and quality of crystals. In this instance, microcrystals are crushed to yield a stock seed solution. The stock seed solution is diluted in series. Using a needle, glass rod, micro-pipet, micro-loop or strand of hair, a small sample from each diluted solution is added to a set of equilibrated drops containing a protein concentration equal to or less than a concentration needed to create crystals without the presence of seeds. The aim is to end up with a single seed crystal that will act to nucleate crystal growth in the drop.

It would be readily apparent to one of skill in the art to vary the crystallization conditions disclosed above to identify other crystallization conditions that would produce crystals of CARM1 protein, CARM1 protein complex, CARM1 domain protein complex or homologue thereof, or CARM1 domain homologue. Such variations include, but are not limited to, adjusting pH, protein concentration and/or crystallization temperature, changing the identity or concentration of salt and/or precipitant used, using a different method for crystallization, or introducing additives such as detergents (e.g., TWEEN 20 (monolaurate), LDOA, Brji 30 (4 lauryl ether)), sugars (e.g., glucose, maltose), organic compounds (e.g., dioxane, dimethylformamide), lanthanide ions, or poly-ionic compounds that aid in crystallizations. High throughput crystallization assays may also be used to assist in finding or optimizing the crystallization condition.

In certain embodiments, the crystal comprising a domain of a CARM1 protein or a homologue thereof diffract X-rays to a resolution of at least 2.0 Å. In other embodiments, the crystal comprising a domain of a CARM1 domain, a homologue, or a CARM1 domain protein or homologue complex diffract X-rays to a resolution of at least 5.0 Å, at least 3.5 Å, at least 3.0 Å, at least 2.5 Å, or at least 2.2 Å.

In certain embodiments, the crystal comprising a domain of a CARM1 protein, a homologue thereof, or complexes thereof can produce an electron density map having resolution of at least 2.0 Å. In other embodiments, the crystal comprising a domain of a CARM1 domain, a homologue, or a CARM1 domain protein or homologue complex can produce an electron density map having resolution of at least 5.0 Å, at least 3.5 Å, at least 3.0 Å, at least 2.5 Å, or at least 2.2 Å.

In certain embodiments, the electron density map produced above is sufficient to determine the atomic coordinates a domain of a CARM1 protein or a homologue thereof.

Binding Pockets of CARM1 Protein or its Homologues

As disclosed herein, applicants have provided the first three-dimensional X-ray structure of CARM1. The atomic coordinate data is presented in FIG. 1A.

To use the structure coordinates generated for the CARM1 domain or one of its binding pockets or a CARM1-like binding pocket, it may be necessary to convert the structure coordinates, or portions thereof, into a three-dimensional shape (i.e., a three-dimensional representation of these proteins and binding pockets). This is achieved through the use of a computer comprising commercially available software that is capable of generating three-dimensional representations or structures of molecules or molecular complexes, or portions thereof, from a set of structure coordinates. These three-dimensional representations may be displayed on a computer screen.

Binding pockets, also referred to as binding sites in the present invention, are of significant utility in fields such as drug discovery. The association of natural ligands or substrates with the binding pockets of their corresponding receptors or enzymes is the basis of many biological mechanisms of action. Similarly, many drugs exert their biological effects through association with the binding pockets of receptors and enzymes. Such associations may occur with all or part of the binding pocket. An understanding of such associations will help lead to the design of drugs having more favorable associations with their target receptor or enzyme, and thus, improved biological effects. Therefore, this information is valuable in designing potential binders of the binding pockets of biologically important targets. The binding pockets of this invention are useful and important for drug design.

The conformations of CARM1 and other proteins at a particular amino acid site, along the polypeptide backbone, can be compared using well-known procedures for performing sequence alignments of the amino acids. Such sequence alignments allow for the equivalent sites on these proteins to be compared. Such methods for performing sequence alignment include, but are not limited to, the “bestfit” program and CLUSTAL W Alignment Tool, Higgins et al., supra.

The SAM binding pocket comprises the amino acid residues found within the near vicinity of SAH bound to CARM1.

In one embodiment, the SAM binding pocket comprises amino acid residues F137, R140, Y149, F150, Y153, Q159, M162, M163, R168, D190, V191, G192, C193, G194, S195, G196, I197, L198, S199, V213, E214, A215, S216, M218, G240, K241, V242, E243, S256, E257, P258, E266, M268, and S271, according to the structure of CARM1 in FIG. 1A. The above-identified amino acid residues were within 5 Å (“5 Å sphere amino acids”) of SAH bound to CARM1. These residues were identified using the program Sybyl (Tripos Associates, St. Louis, Mo.), which allow the display of the structure, and a software program to calculate the residues within 5 Å of SAH bound to CARM1. QUANTA (Molecular Simulations, Inc., San Diego, Calif. ©1998, 2000; Accelrys ©2001, 2002), O (T. A. Jones et al., Acta Cryst., A47: 110-119 (1991)) and RIBBONS (Carson, J. Appl. Cryst., 24: 958-961 (1991)) may also be used to obtain the above residues.

In another embodiment, the SAM binding pocket comprises amino acids V136, F137, S138, R140, T141, A146, Y149, F150, N151, F152, Y153, G154, Y155, Q158, Q159, Q160, N161, M162, M163, Q164, D165, R168, T169, Y172, I176, L189, D190, V191, G192, C193, G194, S195, G196, I197, L198, S199, F200, F201, A212, V213, E214, A215, S216, T217, M218, A219, A222, I238, P239, G240, K241, V242, E243, E244, V245, I255, S256, E257, P258, M259, G260, E266, R267, M268, L269, E270, S271, Y272, H274, A275, H414, W415, and N446 according to the structure of CARM1 protein in FIG. 1A. These amino acid residues were within 8 Å (“8 Å sphere amino acids”) of SAH bound to CARM1. These residues were identified using the program Sybyl (Tripos Associates, St. Louis, Mo.). QUANTA, O and RIBBONS, supra may also be used to obtain the above residues.

In another embodiment, the SAM binding pocket comprises amino acids F137, R140, Y149, F150, Y153, Q159, M162, M163, R168, G192, C193, G194, S195, I197, L198, V213, E214, A215, S216, G240, K241, V242, E243, E257, M268, and S271 according to the structure of CARM1 protein in FIG. 1A. These amino acid residues are within 3.8 Å of SAH bound to CARM1. These residues were identified using the program Sybyl (Tripos Associates, St. Louis, Mo.).

In another embodiment, the SAM binding pocket comprises amino acids F150, R168, D190, C193, L198, A212, E214, V242 and E243 according to the structure of CARM1 protein in FIG. 1A. These amino acid residues make contacts less than 3.8 Å in length with SAH bound to CARM1 (F150 makes primarily hydrophobic interactions or van der Waals contacts; and R168, D190, C193, L198, A212, E214, V242 and E243 form direct or indirect hydrogen bonds). These residues were identified using the program Sybyl (Tripos Associates, St. Louis, Mo.).

In another embodiment, the SAM binding pocket comprises amino acids F137, R140, Y149, Y153, Q159, M162, M163, G192, G194, I197, V213, A215, S216, G240, E257, M268, and S271 according to the structure of CARM1 protein in FIG. 1A.

In another embodiment, the SAM binding pocket comprises amino acids Y149, Y153, M162, M259, Y261, E266, H414 and W415 according to the structure of CARM1 protein in FIG. 1A.

In another embodiment, the SAM binding pocket comprises amino acids R168, E214, and E243 according to the structure of CARM1 protein in FIG. 1A.

It will be readily apparent to those of skill in the art that the numbering of amino acid residues in homologues of human CARM1 may be different than that set forth for human CARM1. Corresponding amino acid residues in homologues of CARM1 are easily identified by visual inspection of the amino acid sequences or by using commercially available homology software programs. Homologues of CARM1 include, for example, CARM1 from other species, such as non-humans primates, mouse, rat, etc.

Those of skill in the art understand that a set of structure coordinates for an enzyme or an enzyme-complex, or a portion thereof, is a relative set of points that define a shape in three dimensions. Thus, it is possible that an entirely different set of coordinates could define a similar or identical shape. Moreover, slight variations in the individual coordinates will have little effect on overall shape. In terms of binding pockets, these variations would not be expected to significantly alter the nature of ligands that could associate with those pockets.

The variations in coordinates discussed above may be generated because of mathematical manipulations of the CARM1 structure coordinates. For example, the structure coordinates set forth in FIG. 1A could undergo crystallographic permutations, fractionalization, integer additions or subtractions, inversion, or any combination of the above.

Alternatively, modifications in the crystal structure due to mutations, additions, substitutions, and/or deletions of amino acids, or other changes in any of the components that make up the crystal could also account for variations in structure coordinates. If such variations are within a certain root mean square deviation as compared to the original coordinates, the resulting three-dimensional shape is considered encompassed by this invention. Thus, for example, a ligand that bound to the binding pocket of CARM1 would also be expected to bind to another binding pocket whose structure coordinates defined a shape that fell within the acceptable root mean square deviation.

Various computational analyses may be necessary to determine whether a molecule or the binding pocket or portion thereof is sufficiently similar to the CARM1 binding pockets described above. Such analyses may be carried out using well known software applications, such as ProFit (A. C. R. Martin, SciTech Software, ProFit version 1.8, University College London, http://www.bioinf.org.uk/software), Swiss-Pdb Viewer (Guex et al., Electrophoresis, 18: 2714-2723 (1997)), the Molecular Similarity application of QUANTA (Molecular Simulations, Inc., San Diego, Calif. ©1998, 2000; Accelrys ©2001, 2002) and as described in the accompanying User's Guide, which are incorporated herein by reference.

The above programs permit comparisons between different structures, different conformations of the same structure, and different parts of the same structure. The procedure used in QUANTA (Molecular Simulations, Inc., San Diego, Calif. ©1998, 2000; Accelrys ©2001, 2002) and Swiss-Pdb Viewer to compare structures is divided into four steps: 1) load the structures to be compared; 2) define the atom equivalences in these structures; 3) perform a fitting operation on the structures; and 4) analyze the results.

The procedure used in ProFit to compare structures includes the following steps: 1) load the structures to be compared; 2) specify selected residues of interest; 3) define the atom equivalences in the selected residues; 4) perform a fitting operation on the selected residues; and 5) analyze the results.

Each structure in the comparison is identified by a name. One structure is identified as the target (i.e., the fixed structure); all remaining structures are working structures (i.e., moving structures). Since atom equivalency within QUANTA (Molecular Simulations, Inc., San Diego, Calif. ©1998, 2000; Accelrys ©2001, 2002) is defined by user input, for the purpose of this invention we will define equivalent atoms as protein backbone atoms N, C, O and Cα or all corresponding amino acids between the two structures being compared.

The corresponding amino acids may be identified by sequence alignment programs such as the “bestfit” program available from the Genetics Computer Group which uses the local homology algorithm described by Smith and Waterman in Advances in Applied Mathematics 2, 482-489 (1981), which is incorporated herein by reference. A suitable amino acid sequence alignment will require that the proteins being aligned share a minimum percentage of identical amino acids. Generally, a first protein being aligned with a second protein should share in excess of about 35% identical amino acids (Hanks, S. K., et al., Science, 241, 42-52 (1988); Hanks, S. K. and Quinn, A. M. Methods in Enzymology, 200: 38-62 (1991)). The identification of equivalent residues can also be assisted by secondary structure alignment, for example, aligning the α-helices, β-sheets in the structure. The program Swiss-Pdb Viewer has its own best fit algorithm that is based on secondary sequence alignment.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by the above programs. The Swiss-Pdb Viewer program sets an RMSD cutoff for eliminating pairs of equivalent atoms that have high RMSD values. An RMSD cutoff value can be used to exclude pairs of equivalent atoms with extreme individual RMSD values. In the program ProFit, the RMSD cutoff value can be specified by the user.

For the purpose of this invention, any molecule, molecular complex, binding pocket, motif, domain thereof or portion thereof that is within a root mean square deviation for backbone atoms (N, Cα, C, O) when superimposed on the relevant backbone atoms described by structure coordinates listed in FIG. 1A are encompassed by this invention.

One embodiment of this invention provides a crystalline molecule comprising a protein defined by structure coordinates of a set of amino acid residues that are identical to CARM1 amino acid residues according to FIG. 1A, wherein the RMSD between said set of amino acid residues and said CARM1 amino acid residues is not more than about 5.0 Å. In other embodiments, the RMSD between said set of amino acid residues and said CARM1 amino acid residues is not greater than about 4.0 Å, not greater than about 3.0 Å, not greater than about 2.0 Å, not greater than about 1.5 Å, not greater than about 1.0 Å, or not greater than about 0.5 Å.

In one embodiment, the present invention provides a crystalline molecule comprising all or part of a binding pocket defined by a set of amino acid residues comprising at least six amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the RMSD of the backbone atoms between said CARM1 amino acid residues and said at least six amino acid residues which are identical is not greater than about 3.0 Å. In other embodiments, the RMSD is not greater than about 2.0 Å, 1.0 Å, 0.8, 0.5 Å, 0.3 Å, or 0.2 Å. In other embodiments, the binding pocket is defined by a set of amino acid residues comprising at least four, six, eight, twelve, or fifteen amino acid residues which are identical to said CARM1 amino acid residues.

In one embodiment, the present invention provides a crystalline molecule comprising all or part of a binding pocket defined by a set of amino acid residues which are identical to human CARM1 amino acid residues F150, R168, D190, C193, L198, A212, E214, V242 and E243 according to FIG. 1A, wherein the RMSD of the backbone atoms between said CARM1 amino acid residues and said set of amino acid residues which are identical is not greater than about 3.0 Å. In other embodiments, the RMSD is not greater than about 2.0 Å, 1.0 Å, 0.8, 0.5 Å, 0.3 Å, or 0.2 Å. In other embodiments, the binding pocket is defined by a set of amino acid residues comprising at least four, five, six, or seven amino acid residues identical to said CARM1 amino acid residues.

In one embodiment, the present invention provides a crystalline molecule comprising all or part of a binding pocket defined by a set of amino acid residues comprising a set of amino acid residues which are identical to human CARM1 amino acid residues R168, E214, and E243 according to FIG. 1A, wherein the RMSD of the backbone atoms between said CARM1 amino acid residues and said set of amino acid residues which are identical is not greater than about 3.0 Å. In other embodiments, the RMSD is not greater than about 2.0 Å, 1.0 Å, 0.8, 0.5 Å, 0.3 Å, or 0.2 Å.

In one embodiment, the above molecule is CARM1 protein, CARM1 domain or homologues thereof. In another embodiment, the above molecules are in crystalline form. A CARM1 protein may be human CARM1. Homologues of human CARM1 can be CARM1 from another species, such as a mouse, a rat or a non-human primate.

Computer Systems

According to another embodiment, this invention provides a machine-readable data storage medium, comprising a data storage material encoded with machine-readable data, wherein said data defines the above-mentioned molecules or molecular complexes or binding pockets thereof. In one embodiment, the data defines the above-mentioned binding pockets by comprising the structure coordinates of said amino acid residues according to FIG. 1A. To use the structure coordinates generated for CARM1, homologues thereof, or one of its binding pockets, it is at times necessary to convert them into a three-dimensional shape or to extract three-dimensional structural information from them. This is achieved through the use of commercially or publicly available software that is capable of generating a three-dimensional structure or a three-dimensional representation of molecules or portions thereof from a set of structure coordinates. In one embodiment, three-dimensional structure or representation may be displayed graphically.

Therefore, according to another embodiment, this invention provides a machine-readable data storage medium comprising a data storage material encoded with machine-readable data. In one embodiment, a machine programmed with instructions for using said data is capable of generating a three-dimensional structure or three-dimensional representation of any of the molecules, or molecular complexes or binding pockets thereof, which are described herein.

This invention also provides a computer comprising:

(a) a machine-readable data storage medium, comprising a data storage material encoded with machine-readable data, wherein said data defines any one of the above molecules or molecular complexes;

(b) a working memory for storing instructions for processing said machine-readable data;

(c) a central processing unit (CPU) coupled to said working memory and to said machine-readable data storage medium for processing said machine readable data and means for generating three-dimensional structural information of said molecule or molecular complex; and

(d) output hardware coupled to said central processing unit for outputting three-dimensional structural information of said molecule or molecular complex, or information produced by using said three-dimensional structural information of said molecule or molecular complex.

In one embodiment, the data defines the binding pocket of the molecule or molecular complex.

Three-dimensional data generation may be provided by an instruction or set of instructions, such as a computer program or commands for generating a three-dimensional structure or graphical representation from structure coordinates, or by subtracting distances between atoms, calculating chemical energies for a CARM1 molecule or molecular complex or homologues thereof, or calculating or minimizing energies for an association of a CARM1 molecule or molecular complex or homologues thereof to a chemical entity. The graphical representation can be generated or displayed by commercially available software programs. Examples of software programs include but are not limited to QUANTA (Accelrys ©2001, 2002), O (Jones et al., Acta Crystallogr. A47: 110-119 (1991)) and RIBBONS (Carson, J. Appl. Crystallogr., 24: 9589-961 (1991)), which are incorporated herein by reference. Certain software programs may imbue this representation with physico-chemical attributes which are known from the chemical composition of the molecule, such as residue charge, hydrophobicity, torsional and rotational degrees of freedom for the residue or segment, etc. Examples of software programs for calculating chemical energies are described in the Rational Drug Design section.

Information about said binding pocket or information produced by using said binding pocket can be outputted through display terminals, touchscreens, facsimile machines, modems, CD-ROMs, printers, a CD or DVD recorder, ZIP™ or JAZ™ drives or disk drives. The information can be in graphical or alphanumeric form.

In one embodiment, the computer is executing an instruction such as a computer program for generating three-dimensional structure or docking. In another embodiment, the computer further comprises a commercially available software program to display the information as a graphical representation. Examples of software programs include but as not limited to, QUANTA (Accelrys ©2001, 2002), O (Jones et al., Acta Crystallogr. A47: 110-119 (1991)) and RIBBONS (Carson, J. Appl. Crystallogr., 24: 9589-961 (1991)), all of which are incorporated herein by reference.

FIG. 5 demonstrates one version of these embodiments. System (10) includes a computer (11) comprising a central processing unit (“CPU”) (20), a working memory (22) which may be, e.g., RAM (random-access memory) or “core” memory, mass storage memory (24) (such as one or more disk drives, CD-ROM drives or DVD-ROM drives), one or more cathode-ray tube (“CRT”), LCD, or plasma display terminals (26), one or more keyboards (28), one or more input lines (30), and one or more output lines (40), all of which are, interconnected by a conventional bi-directional system bus (50).

Input hardware (35), coupled to computer (11) by input lines (30), may be implemented in a variety of ways. Machine-readable data of this invention may be inputted via the use of a modem or modems (32) connected by a telephone line or dedicated data line (34). Alternatively or additionally, the input hardware (35) may comprise CD-ROM or DVD-ROM drives or disk drives (24). In conjunction with display terminal (26), keyboard (28) may also be used as an input device.

Output hardware (46), coupled to computer (11) by output lines (40), may similarly be implemented by conventional devices. By way of example, output hardware (46) may include a CRT, LCD or plasma display terminal (26) for displaying a graphical representation of a binding pocket of this invention using a program such as QUANTA (Molecular Simulations, Inc., San Diego, Calif. ©1998, 2000; Accelrys ©2001, 2002) as described herein. Output hardware may also include a printer (42), so that hard copy output may be produced, or a disk drive (24), to store system output for later use. Output hardware may also include a display terminal, touchscreens, facsimile machines, modems, a CD or DVD recorder, ZIP™ or JAZ™ drives, disk drives, or other machine-readable data storage device.

In operation, CPU (20) coordinates the use of the various input and output devices (35), (46), coordinates data accesses from mass storage (24) and accesses to and from working memory (22), and determines the sequence of data processing steps. A number of programs may be used to process the machine-readable data of this invention. Such programs are discussed in reference to the computational methods of drug discovery as described herein. Specific references to components of the hardware system (10) are included as appropriate throughout the following description of the data storage medium.

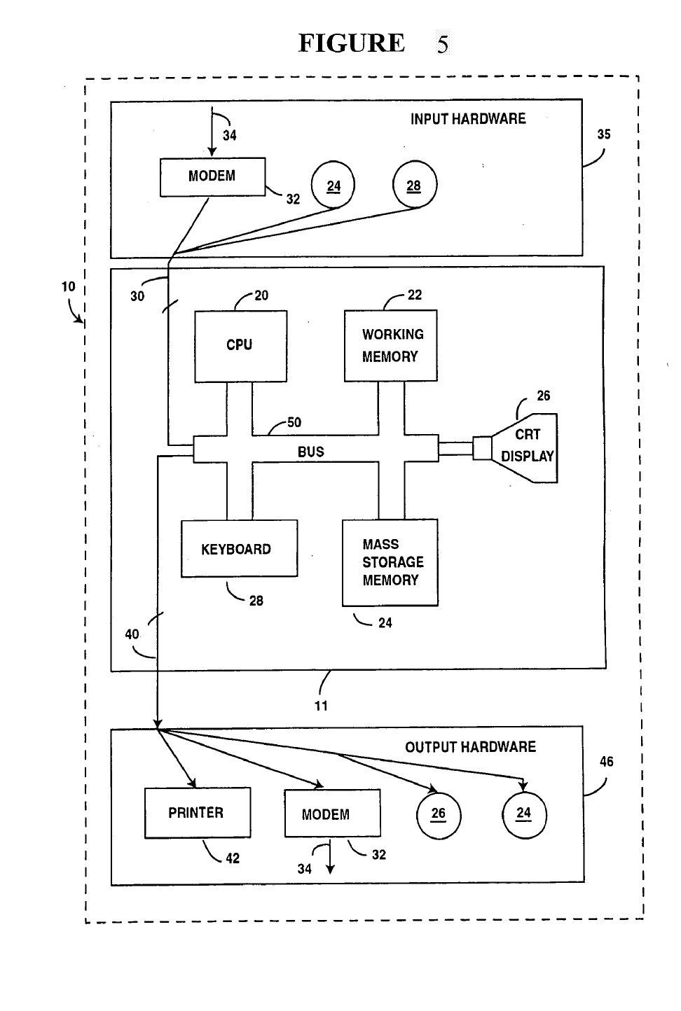

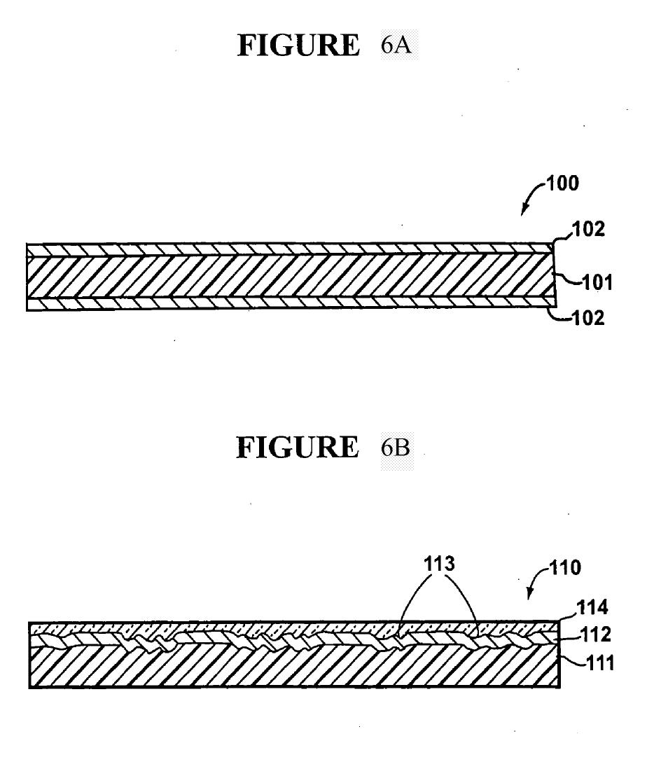

FIG. 6A shows a cross section of a magnetic data storage medium (100) that can be encoded with a machine-readable data that can be carried out by a system such as system (10) of FIG. 5. Medium (100) can be a conventional floppy diskette or hard disk, having a suitable substrate (101), which may be conventional, and a suitable coating (102), which may be conventional, on one or both sides, containing magnetic domains (not visible) whose polarity or orientation can be altered magnetically. Medium (100) may also have an opening (not shown) for receiving the spindle of a disk drive or other data storage device (24).

The magnetic domains of coating (102) of medium (100) are polarized or oriented so as to encode in manner which may be conventional, machine readable data such as that described herein, for execution by a system such as system (10) of FIG. 5.

FIG. 6B shows a cross section of an optically-readable data storage medium (110) which also can be encoded with such a machine-readable data, or set of instructions, which can be carried out by a system such as system (10) of FIG. 5. Medium (110) can be a conventional compact disk read only memory (CD-ROM) or a rewritable medium such as a magneto-optical disk which is optically readable and magneto-optically writable. Medium (100) preferably has a suitable substrate (111), which may be conventional, and a suitable coating (112), which may be conventional, usually of one side of substrate (111).

In the case of CD-ROM, as is well known, coating (112) is reflective and is impressed with a plurality of pits (113) to encode the machine-readable data. The arrangement of pits is read by reflecting laser light off the surface of coating (112). A protective coating (114), which preferably is substantially transparent, is provided on top of coating (112).

In the case of a magneto-optical disk, as is well known, coating (112) has no pits (113), but has a plurality of magnetic domains whose polarity or orientation can be changed magnetically when heated above a certain temperature, as by a laser (not shown). The orientation of the domains can be read by measuring the polarization of laser light reflected from coating (112). The arrangement of the domains encodes the data as described above.

In one embodiment, the structure coordinates of said molecules or molecular complexes or binding pockets are produced by homology modeling of at least a portion of the structure coordinates of FIG. 1A. Homology modeling can be used to generate structural models of CARM1 homologues or other homologous proteins based on the known structure of CARM1 domain. This can be achieved by performing one or more of the following steps: performing sequence alignment between the amino acid sequence of a molecule (possibly an unknown molecule) against the amino acid sequence of CARM1; identifying conserved and variable regions by sequence or structure; generating structure coordinates for structurally conserved residues of the unknown structure from those of CARM1; generating conformations for the structurally variable residues in the unknown structure; replacing the non-conserved residues of CARM1 with residues in the unknown structure; building side chain conformations; and refining and/or evaluating the unknown structure.

Software programs that are useful in homology modeling include XALIGN (Wishart, D. S., et al., Comput. Appl. Biosci., 10: 687-88 (1994)) and CLUSTAL W Alignment Tool, Higgins et al., supra. See also, U.S. Pat. No. 5,884,230. These references are incorporated herein by reference.

To perform the sequence alignment, programs such as the “bestfit” program available from the Genetics Computer Group (Waterman in Advances in Applied Mathematics 2, 482 (1981), which is incorporated herein by reference) and CLUSTAL W Alignment Tool (Higgins et al., supra, which is incorporated by reference) can be used. To model the amino acid side chains of homologous molecules, the amino acid residues in CARM1 can be replaced, using a computer graphics program such as “O” (Jones et al, (1991) Acta Cryst. Sect. A, 47: 110-119), by those of the homologous protein, where they differ. The same orientation or a different orientation of the amino acid can be used. Insertions and deletions of amino acid residues may be necessary where gaps occur in the sequence alignment. However, certain portions of the active site of CARM1 and its homologues are highly conserved with essentially no insertions and deletions.

Homology modeling can be performed using, for example, the computer programs SWISS-MODEL available through Glaxo Wellcome Experimental Research in Geneva, Switzerland; WHATIF available on EMBL servers; Schnare et al., J. Mol. Biol, 256: 701-719 (1996); Blundell et al., Nature 326: 347-352 (1987); Fetrow and Bryant, Bio/Technology 11:479-484 (1993); Greer, Methods in Enzymology 202: 239-252 (1991); and Johnson et al, Crit. Rev. Biochem. Mol. Biol. 29:1-68 (1994). An example of homology modeling can be found, for example, in Szklarz G. D., Life Sci. 61: 2507-2520 (1997). These references are incorporated herein by reference.

Thus, in accordance with the present invention, data capable of generating the three-dimensional structure or three-dimensional representation of the above molecules or molecular complexes, or binding pockets thereof, can be stored in a machine-readable storage medium, which is capable of displaying structural information or a graphical three-dimensional representation of the structure. In one embodiment, means of generating three-dimensional information is provided by means for generating a three-dimensional structural representation of the binding pocket or protein or protein complex.

Rational Drug Design

The CARM1 structure coordinates or the three-dimensional graphical representation generated from these coordinates may be used in conjunction with a computer for a variety of purposes, including drug discovery.

For example, the structure encoded by the data may be computationally evaluated for its ability to associate with chemical entities. Chemical entities that associate with CARM1 may inhibit or activate CARM1 or its homologues, and are potential drug candidates. Alternatively, the structure encoded by the data may be displayed in a graphical three-dimensional representation on a computer screen. This allows visual inspection of the structure, as well as visual inspection of the structure's association with chemical entities.

In one embodiment, the invention provides a method of using a computer for selecting an orientation of a chemical entity that interacts favorably with a binding pocket or domain comprising the steps of:

(a) providing the structure coordinates of said binding pocket or domain on a computer comprising means for generating three-dimensional structural information from said structure coordinates;

(b) employing computational means to dock a first chemical entity in the binding pocket or domain;

(c) quantifying the association between said chemical entity and all or part of the binding pocket or domain for different orientations of the chemical entity; and

(d) selecting the orientation of the chemical entity with the most favorable interaction based on said quantified association.

In one embodiment, the docking is facilitated by said quantified association.

In one embodiment, the above method further comprises the following steps before step (a):

(e) producing a crystal of a molecule or molecular complex comprising a CARM1 domain or homologue thereof;

(f) determining the three-dimensional structure coordinates of the molecule or molecular complex by X-ray diffraction of the crystal; and

(g) identifying all or part of a binding pocket that corresponds to said binding pocket

Three-dimensional structural information in step (a) may be generated by instructions such as a computer program or commands that can generate a three-dimensional representation; subtract distances between atoms; calculate chemical energies for a CARM1 molecule, molecular complex or homologues thereof; or calculate or minimize the chemical energies of an association of CARM1 molecule, molecular complex or homologues thereof to a chemical entity. These types of computer programs are known in the art. The graphical representation can be generated or displayed by commercially available software programs. Examples of software programs include but are not limited to QUANTA (Molecular Simulations, Inc., San Diego, Calif. ©1998, 2000; Accelrys ©2001, 2002), O (Jones et al., Acta Crystallogr. A47: 110-119 (1991)) and RIBBONS (Carson, J. Appl. Crystallogr., 24: 9589-961 (1991)), which are incorporated herein by reference. Certain software programs may imbue this representation with physico-chemical attributes which are known from the chemical composition of the molecule, such as residue charge, hydrophobicity, torsional and rotational degrees of freedom for the residue or segment, etc. Examples of software programs for calculating chemical energies are described below.

The above method may further comprise the following step after step (d): outputting said quantified association to a suitable output hardware, such as a CRT, LCD or plasma display terminal, a CD or DVD recorder, ZIP™ or JAZ™ drive, a disk drive, or other machine-readable data storage device, as described previously. The method may further comprise generating a three-dimensional structure, graphical representation thereof, or both, of the protein, binding pocket, molecule or molecular complex prior to step (b).

One embodiment of this invention provides the above method, wherein energy minimization, molecular dynamics simulations, rigid body minimizations combinations thereof, or similar induced-fit manipulations are performed simultaneously with or following step (b).

The above method may further comprise the steps of:

(e) repeating steps (b) through (d) with a second chemical entity; and

(f) selecting at least one of said first or second chemical entity that interacts more favorably with said-binding pocket or domain based on said quantified association of said first or second chemical entity.

In another embodiment, the invention provides the method of using a computer for selecting an orientation of a chemical entity with a favorable shape complementarity in a binding pocket comprising the steps of:

(a) providing the structure coordinates of said binding pocket and all or part of the SAM binding motif bound therein on a computer comprising means for generating three-dimensional structural information from said structure coordinates;

(b) employing computational means to dock a first chemical entity in the binding pocket;

(c) quantitating the contact score of said chemical entity in different orientations in the binding pocket; and

(d) selecting an orientation with the highest contact score.

In one embodiment, the docking is monitored and directed or facilitated by the contact score.

The method above may further comprise the step of generating a three-dimensional graphical representation of the binding pocket and all or part of the SAM binding motif bound therein prior to step (b).

The method above may further comprise the steps of:

(e) repeating steps (b) through (d) with a second chemical entity; and

(f) selecting at least one of said first or second chemical entity that has a higher contact score based on said quantitated contact score of said first or second chemical entity.

In another embodiment, the invention provides a method for screening a plurality of chemical entities to associate at a deformation energy of binding of no greater than 7 kcal/mol with said binding pocket:

(a) employing computational means, which utilize said structure coordinates to dock one of said chemical entities from the plurality of chemical entities and said binding pocket;

(b) quantifying the deformation energy of binding between the chemical entity and the binding pocket;

(c) repeating steps (a) and (b) for each remaining chemical entity; and

(d) outputting a set of chemical entities that associate with the binding pocket at a deformation energy of binding of not greater than 7 kcal/mol to a suitable output hardware.

In another embodiment, the method comprises the steps of:

(a) constructing a computer model of a binding pocket of a molecule or molecular complex;

(b) selecting a chemical entity to be evaluated by a method selected from the group consisting of assembling said chemical entity; selecting a chemical entity from a small molecule database; de novo ligand design of said chemical entity; and modifying a known agonist or binder, or a portion thereof, of a CARM1 protein, or homologue thereof to produce said chemical entity;

(c) employing computational means to dock said chemical entity to be evaluated in said binding pocket in order to provide an energy-minimized configuration of said chemical entity in the binding pocket; and

(d) evaluating the results of said docking to quantify the association between said chemical entity and the binding pocket

Alternatively, the structure coordinates of the CARM1 binding pockets may be utilized in a method for identifying a candidate binder of a molecule or molecular complex comprising a binding pocket of CARM1. This method comprises the steps of:

(a) using a three-dimensional structure of the binding pocket or domain of CARM1 to design, select or optimize a plurality of chemical entities;

(b) contacting each chemical entity with the molecule and molecular complex;

(c) monitoring the inhibition to the catalytic activity of the molecule or molecular complex by the chemical entity; and

(d) selecting a chemical entity based on the effect of the chemical entity on the activity of the molecule or molecular complex.

Monitoring the inhibition to the CARM1 catalytic activity can be performed by any CARM1 assay known in the art (e.g. see United States published Application 2005/0196753, or International Patent Publication No. WO 03/102143), or any of the CARM1 assays described herein.

In one embodiment, step (a) is carried out using a three-dimensional structure of the binding pocket or domain or portion thereof of the molecule or molecular complex. In another embodiment, the three-dimensional structure is displayed as a graphical representation.

In another embodiment, the method comprises the steps of:

(a) constructing a computer model of a binding pocket of the molecule or molecular complex;

(b) selecting a chemical entity to be evaluated by a method selected from the group consisting of assembling said chemical entity; selecting a chemical entity from a small molecule database; de novo ligand design of said chemical entity; and modifying a known binder, or a portion thereof, of a CARM1 protein or homologue thereof to produce said chemical entity;

(c) employing computational means to dock said chemical entity to be evaluated and said binding pocket in order to provide an energy-minimized configuration of said chemical entity in the binding pocket; and

(d) evaluating the results of said docking to quantify the association between said chemical entity and the binding pocket;

(e) synthesizing said chemical entity; and

(f) contacting said chemical entity with said molecule or molecular complex to determine the ability of said chemical entity to activate or inhibit said molecule.

In one embodiment, the invention provides a method of designing a compound or complex that associates with all or part of the binding pocket of a domain of a CARM1 protein comprising the steps of:

(a) providing the structure coordinates of said binding pocket or domain on a computer comprising means for generating three-dimensional structural information from said structure coordinates;

(b) using the computer to dock a first chemical entity in part of the binding pocket or domain;

(c) docking a second chemical entity in another part of the binding pocket or domain;

(d) quantifying the association between the first and second chemical entity and part of the binding pocket or domain;

(e) repeating steps (b) to (d) with another first and second chemical entity and selecting a first and a second chemical entity based on said quantified association of all of said first and second chemical entity;

(f) optionally, visually inspecting the relationship of the first and second chemical entity to each other in relation to the binding pocket or domain on a computer screen using the three-dimensional graphical representation of the binding pocket or domain and said first and second chemical entity; and

(g) assembling the first and second chemical entity into a compound or complex that interacts with said binding pocket by model building.

For the first time, the present invention permits the use of molecular design techniques to identify, select and design chemical entities, including compounds, capable of binding to CARM1 or CARM1-like binding pockets and domains.

Applicants' elucidation of binding pockets of CARM1 provides the necessary information for designing new chemical entities and compounds that may interact with CARM1 substrate, active site, SAM binding pockets or CARM1-like substrate, active site or SAM binding pockets, in whole or in part.

Throughout this section, discussions about the ability of a chemical entity to bind to, interact with or inhibit CARM1 binding pockets refer to features of the entity alone.

The design of compounds that bind to or inhibit CARM1 binding pockets according to this invention generally involves consideration of two factors. First, the chemical entity must be capable of physically and structurally associating with parts or all of the CARM1 binding pockets. Non-covalent molecular interactions important in this association include hydrogen bonding, van der Waals interactions, hydrophobic interactions and electrostatic interactions.

Second, the chemical entity must be able to assume a conformation that allows it to associate with the CARM1 binding pockets directly. Although certain portions of the chemical entity will not directly participate in these associations, those portions of the chemical entity may still influence the overall conformation of the molecule. This, in turn, may have a significant impact on potency. Such conformational requirements include the overall three-dimensional structure and orientation of the chemical entity in relation to all or a portion of the binding pocket, or the spacing between functional groups of a chemical entity comprising several chemical entities that directly interact with the CARM1 or CARM1-like binding pockets.

The potential effect of a chemical entity on CARM1 binding pockets may be analyzed prior to its actual synthesis and testing by the use of computer modeling techniques. If the theoretical structure of the given entity suggests insufficient interaction and association between it and the CARM1 binding pockets, testing of the entity is obviated. However, if computer modeling indicates a strong interaction, the molecule may then be synthesized and tested for its ability to bind to a CARM1 binding pocket This may be achieved by testing the ability of the molecule to inhibit CARM1 using the assays described herein.

A potential binder of a CARM1 binding pocket may be computationally evaluated by means of a series of steps in which chemical entities or fragments are screened and selected for their ability to associate with the CARM1 binding pockets.

One skilled in the art may use one of several methods to screen chemical entities or fragments or moieties thereof for their ability to associate with the binding pockets described herein. This process may begin by visual inspection of, for example, any of the binding pockets on the computer screen based on the CARM1 structure coordinates FIG. 1A, or other coordinates which define a similar shape generated from the machine-readable storage medium. Selected chemical entities, or fragments or moieties thereof may then be positioned in a variety of orientations, or docked, within that binding pocket as defined supra. Docking may be accomplished using software such as QUANTA (Accelrys ©2001, 2002) and Sybyl (Tripos Associates, St. Louis, Mo.), followed by, or performed simultaneously with, energy minimization, rigid-body minimization (Gshwend, supra) and molecular dynamics with standard molecular mechanics force fields, such as CHARMM and AMBER.

Specialized computer programs may also assist in the process of selecting fragments or chemical entities. These include:

1. GRID (Goodford, P. J., “A Computational Procedure for Determining Energetically Favorable Binding Sites on Biologically Important Macromolecules”, J. Med. Chem., 28: 849-857 (1985)). GRID is available from Oxford University, Oxford, UK.

2. MCSS (Miranker, A., et al., “Functionality Maps of Binding Sites: A Multiple Copy Simultaneous Search Method.” Proteins Struct. Funct. Genet, 11: 29-34 (1991)). MCSS is available from Accelrys, San Diego, Calif.

3. AUTODOCK (Goodsell, D. S., et al., “Automated Docking of Substrates to Proteins by Simulated Annealing”, Proteins Struct., Funct., and Genet, 8: 195-202 (1990)). AUTODOCK is available from Scripps Research Institute, La Jolla, Calif.

4. DOCK (Kuntz, I. D., et al., “A Geometric Approach to Macromolecule-Ligand Interactions”, J. Mol. Biol., 161: 269-288 (1982)). DOCK is available from University of California, San Francisco, Calif.

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of CARM1. This would be followed by manual model building using software such as QUANTA (Accelrys ©2001, 2002) or Sybyl (Tripos Associates, St. Louis, Mo.).

Useful programs to aid one of skill in the art in connecting the individual chemical entities or fragments include:

1. CAVEAT (Bartlett, P. A., et al., “CAVEAT: A Program to Facilitate the Structure-Derived Design of Biologically Active Molecules”, in Molecular Recognition in Chemical and Biological Problems, S. M. Roberts, Ed., Royal Society of Chemistry, Special Publication No. 78: pp. 182-196 (1989); Lauri, G. and Bartlett, P. A., “CAVEAT: A Program to Facilitate the Design of Organic Molecules”, J. Comp. Aid. Molec. Design, 8: 51-66 (1994)). CAVEAT is available from the University of California, Berkeley, Calif.

2. 3D Database systems such as ISIS (MDL Information Systems, San Leandro, Calif.). This area is reviewed in Martin, Y. C., “3D Database Searching in Drug Design”, J. Med. Chem., 35: 2145-2154 (1992).

3. HOOK (Eisen, M. B., et al., “HOOK: A Program for Finding Novel Molecular Architectures that Satisfy the Chemical and Steric Requirements of a Macromolecule Binding Site”, Proteins Struct., Funct., Genet, 19: 199-221 (1994)). HOOK is available from Accelrys, San Diego, Calif.

Instead of proceeding to build of a CARM1 binding pocket in a step-wise fashion one fragment or chemical entity at a time as described above, other CARM1 binding compounds may be designed as a whole or “de novo” using either an empty binding pocket or optionally including some portion(s) of a known binder(s). There are many de novo ligand design methods including:

1. LUDI (Bohm, H.-J., “The Computer Program LUDI: A New Method for the De Novo Design of Enzyme Binders”, J. Comp. Aid. Molec. Design, 6: pp. 61-78 (1992)). LUDI is available from Accelrys, San Diego, Calif.

2. LEGEND (Nishibata, Y., et al., Tetrahedron, 47: 8985-8990 (1991)). LEGEND is available from Accelrys, San Diego, Calif.

3. LeapFrog (available from Tripos Associates, St. Louis, Mo.).

4. SPROUT (Gillet, V., et al., “SPROUT: A Program for Structure Generation)”, J. Comp. Aid. Molec. Design, 7: 127-153 (1993)). SPROUT is available from the University of Leeds, UK.

Other molecular modeling techniques may also be employed in accordance with this invention (see, e.g., Cohen, N. C., et al., “Molecular Modeling Software and Methods for Medicinal Chemistry, J. Med. Chem., 33: 883-894 (1990); see also, Navia, M. A. and Murcko, M. A., “The Use of Structural Information in Drug Design”, Current Opinions in Structural Biology, 2: 202-210 (1992); Balbes, L. M., et al., “A Perspective of Modern Methods in Computer-Aided Drug Design”, in Reviews in Computational Chemistry, K. B. Lipkowitz and D. B. Boyd, Eds., VCH Publishers, New York, 5: pp. 337-379 (1994); see also, Guida, W. C., “Software For Structure-Based Drug Design”, Curr. Opin. Struct. Biology, 4: 777-781 (1994)); Sherman, W., et al., “Novel Procedure for Modeling Ligand/Receptor Induced Fit Effects”, J. Med. Chem., 49: 534-553 (2006)).

Once a chemical entity has been designed or selected by the above methods, the efficiency with which that entity may bind to any of the above binding pockets may be tested and optimized by computational evaluation. For example, an effective binding pocket binder must preferably demonstrate a relatively small difference in energy between its bound and free states (i.e., a small deformation energy of binding). Thus, the most efficient binding pocket binders should preferably be designed with a magnitude of deformation energy of binding of not greater than about 10 kcal/mole, more preferably, not greater than 7 kcal/mole. Binding pocket binders may interact with the binding pocket in more than one conformation that is similar in overall binding energy. In those cases, the deformation energy of binding is taken to be the difference between the energy of the free entity and the average energy of the conformations observed when the binder binds to the protein.

A chemical entity designed or selected as binding to any one of the above binding pockets may be further computationally optimized so that in its bound state it would preferably lack repulsive electrostatic interaction with the target enzyme and with the surrounding water molecules. Such non-complementary electrostatic interactions include repulsive charge-charge, dipole-dipole and charge-dipole interactions.

Specific computer software is available in the art to evaluate compound deformation energy and electrostatic interactions. Examples of programs designed for such uses include: Gaussian 94, revision C (M. J. Frisch, Gaussian, Inc., Pittsburgh, Pa. ©1995); AMBER, version 4.1 (P. A. Kollman, University of California at San Francisco, ©1995); QUANTA/CHARMM (Accelrys ©2001, 2002); Insight II/Discover (Accelrys., San Diego, Calif. ©1998); DelPhi (Accelrys, Inc., San Diego, Calif. 1998); and AMSOL (Quantum Chemistry Program Exchange, Indiana University). These programs may be implemented, for instance, using a Silicon Graphics workstation such as an Indigo2 with “IMPACT” graphics. Other hardware systems and software packages will be known to those skilled in the art.

Another approach enabled by this invention is the computational screening of small molecule databases for chemical entities or compounds that can bind in whole, or in part, to any of the above binding pocket. In this screening, the quality of fit of such entities to the binding pocket may be judged either by shape complementarity or by estimated interaction energy (Meng, E. C., et al., J. Comp. Chem., 13: 505-524 (1992)).

According to another embodiment, the invention provides chemical entities that associate with a CARM1 binding pocket produced or identified by the method set forth above.

Another particularly useful drug design technique enabled by this invention is iterative drug design. Iterative drug design is a method for optimizing associations between a protein and a chemical entity by determining and evaluating the three-dimensional structures of successive sets of protein/chemical entity complexes.

In iterative drug design, crystals of a series of protein or protein complexes are obtained and then the three-dimensional structures of each crystal is solved. Such an approach provides insight into the association between the proteins and compounds of each complex. This is accomplished by selecting compounds with binding capacity, obtaining crystals of this new protein/compound complex, solving the three-dimensional structure of the complex, and comparing the associations between the new protein/compound complex and previously solved protein/compound complexes. By observing how changes in the compound affected the protein/compound associations, these associations may be optimized.

In some cases, iterative drug design is carried out by forming successive protein-compound complexes and then crystallizing each new complex. High throughput crystallization assays may be used to find a new crystallization condition or to optimize the original protein crystallization condition for the new complex. Alternatively, a pre-formed protein crystal may be soaked in the presence of a binder, thereby forming a protein/compound complex and obviating the need to crystallize each individual protein/compound complex.

Any of the above methods may be used to design peptide or small molecule mimics of the SAM binding motif which may have effects on the activity of full-length CARM1 protein or fragments thereof, or on the activity of full-length but mutated CARM1 protein or fragments of the mutated protein thereof.

In one embodiment, the present invention provides a method for identifying a candidate binder that interacts with a binding site of a CARM1 protein or a homologue thereof, comprising the steps of:

(a) obtaining a crystal comprising a domain of said CARM1 protein or said homologue thereof, wherein the crystal is characterized with space group P_{21 21 2} and has unit cell parameters of a=74.852, b=98.629 Å, c=207.316 Å;

(b) obtaining the structure coordinates of amino acids of the crystal of step (a), wherein the structure coordinates are set forth in FIG. 1A-1 to 1A-240;

(c) generating a three-dimensional model of the domain of said CARM1 protein or said homologue thereof using the structure coordinates of the amino acids generated in step (b), a root mean square deviation from backbone atoms of said amino acids of not more than ±2.0 Å;

(d) determining a binding site of the domain of said CARM1 protein or said homologue thereof from said three-dimensional model; and

(e) performing computer fitting analysis to identify the candidate binder which interacts with said binding site.

In one embodiment, the present invention provides the method for identifying a candidate binder that interacts with a binding site of a CARM1 protein or a homologue thereof, further comprising the step of: (f) contacting the identified candidate binder with the domain of said CARM1 protein or said homologue thereof in order to determine the effect of the binder on CARM1 protein activity.

In one embodiment, the present invention provides the method for identifying a candidate binder that interacts with a binding site of a CARM1 protein or a homologue thereof, wherein the binding site of the domain of said CARM1 protein or said homologue thereof determined in step (d) comprises the structure coordinates according to FIG. 1A-1 to 1A-240 of amino acid residues R168, E214, and E243, wherein the root mean square deviation from the backbone atoms of said amino acids is not more than ±2.0 Å.

In one embodiment, the present invention provides the method for identifying a candidate binder that interacts with a binding site of a CARM1 protein or a homologue thereof, wherein the binding site of the domain of said CARM1 protein or said homologue thereof determined in step (d) comprises the structure coordinates according to FIG. 1A-1 to 1A-240 of amino acid residues F150, R168, D190, C193, L198, A212, E214, V242 and E243, wherein the root mean square deviation from the backbone atoms of said amino acids is not more than ±2.0 Å.

In one embodiment, the present invention provides the method for identifying a candidate binder that interacts with a binding site of a CARM1 protein or a homologue thereof, wherein the binding site of the domain of said CARM1 protein or said homologue thereof determined in step (d) comprises the structure coordinates according to FIG. 1A-1 to 1A-240 of amino acid residues F621, K644, A657, E661, M664, L802, S806, C807, V808, H809, R810, D811, D829, and L832, wherein the root mean square deviation from the backbone atoms of said amino acids is not more than ±2.0 Å.

In one embodiment, the present invention provides a method for identifying a candidate binder that interacts with a binding site of a domain of a CARM1 protein or a homologue thereof, comprising the steps of:

(a) obtaining a crystal comprising the domain of said CARM1 protein or said homologue thereof, wherein the crystal is characterized with space group P_{21 21 2} and has unit cell parameters of a=74.852, b=98.629 Å, c=207.316 Å;

(b) obtaining the structure coordinates of amino acids of the crystal of step (a);

(c) generating a three-dimensional model of said CARM1 protein or said homologue thereof using the structure coordinates of the amino acids generated in step (b), a root mean square deviation from backbone atoms of said amino acids of not more than ±2.0 Å;

(d) determining a binding site of the domain of said CARM1 protein or said homologue thereof from said three-dimensional model; and

(e) performing computer fitting analysis to identify the candidate binder which interacts with said binding site. In one embodiment, the step of obtaining a crystal is optional.

In one embodiment, the present invention provides the method for identifying a candidate binder that interacts with a binding site, further comprising the step of:

(f) contacting the identified candidate binder with the domain of said CARM1 protein or said homologue thereof in order to determine the effect of the binder on CARM1 activity.

One embodiment of this invention provides the method for identifying a candidate binder that interacts with a binding site, wherein the binding site of the domain of said CARM1 protein or said homologue thereof determined in step (d) comprises the structure coordinates according to FIG. 1A-1 to 1A-240 of amino acid residues R168, E214, and E243, wherein the root mean square deviation from the backbone atoms of said amino acids is not more than ±2.0 Å.

One embodiment of this invention provides the method for identifying a candidate binder that interacts with a binding site, wherein the binding site of the domain of said CARM1 protein or said homologue thereof determined in step (d) comprises the structure coordinates according to FIG. 1A-1 to 1A-240 of amino acid residues F150, R168, D190, C193, L198, A212, E214, V242 and E243, wherein the root mean square deviation from the backbone atoms of said amino acids is not more than ±2.0 Å.

In one embodiment, the present invention provides the method for identifying a candidate binder that interacts with a binding site, wherein the binding site of the domain of said CARM1 protein or said homologue thereof determined in step (d) comprises the structure coordinates according to FIG. 1A-1 to 1A-240 of amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415, wherein the root mean square deviation from the backbone atoms of said amino acids is not more than ±2.0 Å.

In one embodiment, the present invention provides a method for identifying a candidate binder that interacts with a binding site of a domain of a CARM1 protein or a homologue thereof, comprising the step of determining a binding site of the domain of said CARM1 protein or the homologue thereof from a three-dimensional model to design or identify the candidate binder which interacts with said binding site.

In one embodiment, the present invention provides the method for identifying a candidate binder that interacts with a binding site of a domain of a CARM1 protein or a homologue thereof, wherein the binding site of the domain of said CARM1 protein or said homologue thereof determined comprises the structure coordinates according to FIG. 1A-1 to 1A-240 of amino acid residues R168, E214, and E243, wherein the root mean square deviation from the backbone atoms of said amino acids is not more than ±2.0 Å.

In one embodiment, the present invention provides the method for identifying a candidate binder that interacts with a binding site of a domain of a CARM1 protein or a homologue thereof, wherein the binding site of the domain of said CARM1 protein or said homologue thereof determined comprises the structure coordinates according to FIG. 1A-1 to 1A-240 of amino acid residues F150, R168, D190, C193, L198, A212, E214, V242 and E243, wherein the root mean square deviation from the backbone atoms of said amino acids is not more than ±2.0 Å.

In one embodiment, the present invention provides the method for identifying a candidate binder that interacts with a binding site of a domain of a CARM1 protein or a homologue thereof, wherein the binding site of the domain of said CARM1 protein or said homologue thereof determined comprises the structure coordinates according to FIG. 1A-1 to 1A-240 of amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415, wherein the root mean square deviation from the backbone atoms of said amino acids is not more than ±2.0 Å.

One embodiment of this invention provides a method for identifying a candidate binder of a molecule or molecular complex comprising a binding pocket or domain selected from the group consisting of:

(i) a set of amino acid residues which are identical to human CARM1 amino acid residues R168, E214, and E243 according to FIG. 1A, wherein the root mean square deviation of the backbone atoms between the set of amino acid residues and the CARM1 amino acid residues is not greater than about 2.0 Å;

(ii) a set of amino acid residues comprising at least three amino acid residues which are identical to human CARM1 amino acid residues F150, R168, DI 90, C193, L198, A212, E214, V242 and E243 according to FIG. 1A, wherein the root mean square deviation of the backbone atoms between the at least three amino acid residues and the CARM1 amino acid residues which are identical is not greater than about 2.0 Å;

(iii) a set of amino acid residues comprising at least five amino acid residues which are identical to human CARM1 amino acid residues F150, R168, D190, C193, L198, A212, E214, V242 and E243 according to FIG. 1A, wherein the root mean square deviation of the backbone atoms between the at least five amino acid residues and the CARM1 amino acid residues which are identical is not greater than about 2.0 Å;

(iv) a set of amino acid residues comprising at least five amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least five amino acid residues and the CARM1 amino acid residues which are identical is not greater than about 2.0 Å;

(v) a set of amino acid residues comprising at least six amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least six amino acid residues and the CARM1 amino acid residues which are identical is not greater than about 2.0 Å; and

(vi) a set of amino acid residues that are identical to CARM1 amino acid residues according to FIG. 1A, wherein the root mean square deviation between the set of amino acid residues and the CARM1 amino acid residues is not more than about 2.0 Å;

(vii) a set of amino acid residues that are identical to CARM1 amino acid residues according to FIG. 1A, wherein the root mean square deviation between the set of amino acid residues and the CARM1 amino acid residues is not more than about 3.0 Å;

comprising the steps of:

(a) using a three-dimensional structure of the binding pocket or domain to design, select or optimize a plurality of chemical entities; and

(b) selecting said candidate binder based on the effect of said chemical entities on said domain of said CARM1 protein or said domain of said CARM1 protein homologue on the catalytic activity of the molecule.

In one embodiment, the present invention provides a method of using a crystal of a domain of said CARM1 protein or a homologue in a binder screening assay comprising:

(a) selecting a potential binder by performing rational drug design with a three-dimensional structure determined for the crystal, wherein said selecting is performed in conjunction with computer modeling;

(b) contacting the potential binder with a methyltransferase; and

(c) detecting the ability of the potential binder to modulate the activity of the methyltransferase.

In certain embodiments, the ability of the potential binder for modulating the kinase is assessed using an enzyme inhibition assay. In other embodiments, the ability of the potential binder for inhibiting the kinase is performed using a cellular-based assay.

In one embodiment, the present invention provides a method for identifying a candidate binder that interacts with a binding site of a CARM1 protein or a homologue thereof comprising:

(a) obtaining a crystal of a CARM1 protein or a homologue thereof;

(b) obtaining the atomic coordinates of the crystal; and

(c) using the atomic coordinates and one or more molecular modeling techniques to identify the candidate binder that interacts with a binding site of a CARM1 protein or a homologue thereof. In certain embodiments, the crystal comprises a domain of a CARM1 protein or a homologue thereof. In one embodiment, the step of obtaining a crystal is optional.

In one embodiment, the present invention provides the method for identifying a candidate binder that interacts with a binding site of a CARM1 protein or a homologue thereof, wherein the one or more molecular modeling techniques are selected from the group consisting of graphic molecular modeling and computational chemistry.

In one embodiment, the present invention provides the method for identifying a candidate binder that interacts with a binding site of a CARM1 protein or a homologue thereof, further comprising the candidate binder with the CARM1 protein or the homologue and detecting binding of the candidate binder to the CARM1 protein or the homologue.

In one embodiment, the present invention provides a method of structure-based identification of candidate compounds for binding to a CARM1 protein or a homologue thereof, comprising:

(a) constructing a three-dimensional structure of the CARM1 protein or a homologue thereof,

(b) performing computer-assisted structure-based drug design with said structure of the CARM1 protein or a homologue; and

(c) identifying at least one candidate binder that is predicted to have a compatible conformation with a binding site of the structure of the CARM1 protein or a homologue.

In certain embodiments, the present invention provides for methods wherein the three-dimensional structure is visualized as a computer image generated when said atomic coordinates determined by X-ray diffraction are analyzed on a computer using a graphical display software program to create an electronic file of the image and visualizing the electronic file on a computer capable of representing the electronic file as a three-dimensional image.

Structure Determination of Other Molecules

The structure coordinates set forth in FIG. 1A can also be used in obtaining structural information about other crystallized molecules or molecular complexes. This may be achieved by any of a number of well-known techniques, including molecular replacement.

According to one embodiment, the machine-readable data storage medium comprises a data storage material encoded with a first set of machine readable data which comprises the Fourier transform of at least a portion of the structure coordinates set forth in FIG. 1A or homology model thereof, and which, when using a machine programmed with instructions for using said data, can be combined with a second set of machine readable data comprising the X-ray diffraction pattern of a molecule or molecular complex to determine at least a portion of the structure coordinates corresponding to the second set of machine readable data.

In another embodiment, the invention provides a computer for determining at least a portion of the structure coordinates corresponding to X-ray diffraction data obtained from a molecule or molecular complex having an unknown structure, wherein said computer comprises:

(a) a machine-readable data storage medium comprising a data storage material encoded with machine-readable data, wherein said data comprises at least a portion of the structure coordinates of CARM1 according to FIG. 1A or a homology model thereof;

(b) a machine-readable data storage medium comprising a data storage material encoded with machine-readable data, wherein said data comprises X-ray diffraction data obtained from said molecule or molecular complex having an unknown structure; and

(c) instructions for performing a Fourier transform of the machine-readable data of (a) and for processing said machine-readable data of (b) into structure coordinates.

For example, the Fourier transform of at least a portion of the structure coordinates set forth in FIG. 1A or homology model thereof may be used to determine at least a portion of the structure coordinates of the molecule or molecular complex.

Therefore, another embodiment this invention provides a method of utilizing molecular replacement to obtain structural information about a molecule or a molecular complex of unknown structure wherein the molecule or molecular complex is sufficiently homologous to CARM1, comprising the steps of:

(a) crystallizing said molecule or molecular complex of unknown structure;

(b) generating an X-ray diffraction pattern from said crystallized molecule or molecular complex;

(c) applying at least a portion of the CARM1 structure coordinates set forth in one of FIG. 1A or a homology model thereof to the X-ray diffraction pattern to generate a three-dimensional electron density map of at least a portion of the molecule or molecular complex whose structure is unknown; and

(d) generating a structural model of the molecule or molecular complex from the three-dimensional electron density map.

In one embodiment, the method is performed using a computer. In another embodiment, the molecule is selected from the group consisting of CARM1 protein and CARM1 domain homologues. In another embodiment, the molecular complex is CARM1 domain complex or homologue thereof.

By using molecular replacement, all or part of the structure coordinates of CARM1 as provided by this invention (and set forth in FIG. 1A) can be used to determine the structure of a crystallized molecule or molecular complex whose structure is unknown more quickly and efficiently than attempting to determine such information ab initio.

Molecular replacement provides an accurate estimation of the phases for an unknown structure. Phases are a factor in equations used to solve crystal structures that cannot be determined directly. Obtaining accurate values for the phases, by methods other than molecular replacement, is a time-consuming process that involves iterative cycles of approximations and refinements and greatly hinders the solution of crystal structures. However, when the crystal structure of a protein containing at least a homologous portion has been solved, the phases from the known structure may provide a satisfactory estimate of the phases for the unknown structure.

Thus, this method involves generating a preliminary model of a molecule or molecular complex whose structure coordinates are unknown, by orienting and positioning the relevant portion of CARM1 protein according to FIG. 1A within the unit cell of the crystal of the unknown molecule or molecular complex so as best to account for the observed X-ray diffraction pattern of the crystal of the molecule or molecular complex whose structure is unknown. Phases can then be calculated from this model and combined with the observed X-ray diffraction pattern amplitudes to generate an electron density map of the structure whose coordinates are unknown. This, in turn, can be subjected to any well-known model building and structure refinement techniques to provide a final, accurate structure of the unknown crystallized molecule or molecular complex (E. Lattman, “Use of the Rotation and Translation Functions”, in Meth. Enzymol., 115: 55-77 (1985); M. G. Rossmann, ed., “The Molecular Replacement Method”, Int. Sci. Rev. Ser., No. 13, Gordon & Breach, New York (1972)).

The structure of any portion of any crystallized molecule or molecular complex that is sufficiently homologous to any portion of the structure of human CARM1 protein can be resolved by this method.

In one embodiment, the method of molecular replacement is utilized to obtain structural information about a CARM1 homologue. The structure coordinates of CARM1 as provided by this invention are particularly useful in solving the structure of CARM1 complexes that are bound by ligands, substrates and binders.

Furthermore, the structure coordinates of CARM1 as provided by this invention are useful in solving the structure of CARM1 proteins that have amino acid substitutions, additions and/or deletions (referred to collectively as “CARM1 mutants”, as compared to naturally occurring CARM1). These CARM1 mutants may optionally be crystallized in co-complex with a chemical entity. The crystal structures of a series of such complexes may then be solved by molecular replacement and compared with that of wild-type CARM1. Potential sites for modification within the various binding pockets of the enzyme may thus be identified. This information provides an additional tool for determining the most efficient binding interactions, for example, increased hydrophobic interactions, between CARM1 and a chemical entity or compound.

The structure coordinates are also particularly useful in solving the structure of crystals of the domain of CARM1 or homologues co-complexed with a variety of chemical entities. This approach enables the determination of the optimal sites for interaction between chemical entities, including candidate CARM1 binders. For example, high resolution X-ray diffraction data collected from crystals exposed to different types of solvent allows the determination of where each type of solvent molecule resides. Small molecules that bind tightly to those sites can then be designed and synthesized and tested for their CARM1 inhibition activity.

All of the molecules and complexes referred to above may be studied using well-known X-ray diffraction techniques and may be refined using 1.5-3.4 Å resolution X-ray data to an R value of about 0.30 or less using computer software, such as X-PLOR (Yale University, ©1992, distributed by Accelrys.; see, e.g., Blundell & Johnson, supra; Meth. Enzymol., vol. 114 & 115, H. W. Wyckoff et al., eds., Academic Press (1985)) or CNS (Brunger et al., Acta Cryst., D54: 905-921, (1998)).

The present invention provides a method for determining the intracellular activity of CARM1 methyltransferase comprising, providing a sample of cells to be tested for CARM1 methyltransferase activity, wherein the cells have been engineered to express a CARM1 methyltransferase peptide substrate that is specific for CARM1 methyltransferase, determining the degree of methylation of the peptide substrate by CARM1 methyltransferase in the sample, and thus determining the intracellular activity of CARM1 methyltransferase in the sample of cells. In one embodiment of this invention the sample of cells is incubated for a period of between 12 and 24 hours prior determining the degree of methylation of the peptide substrate by CARM1 methyltransferase.

The invention further provides a method for identifying an agent that inhibits the intracellular activity of CARM1 methyltransferase comprising, providing a sample of cells having CARM1 methyltransferase activity, wherein the cells have been engineered to express a CARM1 methyltransferase peptide substrate that is specific for CARM1 methyltransferase, determining the degree of reduction of methylation of the peptide substrate by CARM1 methyltransferase by contacting the sample of cells with a test agent and comparing the peptide substrate methylation level with the methylation level of peptide substrate in an identical control sample of cells that was not contacted with the test agent, determining the degree of inhibition of intracellular activity of CARM1 methyltransferase in the sample of cells contacted with the agent, and thus determining whether the test agent is an agent that inhibits the intracellular activity of CARM1 methyltransferase. In one embodiment of this invention the contacting with the test agent is performed over a period of between 12 and 24 hours

In embodiments of the above inventions in which the intracellular activity of CARM1 methyltransferase is determined, the sample of engineered cells comprises a stable cell line with an inducible promoter controlling expression of the CARM1 methyltransferase peptide substrate. In other embodiments the sample of engineered cells comprises cells that are transiently transfected with a plasmid that expresses the CARM1 methyltransferase peptide substrate. The CARM1 methyltransferase peptide substrate in any of the above methods is for example any of poly A binding protein 1 (PABP1; GenBank Accession No. NP 002559), histone H3 (e.g. GenBank Accession No. NP 003484), or any peptides derived from these substrates that possess a site that is methylated by CARM1, e.g. STGGKAPRKQLATKAARK from the N-terminus of histone H3, or QNMPGAIRPAAPRPPFSTMRK from PABP1. These substrates can be optionally modified to improve stability, solubility, ability to isolate methylated product etc. by fusion to other peptide sequences. For example, the substrate can be optionally modified with an epitope that permits it to be readily isolated from a reaction mix, e.g. a FLAG sequence. Examples of such substrates are STGGKAPRKQLATKAARK-(FLAG sequence) or QNMPGAIRPAAPRPPFSTMRK-(FLAG sequence). In additional embodiments of the above methods, the degree of methylation of the peptide substrate can be determined by isolation of the substrate and then determination of the degree of methylation at the site modified by CARM1. This can be done for example by utilizing an immunoprecipitation procedure to isolate the substrate, for example by using an anti-FLAG antibody, followed by SDS-polyacrylamide gel electrophoresis, Western blotting, and detection of the methylated substrate using antibodies specific to the methylated form of the substrate.

In further embodiments of the above inventions in which the intracellular activity of CARM1 methyltransferase is determined, the sample of engineered cells can be optionally engineered to express CARM1, either on the same plasmid as the CARM1 substrate, or on a separate plasmid. In an alternative embodiment, the CARM1 and its peptide substrate are produced as a fusion protein, thus improving the efficiency of the methylation reaction. In the latter case either full length CARM1 or an active catalytic fragment can be used as part of the fusion protein. The fusion protein can be optionally fused to an epitope (e.g. FLAG protein) to assist in its isolation, for example by immunoprecipitation. Thus, in one potential embodiment the catalytic C-terminus of CARM1 is fused to an amino terminal peptide from histone H3 (containing the Arg17 methylation site) and a FLAG sequence to form the fusion protein CARM1-H3peptide-FLAG.

This invention will be better understood from the Experimental Details that follow. However, one skilled in the art will readily appreciate that the specific methods and results discussed are merely illustrative of the invention as described more fully in the claims which follow thereafter, and are not to be considered in any way limited thereto.

### EXPERIMENTAL DETAILS

This invention relates to CARM1, CARM1 binding pockets, or CARM1-like binding pockets. The invention relates to a computer comprising a data storage medium encoded with the structure coordinates of such binding pockets. The invention also relates to methods of using the structure coordinates to solve the structure of homologous proteins or protein complexes. The invention relates to methods of using the structure coordinates to screen for and design compounds that bind to CARM1 protein, complexes of CARM1 protein, homologs thereof, or CARM1-like protein or protein complexes. The invention also relates to crystallizable compositions and crystals comprising a CARM1-like protein or homologs thereof. The invention also relates to methods of identifying binders of CARM1-like proteins.

Materials and Methods

CARM1 Assays

CARM1 biochemical Assay. (e.g. for Determining IC50 Values).

A scintillation proximity assay (SPA) was used for measuring the enzymatic activity of CARM1 and for screening for compounds that specifically inhibit CARM1-dependent methylation of histone H3 and PABP1. A fusion protein of CARM1 to MBP (Maltose Binding protein), expressed and purified from E. coli, was used to methylate peptides derived from either the N-terminus of histone H3 (acetyl-STGGKAPRKQLATKAARK-biotin) or from PABP1 (acetyl-QNMPGAIRPAAPRPPFSTMRK-biotin). The H3 peptide has two residues R17 and R26 that have been reported to be methylated by CARM1 (Brandon et al., Biochemistry, 2001, 40(19):5747-5756). The PABP1 peptide also contains two arginine residues, R455 and R460, similarly methylated by CARM1 (Lee and Bedford, EMBO Rep. 2002, 3(3):268-73). The methylation reaction was conducted in the presence of tritiated S-Adenosyl-L-Methionine (3H-SAM), 1 μg MBP-CARM1, 250 nM peptide substrate, and assay buffer (50 mM Tris pH 8.0, 0.03% BSA, 3 mM DTT). The reaction was allowed to proceed at room temperature for 75 minutes before being stopped by Stop buffer (25 mM Tris pH 7.4, 100 mM EDTA, 1% Tween 20) and Streptavidin-coated SPA beads (2 mg/ml) (GE Healthcare). The beads were allowed to settle overnight before the signal was counted in a TOPCOUNT. The final SAM concentration in the reaction and the ratio of tritiated to unlabelled SAM was adjusted to give a good signal/noise.

Results and Discussion

### Example 1

### CARM1 Expression and Purification

A CARM1 protein of amino acid residues 128 to 480 was cloned and expressed using standard techniques. The expressed 128-480 residue CARM1 protein had 3 amino acids added to its N-terminal end (MetAlaLeu) and 8 amino acids added to the C-terminal end (GluGlyHisHisHisHisHisHis). Plasmids containing ligated inserts were transformed into chemically competent TOP10 cells. Colonies were then screened for inserts in the correct orientation and small DNA amounts were purified using a “miniprep” procedure from 2 ml cultures, using a standard kit, following the manufacturer's instructions. For standard molecular biology protocols followed here, see also, for example, the techniques described in Sambrook et al., Molecular Cloning: A Laboratory Manual, Cold Spring Harbor Laboratory, NY, 2001, and Ausubel et al., Current Protocols in Molecular Biology, Greene Publishing Associates and Wiley Interscience, NY, 1989. The miniprep DNA was transformed into BL21 (DE3) cells and plated onto petri dishes containing selective LB medium agar with 30 mg/ml of kanamycin. Isolated, single colonies were grown to mid-log phase and stored at −80° C. in LB containing 15% glycerol.

The bacterial fermentation of this construct is carried out in a T7 E. coli expression system utilizing LB media. Cells are grown at 32° C. overnight to generate a seed culture. The seed culture is then used to inoculate 2 L baffled shake flasks containing LB media. Growth was carried at 37° C. until an OD600=0.8 was reached, at which time 0.4 mM IPTG is added to induce the culture. Temperature was immediately shifted to 22° C. for a 16 hours overnight induction. Cells were collected by centrifugation and frozen pellets were used for purification of the CARM1 protein.

Frozen cells were lysed in buffer (50 mM Tris-HCl pH7.5, 500 mm NaCl, 20 mM Imidazole, 0.1% Tween 20 with protease inhibitor cocktail (Sigma-Aldrich, Cat. #P8849) by sonication at 4° C. for eight bursts of 15 seconds with 2 minutes cooling between bursts and centrifuged to remove cell debris. The soluble fraction was purified over an IMAC column charged with nickel (GE Healthcare, NJ), and eluted under native conditions with a step gradient of 10 mM, then 500 mM imidazole. The protein was desalted with a desalting column (GE Healthcare, NJ), into 50 mM Bis-Tris pH 8.0, 25 mM Tris pH 8.0, mM methionine, 5 mM DTT. Protein was pooled based on A280 measurements. The protein was then further purified by gel filtration using a Superdex 200 column (GE Healthcare, NJ), into 10 mM HEPES pH7.5, 150 mM NaCl, 10% Glycerol, 10 mM methionine, 5 mM DTT. Protein was pooled based on SDS-PAGE analysis of fractions and concentrated to 11 mg/ml.

### Example 2

### Protein Crystallization for Native CARM1

It was found that a hanging drop or sitting drop containing 1.0111 of protein 11 mg/mL in 10 mM HEPES pH7.5, 150 mM NaCl, 10% Glycerol, 10 mM Methionine, 5 mM DTT and 2 mM SAH and 1.0 μL reservoir solution: 100 mM Tris HCl pH 8.5 and 2.2M ammonium sulfate in a sealed container containing 500 μL reservoir solution, incubated overnight at 21° C. provided diffraction quality crystals. Alternatively, crystals were also grown with a reservoir solution of 100 mM Hepes pH 8.5 and 2.2M Ammonium Sulfate.

### Example 3

### X-ray Diffraction and Structure Determination of CARM1

The crystals were individually harvested from their trays and transferred to a cryoprotectant consisting of 80% reservoir solution plus 20% glycerol. The crystals were collected and transferred into liquid nitrogen. The crystals frozen in liquid nitrogen were transferred to the Advanced Photon Source (Argonne National Laboratory) where data from a single wavelength experiment was collected. Table 1 summarizes information about the data collection.

X-ray diffraction data were indexed and integrated using the program MOSFLM (Collaborative Computational Project, Number 4 (1994) Acta. Cryst. D50, 760-763; http://www.ccp4.ac.uk/main.html) and then merged using the program SCALA (Collaborative Computational Project, Number 4 (1994) Acta. Cryst. D50, 760-763; http://www.ccp4.ac.uk/main.html). The subsequent conversion of intensity data to structure factor amplitudes was carried out using the program TRUNCATE (Collaborative Computational Project, Number 4 (1994) Acta. Cryst. D50, 760-763; http://www.ccp4.ac.uk/main.html). A molecular replacement solution was obtained with the program MOLREP (Collaborative Computational Project, Number 4 (1994) Acta. Cryst. D50, 760-763; http://www.ccp4.ac.uk/main.html) and using the PDB coordinates for the PRMT1 protein arginine methyltransferase (1ORI) as a search model. This model was refined using the program REFMAC (Collaborative Computational Project, Number 4 (1994) Acta. Cryst. D50, 760-763; http://www.ccp4.ac.uk/main.html) with interactive refitting carried out using the program XTALVIEW/XFIT (McRee, D. E. J. Structural Biology (1993) 125:156-65; available from CCMS (San Diego Super Computer Center) CCMS-request@sdsc.edu).

The electron density corresponding to side chains absent from the search model was generally clear and unambiguous in the methyltransferase domain.

The final CARM1 structure contains four copies of the methyltransferase domain (putatively residues 183 to 258), with one SAH molecule bound in each, and 50 water molecules in the unit cell. During the course of the refinement, the electron density corresponding to residues 128-135 in all four copies (chains A-D) and residues 475-480 in chains B and C, 476-480 in chain D, and 477-480 in chain A, was poor and did not improve. Consequently, these residues that reside were removed from the final model. Crystallographic refinement statistics are provided in Table 1.

TABLE 1
CARM1 Data Collection Statistics

| Space group | P 21 21 2 |
|---|---|
| Cell dimensions | a = 75.85 Å |
| | b = 98.63 Å |
| | c = 207.32 Å |
| | a. = 90° |
| | i. = 90° |
| | γ = 90° |
| Wavelength λ | 0.9794 Å |
| Overall Resolution limits | 37.42 Å |
| | 2.45 Å |
| Number of reflections | 403249 |
| collected | |
| Number of unique | 56408 |
| reflections | |
| Overall Redundancy of | 7.1 |
| data | |
| Overall Completeness of | 98.7% |
| data | |
| Completeness of data in last | 92.2% |
| data shell | |
| Overall R_{SYM} | 0.113 |
| R_{SYM} in last resolved shell | 0.346 |
| Overall I/sigma(I) | 11.9 |
| I/sigma(I) in last shell | 4.8 |

### Example 4

### Overview of CARM1 Structure

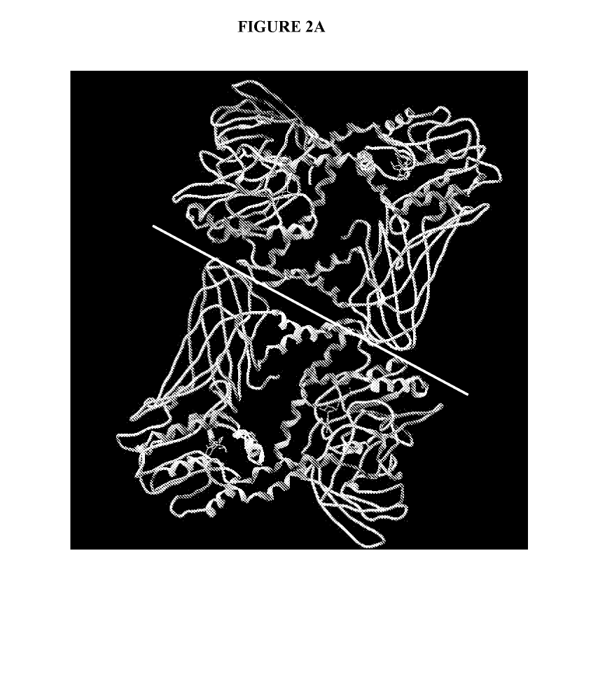

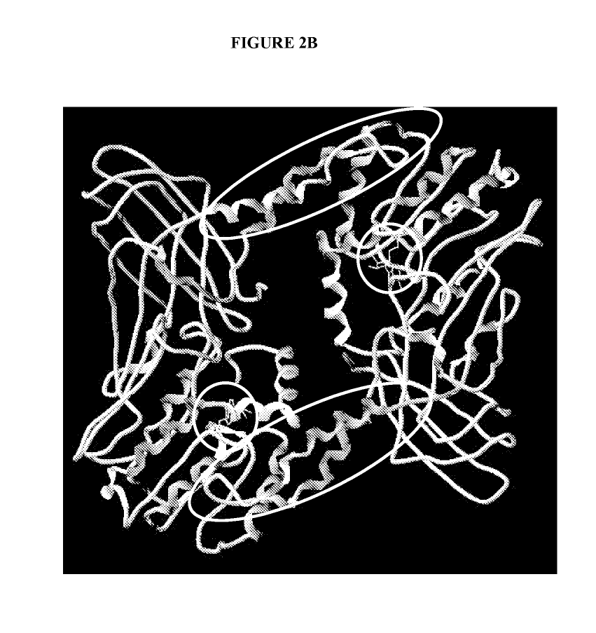

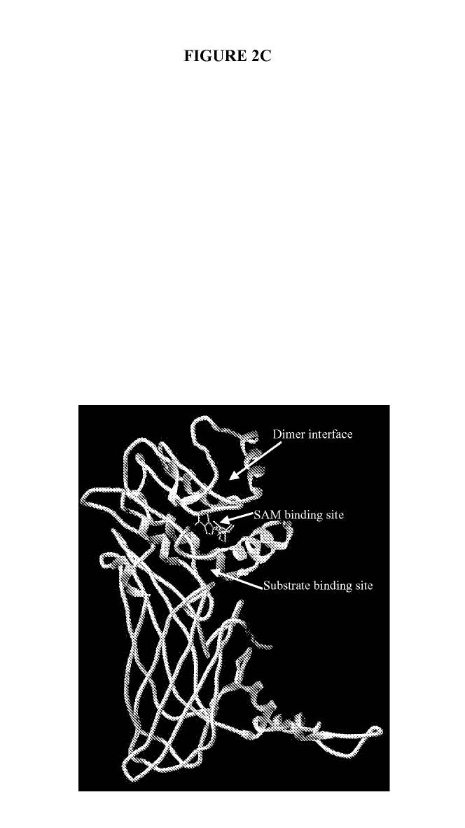

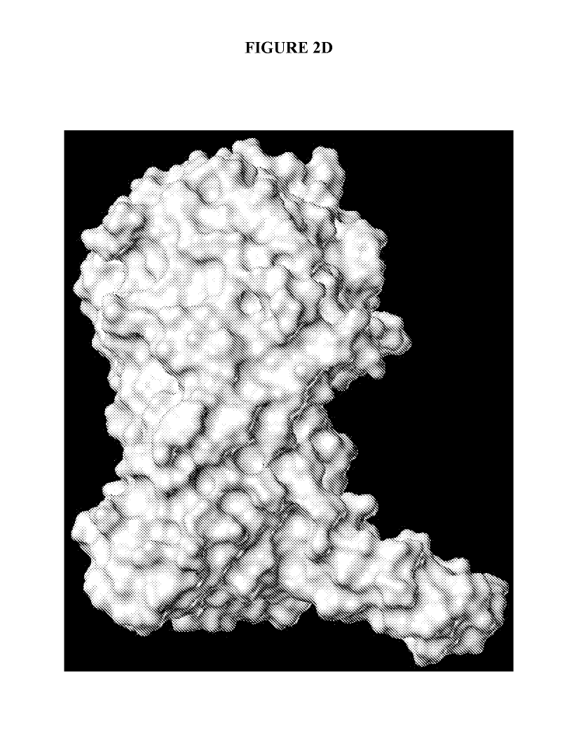

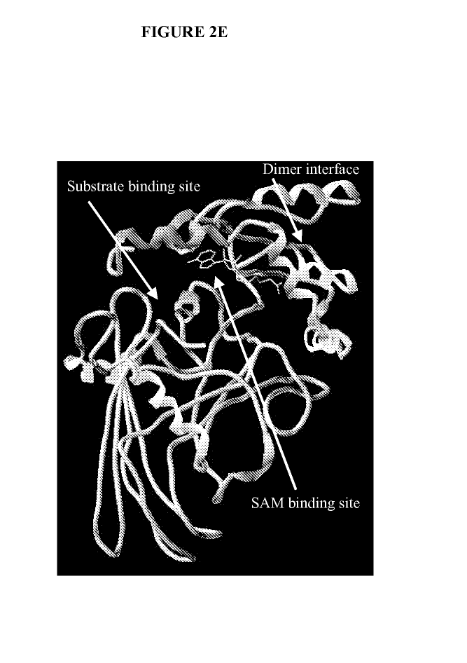

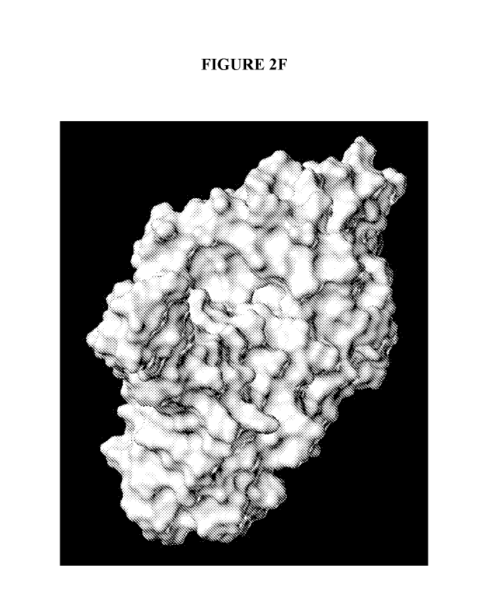

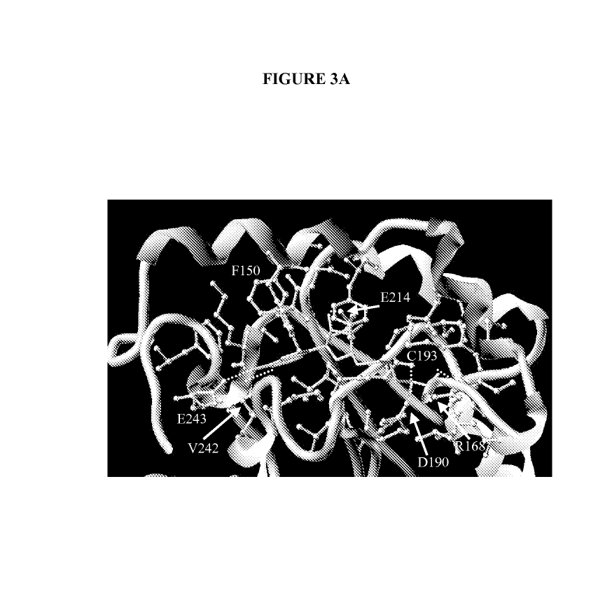

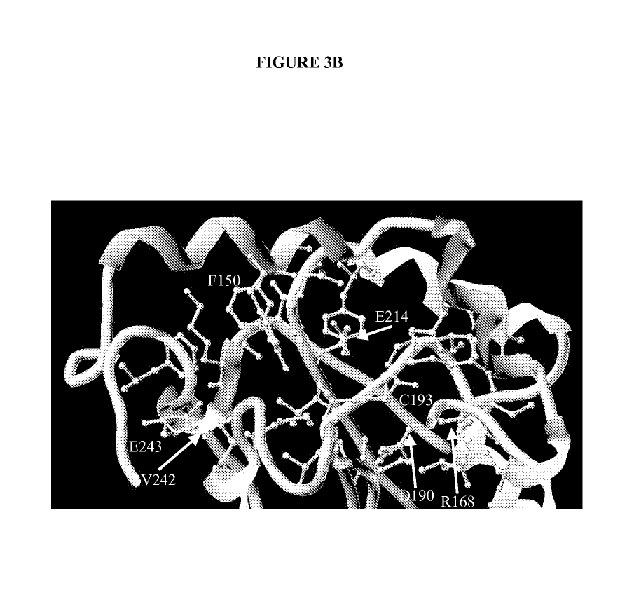

The principal features of the CARM1 structures include a dimer of CARM1 dimers (FIG. 2). The dimer structure is similar to that of PRMT1, with a globular methyltransferase domain and a helix-turn-helix arm that extends to the dimerization partner. Dimerization is anti-parallel. The helical arms from one protein are backed by 8 anti-parallel strands of a β-sandwich and contact a set of 4 helices of the other protein distal to the SAM binding site. The β-sandwich sits below the putative substrate binding cleft. SAH rests within the completely buried SAM binding pocket. Key hydrogen bonds exist between SAH and the pocket. For example, the 6-amino of SAH donates a proton to the side chain of E243. The NI position of SAH accepts a proton from the backbone N of V242. The side chain of E214 can accept protons from either of the ribose hydroxyls. The backbone carbonyl of C193 accepts a proton from the basic amine of SAH and a bridging water also connects that basic amine to the side chain of D190. The side chain of R168 donates two protons to the carboxylate of SAH. In addition, the phenyl ring of F150 makes a pi-edge aromatic-aromatic interaction with the purine ring system. These buried interactions feature low desolvation costs, suggesting a potent binding mode, consistent with experiment. The opening to the substrate binding cleft is maintained, even in the absence of the substrate, facilitating the design of binders to the peptide binding site if so desired.

TABLE 2
CARM1: Secondary structure elements

| Secondary | Starting | Ending |
|---|---|---|
| Structure Type | residue | residue |
| HELIX | PHE137 | ARG140 |
| HELIX | GLU143 | TYR153 |
| HELIX | LEU156 | GLN164 |
| HELIX | TYR166 | LEU177 |
| HELIX | ASN179 | PHE183 |
| HELIX | ILE197 | GLN204 |
| HELIX | MET218 | SER228 |
| HELIX | MET268 | HIS274 |
| HELIX | GLU300 | THR308 |
| HELIX | ALA310 | GLN315 |
| HELIX | LEU323 | ALA325 |
| HELIX | ARG327 | PHE335 |
| HELIX | ASP344 | ILE347 |
| HELIX | LYS363 | LEU367 |
| SHEET | ILE187 | VAL191 |
| SHEET | LYS209 | GLU214 |
| SHEET | ILE235 | PRO239 |
| SHEET | VAL251 | SER256 |
| SHEET | LEU279 | PHE286 |
| SHEET | ILE289 | PHE297 |
| SHEET | VAL339 | ASP341 |
| SHEET | VAL353 | ASN358 |
| SHEET | ARG369 | HIS377 |
| SHEET | GLY382 | ILE396 |
| SHEET | THR401 | SER405 |
| SHEET | GLN417 | ALA428 |
| SHEET | THR433 | ALA442 |
| SHEET | TYR448 | VAL456 |
| SHEET | LYS462 | ASP468 |
| SHEET | PHE473 | PHE474 |

### Example 5

### Docking to the CARM1 Structure

In order to establish the utility of the structure to find chemical matter capable of binding to CARM1, a collection of commercially available compounds were screened on a cluster of Linux boxes using the structure from FIG. 1A in the software FlexX (BioSolveIT, GmbH, Sankt Augustin, Germany) with default parameters. Compounds were ranked according to their FlexX scores. The top 10,000 compounds were grouped by vendor. Sets of compounds with no pricing available or with fewer than 100 compounds from the same vendor were excluded. The approximately 150 remaining compounds were acquired and tested in the CARM1 biochemical assay described herein, yielding the following hit:

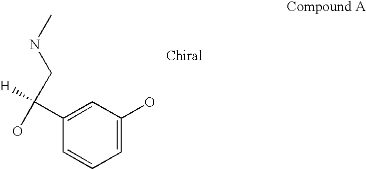

This compound had a low micromolar IC₅₀ at saturating SAM concentrations and was demonstrated to be SAM competitive. Inspection of the predicted binding mode of this hit in the active site suggested key modifications or extensions to this hit would be tolerated by the site. Searches based off a new query based only on the key pharmacophoric elements led to identification of two additional low micromolar hits:

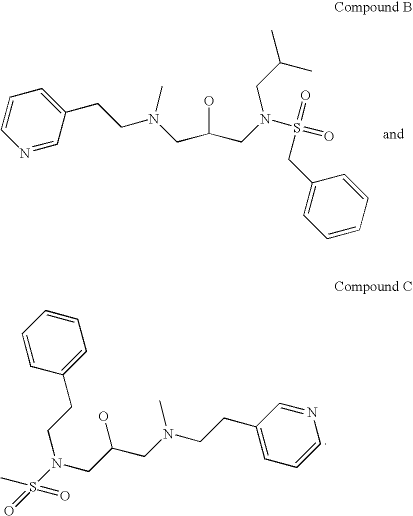

### Example 6

### CARM1 Assays

A critical element for a successful methyltransferase mechanistic assay is to monitor de novo methylation of a substrate. This is necessitated by the fact that the methylation mark on most substrates examined to date is quite stable and its diminution in the presence of an inhibitor would depend on the rate of degradation of the protein and that of new protein synthesis. Monitoring de novo methylation has been achieved previously by incubating cells with L-[methyl-3H]methionine in the presence of the protein synthesis inhibitor, cycloheximide. Since no new protein synthesis occurs methylated proteins get labeled after the tritiated methionine gets converted into the methyl donor S-Adenosyl Methionine. The methylated protein is then immunoprecipitated from the labeled cell extracts and subjected to fluorography and western blotting. The shortcoming of this approach is that it detects total protein methylation but is unable to detect the methylation on a specific amino acid.

One aspect of the invention described herein is a method for monitoring the effect of compounds on substrate methylation that relies on generating a cell line that has the substrate (tagged with a capture/purification tag) under the control of an inducible promoter. Induction and compound addition can then be done simultaneously and protein methylation monitored after an appropriate period of incubation. An alternative to the inducible system that was also used relies on transient transfection of an expression plasmid for the substrate followed by removal of transfection reagent 3-4 hours later, addition of compounds, and incubation for 12-24 hours before cell lysis. In either case, the substrate is immunoprecipitated by an antibody specific to the fused tag then examined for methylation by an antibody raised against the specific methyl-arginine epitope in the substrate.

Our experience with CARM1 demonstrates that if de novo methylation of a transfected or induced substrate is not efficient then engineering a system for ‘tethered catalysis’ might solve the problem. This system has been utilized previously in a yeast two-hybrid approach in which creating a physical linkage between an enzyme and its protein substrate ensures constitutive modification of the substrate. The physical linkage of the two proteins results in more efficient catalysis than a co-expression situation (Guo et al., 2004, Nat Biotechnol. 22(7):888-92). An example would be the linkage of an amino terminal peptide from histone H3 (containing Arg 17) to the C-terminus of the CARM1 coding sequence. An additional sequence coding for the Flag epitope is fused to the amino terminus of CARM1. The expression of the resulting protein Flag-CARM1-H3pep is induced simultaneously with the addition to cells of potential CARM1 inhibitors. Afterwards, Flag-CARM1-H3pep is captured from cell lysates with an anti-Flag antibody and methylation of the tethered H3 peptide detected by anti-me-Arg17-H3 antibody.

The gene for a CARM1 protein substrate X (PrX) is cloned as a fusion to a purification tag (e.g Flag tag) in an expression vector under the control of an inducible promoter. Example of such a promoter is the Tet-inducible promoter of the plasmid pcDNA5/TO [Invitrogen].

The expression plasmid containing the gene for Flag-PrX is transfected into a Tet system-compatible cell lines (such as HEK293 T-Rex or HeLa T-Rex from Invitrogen) and clones are selected in the presence of a selection agent (e.g. Hygromycin for pcDNA5/TO). Stable transfectant clones that demonstrate Tet-inducible expression of Flag-PrX are chosen.

A stable cell clone is used for monitoring the cellular activity of small molecule CARM1 inhibitors. An inhibitor is added to the cells at different concentration simultaneously with the addition of Tetracycline. The inhibitor if active will inhibit the de novo methylation of protein X synthesized from the Tet-inducible promoter.

After an incubation period of an appropriate period of time (usually 8-24 hours), cells are lysed, Flag-PrX is either immunoprecipitated with an anti-Flag antibody linked to beads or captured on an ELISA plate coated with anti-Flag antibody.

Immunoprecipitated Flag-PrX is then run on a SDS-PAGE gel, blotted to a membrane, and detected simultaneously with two antibodies, anti-Flag antibody and anti-Methyl-Arg-PrX antibody. The two antibodies are derived from different species and hence can be detected by different dye-conjugated secondary antibodies that allow quantitation with a LI-COR instrument.

Alternatively, the methylation status of a specific CARM1-modified arginine residue on the Flag-PrX captured on ELISA plate can be detected by incubation with an anti-Methyl-Arg-PrX antibody and an HRP-conjugated secondary antibody.

A variation of the approach detailed above is to tether a CARM1 peptide substrate (such as an amino-terminal peptide from histone H3) to the C-terminus of Flag-CARM1. The resulting Flag-CARM1-H3pep is captured by anti-Flag antibody. Methylation of Arg17-H3 is then detected by an anti-me-Arg17-H3 antibody.

The cellular methylation inhibitors sinefungin, 5-Deoxy-5-Methylthioadenosine (MTA), and periodate-oxidized adenosine (AdOx) are used a controls to validate the different substrate/methyl-specific antibody combinations. These inhibit most known methyltransferase enzymes within the cell, the first two through competitive inhibition of SAM binding and AdOx through inhibiting S-adenosylhomocysteine hydrolase. S-adenosylhomocysteine hydrolase inhibition causes the accumulation of S-adenosylhomocysteine, a product of the methyl transfer reaction and a potent inhibitor of most methyltransferase enzymes.

Flag-PABP1 Tet Induction and Methylation Assay.

Detailed Assay Protocol:

Hek-293 T-REX with a stable integration of the pcDNA5-TO-3xFlag-PABP1 plasmid were plated at 0.4×10⁶ cells/well onto a collagen-coated six well plate in DMEM supplemented with 10% FCS. The pcDNA5-TO-3xFlag-PABP1 plasmid has the PABP1 gene (polyA binding protein 1) fused to a 3xFlag tag and under the control of tetracycline operator (TO) DNA elements.

The following day a serial dilution of each compound to be tested was added to wells (20, 10, 5, 2.5, . . . μM) and the expression of Flag-PABP1 was induced by the addition of 110 g/ml tetracycline. Twenty four hours later cells were harvested via scraping and collected by spinning for 5 minutes @ 4° C. in a 15 ml tube. Cells were then washed with 1 ml PBS, transferred to Eppendorf tubes and re-spun for 5 minutes. The supernatant was aspirated and the pellet lysed for 20 min on ice. The cells were again spun for 5 minutes @ 4° C. to remove cell debris and the supernatant transferred to a fresh Eppendorf tube. Protein quantity was determined using a BCA kit (Pierce). For immunoprecipitation 20 μl of Sigma EZ view Flag Affinity gel (Sigma,) was used to immunoprecipitate Flag-PABP1 from ˜150 μg of total cell lysate (overnight incubation @4° C. with constant rotation). The following day the immunoprecipitates (IPs) were washed with 3×1 ml/wash in lysis buffer. 30 μl/IP of 4×SDS-PAGE gel loading buffer was added, samples were boiled 5 minutes and spun down. 15 μl/lane was loaded onto 4-12% Bis Tris gels (Invitrogen) and run for 2 hrs @ 125 volts. Proteins were transferred from the gel to Nitrocellulose for an additional 2 hrs at 25 volts. The membranes were blocked for 1 hr in PBS/0.5% Tween 20/5% Milk. Two primary antibodies were added to the membrane and left overnight @ 4° C.: (a) Rabbit anti-Methyl-PABP1 [R455, R460] @ 1:2000, and (b) Mouse anti-Flag M2 (Sigma F3165) used @ 1:5000. The following day blots were washed 3×5 minutes with PBST. Secondary antibodies were added for 1 hr @ room temperature (1:1000 PBST/5% milk—with the membrane being kept in the dark): (a) Alex-Fluor 680 (Molecular Probes) Goat anti-Mouse IgG and (b) IR Dye 8000 CW Conjugated anti-Rabbit IgG (Rockland). Blots were washed for 4×15 minutes with PBST. Blots were scanned on LI-COR and percent inhibition of methylation was quantitated relative to the ratio of methyl-PABP1/Flag signal of untreated samples.

CARM1-pep-Flag Transient Transfection Assay

Cells were plated in 6-well dishes and allowed to adhere and grow overnight such that they were 80% confluent at the time of transfection. Transfections were performed using Lipofectamine 2000 (Invitrogen) and OptiMEM media. The total amount of DNA transfected was held constant within experiments. Four hours post-transfection the Lipofectamine-DNA mix was removed and replaced with fresh media containing 10% serum. Compounds were added at this time. CARM1-pep-Flag was immunoprecipitated (EZ view Flag affinity gel) from 150 μg total lysate, resolved on a 4-12% Tris-glycine gel, transferred to Nitrocellulose, and the resulting blots were probed with rabbit anti-Methyl-H3 R17 antibody (Upstate, used (1:1000) and mouse anti-Flag M2 Monoclonal antibody (Sigma F3165, used (1:5000). The blots were scanned, signals detected by the methyl-specific and Flag antibodies, and quantitated on the LI-COR machine.

Detailed Protocol:

HCT 116 cells were plated at 0.4×10⁶ c/well onto a six well plate in McCoy's supplemented with 10% FCS. The following day the media in each well was removed via aspiration and replaced with 1.5 ml fresh McCoy's medium supplemented with 10% FCS. CARM1-pep-Flag was transfected at 2 μg/well using lipofectamine 2000 at 5 μl/well in a volume of 0.5 ml OptiMEM/well added dropwise to the cells. After 4 hours the media was removed via aspiration. Compounds were added to wells in a final volume of 2 ml in McCoy's medium supplemented with 10% FCS. Cells were harvested via scraping after 24 hrs and collected in 15 ml tubes. Cells were then spun for 5 minutes at 4° C., washed with 1 ml PBS, transferred to Eppendorf tubes, and re-spun for 5 minutes. The supernatants were aspirated and the pellets lysed for 20 min on ice. The cell lysates were spun for 5 minutes at 4° C. and the supernatants transferred to a fresh Eppendorf tubes. Protein quantity was determined using the BCA kit (Pierce). For immunoprecipitation, 20 μl Flag affinity gel was added per 150 μg of total lysate and the volume was brought up to 500 μl with lysis buffer. Immunoprecipitates were rotated overnight at 4° C. The following day the immunoprecipitates were washed with 3×1 ml/wash in lysis buffer. 4×SDS-PAGE loading buffer was added at 30 μl per immunoprecipitate, and samples were boiled for 5 minutes and spun down. 15 μl/lane was loaded onto 4-12% Bis-Tris gels (Invitrogen) and run for 2 hrs at 125 volts (using duplicate gels). The gels were transferred to nitrocellulose, for 2 hrs at 25 volts. The membranes were blocked for 1 hr in PBS/0.5% Tween 20/5% milk. The primary antibodies were added for the overnight incubation at 4° C. (in PBST/5% Milk), i.e. Anti-Methyl-H3 R17 (Upstate) used @ 1:1000, and Anti-Flag M2 Monoclonal Ab (Sigma F3165) used at 1:5000 dilution. The following day blots were washed for 3×5 minutes in PBST. Secondary antibodies were added for 1 hr at room temperature (1:1000 PBST/5% milk—the membrane was kept in the dark), i.e. Alex-Fluor 680 (Molecular Probes) Goat anti-Mouse IgG. And IR Dye 8000 CW Conjugated anti-Rabbit IgG (Rockland). Blots were washed for 4×15 minutes in PBST. Blots are scanned on LI-COR and percent inhibition of methylation was quantitated relative to the ratio of methyl-PABP1/Flag signal of untreated samples.

### ABBREVIATIONS

PRMT=Protein Arginine Methyltransferase; RCSB=Research Collaboratory for Structural Bioinformatics

### INCORPORATION BY REFERENCE

All patents, published patent applications and other references disclosed herein are hereby expressly incorporated herein by reference.

### EQUIVALENTS

Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A crystal comprising a domain of a CARM1-like methyltransferase protein or a homologue thereof, wherein said domain of said CARM1-like methyltransferase protein is selected from the group consisting of amino acid residues X-Y of SEQ ID NO:1, where X is one of 27, 60, 93, 128, 133, or 140, and Y is one of 472, 480, 521, or 608, and optionally additional chemical entities are present.

2. The crystal of claim 1, wherein said domain of said CARM1-like methyltransferase comprises amino acid residues 128-480 of SEQ ID NO:1, and optionally other chemical entities are present.

3. A crystallizable composition comprising a domain of a CARM1-like methyltransferase protein or a homologue thereof, wherein said domain of said CARM1-like methyltransferase is selected from the group consisting of amino acid residues X-Y of SEQ ID NO:1, where X is one of 27, 60, 93, 128, 133, or 140, and Y is one of 472, 480, 521, or 608 of SEQ ID NO:1.

4. The crystallizable composition of claim 3, wherein said domain of said CARM1-like methyltransferase protein comprises amino acid residues 128-480 of SEQ ID NO:1.

5. A computer comprising:
(a) a machine-readable data storage medium, comprising a data storage material encoded with machine-readable data, wherein said data defines a binding pocket or domain selected from the group consisting of:
(i) a set of amino acid residues which are identical to human CARM1 amino acid residues R168, E214, and E243 according to FIG. 1A, wherein the root mean square deviation of the backbone atoms between the set of amino acid residues and the CARM1 amino acid residues is not greater than about 2.0 Å;
(ii) a set of amino acid residues comprising at least three amino acid residues which are identical to human CARM1 amino acid residues F150, R168, D190, C193, L198, A212, E214, V242 and E243 according to FIG. 1A, wherein the root mean square deviation of the backbone atoms between the at least three amino acid residues and the CARM1 amino acid residues which are identical is not greater than about 2.0 Å;
(iii) a set of amino acid residues comprising at least five amino acid residues which are identical to human CARM1 amino acid residues F150, R168, D190, C193, L198, A212, E214, V242 and E243 according to FIG. 1A, wherein the root mean square deviation of the backbone atoms between the at least five amino acid residues and the CARM1 amino acid residues which are identical is not greater than about 2.0 Å;
(iv) a set of amino acid residues comprising at least five amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least five amino acid residues and the CARM1 amino acid residues which are identical is not greater than about 2.0 Å;
(v) a set of amino acid residues comprising at least six amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least six amino acid residues and the CARM1 amino acid residues which are identical is not greater than about 2.0 Å; and
(vi) a set of amino acid residues that are identical to CARM1 amino acid residues according to FIG. 1A, wherein the root mean square deviation between the set of amino acid residues and the CARM1 amino acid residues is not more than about 2.0 Å;
(vii) a set of amino acid residues that are identical to CARM1 amino acid residues according to FIG. 1A, wherein the root mean square deviation between the set of amino acid residues and the CARM1 amino acid residues is not more than about 3.0 Å;
(b) a working memory for storing instructions for processing said machine-readable data;
(c) a central processing unit coupled to said working memory and to said machine-readable data storage medium for processing said machine-readable data and a means for generating three-dimensional structural information of said binding pocket or domain; and
(d) output hardware coupled to said central processing unit for outputting said three-dimensional structural information of said binding pocket or domain, or information produced using said three-dimensional structural information of said binding pocket or domain.

6. The computer of claim 5, wherein the binding pocket is produced by homology modeling of the structure coordinates of said CARM1-like methyltransferase amino acid residues according to the associated crystal structure.

7. The computer of claim 5, wherein said means for generating three-dimensional structural information is provided by means for generating a three-dimensional graphical representation of said binding pocket or domain.

8. The computer of claim 5, wherein said output hardware is a display terminal, a printer, CD or DVD recorder, ZIP™ or JAZ™ drive, a disk drive, or other machine-readable data storage device.

9. A method of using a computer for selecting an orientation of a chemical entity that interacts favorably with a binding pocket or domain selected from the group consisting of:
(i) a set of amino acid residues which are identical to human CARM1 amino acid residues R168, E214, and E243 according to FIG. 1A, wherein the root mean square deviation of the backbone atoms between the set of amino acid residues and the CARM1 amino acid residues is not greater than about 2.0 Å;
(ii) a set of amino acid residues comprising at least three amino acid residues which are identical to human CARM1 amino acid residues F150, R168, D190, C193, L198, A212, E214, V242 and E243 according to FIG. 1A, wherein the root mean square deviation of the backbone atoms between the at least three amino acid residues and the CARM1 amino acid residues which are identical is not greater than about 2.0 Å;
(iii) a set of amino acid residues comprising at least five amino acid residues which are identical to human CARM1 amino acid residues F150, R168, D190, C193, L198, A212, E214, V242 and E243 according to FIG. 1A, wherein the root mean square deviation of the backbone atoms between the at least five amino acid residues and the CARM1 amino acid residues which are identical is not greater than about 2.0 Å;
(iv) a set of amino acid residues comprising at least five amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least five amino acid residues and the CARM1 amino acid residues which are identical is not greater than about 2.0 Å;
(v) a set of amino acid residues comprising at least six amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least six amino acid residues and the CARM1 amino acid residues which are identical is not greater than about 2.0 Å; and
(vi) a set of amino acid residues that are identical to CARM1 amino acid residues according to FIG. 1A, wherein the root mean square deviation between the set of amino acid residues and the CARM1 amino acid residues is not more than about 2.0 Å;
(vii) a set of amino acid residues that are identical to CARM1 amino acid residues according to FIG. 1A, wherein the root mean square deviation between the set of amino acid residues and the CARM1 amino acid residues is not more than about 3.0 Å;
said method comprising the steps of:
(a) providing the structure coordinates of said binding pocket or domain on a computer comprising means for generating three-dimensional structural information from said structure coordinates;
(b) employing computational means to dock a first chemical entity in the binding pocket or domain;
(c) quantifying the association between said chemical entity and all or part of the binding pocket or domain for different orientations of the chemical entity; and
d) selecting the orientation of the chemical entity with the most favorable interaction based on said quantified association.

10. The method of claim 9, further comprising the step of: (e) generating a three-dimensional graphical representation of the binding pocket or domain prior to step (b).

11. The method of claim 9, wherein energy minimization, molecular dynamics simulations, rigid-body minimizations, combinations thereof, or similar induced-fit manipulations are performed simultaneously with or following step (b).

12. The method of claim 9, further comprising the steps of: (e) repeating steps (b) through (d) with a second chemical entity; and (f) selecting at least one of said first or second chemical entity that interacts more favorably with said-binding pocket or domain based on said quantified association of said first or second chemical entity.

13. A method of using a computer for selecting an orientation of a chemical entity with a favorable shape complementarity in a binding pocket selected from the group consisting of:
(i) a set of amino acid residues which are identical to human CARM1 amino acid residues R168, E214, and E243 according to FIG. 1A, wherein the root mean square deviation of the backbone atoms between the set of amino acid residues and the CARM1 amino acid residues is not greater than about 2.0 Å;
(ii) a set of amino acid residues comprising at least three amino acid residues which are identical to human CARM1 amino acid residues F150, R168, D190, C193, L198, A212, E214, V242 and E243 according to FIG. 1A, wherein the root mean square deviation of the backbone atoms between the at least three amino acid residues and the CARM1 amino acid residues which are identical is not greater than about 2.0 Å;
(iii) a set of amino acid residues comprising at least five amino acid residues which are identical to human CARM1 amino acid residues F150, R168, D190, C193, L198, A212, E214, V242 and E243 according to FIG. 1A, wherein the root mean square deviation of the backbone atoms between the at least five amino acid residues and the CARM1 amino acid residues which are identical is not greater than about 2.0 Å;
(iv) a set of amino acid residues comprising at least five amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least five amino acid residues and the CARM1 amino acid residues which are identical is not greater than about 2.0 Å;
(v) a set of amino acid residues comprising at least six amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least six amino acid residues and the CARM1 amino acid residues which are identical is not greater than about 2.0 Å; and
(vi) a set of amino acid residues that are identical to CARM1 amino acid residues according to FIG. 1A, wherein the root mean square deviation between the set of amino acid residues and the CARM1 amino acid residues is not more than about 2.0 Å;
(vii) a set of amino acid residues that are identical to CARM1 amino acid residues according to FIG. 1A, wherein the root mean square deviation between the set of amino acid residues and the CARM1 amino acid residues is not more than about 3.0 Å;
said method comprising the steps of:
(a) providing the structure coordinates of said binding pocket and all or part of the putative substrate binding pocket bound therein on a computer comprising means for generating three-dimensional structural information from said structure coordinates;
(b) employing computational means to dock a first chemical entity in the binding pocket;
(c) quantitating the contact score of said chemical entity in different orientations; and
(d) selecting an orientation with the highest contact score.

14. The method of claim 13, further comprising the step of: (e) generating a three-dimensional graphical representation of the binding pocket and all or part of the putative substrate binding pocket bound therein prior to step (b).

15. The method of claim 13, further comprising the steps of: (e) repeating steps (b) through (d) with a second chemical entity; and (f) selecting at least one of said first or second chemical entity that interacts more favorably with said-binding pocket or domain based on said quantified association of said first or second chemical entity.

16. A method for identifying a candidate binder of a molecule or molecular complex comprising a binding pocket or domain selected from the group consisting of:
(i) a set of amino acid residues which are identical to human CARM1 amino acid residues R168, E214, and E243 according to FIG. 1A, wherein the root mean square deviation of the backbone atoms between the set of amino acid residues and the CARM1 amino acid residues is not greater than about 2.0 Å;
(ii) a set of amino acid residues comprising at least three amino acid residues which are identical to human CARM1 amino acid residues F150, R168, D190, C193, L198, A212, E214, V242 and E243 according to FIG. 1A, wherein the root mean square deviation of the backbone atoms between the at least three amino acid residues and the CARM1 amino acid residues which are identical is not greater than about 2.0 Å;
(iii) a set of amino acid residues comprising at least five amino acid residues which are identical to human CARM1 amino acid residues F150, R168, D190, C193, L198, A212, E214, V242 and E243 according to FIG. 1A, wherein the root mean square deviation of the backbone atoms between the at least five amino acid residues and the CARM1 amino acid residues which are identical is not greater than about 2.0 Å;
(iv) a set of amino acid residues comprising at least five amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least five amino acid residues and the CARM1 amino acid residues which are identical is not greater than about 2.0 Å;
(v) a set of amino acid residues comprising at least six amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least six amino acid residues and the CARM1 amino acid residues which are identical is not greater than about 2.0 Å; and
(vi) a set of amino acid residues that are identical to CARM1 amino acid residues according to FIG. 1A, wherein the root mean square deviation between the set of amino acid residues and the CARM1 amino acid residues is not more than about 2.0 Å;
(vii) a set of amino acid residues that are identical to CARM1 amino acid residues according to FIG. 1A, wherein the root mean square deviation between the set of amino acid residues and the CARM1 amino acid residues is not more than about 3.0 Å;
comprising the steps of:
(a) using a three-dimensional structure of the binding pocket or domain to design, select or optimize a plurality of chemical entities;
(b) contacting each chemical entity with the molecule or the molecular complex;
(c) monitoring an effect on the catalytic activity of the molecule or molecular complex by each chemical entity; and
(d) selecting a chemical entity based on the effect of the chemical entity on the catalytic activity of the molecule or molecular complex.

17. A method of designing a compound or complex that interacts with a binding pocket or domain selected from the group consisting of:
(i) a set of amino acid residues which are identical to human CARM1 amino acid residues R168, E214, and E243 according to FIG. 1A, wherein the root mean square deviation of the backbone atoms between the set of amino acid residues and the CARM1 amino acid residues is not greater than about 2.0 Å;
(ii) a set of amino acid residues comprising at least three amino acid residues which are identical to human CARM1 amino acid residues F150, R168, D190, C193, L198, A212, E214, V242 and E243 according to FIG. 1A, wherein the root mean square deviation of the backbone atoms between the at least three amino acid residues and the CARM1 amino acid residues which are identical is not greater than about 2.0 Å;
(iii) a set of amino acid residues comprising at least five amino acid residues which are identical to human CARM1 amino acid residues F150, R168, D190, C193, L198, A212, E214, V242 and E243 according to FIG. 1A, wherein the root mean square deviation of the backbone atoms between the at least five amino acid residues and the CARM1 amino acid residues which are identical is not greater than about 2.0 Å;
(iv) a set of amino acid residues comprising at least five amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least five amino acid residues and the CARM1 amino acid residues which are identical is not greater than about 2.0 Å;
(v) a set of amino acid residues comprising at least six amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least six amino acid residues and the CARM1 amino acid residues which are identical is not greater than about 2.0 Å; and
(vi) a set of amino acid residues that are identical to CARM1 amino acid residues according to FIG. 1A, wherein the root mean square deviation between the set of amino acid residues and the CARM1 amino acid residues is not more than about 2.0 Å;
(vii) a set of amino acid residues that are identical to CARM1 amino acid residues according to FIG. 1A, wherein the root mean square deviation between the set of amino acid residues and the CARM1 amino acid residues is not more than about 3.0 Å;
comprising the steps of:
(a) providing the structure coordinates of said binding pocket or domain on a computer comprising means for generating three-dimensional structural information from said structure coordinates;
(b) using the computer to dock a first chemical entity in part of the binding pocket or domain;
(c) docking at least a second chemical entity in another part of the binding pocket or domain;
(d) quantifying the association between the first or second chemical entity and part of the binding pocket or domain;
(e) repeating steps (b) to (d) with another first and second chemical entity;
(f) selecting a first and a second chemical entity based on said quantified association of both said first and second chemical entity;
(g) optionally, visually inspecting the relationship of the first and second chemical entity to each other in relation to the binding pocket or domain on a computer screen using the three-dimensional graphical representation of the binding pocket or domain and said first and second chemical entity; and
(h) assembling the first and second chemical entity into a compound or complex that interacts with said binding pocket or domain by model building.

18. A method of utilizing molecular replacement to obtain structural information about a molecule or a molecular complex of unknown structure, wherein the molecule is sufficiently homologous to a domain of a CARM1 methyltransferase protein or a homologue thereof, comprising the steps of:
(a) crystallizing said molecule or molecular complex;
(b) generating an X-ray diffraction pattern from said crystallized molecule or molecular complex;
(c) applying at least a portion of the structure coordinates set forth in the associated crystal structure or a homology model thereof to the X-ray diffraction pattern to generate a three-dimensional electron density map of at least a portion of the molecule or molecular complex of unknown structure; and
(d) generating a structural model of the molecule or molecular complex from the three-dimensional electron density map.

19. The method of claim 18, wherein the molecule is selected from the group consisting of said domain of said CARM1-like methyltransferase protein, and said domain of said CARM1-like methyltransferase protein homologue.

20. The method of claim 18, wherein the molecular complex is selected from the group consisting of said domain of said CARM1-like methyltransferase protein complex and said domain of said CARM1-like methyltransferase protein homologue complex.

21. A method for identifying a candidate binder that interacts with a binding site of a CARM1-like methyltransferase protein or a homologue thereof, comprising the steps of:
(a) obtaining a crystal comprising a domain of said CARM1-like methyltransferase protein or said homologue thereof, wherein the crystal is characterized with space group P_{21 21 2} and has unit cell parameters of a=74.852, b=98.629 Å, c=207.316 Å;
(b) obtaining the structure coordinates of amino acids of the crystal of step (a), wherein the structure coordinates are set forth in the associated crystal structure;
(c) generating a three-dimensional model of the domain of said CARM1-like methyltransferase protein or said homologue thereof using the structure coordinates of the amino acids obtained in step (b), a root mean square deviation from backbone atoms of said amino acids of not more than ±2.0 Å;
(d) determining a binding site of the domain of said CARM1-like methyltransferase protein or said homologue thereof from said three-dimensional model; and
(e) performing computer fitting analysis to identify the candidate binder which interacts with said binding site.

22. The method of claim 21, further comprising the step of: (f) contacting the identified candidate binder with the domain of said CARM1-like methyltransferase protein or said homologue thereof in order to determine the effect of the binder on CARM1-like methyltransferase protein activity.

23. The method of claim 21, wherein the binding site of the domain of said CARM1-like methyltransferase protein or said homologue thereof determined in step (d) comprises the structure coordinates according to the associated crystal structure of amino acid residues R168, E214, and E243, wherein the root mean square deviation from the backbone atoms of said amino acids is not more than ±2.0 Å.

24. The method of claim 21, wherein the binding site of the domain of said CARM1-like methyltransferase protein or said homologue thereof determined in step (d) comprises the structure coordinates according to the associated crystal structure of amino acid residues F150, R168, D190, C193, L198, A212, E214, V242 and E243, wherein the root mean square deviation from the backbone atoms of said amino acids is not more than ±2.0 Å.

25. The method of claim 21, wherein the binding site of the domain of said CARM1-like methyltransferase protein or said homologue thereof determined in step (d) comprises the structure coordinates according to the associated crystal structure of amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415, wherein the root mean square deviation from the backbone atoms of said amino acids is not more than ±2.0 Å.

26. A method for identifying a candidate binder that interacts with a binding site of a domain of a CARM1-like methyltransferase protein or a homologue thereof, comprising the steps of:
(a) obtaining a crystal comprising the domain of said CARM1-like methyltransferase protein or said homologue thereof, wherein the crystal is characterized with space group P_{21 21 2} and has unit cell parameters of a=74.852, b=98.629 Å, c=207.316 Å;
(b) obtaining the structure coordinates of amino acids of the crystal of step (a);
(c) generating a three-dimensional model of said CARM1-like methyltransferase protein or said homologue thereof using the structure coordinates of the amino acids generated in step (b), a root mean square deviation from backbone atoms of said amino acids of not more than ±2.0 Å;
(d) determining a binding site of the domain of said CARM1-like methyltransferase protein or said homologue thereof from said three-dimensional model; and
(e) performing computer fitting analysis to identify the candidate binder which interacts with said binding site.

27. The method of claim 26, further comprising the step of: (f) contacting the identified candidate binder with the domain of said CARM1-like methyltransferase protein or said homologue thereof in order to determine the effect of the binder on CARM1-like methyltransferase protein activity.

28. The method of claim 26, wherein the binding site of the domain of said CARM1-like methyltransferase protein or said homologue thereof determined in step (d) comprises the structure coordinates according to the associated crystal structure of amino acid residues R168, E214, and E243, wherein the root mean square deviation from the backbone atoms of said amino acids is not more than ±2.0 Å.

29. The method of claim 26, wherein the binding site of the domain of said CARM1-like methyltransferase protein or said homologue thereof determined in step (d) comprises the structure coordinates according to the associated crystal structure of amino acid residues F150, R168, D190, C193, L198, A212, E214, V242 and E243, wherein the root mean square deviation from the backbone atoms of said amino acids is not more than ±2.0 Å.

30. The method of claim 26, wherein the binding site of the domain of said CARM1-like methyltransferase protein or said homologue thereof determined in step (d) comprises the structure coordinates according to the associated crystal structure of amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415, wherein the root mean square deviation from the backbone atoms of said amino acids is not more than ±2.0 Å.

31. A method for identifying a candidate binder that interacts with a binding site of a domain of a CARM1-like methyltransferase protein or a homologue thereof, comprising the step of determining a binding site of the domain of said CARM1-like methyltransferase protein or the homologue thereof from a three-dimensional model to design or identify the candidate binder which interacts with said binding site.

32. The method of claim 31, wherein the binding site of the domain of said CARM1-like methyltransferase protein or said homologue thereof determined comprises the structure coordinates according to the associated crystal structure of amino acid residues R168, E214, and E243, wherein the root mean square deviation from the backbone atoms of said amino acids is not more than ±2.0 Å.

33. The method of claim 31, wherein the binding site of the domain of said CARM1-like methyltransferase protein or said homologue thereof determined comprises the structure coordinates according to the associated crystal structure of amino acid residues F150, R168, D190, C193, L198, A212, E214, V242 and E243, wherein the root mean square deviation from the backbone atoms of said amino acids is not more than ±2.0 Å.

34. The method of claim 31, wherein the binding site of the domain of said CARM1-like methyltransferase protein or said homologue thereof determined comprises the structure coordinates according to the associated crystal structure of amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415, wherein the root mean square deviation from the backbone atoms of said amino acids is not more than ±2.0 Å.

35. A method for identifying a candidate binder of a molecule or molecular complex comprising a binding pocket or domain selected from the group consisting of:
(i) a set of amino acid residues which are identical to human CARM1 amino acid residues R168, E214, and E243 according to FIG. 1A, wherein the root mean square deviation of the backbone atoms between the set of amino acid residues and the CARM1 amino acid residues is not greater than about 2.0 Å;
(ii) a set of amino acid residues comprising at least three amino acid residues which are identical to human CARM1 amino acid residues F 150, R168, D190, C193, L198, A212, E214, V242 and E243 according to FIG. 1A, wherein the root mean square deviation of the backbone atoms between the at least three amino acid residues and the CARM1 amino acid residues which are identical is not greater than about 2.0 Å;
(iii) a set of amino acid residues comprising at least five amino acid residues which are identical to human CARM1 amino acid residues F150, R168, D190, C193, L198, A212, E214, V242 and E243 according to FIG. 1A, wherein the root mean square deviation of the backbone atoms between the at least five amino acid residues and the CARM1 amino acid residues which are identical is not greater than about 2.0 Å;
(iv) a set of amino acid residues comprising at least five amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least five amino acid residues and the CARM1 amino acid residues which are identical is not greater than about 2.0 Å;
(v) a set of amino acid residues comprising at least six amino acid residues which are identical to human CARM1 amino acid residues F137, R140, Y149, F150, Y153, Q159, M162, M163, R168, D190, G192, C193, G194, S195, I197, L198, A212, V213, E214, A215, S216, G240, K241, V242, E243, E257, P258, M259, G260, Y261, N265, E266, M268, S271, and W415 according to FIG. 1A, wherein the root mean square deviation of the backbone atoms between the at least six amino acid residues and the CARM1 amino acid residues which are identical is not greater than about 2.0 Å; and
(vi) a set of amino acid residues that are identical to CARM1 amino acid residues according to FIG. 1A, wherein the root mean square deviation between the set of amino acid residues and the CARM1 amino acid residues is not more than about 2.0 Å;
(vii) a set of amino acid residues that are identical to CARM1 amino acid residues according to FIG. 1A, wherein the root mean square deviation between the set of amino acid residues and the CARM1 amino acid residues is not more than about 3.0 Å;
comprising the steps of:
(a) using a three-dimensional structure of the binding pocket or domain to design, select or optimize a plurality of chemical entities; and
(b) selecting said candidate binder based on the effect of said chemical entities on a domain of a CARM1-like methyltransferase protein or a domain of a CARM1-like methyltransferase protein homologue on the catalytic activity of the molecule or molecular complex.

36. A method of using the crystal according to claim 1 or 2 in a screening assay comprising: (a) selecting a potential binder by performing rational drug design with a three-dimensional structure determined for the crystal, wherein said selecting is performed in conjunction with computer modeling; (b) contacting the potential binder with a methyltransferase; and (c) detecting the ability of the potential binder to modulate the activity of the methyltransferase.

37. A set of coordinates defining the 3-dimensional structure of the protein CARM1 with the amino acid sequence 128-420.

38. A method for determining the intracellular activity of CARM1 methyltransferase comprising, providing a sample of cells to be tested for CARM1 methyltransferase activity, wherein the cells have been engineered to express a CARM1 methyltransferase peptide substrate that is specific for CARM1 methyltransferase, determining the degree of methylation of the peptide substrate by CARM1 methyltransferase in the sample, and thus determining the intracellular activity of CARM1 methyltransferase in the sample of cells.

39. A method for identifying an agent that inhibits the intracellular activity of CARM1 methyltransferase comprising, providing a sample of cells having CARM1 methyltransferase activity, wherein the cells have been engineered to express a CARM1 methyltransferase peptide substrate that is specific for CARM1 methyltransferase, determining the degree of reduction of methylation of the peptide substrate by CARM1 methyltransferase by contacting the sample of cells with a test agent and comparing the peptide substrate methylation level with the methylation level of peptide substrate in an identical control sample of cells that was not contacted with the test agent, determining the degree of inhibition of intracellular activity of CARM1 methyltransferase in the sample of cells contacted with the agent, and thus determining whether the test agent is an agent that inhibits the intracellular activity of CARM1 methyltransferase.

40. A composition comprising a compound having the formula: 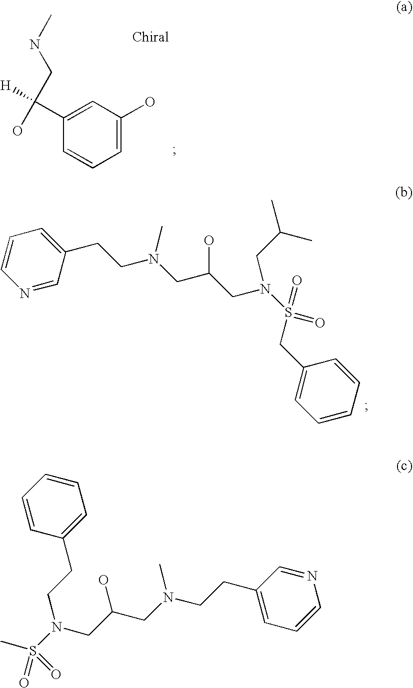
or a salt thereof.

41. A method of treating a CARM1 associated disorder comprising administering to a subject in need thereof the composition of claim 40.

42. The method of claim 41, wherein said CARM1 associated disorder is inflammation, cancer, diabetes, heart disease, schizophrenia, wound healing, or a parasitic infection.

43. A method of decreasing CARM1 activity in a cell comprising contacting said cell with the composition of claim 40.


SEQUENCE LISTING (1 sequence)

SEQ ID NO: 1 - protein, 608 aa, Homo sapiens
MAAAAAAVGPGAGGAGSAVPGGAGPCATVSVFPGARLLTIGDANGEIQRHAEQQALRLEVRAGPDSAGIALYSHEDVCVFKCSVSRETECSRVGKQSFIITLGCNSVLIQFATPNDFCSFYNILKTCRGHTLERSVFSERTEESSAVQYFQFYGYLSQQQNMMQDYVRTGTYQRAILQNHTDFKDKIVLDVGCGSGILSFFAAQAGARKIYAVEASTMAQHAEVLVKSNNLTDRIVVIPGKVEEVSLPEQVDIIISEPMGYMLFNERMLESYLHAKKYLKPSGNMFPTIGDVHLAPFTDEQLYMEQFTKANFWYQPSFHGVDLSALRGAAVDEYFRQPVVDTFDIRILMAKSVKYTVNFLEAKEGDLHRIEIPFKFHMLHSGLVHGLAFWFDVAFIGSIMTVWLSTAPTEPLTHWYQVRCLFQSPLFAKAGDTLSGTCLLIANKRQSYDISIVAQVDQTGSKSSNLLDLKNPFFRYTGTTPSPPPGSHYTSPSENMWNTGSTYNLSSGMAVAGMPTAYDLSSVIASGSSVGHNNLIPLANTGIVNHTHSRMGSIMSTGIVQGSSGAQGSGGGSTSAHYAVNSQFTMGGPAISMASPMSIPTNTMHYGS